(19) United States
(12) Patent Application Publication
(10) Pub. No.: US 2024/0360466 A1
(43) Pub. Date: Oct. 31, 2024

(54) ELEVATED RESISTANCE TO INSECTS AND PLANT PATHOGENS WITHOUT COMPROMISING SEED PRODUCTION

(71) Applicant: Board of Trustees of Michigan State University, East Lansing, MI (US)

(72) Inventors: Gregg A. Howe, East Lansing, MI (US); Qiang Guo, East Lansing, MI (US)

(21) Appl. No.: 18/584,801

(22) Filed: Feb. 22, 2024

Related U.S. Application Data
(62) Division of application No. 17/071,555, filed on Oct. 15, 2020, now Pat. No. 11,939,588.
(60) Provisional application No. 62/916,609, filed on Oct. 17, 2019.

(51) Int. Cl. C12N 15/82

(52) U.S. Cl. CPC C12N 15/8279; C12N 15/8286

(57) ABSTRACT

Described herein are modified plants, plant cells, and plant seeds that have at least one mutant loss-of-function JAZ gene and at least one loss-of-function cdk8 gene. Such plants are highly resistant to pests and environmental stress and have restored growth and increased seed yield compared to plant lines with a jazD genetic background.

This application is a divisional of U.S. application Ser. No. 17/071,555, filed Oct. 15, 2020 which claims benefit of priority to the filing date of U.S. Provisional Application Ser. No. 62/916,609, filed Oct. 17, 2019, the contents of which are specifically incorporated herein by reference in their entirety.

### GOVERNMENT FUNDING

This invention was made with government support under DE-FG02-91ER20021 awarded by the U.S. Department of Energy. The government has certain rights in the invention.

### Sequence Listing XML

The instant application contains a sequence listing, which has been submitted in XML file format by electronic submission and is hereby incorporated by reference in its entirety. The XML file, created on Jul. 16, 2024, is named P14830US02.xml and is 153,352 bytes in size.

### BACKGROUND OF THE INVENTION

Currently, a main control strategy for insect pests is the application of insecticides, aimed at killing adults, juveniles and eggs. Besides the substantial costs of insecticide application this practice has a severe environmental impact. Emerging resistance to insecticides makes control of insect pests difficult.

Sustainable food production for an increasing world population likely depends on the next generation of “designer” crops that exhibit both superior yield and resilience to harsh environmental conditions, including environmental and biotic stresses. Such environmental and biotic stresses include drought, insects, and salt stresses. High yield/growth potential, however, is typically associated with reduced plant immunity, and vice versa; this phenomenon is often referred to as the plant “dilemma” to grow or defend.

There is a need for new ways of controlling crop damage and losses due to plant insect pests, both in field-grown and greenhouse-grown crops without adversely affecting plant immunity.

### SUMMARY

Described herein are plants and methods providing improved defenses to increased resistance to pests and environmental stresses. The plants and method involve jaz mutations to reduce JAZ repressors of defense (that can reduce plant growth) combined with CYCLIN-DEPENDENT KINASE 8 (CDK8) mutations that restore growth of the jaz mutant plants without compromising the elevated pest defense.

Plants with reduced JAZ expression and/or reduced JAZ functioning have reduced growth, and a smaller stature. However, as shown herein, combining loss of JAZ with loss of CDK8 functioning can lead to plants that exhibit good vegetative growth stature while simultaneously maintaining strong biotic stress resistance to insects and pathogens. One example of a plant line with reduced JAZ functioning is the jazD plant line. Mutation of CDK8 in the jazD genetic background improved the reproductive output of jazD, achieving seed yields that were comparable to or even greater than wild type plants. Therefore, described herein is a useful strategy to promote strong pest and biotic stress resistance while improving seed production and growth.

The plants can have one or more loss of function mutations in at least one JAZ gene. For example, plants, and seeds described herein have endogenous jazD mutations that include mutations in the genes encoding JAZ1, JAZ2, JAZ3, JAZ4, JAZ5, JAZ6, JAZ7, JAZ9, JAZ10, and/or JAZ13 proteins. Such mutations have reduced JAZ1, JAZ2, JAZ3, JAZ4, JAZ5, JAZ6, JAZ7, JAZ9, JAZ10, and/or JAZ13 activity. For example, in some cases the expression of JAZ1, JAZ2, JAZ3, JAZ4, JAZ5, JAZ6, JAZ7, JAZ9, JAZ10, and/or JAZ13 proteins is undetectable. Similarly, in mutant cdk8 plant cells, mutant cdk8 plants, and/or mutant cdk8 seeds the endogenous CDK8 proteins have reduced activity or their expression is undetectable. However, in some cases endogenous JAZ8, JAZ11, and JAZ12 genes are not modified or mutated in the jaz cdk8 plant cells, plants and plant seeds. Hence, endogenous JAZ8, JAZ11, and JAZ12 proteins can still be active in some cells and can be expressed in the mutant Jaz cdk8 plant cells, plants and/or plant seeds.

In some cases, the plants or a plant grown from the seeds described herein have at least 5% less leaf damage from insect feeding than a wild type plant of the same species grown under the same conditions. In some cases, the plants or a plant grown from the seeds described herein have the same or at least about 10% more seed yield than a wild type plant of the same species grown under the same conditions.

Methods of generating such plants, seed, and plant cells as well as methods of cultivating such plant seeds and plants are also described herein.

### DESCRIPTION OF THE FIGURES

FIG. 1A-1D illustrate the pedigree and structure mutated JAZ genes in the Arabidopsis jazD plant line. FIG. 1A-1 to 1A-4 illustrate the jazD pedigree. The jazD plants have loss-of-function mutations in ten JAZ genes: Jaz1, Jaz2, Jaz3, Jaz4, Jaz5, Jaz6, Jaz7, Jaz9, Jaz10, and Jaz13. The ‘x’ and the term ‘self’ indicate cross-pollination and self-pollination, respectively. The Jaz single mutants in boxes were previously characterized. FIG. 1A-1 illustrates crosses for generating jaz3-4 jaz3-3 jaz4-1 jaz9-41+gl1-2+ genotypes. FIG. 1A-2 illustrates crosses for generating jaz5-1+ jaz10-1+gl1-2+ genotypes and the jaz1-2 gl1-2 genotypes. FIG. 1A-3 illustrates crosses for generating jaz1-2 jaz4-1+ jaz9-4+ jaz3-4+ jaz10-1 gl1-2+ genotypes, and jaz-5-1 jaz1-2 jaz4-1 jaz9-4 jaz3-4 jaz0-1 jaz7-1+ gla-2+ genotypes, and jaz3-4+ jaz13-11+ genotypes. FIG. 1A-4 illustrates crosses for generating the jazD genotype (jaz5-1jaz1-2 jaz4-1 jaz9-4 jaz6-4 jaz2-3 jaz7-1 jaz3-4 jaz13-1 jaz0-1). The jaz2-1, jaz3-3, jaz9-1 alleles were characterized by Thines et al. Nature 448:661-665 (2007); and Chini et al. Nature 448:666-671 (2007). The jaz6-Wisc was characterized by the inventors, but jaz2-3, jaz3-4, jaz6-4 and jaz9-4 were later selected as alternative alleles for construction of jazD. gl1-2 was included to study trichome development. Male sterility of coil-1 mutants was exploited to assist in selection of rare recombination events between closely linked loci (Barth & Jander Plant J 46:549-562 (2006)). FIG. 1B illustrates a phylogenetic tree of thirteen JAZ proteins in Arabidopsis. Black and open (white) asterisks denote JAZ genes that contain insertion mutations in jazQ and jazD, respectively. FIG. 1C shows schematic diagrams of insertion mutations used for construction of jazQ and jazD. The jazQ plant lines have loss-of-function mutations in the Jaz1, Jaz3, Jaz4, Jaz9, and Jaz10. White boxes represent untranslated regions (UTRs), while shaded boxes represent exons. The identity and position of each insertion mutation is shown. Arrows beneath the exons show the position of primers used to assess expression of JAZ genes by RT-PCR. FIG. 1D illustrates expression of JAZ genes in wild-type Col-0 (WT), jazQ, and jazD in Arabidopsis plant leaves as evaluated by RT-PCR analysis. RNA was extracted from rosette leaves of 23-day-old plants grown under long-day conditions. The ACTIN1 gene (At2g37620) was used as a positive control. Arrows denote PCR products that have the predicted size of full-length JAZ transcripts. Note that some bands in the Jaz4 gel are artefacts and do not indicate that a Jaz4 transcript was expressed.

FIG. 2A-2E illustrate that a jaz decuple mutant (jazD) is highly sensitive to jasmonate (e.g. methyljasmonate, MeJA) and exhibits reduced growth and fertility. FIG. 2A graphically illustrates root length of 8-day-old wild type Col-0 (WT), jazQ, and jazD seedlings grown in the presence of 0, 5, or 25 μM methyljasmonate (MeJA). The data show the mean±SD of 30 plants per genotype at each concentration. Capital letters denote significant differences according to Tukey's honest significant difference (HSD) test (P<0.05). FIG. 2B shows that jazD leaves are hypersensitive to coronatine (COR). The eighth leaf of 40-day-old plants from different plant types grown under 12-hour light/12-hour dark photoperiods was treated with 5 μL water (mock) or 50 μM coronatine (COR). Leaves were excised and photographed after 2 or 4 days of treatment. Arrows denote location of visible anthocyanin accumulation at the site of coronatine application. The images to the right are enlargements of photograph of the COR-treated jazD leaves. (Scale bars: 1 cm.) FIG. 2C graphically illustrates the relative growth rate (RGR) of soil-grown wild type (WT), jazQ, and jazD plants. FIG. 2D graphically illustrates the total fatty acid content in wild type (WT), jazQ, and jazD seeds. Data show the mean±SD of seeds obtained from five plants per genotype. FIG. 2E graphically illustrates the time course of seed germination. Bars indicate the percentage of germinated seeds at various times after sowing on water agar: lowest stippled portion, day 1; open portion, day 2; striped portion, day 3; and all later times: top hatched portion, nongerminated seeds.

FIG. 3A-3F illustrates that jazD plants are highly resistant to insect herbivores and necrotrophic pathogens. FIG. 3A shows images of representative short-day grown wild type Col-0 (WT), jazQ, and jazD plants before and after challenge with four T. ni larvae for 12 days (scale bar: 3 cm). FIG. 3B graphically illustrates weight gain of T. ni larvae reared on plants shown in FIG. 3A. Data shown are the mean±SD of at least 30 larvae per genotype. Capital letters denote significant differences according to Tukey's HSD test (P<0.05). FIG. 3C is a heat map displaying the expression level of various jasmonate/ethylene-responsive genes in leaves of jazQ and jazD normalized to wild type. ACT, agmatine coumaroyl transferase (accession no. At5g61160). FIG. 3D shows images of representative leaves illustrating symptoms following 5 days of treatment with B. cinerea spores or mock solution (scale bars: 2 cm). FIG. 3E graphically illustrates disease lesion size on leaves of the indicated genotypes following 5 days of treatment with B. cinerea spores for the indicated plant lines. Data show the mean±SD of at least 19 leaves per genotype. Capital letters denote significant differences (Tukey's HSD test, P<0.05). FIG. 3F graphically illustrates apical hook angles of seedlings grown in the presence of various concentrations of the ethylene precursor 1-aminocyclopropane-1-carboxylic acid (ACC). Data shown are the mean±SD of at least 21 seedlings per genotype. Asterisks denote significant difference compared with WT (Tukey's HSD test, *P<0.05).

FIG. 4A-4G illustrate reconfiguration of primary and secondary metabolism in jazD plants. FIG. 4A schematically illustrates that mapping of differentially regulated genes in jazD to various metabolic pathways results in elevated production of defense metabolites derived from amino acids. Mapped pathways include photosynthesis (1), pentose phosphate pathway (2), shikimate pathway (3), amino acids from pentose phosphate intermediates (4), glycolysis (5), amino acids from glycolysis intermediates (6), TCA cycle (7), amino acids from TCA intermediates (8), sulfur metabolism (9), and defense metabolites from amino acids (10). Shading on the arrows denotes the average fold-change of differentially expressed transcripts mapping to a particular pathway (P<0.05). FIG. 4B shows a schematic of the tryptophan biosynthetic pathway from erythrose 4-phosphate (E4P), phosphoenolpyruvate (PEP), and 3-phosphoglycerate (3PG) showing up-regulation of genes and proteins in jazD. Each arrow represents an enzymatic reaction in the pathway. Boxes represent individual genes with at least 2-fold-change for jazD relative to wild type according to RNA-seq data, whereas genes without boxes denote genes with no significant change in expression. Gene names within boxes denote significantly increased protein levels according to proteomics data. Gene abbreviations: AnPRT, anthranilate phosphoribosyltransferase; AS, anthranilate synthase; CS, chorismate synthase; DHQS, 3-dehydroquinate synthase; DHS, 3-deoxy-7-phosphoheptulonate synthase; DQD/SDH, 3-dehydroquinate dehydratase/shikimate dehydrogenase; EPSP, 5-enolpyruvylshikimate-3-phosphate synthase; IGPS, indole-3-glycerol-phosphate synthase; IGs, indole glucosinolates; OAS, 0-acetylserine lyase; PAI, phosphoribosylanthranilate isomerase; PGDH, phosphoglycerate dehydrogenase; PSAT, phosphoserine aminotransferase; PSP, phosphoserine phosphatase; SAT, serine acetyltransferase; SK, shikimate kinase; TSA, tryptophan synthase alpha subunit; TSB, tryptophan synthase β-subunit. FIG. 4C graphically illustrates indole glucosinolate levels in jazD leaves relative to indole glucosinolate levels in wild type leaves. Asterisks denote significant differences in comparison with WT (Student's t test, *P<0.05). Abbreviations: I3M, indol-3-ylmethyl (glucobrassicin); OH-13M, 4-hydroxyindol-3-ylmethyl (hydroxyglucobrassicin); 4MOI3M, 4-methoxyindol-3-ylmethyl (methoxyglucobrassicin); 1MOI3M, 1-methoxyindol-3-ylmethyl (neoglucobrassicin). FIG. 4D graphically illustrates net gas exchange rates in wild type and jazD rosette leaves measured at 400 μmol CO₂ and 20° C. after acclimation in 500 μmol m⁻² s⁻¹ in light. FIG. 4E graphically illustrates net gas exchange rate in wild type and jazD rosette leaves measured at 400 μmol CO₂ and 20° C. after acclimation in 500 μmol m⁻² s⁻¹ in the dark. FIG. 4F graphically illustrates daytime respiration on a leaf area basis. Daytime respiration was determined from the intersection of CO₂ response curves measured at sub-saturating light intensities. FIG. 4G graphically illustrates nighttime dark respiration. Data shown for FIG. 4F-4G are the mean±SD of four replicates per genotype.

FIG. 5A-5F illustrate that jazD plants exhibit symptoms of carbon starvation. FIG. 5A shows a time course of starch levels in wild type Col-0 (WT) and jazD plants during a long day photoperiod. FIG. 5B shows a time course of sucrose levels in wild type Col-0 (WT) and jazD plants during a long day photoperiod. Asterisks in FIG. 5A-5B show that significant differences exist in comparison with WT (Student's t test, *P<0.05). FIG. 5C shows a heat map illustrating the expression level of sugar starvation marker (SSM) genes in jazQ and jazD leaves. Gene-expression levels determined by RNA-seq are represented as fold-change (log 2) over WT. FIG. 5D shows a photograph of 16-day-old wild type, jazQ, and jazD seedlings grown horizontally on MS medium containing the indicated concentration of sucrose (scale bar: FIG. 5D, 0.5 cm). FIG. 5E graphically illustrates dry weight (DW) of 16-day-old wild type, jazQ, and jazD seedlings grown horizontally on MS medium containing one of the concentrations of sucrose indicated in the key above graph. FIG. 5F graphically illustrates the root length of 11-day-old wild type, jazQ, and jazD seedlings grown vertically on MS medium lacking sucrose (open bar) or containing 23 mM sucrose (filled bar). Two-way ANOVA was used to test the effect of sucrose on growth (FIGS. 5E and 5F) and showed that, whereas genotype (P<0.001 for both WT vs. jazQ and WT vs. jazD) and sucrose (P<0.001 for both WT vs. jazQ and WT vs. jazD) significantly affect shoot and root growth, the genotype×sucrose interaction was significant only for jazD comparisons.

FIG. 6A-6C illustrate that genetic combination of jaz8 and jazD mutations reduces root lengths and nearly abolishes seed production in the resulting undecuple mutant. The jazD mutations eliminate transcription from Jaz1, Jaz2, Jaz3, Jaz4, Jaz5, Jaz6, Jaz7, Jaz9, Jaz10 and Jaz13 genes, while the jaz undecuple (jazU) mutations are homozygous for mutations in Jaz1-Jaz10 and Jaz13. Hence, the jazU plant line has a mutant jaz8 gene whereas the jazD plant line has a wild type Jaz8 gene. FIG. 6A illustrates root length of 10-day-old wild type Col-0 (WT), jazD, and jazU seedlings grown in the presence of 0, 0.2, or 1 μM MeJA. Data show the mean±SD of 14-20 seedlings per genotype at each concentration. Capital letters denote significant differences according to Tukey's HSD test (P<0.05). FIG. 6B shows a photograph of WT, jazQ, jazD, and jazU rosettes of 28-d-old plants. FIG. 6C shows a photograph of WT, jazD, and jazU inflorescence of 8-week-old plants.

FIG. 7A-7C illustrate that loss of function of cdk8 restores growth and reproductive output when included in a jazD genetic background while the jazD cdk8 plants maintain anti-insect defenses. FIG. 7A graphically illustrates total rosette biomass of short day-grown plants of the indicated genotypes. FIG. 7B graphically illustrates total seed yield of plants of the indicated genotypes. FIG. 7C graphically illustrates resistance to insect feeding by Trichoplusia ni insect larvae on plants of the indicated genotypes. Data points show the mean±SD of at least five plants per genotype. As shown, jazD plants strongly defend against larval infestation and the cdk8 loss of function mutation helps jazD plants maintain good growth improved seed production.

FIG. 8A-8D illustrate increased growth and improved defenses against insects by the cdk8 loss-of-function mutant line (sjd56), which has the cdk8 loss-of-function mutation in the jazD genetic background. The results for the sjd56 plants are compared to those for the jazD and wild type plants. FIG. 8A graphically illustrates rosette fresh weight of 58-day-old wild type Col-0 (WT), jazD and sjd56 plants grown under short-day (8-h-light/16-h-dark) conditions. FIG. 8B graphically illustrates projected leaf area of 58-day-old wild type Col-0 (WT), jazD and sjd56 plants grown under short-day (8-h-light/16-h-dark) conditions. Data shown for FIGS. 8A-8B are the mean±SD of five plants per genotype. Letters denote significant difference according to Tukey's HSD test (P<0.05). Scar bar, 2 cm. FIG. 8C graphically illustrates anthocyanin levels in leaves of 23-day-old WT, jazD and sjd56 plants grown under long-day (16-h-light/8-h-dark) conditions. Data show the mean±SD of five plants per genotype. FIG. 8D graphically illustrates Trichoplusia ni (T. ni) weight after feeding on WT, jazD and sjd56 plants for ten days. Plants were grown under the photoperiods of 16-h-light/8-h-dark for 67 days. Data show the mean±SD of at least ten larvae per genotype.

FIG. 9A-9H illustrate that cdk8 mutations largely restore the growth and reproduction while delaying vegetative and reproductive transitions of jazD. FIG. 9A illustrates the growth and rosette fresh weights (numbers under the images) of representative Col-0 (WT), cdk8-1, cdk8-2, jazD, jazD cdk8-1 and jazD cdk8-2 plants. Plants were grown under short-day conditions (8-h-ligh/16-h-dark) for 58 days. Data show the mean±SD of five plants per genotype. Letters denote significant difference according to Tukey's HSD test (P<0.05). Scale bar, 2 cm. FIG. 9B graphically illustrates the number of days until the first flower opens for WT, cdk8-1, cdk8-2, jazD, jazD cdk8-1 and jazD cdk8-2 plants. FIG. 9C graphically illustrates the number of rosette leaves at bolting for WT, cdk8-1, cdk8-2, jazD, jazD cdk8-1 and jazD cdk8-2 plants. For FIGS. 9B-9C, plants were grown under long-day (16-h-light/8-h-dark) conditions in soil. Data show the mean±SD of ten plants per genotype. Letters denote significant difference according to Tukey's HSD test (P<0.05). FIG. 9D graphically illustrates the seed yield from WT, cdk8-1, cdk8-2, jazD, jazD cdk8-1 and jazD cdk8-2 plants FIG. 9E graphically illustrates the average seed mass of seeds from WT, cdk8-1, cdk8-2, jazD, jazD cdk8-1 and jazD cdk8-2 plants. Seed yield was determined by collecting all seeds from individual plants. Average seed mass was determined by weighing batches of 100 seeds. Data show the mean±SD of at least five plants per genotype. Letters denote significant difference compared with WT plants according to Tukey's HSD test (P<0.05). FIG. 9F graphically illustrates projected leaf area of different plant types, showing that loss of cdk8 positively impacts the growth of jazD. The leaf area of Col-0 (WT), cdk8-1, cdk8-2, jazD, jazD cdk8-1 and jazD cdk8-2 plants was measured after growth under short-day (8-h-ligh/16-h-dark) conditions for 58-days. Data show the mean±SD of five plants per genotype. Letters denote significant difference according to Tukey's HSD test (P<0.05). FIG. 9G graphically illustrates the rosette diameter of Col-0 (WT), cdk8-1, cdk8-2, jazD, jazD cdk8-1 and jazD cdk8-2 plants measured after growth under short-day (8-h-ligh/16-h-dark) conditions for 58-days. Data show the mean±SD of five plants per genotype. Letters denote significant difference according to Tukey's HSD test (P<0.05). FIG. 9H graphically illustrates silique number per plant for WT, cdk8-1, cdk8-2, jazD, jazD cdk8-1 and jazD cdk8-2 plants grown under long-day (16-h-light/8-h-dark) conditions in soil. Fully elongated 7th, 9th, and 11th siliques were collected for measurements of silique traits (silique length and number of seeds per silique). These traits were used with total seed yield to calculate the number of siliques per plant. Letters denote significant difference according to Tukey's HSD test (P<0.05).

FIG. 10A-10B shows that cdk8 mutations partially recover the defense phenotypes of jazD. FIG. 10A shows representative images of Trichoplusia ni (T. ni) larvae after feeding on short-day-grown (8-h-light/16-h-dark) WT Col-0 (WT), cdk8-1, cdk8-2, jazD, jazD cdk8-1 and jazD cdk8-2 plants for nine days. Scale bar, 1 cm. FIG. 10B graphically illustrates the larval weight of T. ni shown in FIG. 10A. Data shown are the mean±SD of at least 18 larvae per genotype. Letters denote significant difference according to Tukey's HSD test (P<0.05).

FIG. 11A-11F illustrate that the increased production of defense compounds in jazD plants is partially regulated by CDK8. FIG. 11A graphically illustrates anthocyanin levels in leaves of 25-day-old WT Col-0 (WT), cdk8, jazD and jazD cdk8 plants. Plants were grown under long-day conditions (16-h-light/8-h-dark) in soil. Data show the mean±SD of three plants per genotype. Letters denote significant differences according to Tukey's HSD test (P<0.05). FIG. 11B graphically illustrates comparison of indole glucosinolates levels in WT, cdk8, jazD and jazD cdk8 leaves. FIG. 11C graphically illustrates Nδ-acetylornithine levels in WT, cdk8, jazD and jazD cdk8 leaves. FIG. 11D graphically illustrates hydroxycinnamic acid amides (HCAAs) levels in WT, cdk8, jazD and jazD cdk8-1 leaves. Defense compounds were extracted from leaves of 23-day-old plants grown under long-day conditions (16-h-light/8-h-dark). Peak area for the indicated compound in the WT sample was set to “1” and the peak area of the same compound in other genotypes was normalized to the WT sample. Abbreviations: I3M: indol-3-ylmethyl, glucobrassicin; OH-13M: 4-hydroxyindol-3-ylmethyl, hydroxyglucobrassicin; 4MOI3M: 4-methoxyindol-3-ylmethyl, methoxyglucobrassicin; 1MOI3M: 1-methoxyindol-3-ylmethyl, neoglucobrassicin. Data show the mean±SD of three biological replicates per genotype. Letters denote significant differences according to Tukey's HSD test (P<0.05). FIG. 11E graphically illustrates relative expression levels of VEGETATIVE STORAGE PROTEIN 2 (VSP2, accession no. AT5G24770) while FIG. 11F graphically illustrates relative expression levels of PLANT DEFENSIN 1.2 (PDF1.2, accession no. AT5G44420) in leaves of 25-day-old WT, cdk8, jazD and jazD cdk8 plants grown under long-day conditions (16-h-light/8-h-dark). PP2A (AT1g13320) was used for qPCR normalization. Data show the mean±SD of three biological replicates per genotype. Letters denote significant differences according to Tukey's HSD test (P<0.05).

FIG. 12 graphically illustrates that CDK8 loss promotes the production of many aliphatic glucosinolates in jazD cdk8 plants as shown by the aliphatic glucosinolate levels in WT, cdk8, jazD and jazD cdk8 leaves. Aliphatic glucosinolates were extracted from leaves of 23-day-old plants grown under long-day conditions (16-h-light/8-h-dark). Peak area for the indicated compound in the WT sample was set to “1” and the peak area of the same compound in other genotypes was normalized to the WT sample. Abbreviations for the compounds detected were: 3MSOP: 3-methylsulphinylpropyl glucosinolate, glucoiberin; 4MSOB: 4-methylsulphinylbutyl glucosinolate, glucoraphanin; 5MSOP: 5-methylsulphinylpentyl glucosinolate, glucoalyssin; 6MSOH: 6-methylsulphinylhexyl glucosinolate, glucohesperin; 7MSOH: 7-methylsulphinylheptyl glucosinolate, glucoibarin; 3MTP: 3-methylthiopropyl glucosinolate, glucoiberverin; 8MSOO: 8-methylsulphinyloctyl glucosinolate, glucohirsutin; 4MTB: 4-methylthiobutyl glucosinolate, glucoerucin; 5MTP: 5-methylthiopentyl glucosinolate, glucoberteroin; and 7MTH: 7-methylthioheptyl glucosinolate. The data shown are the mean±SD of three biological replicates per genotype. Letters denote significant differences according to Tukey's HSD test (P<0.05).

FIG. 13A-13B illustrate that the increased resistance of jazD to 5-methyl-tryptophan (5-MT) is partially dependent on CDK8. FIG. 13A is a schematic of tryptophan biosynthesis from chorismate. Tryptophan feedback inhibits the activity of anthranilate synthase (AS). Although 5-methyl-tryptophan (5-MT) inhibits anthranilate synthase activity, it cannot be used for the production of proteins. The abbreviations used in FIG. 13A are: TRP, anthranilate phosphoribosyltransferase; PAI, phosphoribosyl-anthranilate isomerase; IGPS, indole-3-glycerol-phosphate synthase; TSA, tryptophan synthase alpha subunit; TSB, tryptophan synthase beta subunit. FIG. 13B graphically illustrates root length of WT, cdk8-1, jazD, and jazD cdk8-1 10-day-old seedlings grown on medium supplemented with 0 or 15 μM of 5-methyl-tryptophan (5-MT). Data shown are the mean±SD of at least 24 seedlings per genotype at each 5-MT concentration. Letters denote significant differences according to Tukey's HSD test (P<0.05).

### DETAILED DESCRIPTION

Described herein are plants that have loss-of-function jaz decuple (jazD) mutations and loss-of-function CYCLIN-DEPENDENT KINASE 8 (CDK8) mutations. The jazD plants, by comparison to wild-type (WT) and jazQ plants, are highly resistant to both insect herbivores and necrotrophic pathogens but also exhibit reduced vegetative growth and reduced seed yield. However, when the jazD loss-of-function mutations are coupled with CDK8 loss-of-function mutations, plant growth is restored while the plants maintain strong biotic stress resistance to insects and pathogens. Moreover, mutation of CDK8 in the jazD genetic background seemed to improve the reproductive output of jazD, achieving seed yields that were comparable to or even greater than wild type plants.

Hence, described herein are jazD, cdk8 loss-of-function plants and seeds with resistance to insects and pathogens that grow as well as wild type plants and that have seed yields that were comparable to or even greater than wild type plants.

Methods of making such plants and seeds as well as methods of cultivating such plants and seeds are also described herein.

### Mutation Methods

Plants and methods of making such plants are described herein that grow well and are resistant to environmental stresses such as drought and insects. The plants have mutations that reduce or eliminate the expression or function of proteins that modulate jasmonic acid responses (e.g., JAZ genes/proteins). Plants with such mutations are referred to herein as jaz mutants or jaz plants. Such reduction/elimination of jasmonic acid regulatory protein expression and/or function improves the resistance (compared to wild type plants) of jaz mutant plants to insects and biotic stress. An additional mutation that reduces or eliminates the function of the cdk8 gene improves the growth of jazD mutant plants.

Plants with jazD mutations exhibit significantly improved resistance to insects and biotic stress, and when combined with loss-of-function cdk8 mutations, the plants grow reproduce well.

The jazD plants have loss-of-function mutations in ten JAZ genes: JAZ1, JAZ2, JAZ3, JAZ4, JAZ5, JAZ6, JAZ7, JAZ9, JAZ10, and JAZ13. Such jazD plants therefore have three remaining intact JAZ genes: JAZ8, JAZ11, and JAZ12. For example, plants with jazD mutations have transcription and/or translation of JAZ1, JAZ2, JAZ3, JAZ4, JAZ5, JAZ6, JAZ7, JAZ9, JAZ10, and JAZ13 reduced by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have the jazD). In some cases, plants with jazD mutations have transcription and/or translation of JAZ1, JAZ2, JAZ3, JAZ4, JAZ5, JAZ6, JAZ7, JAZ9, JAZ10, and JAZ13 reduced by at least 100%.

The jazD mutations are combined with loss-of-function cdk8 mutations. For example, plants with loss-of-function cdk8 mutations have transcription and/or translation of CDK8 reduced by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have the cdk8 loss-of-function mutation). In some cases, plants with cdk8 mutations have transcription and/or translation of CDK8 proteins reduced by at least 100%.

Non-limiting examples of methods of introducing a modification into the genome of a plant cell can include microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with guide nucleic acid that allows the nuclease to target the genomic JAZ and CDK8 site(s). In some cases of the various aspects described herein, a targeting vector can be used to introduce a deletion or modification of the genomic JAZ and CDK8 chromosomal sites.

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, the genomic JAZ and CDK8 site(s) can be disrupted by insertion of T-DNA. In another example, the foreign DNA to be inserted may encode a selectable marker, such as an antibiotics resistance gene. Examples for suitable selectable markers include chloramphenicol resistance, gentamycin resistance, kanamycin resistance, spectinomycin resistance (SpecR), neomycin resistance gene (NEO) and hygromycin β-phosphotransferase markers (genes). The 5′ flanking region and the 3′ flanking region can be homologous to regions within the gene, or such flanking regions can flank the coding region of gene to be deleted, mutated, or replaced with the unrelated DNA sequence. In some cases, the targeting vector does not comprise a selectable marker. DNA comprising the targeting vector and the native gene of interest are contacted under conditions that favor homologous recombination (e.g., by transforming plant cell(s) with the targeting vector).

A typical targeting vector contains nucleic acid fragments of not less than about 0.1 kb nor more than about 10.0 kb from both the 5′ and the 3′ ends of the genomic locus which encodes the gene to be modified (e.g. the genomic JAZ and/or CDK8 site(s)). These two fragments can be separated by an intervening fragment of nucleic acid that includes the modification to be introduced. When the resulting construct recombines homologously with the chromosome at this locus, it results in the introduction of the modification, e.g. an insertion, substitution, or a deletion of a portion of the genomic JAZ and/or CDK8 site(s).

In some cases, a Cas9/CRISPR system can be used to create a modification in genomic JAZ and/or CDK8 site(s). Clustered regularly interspaced short palindromic repeats (CRISPR)/CRISPR-associated (Cas) systems are useful for, e.g. RNA-programmable genome editing (see e.g., Marraffini & Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is available in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, CA.

In other cases, a cre-lox recombination system of bacteriophage P1, described by Abremski et al. 1983. Cell 32:1301 (1983), Sternberg et al., Cold Spring Harbor Symposia on Quantitative Biology, Vol. XLV 297 (1981) and others, can be used to promote recombination and alteration of the genomic JAZ and/or CDK8 site(s). The cre-lox system utilizes the cre recombinase isolated from bacteriophage P1 in conjunction with the DNA sequences (termed lox sites) it recognizes. This recombination system has been effective for achieving recombination in plant cells (U.S. Pat. No. 5,658,772), animal cells (U.S. Pat. Nos. 4,959,317, 5,801,030), and in viral vectors (Hardy et al., J. Virology 71:1842 (1997).

The plant cells, plants, and plant seeds can have genomic mutations that alter one or more amino acids in the encoded JAZ and/or CDK8 proteins. For example, plant cells, plants, and seeds can be modified so that at least one amino acid of a JAZ and/or CDK8 polypeptide is deleted or mutated to reduce the function of JAZ and/or CDK8 proteins. In some cases, a conserved amino acid or a conserved domain of the JAZ and/or CDK8 polypeptide is modified. For example, a conserved amino acid or several amino acids in a conserved domain of the JAZ and/or CDK8 polypeptide can be modified to change the physical and/or chemical properties of the conserved amino acid(s). For example, to change the physical and/or chemical properties of the conserved amino acid(s), the amino acid(s) can be deleted or replaced by amino acid(s) of another class, where the classes are identified in the following Table 1.

TABLE 1

| Classification | Genetically Encoded | Genetically Non-Encoded |
|---|---|---|
| Hydrophobic | | |
| Aromatic | F, Y, W | Phg, Nal, Thi, Tic, Phe(4-Cl), |
| | | Phe(2-F), Phe(3-F), |
| | | Phe(4-F), Pyridyl Ala, |
| | | Benzothienyl Ala |
| Apolar | M, G, P | |
| Aliphatic | A, V, L, I | t-BuA, t-BuG, MeIle, Nle, |
| | | MeVal, Cha, bAla, MeGly, |
| | | Aib |
| Hydrophilic | | |
| Acidic | D, E | |
| Basic | H, K, R | Dpr, Orn, hArg, Phe(p-NH₂), |
| | | DBU, A₂BU |
| Polar | Q, N, S, T, Y | Cit, AcLys, MSO, hSer |
| Cysteine-Like | C | Pen, hCys, β-methyl Cys |

Different types of amino acids can be in the modified JAZ and/or CDK8 polypeptide(s), such as any of those listed in Table 2.

TABLE 2

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| N-Methylglycine | | MeGly |
| (sarcosine) | | |
| Ornithine | | Orn |
| Citrulline | | Cit |
| N-methylisoleucine | | MeIle |
| Phenylglycine | | Phg |
| Norleucine | | Nle |
| Penicillamine | | Pen |
| Homoarginine | | hArg |
| N-acetyl lysine | | AcLys |
| ρ-Aminophenylalanine | | Phe(pNH₂) |
| N-methylvaline | | MeVal |
| Homocysteine | | hCys |
| Homoserine | | hSer |

For example, modified JAZ and/or CDK8 proteins can have any naturally occurring amino acid within the protein replaced with any of the amino acids listed in Tables 1 or 2.

In some cases, jaz and/or cdk8 mutations are introduced by insertion of foreign DNA into the gene of interest. For example, this can involve the use of either transposable elements (see, e.g., Parinov et al., Plant Cell 11, 2263-2270 (1999)) or T-DNA. The foreign DNA not only disrupts the expression of the gene into which it is inserted but also acts as a marker for subsequent identification of the mutation. Because some plant introns are small, and because there can be very little intergenic material in plant chromosomes, the insertion of a piece of T-DNA on the order of 5 to 25 kb in length generally produces a dramatic disruption of gene function. If a large enough population of T-DNA-transformed lines is available, one has a very good chance of finding a plant carrying a T-DNA insert within any gene of interest.

Mutations that are homozygous lethal can be maintained in the population in the form of heterozygous plants.

Table 3 illustrates jaz mutations that can be combined to generate jazD mutant strains.

TABLE 3
Mutants used for construction of jazD and jazU.

| Mutant | Original name | Source | Accession | Mutagen | Resistance¹ |
|---|---|---|---|---|---|
| jaz1-2 | SM _3.22668 | JIC SM | Col-0 | dSpm transposon | Basta (confirmed) |
| jaz2-3 | RIKEN_13-5433-1 | RIKEN | No-0 | Ds transposon | Hygromycin |
| | | | | | (confirmed) |
| jaz3-4 | GK-097F09 | GABI Kat | Col-0 | T-DNA (pAC161) | Sulfadiazine |
| | | | | | (confirmed) |
| jaz4-1 | SALK_141628 | SALK | Col-0 | T-DNA (pROK2) | Kanamycin |
| | | | | | (silenced) |
| jaz5-1 | SALK_053775 | SALK | Col-0 | T-DNA | Kanamycin |
| | | | | (pROK2) | (confirmed) |
| jaz6-4 | CSHL_ET30 | CSHL | Ler | Ds transposon | Kanamycin |
| | | | | (Enhancer trap GUS) | (confirmed) |
| jaz7-1 | WiscDsLox7H11 | Wisconsin | Col-0 | T-DNA (pWiscDsLox) | Basta |
| | | | | | (not tested) |
| jaz8-V² | N/A | ABRC | Vash-1 | SNP | N/A |
| jaz9-4 | GK_265H05 | GABI kat | Col-0 | T-DNA | Sulfadiazine |
| | | | | (pAC161) | (confirmed) |
| jaz10-1 | SAIL_92_D08 | SAIL | Col-0 | T-DNA | Basta |
| | | | | (pCSA110) | (confirmed) |
| | | | | | GUS |
| jaz13-1 | GK_193G07 | GABI kat | Col-0 | T-DNA | Sulfadiazine |
| | | | | (pAC161) | (not tested) |

¹Resistance of the mutant line to the indicated selectable marker was tested and confirmed.
²The C-to-A nonsense mutation present in JAZ8 from accession Vash-1 was backcrossed four times to Col-0 to generate a line (#28-6-30) that was used for subsequent genetic crosses (Thireault et al., Plant J 82: 669-679 (2015)).
N/A, not applicable.

jazD Mutations

A series of JAZ transcriptional repressor genes can be modified to improve insect and biotic resistance in plants. The JAZ transcriptional repressor genes can encode JAZ1, JAZ2, JAZ3, JAZ4, JAZ5, JAZ6, JAZ7, JAZ9, JAZ10, JAZ13, and/or related proteins. Reduction or deletion of genes that encode JAZ1, JAZ2, JAZ3, JAZ4, JAZ5, JAZ6, JAZ7, JAZ9, JAZ10, JAZ13, and/or related proteins can provide insect and biotic resistance to plants.

JAZ1 proteins are repressors of the jasmonic acid signaling pathway. One example, of an Arabidopsis thaliana jasmonate-zim-domain protein 1 (JAZ1) protein sequence is shown below (SEQ TD NO: 1).

| | |
|---|---|
| 1 | MSLFPCEASN MDSMVQDVKP TNLFPRQPSF SSSSSSLPKE |
| 41 | DVLKMTQTTR SVKPESQTAP LTIFYAGQVI VFNDFSAEKA |
| 81 | KEVINLASKG TANSLAKNQT DIRSNIATIA NQVPHPRKTT |
| 121 | TQEPIQSSPT PLTELPIARR ASLHRFLEKR KDRVTSKAPY |
| 161 | QLCDPAKASS NPQTTGNMSW LGLAAEI |

A chromosomal DNA sequence for the Arabidopsis thaliana jasmonate-zim-domain protein 1 (JAZ1) protein with SEQ ID NO:1 is shown below as SEQ ID NO:2.

| | |
|---|---|
| 1 | ATATTGGAGG TAGGAAGAAG AACTCTGCAA CCAAACCAAC |
| 41 | CAACCCCAAA GCCAAACAAA GTTTTATAGA GACCTTCCAT |
| 121 | TTCTCCCTCT CGTGACAAAC GCAATTTGCA GAGAAGCAAC |
| 201 | AGCAACAACA AGAAGAAGAA GAAAAAGATT TGAGATTACT |
| 241 | TTGTATCGAT TTAGCTATTC GAGAAACTCT TGCCGTTTGA |
| 281 | AAGTTTTAAT TGTTAAAGAT GTCGAGTTCT ATGGAATGTT |
| 321 | CTGAGTTCGT CGGTAGCCGG AGATTTACTG GGAAGAAGCC |
| 361 | TAGCTTCTCA CAGACGTGTA GTCGATTGAG TCAGTATCTA |
| 401 | AAAGAGAACG GTAGCTTTGG AGATCTGAGC TTAGGAATGG |
| 441 | CATGCAAGCC TGATGTCAAT GGTAAGAAAC CTTCTCTTTC |
| 481 | TCCTAGATCC ACTTCTTTTT TCGTTTTCTC TGTTTTTTAT |
| 521 | TTCTTGAATC TTGATCTTGA AAACTTTTCA AGAAAATTTT |
| 561 | GAATCGATTT CAAAGAAATT AGGGAGAGTT AGTTTGCTAA |
| 601 | ATTTTGACAT AGAAAATGAT TGGAGAGAGT TCTAACTTTT |
| 641 | GGATCATATA TATTTGCAGG AACTTTAGGC AACTCACGTC |
| 681 | AGCCGACAAC AACCATGAGT TTATTCCCTT GTGAAGCTTC |
| 721 | TAACATGGAT TCCATGGTTC AAGATGTTAA ACCGACGAAT |
| 761 | CTGTTTCCTA GGCAACCAAG CTTTTCTTCC TCATCTTCCT |
| 801 | CTCTTCCAAA GGAAGATGTT TTGAAAATGA CACAGACTAC |
| 841 | CAGATCTGTG AAACCAGAGT CTCAAACTGC ACCATTGACT |
| 881 | ATATTCTACG CCGGGCAAGT GATTGTATTC AATGACTTTT |
| 921 | CTGCTGAGAA AGCCAAAGAA GTGATCAACT TGGCGAGCAA |
| 961 | AGGCACCGCT AATAGCTTAG CCAAGAATCA AACCGATATC |
| 1001 | AGAAGCAACA TCGCTACTAT CGCAAACCAA GTTCCTCATC |
| 1041 | CAAGAAAAAC CACAACACAA GAGCCAATCC AATCCTCCCC |
| 1081 | AACACCATTG ACAGAACTTC CTATTGCTAG AAGAGCTTCA |
| 1121 | CTTCACCGGT TCTTGGAGAA GAGAAAGGAC AGAGTTACGT |
| 1161 | CAAAGGCACC ATACCAATTA TGCGATCCAG CCAAAGCGTC |
| 1201 | TTCAAACCCT CAAACCACAG GCAACATGTC GTGGCTCGGT |
| 1241 | TTAGCAGCTG AAATATGAAT GCTAACCACC CTCAAGCCGT |
| 1281 | ACCAAGAAAT TCTTTTGACG ACGTTGCTTC AAGACAAGAT |
| 1321 | ATAAAAGCTC CTATCTTCAT GCTTTTTGAT TTAAGATACA |
| 1361 | AACTACTCAA TGATTAGGAA ACTTCATATA TTTGTATGTA |
| 1401 | TTGATTAGTG ATCAATTATT GTTAGTATTC GTTATAGTCT |
| 1441 | GTTTTTCTAC TAGTTATTGT CGCCTGTCTA AATCCCCTTG |
| 1481 | CTATGGGTTA TCTCAAAATT AGTTTCGTAT GTAACTAATT |
| 1521 | TTGTAAGAAC AATAATTTTT GTTGACGAAC CATACTATCA |
| 1561 | AATACTCTAA ATTATATCTT GATAAATCTA CCTATCAGGT |
| 1601 | AAGTAGG |

JAZ2 is a coronatine (COR) and jasmonate isoleucine (JA-Ile) co-receptor, and is constitutively expressed in guard cells and modulates stomatal dynamics during bacterial invasion. It is expressed in cotyledons, hypocotyls, roots, sepals, petal vascular tissue and stigmas of developing flowers. JAZ2 is also expressed in stamen filaments after jasmonic acid treatment. One example, of an Arabidopsis thaliana jasmonate-zim-domain protein 2 (JAZ2) protein sequence is shown below (SEQ ID NO:3).

| | |
|---|---|
| 1 | MSSFSAECWD FSGRKPSFSQ TCTRLSRYLK EKGSFGDLSL |
| 41 | GMTCKPDVNG GSRQPTMMNL FPCEASGMDS SAGQEDIKPK |
| 81 | TMFPRQSSFS SSSSSGTKED VQMIKETTKS VKPESQSAPL |
| 121 | TIFYGGRVMV FDDFSAEKAK EVIDLANKGS AKSFTCFTAE |
| 161 | VNNNHSAYSQ KEIASSPNPV CSPAKTAAQE PIQPNPASLA |
| 201 | CELPIARRAS LHRFLEKRKD RITSKAPYQI DGSAEASSKP |
| 241 | TNPAWLSSR |

The Arabidopsis thaliana jasmonate-zim-domain 2 (JAZ2) gene resides on chromosome 1. A cDNA encoding the protein with SEQ ID NO:3 is shown below as SEQ ID NO:4.

| | |
|---|---|
| 1 | GCAACCAGCG AAAAAAAAGT AATAAAGAGG TCCTCCATTT |
| 41 | CTTCCTCGTG ACAAAACGCA CTTGGCAGAG AAAGATAAAC |
| 81 | AAGAACCCTA AGTTTTTTTA TAAGATTCGA GAAAATTCAA |
| 121 | CAACTCAGGA AGGAAGATCC TTTTGCTCCA ATTTCTCAAT |
| 161 | CGAAACGATT TCAATTTCGG TTTCAACGAT GTCGAGTTTT |
| 201 | TCTGCCGAGT GTTGGGACTT CTCTGGTCGT AAACCGAGCT |
| 241 | TTTCACAAAC ATGTACTCGA TTGAGTCGTT ACCTGAAGGA |
| 281 | GAAGGGTAGT TTTGGAGATC TGAGCTTAGG GATGACATGC |
| 321 | AAGCCCGACG TTAATGGAGG TTCACGTCAG CCTACAATGA |
| 361 | TGAATCTGTT CCCTTGTGAA GCTTCAGGAA TGGATTCTTC |
| 401 | TGCTGGTCAA GAAGACATTA AACCGAAGAC TATGTTTCCG |
| 441 | AGACAATCAA GCTTTTCTTC TTCCTCTTCC TCTGGGACCA |
| 481 | AAGAAGATGT ACAGATGATC AAAGAGACTA CTAAATCTGT |
| 521 | GAAGCCAGAG TCTCAATCTG CTCCGTTGAC TATATTCTAC |
| 561 | GGTGGTCGAG TTATGGTGTT TGATGATTTT TCTGCTGAGA |
| 601 | AAGCTAAAGA AGTCATTGAT TTGGCTAACA AAGGAAGTGC |
| 641 | CAAAAGCTTC ACATGTTTCA CAGCTGAAGT AAACAATAAC |
| 681 | CATAGTGCTT ATTCTCAAAA AGAGATTGCT TCTAGCCCAA |
| 721 | ATCCTGTTTG TAGTCCTGCA AAAACCGCAG CACAAGAGCC |
| 761 | AATTCAGCCT AACCCGGCCT CTTTAGCCTG CGAACTCCCG |
| 801 | ATTGCAAGAA GAGCTTCACT TCATCGGTTC CTTGAGAAGA |
| 841 | GGAAGGATAG GATCACATCA AAGGCACCAT ACCAAATAGA |
| 881 | CGGTTCAGCT GAAGCGTCTT CCAAGCCTAC TAACCCAGCT |
| 921 | TGGCTCAGTT CACGGTAAAC TTCGAGCCTG TCCGACCCAG |
| 961 | AAGGCACAAC TTGAGAGACC TTCTTGTAAG ATTCTTCTGA |
| 1001 | TGCTCCATCG TTACAAATAT CAAGCTGCTC CTCTGTTCAT |
| 1041 | TTTTTCTATA GATTAATTTC ACCCCTAGTA GTTTTGTTTG |
| 1081 | TTTAACTCCC CCGAAAACTC ATTATATTTG TATGAAATCA |
| 1121 | ATATCAATAG TGTTCAATGT TTGCTTCTGG GGTTTAAGTT |
| 1161 | TTAGCCAGTG TGTATAACCC TTTCCTCTGC CGATCTCAAC |
| 1201 | ATTAGCTTGC AACTTTTGTA AGAAACATCA CTTGTGTTTT |
| 1241 | TGTGTTGATG GCCATTAATA TAATCCAAGT TTATTTAATC |
| 1281 | CG |

JAZ3 is also a repressor of jasmonate responses, and it is targeted by the SCF(COI1) complex for proteasome degradation in response to jasmonate. One example, of an Arabidopsis thaliana jasmonate-zim-domain protein 3 (JAZ3) protein sequence is shown below (SEQ ID NO:5).

| | |
|---|---|
| 1 | MERDFLGLGS KNSPITVKEE TSESSRDSAP NRGMNWSFSN |
| 41 | KVSASSSQFL SFRPTQEDRH RKSGNYHLPH SGSFMPSSVA |
| 81 | DVYDSTRKAP YSSVQGVRMF PNSNQHEETN AVSMSMPGFQ |
| 121 | SHHYAPGGRS FMNNNNNSQP LVGVPIMAPP ISILPPPGSI |
| 161 | VGTTDIRSSS KPIGSPAQLT IFYAGSVCVY DDISPEKAKA |
| 201 | IMLLAGNGSS MPQVFSPPQT HQQVVHHTRA SVDSSAMPPS |
| 241 | FMPTISYLSP EAGSSTNGLG ATKATRGLTS TYHNNQANGS |
| 281 | NINCPVPVSC STNVMAPTVA LPLARKASLA RFLEKRKERV |
| 321 | TSVSPYCLDK KSSTDCRRSM SECISSSLSS AT |

A chromosomal DNA sequence for the Arabidopsis thaliana jasmonate-zim-domain protein 3 (JAZ3) protein with SEQ ID NO:5 is shown below as SEQ ID NO:6.

| | |
|---|---|
| 1 | GCGATTTGTT AATAAAACTA GAAATTGCGG TGAATTAACT |
| 41 | TCATTCCACG TTTTTTCATT TTCTCCCTCA AAAGTCTCTG |
| 81 | TTTTTTTTCC TTTTTCCGGC GAAGCTCTAT TTAGCTTGAT |
| 121 | TCCGGCGTTT AACACGCGTT TTAATCGAAA CAGACATTTG |
| 161 | AGATCGAATT AATTTTGTAG CGGGCTGTGT CTTTATTATA |
| 201 | GATGGAGAGA GATTTTCTCG GGTTGGGTTC GAAAAATTCT |
| 241 | CCGATCACTG TCAAGGAGGA AACCAGCGAA AGCTCTAGAG |
| 281 | ATTCAGGTTA TTTATTACTC TTCTCAATTT TTCTGATTCT |
| 321 | GATTGTTTTT AAATCGTAGA TTTGTTTGAT TGATTAGGAG |
| 361 | TTATTAGGAC TACTTGTAGT ATGGAATTTG TTTTTGGATA |
| 401 | GCTGATTTTA TGGCTTGCTC GGGAACTGGA ATTGTCAGTT |
| 441 | TGTTGCTTGG AGCAGAACAT TGTCCTTTGC TTTTCTCGGG |
| 481 | AGATGTAGAA TTTGGATTTG GAAAAACTAG TGTTCTTTTC |
| 521 | CAAAGCCTTG TCTTAAACAT GCTTTCGGTC GGAGAAATTA |
| 561 | ACGAGAACTA ATCTCAAGCT TCTAACATAA TTAAACTCGG |
| 601 | TAAACTTTTT TTTACTAGAG TAAATTTTTT TGTTTTGTTT |
| 641 | GAAGAGTCTT ATAATTGAGA AATACTTTAT TAGTTTATAC |
| 681 | TAAAAAAAAA ACGAATACGT AAAATGTTGG AAAAGAGGGG |
| 721 | ATGTATAGAG ACTGATACAA AAATGATAAA ATAGAGACGG |
| 761 | TTGGTAGTAG GTAGAAAGAT TAAATATACT CAAAAGAGTG |
| 801 | AGTTGGATTA GTTTATAAGA TGATTAACTT CTTGATTGTG |
| 841 | TGAGTTGGAT TAGTTTATGA GATTATTAAA ATATTGATTG |
| 881 | TGTATTTGTG TTGTGTGTTG ATTAAGCGGA ACTTGCGTTA |
| 921 | GAATATTGTT CAAGGTACAA TCTGGAAATA ATAGTTTTCT |
| 961 | CACCACGAGG AATATAATTA TTTCAACTTT GTTTTCTTAT |
| 1001 | CAGCCAAAAC GTGCCACACC ATAAAAGTAG TGCATCAACA |
| 1041 | TGTGGTGTGG TGTGGTGGGG TTAAAGTTTG AATCTCTCTT |
| 1081 | TAATTTAAAC TATTAAAACA AACTTAAATT ATTGGAGTTT |
| 1121 | CGTACAATGA CTTTCAATCA AATGTTTTAG AATTAGACAC |
| 1161 | GGTTTTCGAA AGTGGTTTTC CCTCGTTGAA TTTGTCAACA |
| 1201 | GTATGAGATT CTACATTGTT GGTTACTAAT CTTTTCCTTG |
| 1241 | AAGTAGGTGT TGAATTAATC CTCTGTTGTT TATGTAAGGA |
| 1281 | GATCTCGAGA CATTTATGGT TAACAGTTAA CACTACATGT |
| 1321 | TTGACTTTAA ACTGATTATC TTTTATTCTT TTTCTTTTGT |
| 1361 | AGCTCCCAAC AGAGGAATGA ACTGGTCTTT CTCAAACAAA |
| 1401 | GTATCAGCTT CTTCTTCTCA GTTTCTATCC TTCAGGCCAA |
| 1441 | CTCAAGAAGA TAGACATAGA AAGTCTGGAA ATTATCATCT |
| 1481 | TCCTCACTCT GGTTCCTTCA TGCCATCATC AGTAGCTGAT |
| 1521 | GTTTATGATT CAACCCGCAA AGCTCCTTAC AGTTCTGTAC |
| 1561 | AGGTATTTGT CATCAAAACC TATGTTAACC AAGACCCTTG |
| 1601 | TGTTTTTTTT ATCCTTCGCA AGATAGCTTT AAAAGTGAGC |
| 1641 | CCTGTTTTAT GAGCATATAG TAATTGGTTT TGAGTCTAGT |
| 1681 | TTAGCACAAG TTCATGGCAA TTAGTTTGTG GATCTAATCT |
| 1721 | TGGTTTAATA CTGATTCATT TTAAGTGTAA GCTAAGCTTC |
| 1761 | TCATTTTTGA TAAGTTAGTT CATACAATGC CTCACACCTA |
| 1801 | CTTTATGGCT TGTTACTCTC AGGGAGTGAG GATGTTCCCT |
| 1841 | AATTCCAATC AACACGAAGA AACTAACGCA GTTTCCATGT |
| 1881 | CGATGCCGGG TTTCCAGTCT CATCATTATG CACCAGGAGG |
| 1921 | AAGAAGCTTC ATGAACAATA ACAATAACTC ACAACCTTTG |
| 1961 | GTAGGAGTTC CTATCATGGC ACCTCCAATT TCAATCCTTC |
| 2001 | CTCCTCCAGG TTCCATTGTA GGGACTACTG ATATTAGGTA |
| 2041 | CCCACTAGTC ATCATATCAT ACAGAAACTC TTTCTACATT |
| 2081 | TTCATAGTTG ACTAAAGACT TATTTTTGTC AGATCTTCTT |
| 2121 | CCAAGCCAAT AGGTTCACCT GCGCAGTTGA CGATCTTTTA |
| 2161 | TGCCGGTTCA GTTTGTGTTT ACGATGACAT ATCTCCTGAA |
| 2201 | AAGGTATCTC AATCATTTTC TTCCATATAT GCATCTCTTT |
| 2241 | TACTCGTAAG GTATGGTACT CATTTGCTTT CTTTCATTTC |
| 2281 | TCAGGCAAAG GCGATAATGT TGCTAGCTGG GAACGGTTCC |
| 2321 | TCTATGCCTC AAGTCTTTTC GCCGCCTCAA ACTCATCAAC |
| 2361 | AAGTGGTCCA TCATACTCGT GCCTCTGTCG ATTCTTCAGC |
| 2401 | TATGCCTCCT AGCTTCATGC CTACAATATC TTATCTTAGC |
| 2441 | CCTGAAGCTG GAAGTAGCAC AAACGGACTC GGAGCCACAA |
| 2481 | AAGCGACAAG AGGCTTGACG TCAACATATC ACAACAACCA |
| 2521 | AGCTAATGGA TCCAATATTA ACTGCCCAGT ACCAGTTTCT |
| 2561 | TGTTCTACCA ATGTAATGGC TCCAACAGGT AAAAAACAAA |
| 2601 | GTCAGAGACC TGATACTACA TTCGCCATCT AACTTACTAG |
| 2641 | TATTTTCATG GATGTAACTT CATTCTCGTT CTGTTTCTTA |
| 2681 | TGCAGTGGCA TTACCTCTGG CTCGCAAAGC ATCCCTGGCT |
| 2721 | AGGTTTTTAG AGAAACGCAA AGAAAGGTAC GCAACACTTC |
| 2761 | TTTAGAATAC ACCATTCAAT AGTTTCTTGG GCTAACTCTC |
| 2801 | TTTCTCGCTG TGGGTTTCTC AGGGTCACGA GCGTATCCCC |
| 2841 | ATATTGCTTA GACAAGAAGT CATCGACAGA TTGTCGCAGA |
| 2881 | TCAATGTCTG AATGCATTAG TTCTTCTCTC AGCTCTGCAA |
| 2921 | CCTAATTTCA TCTACAGTAA GAAGGTTGCT TTAGACCACT |
| 2961 | CCACATCCAT ATTTGCATTT CAATGGCGGT CTTTTCAATG |
| 3001 | TCTCAGTTAA TTTTTCCTCA CTCGCCACAC TGAGTTTCTC |
| 3041 | CTTAGCTTTA TATATACGAT AGTGTATACT TTGTTTACAT |
| 3081 | GTTTTTTGGT GGAATGGAAC TTATGAGAGC ATATCAGATA |
| 3121 | TGTACTTGGG AAAATTAGTA GAAACTGTTT GTTTCTTTTT |
| 3161 | TTTTAACTCT GTTCTTTTGT ATATATCACT GAAGCTCGCA |
| 3201 | TATGTATAAT TCATGTAATG GAATTGCATC GCTTCTGTTT |
| 3241 | CCCTAAGTTA TTT |

JAZ4 is also a repressor of jasmonate responses. One example of an Arabidopsis thaliana jasmonate-zim-domain protein 4 (JAZ4) protein sequence is shown below (SEQ ID NO:7).

| | |
|---|---|
| 1 | MERDFLGLGS KLSPITVKEE TNEDSAPSRG MMDWSFSSKV |
| 41 | GSGPQFLSFG TSQQETRVNT VNDHLLSSAA MDQNQRTYFS |
| 81 | SLQEDRVFPG SSQQDQTTIT VSMSEPNYIN SFINHQHLGG |
| 121 | SPIMAPPVSV FPAPTTIRSS SKPLPPQLTI FYAGSVLVYQ |
| 161 | DIAPEKAQAI MLLAGNGPHA KPVSQPKPQK LVHHSLPTTD |
| 201 | PPTMPPSFLP SISYIVSETR SSGSNGVTGL GPTKTKASLA |
| 241 | STRNNQTAAF SMAPTVGLPQ TRKASLARFL EKRKERVINV |
| 281 | SPYYVDNKSS IDCRTLMSEC VSCPPAHHLH |

A chromosomal DNA sequence for the Arabidopsis thaliana jasmonate-zim-domain protein 4 (JAZ4) protein with SEQ ID NO:7 is shown below as SEQ ID NO:8.

| | |
|---|---|
| 1 | ATTAGAGGAA TCATAAATCG GCGGTGTGTG TAACTTCAAC |
| 41 | TCACGTTTTT CATTTCTCTC CAAAGTCCTT CAATTGTTAC |
| 81 | TAATTCTCTC TGATCTCTCA TTTCTTCTCT TCTCCGGTGA |
| 121 | CATTTTTTTT CTCCCCCGCG AAAGCTAAAC CGTTTTTGTA |
| 161 | TTCTCAACGA TTGATAAGCC TGATGGAGAG AGATTTTCTC |
| 201 | GGGCTGGGAT CAAAGTTATC TCCGATAACT GTGAAGGAGG |
| 241 | AAACTAACGA AGATTCAGGT AATTCATCTT CAACATCTTC |
| 281 | CATTATGATC TGATGATTGT GTTTTTCATC TCACTTTTTT |
| 321 | TTGTTTCTAT TTTTGTAATC TCTTTTTTTG TTTATTGTTC |
| 361 | AAGTACATAT ATATTGTTTT TCTAGCTTGA TTGGGAGTCC |
| 401 | TACTGTCTGG TTTTTTCTTG AACAAGAAAT TTTTTCTTCG |
| 441 | TTTTCTCGGG AAGAGAAAAA ATAAATTAGG GTTTCTTTTT |
| 481 | TCTTGATATA TATTTAAGAA ATTAGGTTTT AGTACTATAG |
| 521 | ACAGAAATTT AGCTACTCGA ATTTGTTTGA CGTAGCCGAT |
| 561 | GAAAAAACAC GTTTTGGGAC TCGATAGTTA GAAAATTCAT |
| 601 | ACGTTCACGA TCTACTTTTG AAGTTTTTTT CATTAAATAT |
| 641 | TTTTTGCAAA CTACAAATGT ACAAGTATAC AACTATACAA |
| 681 | GCAAACACCA AACTTGTTGA CGTTAGTAAT TTAACAAGTG |
| 721 | TTAGTATTAT CTTTGAAAAA TAATATTCAG AGAACAAACT |
| 761 | TGATTTTCTA GGTGACTAGG TGATGCATGT TTCTAAAGCT |
| 801 | GTTGGTAATG TTGAGTGTTT TCAAAATAAT TTCGTTTTTT |
| 841 | TCTTCAAACA GCCGACACCG ACAGAACAAA AATGCTATAT |
| 881 | TTTTITTGTT GCTTACAAAA TTGATCAATT GGTTTCAATA |
| 921 | CAATAGTATC TTCTTTAGAA AAGATTGTTT TTTTCAAAGC |
| 1001 | CGGATTGAAT ATTGAGAATT AGAACATTGG CTGGTTATTC |
| 1041 | TTTTTGAAAA GTTTATGCCA TTTTTTAAGG TTTATTAAGC |
| 1081 | AACTTGAATT CTATCAGTAT TATTTAAAAA CGAAGACGTG |
| 1121 | AAATGTTGGG AAAAGAATGC GTTATATAGC GACCGGCTGA |
| 1161 | CGATTAGAGA TTTAACAACA AATGCAAGTT GAATTATATA |
| 1201 | AAAGCAAGAT TGATTGTGAC TTGATTAAGT TTTATTTCTA |
| 1241 | TCCAAGTAGA CTCATTGATT AAGTTAGGAT CATGTTGGGT |
| 1281 | ATTAAATTTA GATCAAGTTA CAATTTGGAT GAATAATTTA |
| 1321 | CTTACCCACG AGGAATTTAA TAGTTAGTTC TTGTCTTTTT |
| 1361 | ATATTCCGAA ACGTGCCATT TCTTGAAAGT ATTTGTATGA |
| 1401 | TCACTATTTT CCCCAGTGTG TTTGGCTTTA TGCAGATTTG |
| 1441 | TTCATTGTTG ATGAATCTAA TGTTAAGAGT CGTCCACTTT |
| 1481 | AGCATAGCTA GATCTGAGTG TTTCCTAGTT TGATAAAATC |
| 1521 | TAAAGACATT TGCTCATGTT TCAGCCCCAA GTAGAGGTAT |
| 1561 | GATGGATTGG TCATTCTCAA GCAAAGTCGG TTCTGGTCCT |
| 1601 | CAGTTTCTTT CTTTTGGGAC ATCCCAACAA GAAACGCGTG |
| 1641 | TAAACACAGT CAATGATCAT TTGCTTTCTT CTGCTGCAAT |
| 1681 | GGATCAAAAC CAGAGAACTT ACTTCAGCTC ACTACAGGTT |
| 1721 | AGGCTATTTC TTGAAAAGAA AAAAAGTAGT GATAAAGTGT |
| 1761 | GATTTAGTGA CCTTGTAAGA AAGCTTGGCA ATTGGTTTAG |
| 1801 | TTTCTTCTGG TCTCAAAATT GATACAAAAT GATCTCAGGA |
| 1841 | AGACAGAGTG TTCCCAGGTT CCAGTCAGCA AGACCAAACA |
| 1881 | ACCATCACAG TCTCCATGTC CGAACCAAAC TACATCAACA |
| 1921 | GTTTCATAAA CCACCAACAT TTAGGAGGAT CTCCTATCAT |
| 1961 | GGCACCTCCA GTTTCAGTAT TTCCTGCTCC AACCACTATT |
| 2001 | AGGCATGCAC TGCATTCTAT CTTCTTCTGT TTAACATCAG |
| 2041 | ATACAGAACC TCTTTACTTC TATAGTTGAC TCGAGCTCCT |
| 2081 | TTATGTTCAT CTCCAGATCT TCTTCAAAAC CACTTCCCCC |
| 2121 | TCAGTTGACA ATCTTTTATG CCGGTTCAGT ATTAGTTTAC |
| 2161 | CAAGACATAG CTCCTGAAAA GGTAACCAAA TTTCCTTCAA |
| 2221 | TATGTGTTAC ATTAGAGTCC AAGCTATCCA CTGACTAAGT |
| 2241 | ATTCAATCAA AGAAATAAGT TTCACGTATA GACATGCTGA |
| 2281 | AGTTATAGAA AGTTACTAAC CTGGTTTCAA CATACAGTAT |
| 2321 | GTTAATGATT CATAGATATG ATAAATCTTT GTCCTTACTT |
| 2361 | CTTCATTTAT TTTGTATTCA TAGGCCCAAG CTATCATGTT |
| 2401 | GCTAGCCGGA AATGGACCTC ATGCTAAACC GGTTTCACAA |
| 2441 | CCTAAACCTC AAAAACTGGT TCATCACTCT CTTCCAACCA |
| 2481 | CTGATCCTCC AACTATGCCT CCTAGTTTCC TGCCTTCCAT |
| 2521 | CTCTTACATT GTCTCTGAAA CCAGAAGTAG TGGATCCAAC |
| 2561 | GGGGTTACTG GACTTGGACC AACAAAAACA AAGGCGAGTT |
| 2601 | TAGCATCCAC GCGCAACAAC CAAACTGCTG CCTTCTCTAT |
| 2641 | GGCTCCAACA GGTTATAAAT GAAGTCTTAA CTCCTATTAA |
| 2681 | TGTTTTGTCA TCAAACTTCT ATCTTAGGTT TAGTTTGTTA |
| 2721 | TAACCAAAAA ATCTTGCTAT GATTTAATAC AGTGGGTTTA |
| 2761 | CCACAAACAC GCAAAGCATC CTTGGCTCGG TTCTTAGAGA |
| 2801 | AACGCAAAGA AAGGTACTGA GCTACAAGAT TATTCACTTA |
| 2841 | TTCACAATAT CAAAACACAG GTTTGCTGTA TATTGGCTTC |
| 2881 | GTTTTCTTGC AGGGTCATTA ACGTATCACC TTATTACGTA |
| 2921 | GACAACAAGT CATCAATAGA CTGTAGAACA CTGATGTCTG |
| 2961 | AATGTGTAAG CTGTCCTCCA GCTCATCATC TGCACTAAAA |
| 3041 | CCAATTTAGA CCCCTCATTG TTCTAAAGGC TTTTTCTTTT |
| 3081 | TTCTCTGGCT CTGTATCCTA TAGACTATAG TATAGTTGTT |
| 3121 | ATAGCTTTTG TTTATTCAGA TTTTAGTACA CTGGGCTTGT |
| 3161 | AAAAGCAAGT TATTTATATA TATCCTATAA ATTTAATTTG |
| 3201 | GATACTGTAT GTTTTGTCTT TACTCTTGCA TGTGTATAAA |
| 3241 | AAACATAAAA GTAAGACTAT TCAAGCT |

JAZ5 is also a repressor of jasmonate responses. One example of an Arabidopsis thaliana jasmonate-zim-domain protein 5 (JAZ5) protein sequence is shown below (SEQ ID NO:9).

| | |
|---|---|
| 1 | MSSSNENAKA QAPEKSDFTR RCSLLSRYLK EKGSFGNIDL |
| 41 | GLYRKPDSSL ALPGKFDPPG KQNAMHKAGH SKGEPSTSSG |
| 81 | GKVKDVADLS ESQPGSSQLT IFFGGKVLVY NEFPVDKAKE |
| 121 | IMEVAKQAKP VTEINIQTPI NDENNNNKSS MVLPDLNEPT |
| 161 | DNNHLTKEQQ QQQEQNQIVE RIARRASLHR FFAKRKDRAV |
| 201 | ARAPYQVNQN AGHHRYPPKP EIVTGQPLEA GQSSQRPPDN |
| 241 | AIGQTMAHIK SDGDKDDIMK IEEGQSSKDL DLRL |

A cDNA sequence for the Arabidopsis thaliana jasmonate-zim-domain protein 5 (JAZ5) protein with SEQ ID NO:9 is shown below as SEQ ID NO:10.

| | |
|---|---|
| 1 | TAATCATGGA TGAAAATTCC TTTCTTCACA CTAGATATAG |
| 41 | TTCTTTAACT AGTTAAAAAT GCATGCGATG GAATATTACT |
| 81 | AAATATGATA TAATCTCATG GCTTTATGTA AGATTTGTTT |
| 121 | TTTGGTTTTT TTGGTTGTTG TTAATAAATT TATTATTGAG |
| 161 | AAGTTTAATT CTATTTTGGT CACAATATAT TGAAATATTT |
| 201 | TTAAGAAACT AAAAAGTTCC TATTTATTTT TGTTTTCATT |
| 241 | AATTTATGAG AGGCTATTAA AGTCACAGAA ACTTATTGGG |
| 281 | TGAATGAGTT TATAAACACA TGAGCTATTG AGCTAGTAGC |
| 321 | CTCTTGTACT CTTCCATTTT ACGCGCAATC CACGCACCAA |
| 361 | CAAAAAGAAA AGAAAAGAAG AGATAAAGAA TATCTTTAAA |
| 401 | AAGTAAGTGT GGAGAATTCT TTCTTCTCAA TAAACAACAA |
| 441 | CATGTCGTCG AGCAATGAAA ATGCTAAGGC ACAAGCGCCG |
| 481 | GAGAAATCTG ACTTTACCCG GAGATGTAGT TTGCTCAGCC |
| 521 | GTTACTTGAA GGAGAAGGGT AGTTTCGGAA ACATTGATCT |
| 561 | TGGCTTATAC CGAAAACCCG ATTCCAGTCT CGCGTTGCCC |
| 601 | GGAAAATTCG ATCCACCAGG GAAACAAAAT GCGATGCATA |
| 641 | AGGCAGGGCA TTCCAAAGGC GAACCCTCTA CCTCATCAGG |
| 681 | AGGCAAAGTC AAAGATGTTG CTGACCTCAG TGAATCACAG |
| 721 | CCAGGAAGTT CGCAGCTGAC CATATTCTTC GGAGGGAAAG |
| 761 | TTTTAGTATA TAATGAGTTC CCCGTAGACA AAGCTAAAGA |
| 801 | GATTATGGAA GTAGCAAAAC AAGCCAAGCC TGTGACTGAG |
| 841 | ATTAACATTC AGACACCAAT CAATGACGAA AACAACAACA |
| 881 | ACAAGAGCAG CATGGTTCTT CCTGATCTCA ATGAGCCTAC |
| 921 | TGATAATAAT CACCTAACAA AGGAACAACA ACAGCAACAA |
| 961 | GAACAAAATC AGATCGTGGA ACGTATAGCA CGTAGAGCTT |
| 1001 | CCCTCCATCG ATTCTTTGCT AAACGGAAAG ACAGAGCTGT |
| 1041 | GGCTAGGGCT CCGTACCAAG TTAACCAAAA CGCAGGTCAT |
| 1081 | CATCGTTATC CTCCCAAGCC AGAGATTGTA ACCGGTCAAC |
| 1121 | CACTAGAGGC AGGACAGTCG TCACAAAGAC CGCCGGATAA |
| 1161 | CGCCATTGGT CAAACCATGG CCCATATCAA ATCAGACGGT |
| 1201 | GATAAAGATG ATATTATGAA GATTGAAGAA GGCCAAAGTT |
| 1241 | CGAAAGATCT CGATCTAAGG CTATAGTAAT ATTTGCTAAA |
| 1281 | TTTCTTGTAG GAACTGAGTT TTTAGATTAA CGTTTCGATT |
| 1321 | TTTCTGACTT ATCTAAGTGA TTTTATTTTG CTTTGTACTA |
| 1361 | CAGTATGTAA TCTTATTCTA ACTTGAATAT TCATTCATAA |
| 1401 | ACACAATAGA CGATAGTAAA GTTATATTAT AATTAGTTAA |
| 1441 | CTACGTACAA CACTTGGGAG TTAAATTACA TAACGTTAAG |
| 1481 | CGAGAAATAG CAAATTAGAC AAGAGGAAGA ATATTTAGGA |
| 1521 | GTTGTGAATT GATCTGACTG CAATAACATG AAGAGGAATC |
| 1561 | TGACTGCAAT CGTAATGCGG GTAAAGATGG TTGAAAGTGA |
| 1601 | TCAGAGCTCC TTTCTAATTT ATTTAGGGTG TAATTTATGA |
| 1641 | AAATGATTAT TATTGGAGTG TATATCAAGT TTTCACTAAA |
| 1681 | CTCAGGGGTG TTTATTGTAA TTAGTTGTCA GGTTCAAGTT |
| 1721 | CATTGAAGGC GTGTCTGATT TGGACAGTGA TTGGGCCTGA |
| 1761 | GCCAT |

JAZ6 is also a repressor of jasmonate responses. One example of an Arabidopsis thaliana jasmonate-zim-domain protein 6 (JAZ6) protein sequence is shown below (SEQ ID NO:11).

| | |
|---|---|
| 1 | MSTGQAPEKS NFSQRCSLLS RYLKEKGSFG NINMGLARKS |
| 41 | DLELAGKFDL KGQQNVIKKV ETSETRPFKL IQKFSIGEAS |
| 81 | TSTEDKAIYI DLSEPAKVAP ESGNSQLTIF FGGKVMVFNE |
| 121 | FPEDKAKEIM EVAKEANHVA VDSKNSQSHM NLDKSNVVIP |
| 161 | DLNEPTSSGN NEDQETGQQH QVVERIARRA SLHRFFAKRK |
| 201 | DRAVARAPYQ VNQHGSHLPP KPEMVAPSIK SGQSSQHIAT |
| 241 | PPKPKAHNHM PMEVDKKEGQ SSKNLELKL |

A cDNA sequence for the Arabidopsis thaliana jasmonate-zim-domain protein 6 (JAZ6) protein with SEQ ID NO:11 is shown below as SEQ ID NO:12.

| | |
|---|---|
| 1 | AAATTAATAG CCTATAATAT GTTTGACCAT AAAAAGAATT |
| 41 | TCTTCTTCTT GAACCATCAT AAGAAAAATG TGTGTTTAGT |
| 81 | CTATTGATCA GTTTTGTGTT GAAAAAAAAA AAAAAAATGT |
| 121 | CTATCCATCA GTTAGGTGTA AAAAAAAAAG TTACAAAACT |
| 161 | CCTGACAAAA ACATTCTATA TTGGACACAC ATCACTGTCA |
| 201 | CTTCAGACTA AATAAAAAAA AAGAACACGT TATTTCGTTT |
| 241 | TCTTTATTTA TTCGGGAGAG GTTAAAAGCC ACAGAAACTT |
| 281 | ATTGGCTAGA ATTGGTTATT TATAACAACA ACACATGAGC |
| 321 | AAAAAGCTCA AAACATCTAC ATACTCTTTG GAATCCTCGA |
| 361 | TTTTTTGTAC GTGTAAAGAA GTCACACAAG AAAATCTTGG |
| 401 | GTTGTTGTAA TCTTCATCAC ACTAGTATGT CAACGGGACA |
| 441 | AGCGCCGGAG AAGTCCAATT TTTCTCAGAG ATGTAGTCTG |
| 481 | CTCAGCCGGT ACTTGAAGGA GAAGGGAAGT TTTGGGAATA |
| 521 | TTAATATGGG GTTGGCTCGA AAATCCGATC TTGAACTCGC |
| 561 | CGGAAAATTC GATCTCAAAG GACAACAAAA TGTGATTAAG |
| 601 | AAGGTAGAGA CCTCAGAAAC TAGACCGTTC AAGTTGATTC |
| 641 | AGAAGTTTTC TATTGGTGAG GCCTCTACTT CTACCGAAGA |
| 681 | CAAAGCCATA TATATTGATC TCAGTGAACC GGCAAAAGTA |
| 721 | GCACCGGAGT CTGGAAATTC ACAGTTGACC ATATTCTTTG |
| 761 | GAGGAAAAGT TATGGTTTTC AACGAGTTTC CTGAAGACAA |
| 801 | AGCTAAGGAG ATAATGGAAG TAGCTAAAGA AGCGAATCAT |
| 841 | GTTGCTGTTG ATTCTAAGAA CAGTCAGAGT CACATGAATC |
| 881 | TTGACAAAAG CAACGTGGTG ATTCCCGATC TTAACGAGCC |
| 921 | AACGAGTTCC GGGAACAATG AAGATCAAGA AACTGGGCAG |
| 961 | CAACATCAGG TTGTGGAACG CATTGCAAGA AGAGCTTCTC |
| 1001 | TTCATCGATT CTTTGCTAAA CGAAAAGACA GGGCTGTGGC |
| 1041 | TAGAGCTCCA TATCAAGTGA ACCAACACGG TAGTCATCTT |
| 1081 | CCTCCCAAGC CAGAGATGGT TGCTCCATCG ATAAAGTCAG |
| 1121 | GCCAATCGTC GCAACACATT GCAACTCCTC CAAAACCAAA |
| 1161 | GGCCCATAAC CATATGCCGA TGGAGGTGGA CAAGAAAGAA |
| 1201 | GGACAATCTT CCAAAAACCT TGAACTCAAG CTTTAGGGCG |
| 1241 | TATAAAATGC ACGATCGAGT TCACGTTTCT AGTTTTCACT |
| 1281 | TATTTAGGAT TTGAACCCAA ATACCCTTTT ATATTTTCTT |
| 1321 | CCATTACTTT TGACCAATTT AAGTTATTTA TAGTACTGTA |
| 1361 | TTACGTAGCT AGTATTTATA TTTCAAAACA TAGATATTTT |
| 1401 | GATACTTGTT TTTTAGATTC TTTAATTAAA ATTGTCATCT |
| 1441 | GGATTACCCT TTATCGAAAT TTTTTAATCA CCTGATATAA |
| 1481 | TCTCACCAGT GATGGATTTG CGTTGTTAGT AATTTTTCTA |
| 1521 | AGTGGCAAAA GTATATTAAC CTATAATAGG TTTCAAAGAT |
| 1561 | ATACATATAA TGTTTCTATC AAAGATATTA GTATAATATT |
| 1601 | TTAC |

JAZ7 is also a repressor of jasmonate responses. One example of an Arabidopsis thaliana jasmonate-zim-domain protein 7 (JAZ7) protein sequence is shown below (SEQ ID NO:13).

| | |
|---|---|
| 1 | MIIIIKNCDK PLLNFKEMEM QTKCDLELRL LTSSYDSDFH |
| 41 | SSLDESSSSE ISQPKQESQI LTIFYNGHMC VSSDLTHLEA |
| 81 | NAILSLASRD VEEKSLSLRS SDGSDPPTIP NNSTRFHYQK |
| 121 | ASMKRSLHSF LQKRSLRIQA TSPYHRYR |

A cDNA sequence for the Arabidopsis thaliana jasmonate-zim-domain protein 7 (JAZ7) protein with SEQ ID NO:13 is shown below as SEQ ID NO:14.

| | |
|---|---|
| 1 | GTTGTGTTCT GTCCAAACTC TGTTCTAATG CCAGCTTTTG |
| 41 | TCTCGTCTTT TCTCTATCCT TATCTTCCCT CTATCTTCGA |
| 81 | TCCCAACACA TACACAACAC GCACACACAC ATATATAAAT |
| 121 | CAACTGACTG ACACATACAA TCATGATCAT CATCATCAAA |
| 161 | AACTGCGACA AGCCTTTACT CAATTTCAAA GAGATGGAGA |
| 201 | TGCAAACAAA ATGCGACTTG GAACTTCGCC TTCTTACTTC |
| 241 | TTCTTATGAT TCTGATTTCC ATAGCTCGTT GGACGAATCA |
| 281 | AGCAGCTCTG AAATTTCACA ACCAAAGCAA GAATCTCAGA |
| 321 | TATTAACCAT TTTCTACAAC GGGCACATGT GTGTTTCTTC |
| 361 | AGATCTTACC CATCTTGAGG CTAACGCTAT ACTATCGCTA |
| 401 | GCGAGTAGAG ATGTGGAAGA GAAATCTTTA TCCTTGAGAA |
| 441 | GTTCAGACGG TTCGGATCCT CCAACAATCC CAAACAATTC |
| 481 | GACTCGATTT CATTATCAAA AGGCCTCTAT GAAGAGATCT |
| 521 | CTTCACAGTT TTCTTCAGAA ACGAAGTCTT CGGATTCAAG |
| 561 | CAACTTCCCC TTACCACCGT TACCGATAGC ACTATCTATT |
| 601 | TGATTTCATT TTTGTGATTC TCTTCAATTT TTTTTTTACT |
| 641 | GTAACATAAT AATCCAATTG TCTTGAATTC TTTTTCTGTG |
| 681 | TGTTTGGATG GATTAGAGAC CTTAATTAGG TAGAGTATTA |
| 721 | AAGTTTCATA ATTTCCAGTA ACTTGTGTTT AGAGTTCAAG |
| 761 | AGGTTGACAA AATTTATCAA CGGTCTCCTA AAATGGGTAA |
| 801 | ACCGAGAAAC TTTTATACGA AAA |

JAZ9 is also a repressor of jasmonate responses. One example of an Arabidopsis thaliana jasmonate-zim-domain protein 9 (JAZ9) protein sequence is shown below (SEQ ID NO:15).

| | |
|---|---|
| 1 | MERDFLGLSD KQYLSNNVKH EVNDDAVEER GLSTKAAREW |
| 41 | GKSKVFATSS FMPSSDFQEA KAFPGAYQWG SVSAANVFRR |
| 81 | CQFGGAFQNA TPLLLGGSVP LPTHPSLVPR VASSGSSPQL |
| 121 | TIFYGGTISV FNDISPDKAQ AIMLCAGNGL KGETGDSKPV |
| 161 | REAERMYGKQ IHNTAATSSS SATHTDNFSR CRDTPVAATN |
| 201 | AMSMIESFNA APRNMIPSVP QARKASLARF LEKRKERLMS |
| 241 | AMPYKKMLLD LSTGESSGMN YSSTSPT |

A chromosomal DNA sequence for the Arabidopsis thaliana jasmonate-zim-domain protein 9 (JAZ9) protein with SEQ ID NO:15 is shown below as SEQ ID NO:16.

| | |
|---|---|
| 1 | GCAAAGAGTT AAATAAGCCT CTCCAAAAGT GTGTCTGTAA |
| 41 | CATTACCAAA ACGAAACCTT CCTTGTGGAT TCCCACTTCT |
| 81 | TTCTTCTGTT TTCTTCTTCC TCTTCTTTAA ATTGGATGTT |
| 121 | TTGGGCAAGA AACAGAGAGA AACACGTTAA TTTGAGAGTT |
| 161 | TGTCATTGAA TATTTGGTTT GCAATGGAAA GAGATTTTCT |
| 201 | GGGTTTGAGC GACAAGCAGT ATCTAAGTAA TAACGTTAAG |
| 241 | CATGAGGTTA ACGATGATGC TGTCGAAGAA CGAGGTTTGT |
| 281 | GTTCTTGTCT CGAGAATCTT TTATTTTAAT GTTTCAAGAA |
| 321 | GAGATCAGTT TTCACTTTTA ACATAGCCGT ATAAAGTTGT |
| 361 | TTATTTAAAT ATAATTTTTC AGATTCCAAA ACTTGAAAAA |
| 401 | AAAAAGATTC CATTAAATCT TTTATAAAAA TGAGATTGGA |
| 441 | TAGATTAGTC AAATTGACGA CCATAAAAAA TGATACTTAT |
| 481 | AGGGTTAAGT ACGAAGGCAG CTAGAGAATG GGGGAAGTCA |
| 521 | AAGGTTTTTG CTACTTCAAG TTTCATGCCT TCTTCAGATT |
| 561 | TCCAGGTTGG TTCATCTTAA AATTTAACTT ACTCTGTATC |
| 601 | AGTTTCAGAT GTTATGGCTA ATCTAATGGT TCTATAAGCT |
| 641 | ACCGCATAAT CATGGTCGTC TTTTAGCATG TGCAAGAGGA |
| 681 | GTACTCAATT ATGGTCTTGA TTAAAAAGAA GAATTTACTT |
| 721 | TCAAATTATG TTAAACACAT CAATCACATA TTTATGAGAA |
| 761 | AAGTTGTTTT CGTAAGAGAT AGCCACCGGA AAATGGTCGG |
| 801 | ATAAATGGCC GAACTTTATC ATTTTTGTGT ATGTGGCCAA |
| 841 | TCATTAACCA GGGAAAAAAA ATTGTTGGAT AAGTGCTAGT |
| 881 | TAAGAGCTGG TAGGGTCGGT CGTCTGCCAG CCGCAAAGTT |
| 921 | AGGGAAAAAA TAATTTAATA TTTTGTGGCG TTTGGTGTTT |
| 961 | GGCGTTTGGA TCACGTTTAT TTCTTGGCAT TTTTCTAAAT |
| 1001 | TTAGAATGTA CAAAAAATTT AAAGACGTTG ACGATTAAAA |
| 1041 | TTTGAATTTA ACAAATTAGG AGGCTAAGGC GTTTCCGGGT |
| 1081 | GCATACCAGT GGGGATCAGT TTCTGOGGCC AATGTTTTCC |
| 1121 | GCAGATGCCA ATTTGGTGGT GCGTTTCAAA ACGCGACGCC |
| 1161 | GCTTTTACTA GGCGGTTCAG TTCCTTTACC AACTCATCCT |
| 1201 | TCTCTTGTTC CACGGTAATT TCCATATTAT GATGCAAAAA |
| 1241 | CATTCAACAA TTTTTTTGCT CTTTTCATAT TTTGATTTGG |
| 1281 | TTATGTGGGT TTGTGGAAAC AGAGTGGCTT CCTCCGGATC |
| 1321 | ATCTCCTCAG CTCACAATCT TTTATGGCGG AACTATAAGC |
| 1361 | GTCTTTAATG ACATATCTCC CGATAAGGTA TATATAATCA |
| 1401 | AGATTCATAC AAATAACATT TACATAACAT TTACATGTTC |
| 1441 | TAAAACGGAC TATTCATGAT ATGTGAGTAG GCTCAAGCCA |
| 1481 | TCATGTTATG CGCCGGGAAC GGTTTGAAAG GTGAAACTGG |
| 1521 | AGATAGCAAA CCGGTTCGAG AAGCTGAAAG AATGTATGGA |
| 1561 | AAACAAATCC ATAACACTGC TGCTACCTCA TCAAGCTCTG |
| 1601 | CCACTCACAC TGATAATTTC TCAAGGTGTA GGGACACACC |
| 1641 | CGTTGCTGCG ACTAATGCAA TGAGCATGAT CGAATCATTC |
| 1681 | TATGCAGCTC CTCGTAACAT GATTCCTTCA GGTATGTGTG |
| 1721 | TCTAATATCA ACATCAAAAC AAAATATAAT CAAGATTTTT |
| 1761 | GCTTCCTCAA ATCATATGTC TAAACTCGAA AATTGCTTTT |
| 1801 | TTCCAGTCCC TCAAGCTCGG AAAGCATCCT TGGCTCGGTT |
| 1841 | CTTGCAGAAG CCCAAAGAGA GGTTTGATTT TGTATTTTTT |
| 1881 | TTCTTTATAG AAAATTTTGA GGTTTTTCAA TTGAATCTAA |
| 1921 | AAGAATTGAT GTTGTTGGTG CAGGCTTATG AGTGCAATGC |
| 1961 | CATACAAGAA GATGCTTCTT GATTTGTCGA CCGGAGAATC |
| 2001 | CAGTGGAATG AATTACTCTT CTACTTCTCC TACATAAAAC |
| 2041 | CTACACTTTT TTTTTTTTTT TTTACAATGG TAATTTGTAA |
| 2081 | TTGTAATCAT TAGATTATGA TTATATAGTT ACCATTTATA |
| 2121 | TTCTTACGAG CAGGAGAAGA CGTTAGGGCG TCTCTGTATT |
| 2161 | TGATCATTGT TTGTAATGCT TTGGTCTGTT TATTGTAGGA |
| 2201 | TTACATTATA ACTTTAAGAA CTAACAGATA TATGTTTGTC |
| 2241 | ATGGACTCAT GTCTGTCAAG AATTTAATAT CAAATAAAAT |
| 2281 | TCACTATAAT TTTTTTT |

JAZ10 is also a repressor of jasmonate responses. One example of an Arabidopsis thaliana jasmonate-zim-domain protein 10 (JAZ10) protein sequence is shown below (SEQ ID NO:17).

| | |
|---|---|
| 1 | MSKATIELDF LGLEKKQTNN APKPKFQKFL DRRRSFRDIQ |
| 41 | GAISKIDPEI IKSLLASTGN NSDSSAKSRS VPSTPREDQP |
| 81 | QIPISPVHAS LARSSTELVS GTVPMTIFYN GSVSVFQVSR |
| 121 | NKAGEIMKVA NEAASKKDES SMETDLSVIL PTTLRPKLFG |
| 161 | QNLEGDLPIA RRKSLQRFLE KRKERLVSTS PYYPTSA |

A chromosomal DNA sequence for the Arabidopsis thaliana jasmonate-zim-domain protein 10 (JAZ10) protein with SEQ ID NO:17 is shown below as SEQ ID NO:18.

| | |
|---|---|
| 1 | AAAAACTCTC ACATGAGAAA TCAGAATCCG TTATTATTCC |
| 41 | TCCATTTATT CATCTCAAAA CCCATATCTC TCTGTCTTGA |
| 81 | TCTCTCTCTC ACTTTCTAAT AAGATCAAAG AAGATGTCGA |
| 121 | AAGCTACCAT AGAACTCGAT TTCCTCGGAC TTGAGAAGAA |
| 161 | ACAAACCAAC AACGCTCCTA AGCCTAAGTT CCAGAAATTT |
| 201 | CTCGATCGCC GTCGTAGTTT CCGAGGTTCG TTTGGTTTTT |
| 241 | AGTCGCTCTC TCTTTTTTTT TTCTTGCGAT AAATCGAATT |
| 281 | TATTCATATG GAACTCCTGC AGATATTCAA GGTGCGATTT |
| 321 | CGAAAATCGA TCCGGAGATT ATCAAATCGC TGTTAGCTTC |
| 361 | CACTGGAAAC AATTCCGATT CATCGGCTAA ATCTCGTTCG |
| 401 | GTTCCGTCTA CTCCGAGGGA AGATCAGCCT CAGATCCCGA |
| 441 | TTTCTCCGGT CCACGCGTCT CTCGCCAGGT ATTTTTGTCT |
| 481 | TTCCGGTAAA GTTTTTTTTT TCTTTCTAAC TTTTTTGGCG |
| 521 | CTACCAGAAA AGACGAAAAA ATTTGAAATT CAAATTTTCA |
| 561 | AAACATTCAT TTTCCTCAGG TCTAGTACCG AACTCGTTTC |
| 601 | GGGAACTGTT CCTATGACGA TTTTCTACAA TGGAAGTGTT |
| 641 | TCAGTTTTCC AAGTGTCTCG TAACAAAGCT GGTGAAATTA |
| 681 | TGAAGGTCGC TAATGAAGCA GCATCTAAGA AAGACGAGTC |
| 721 | GTCGATGGAG ACAGATCTTT CGGTAATTCT TCCGACCACT |
| 761 | CTAAGACCAA AGCTCTTTGG CCAGAATCTA GAAGGAGGTT |
| 801 | AGTATAATAA TAATAAATAT CACTTAGTGC TGGATTCTTC |
| 841 | TAGAATTTTA GTTACATATT ATTGCATGTA GAGATCTAAG |
| 881 | AAGAGTTTGT TGTTAGAGAG GAATTGGTTG CTAATTAGTT |
| 921 | TGCAATTAGA TATCAAAGAG TTAAAGACTA TAGTTTATGT |
| 961 | CTATACGTAT TAATATACGT TATTAATAAA AGTATAAACA |
| 1001 | TGTTGTTTAA TTTCTGATAA GAAACTGGTT TATGCGTGTG |
| 1041 | TATGCAGATC TTCCCATCGC AAGGAGAAAG TCACTGCAAC |
| 1081 | GTTTTCTCGA GAAGCGCAAG GAGAGGTAAT GATTCTTCAA |
| 1121 | CAATCCAAGG ATTTTTACCC CCAAATAATT AAAGAAAGGT |
| 1161 | TTTTATTTTT CTCTCTCTCG ACCTTTTTTT TACTATAAGT |
| 1201 | TATTTAAGAT AGTAATTATG GGTCCTGCCT CTTTTACTCT |
| 1241 | CACATACAAC TTAAGATTCA ACTAGTTTTG TTCAACAACG |
| 1281 | CACATGCTTA TACGTAGATA GATAATGGAG ATCAGTAGTA |
| 1321 | ATATCGGTAT ACGTAGGTTA CTATTGTAAT GGAACTTTTA |
| 1361 | AAAAGCGCGT TGACTTTGAG TCTTTGACTC TAGTTCTGTT |
| 1401 | TGCTACACCG ACAAGTTATA TTTTTCAAAA TGATGAGAAA |
| 1441 | ACGAGGAGAA ACACCGGAAA AAAATTTGAA CTTTTACTTT |
| 1481 | TATCAGACCA TACGGCCAAA GAAAGATCTG TATATTATAT |
| 1521 | AAGTTATCAC AAAACGCGGT TTCACATTTT CTTTTTCGTC |
| 1561 | TTGTTGTGTT TGCAGATTAG TATCAACATC TCCTTACTAT |
| 1601 | CCGACATCGG CCTAAACGAT CTCTTTTTAG ATTGGGACAT |
| 1641 | GGACCAAATT TGTCTTTTTC AATCGGAAGA CATCCATGTT |
| 1681 | CGTTTTTGCA TTTGGCTTAT TTCCAATCTT CTTTTGAAGC |
| 1721 | CTTCTTCGTC GTTGCTAAAT CGTATACTAT TCACGACAAA |
| 1761 | CGTTTTTAGG AGATTACGTT ACCTACTAAG ATTATATATA |
| 1801 | TTGGTTTGTT TTTAAAAATG TCTATTATCT TTATTGTCAT |
| 1841 | TGATAGCTTG ATTTAAGAAG CTCTCTCTTA TCCCGTGACC |
| 1881 | TTCTACTTTT GTTTTATTTT TTAGTATATG GTAAAGAAAA |
| 1921 | TTATAAC |

JAZ13 is also a repressor of jasmonate responses. One example of an Arabidopsis 40 thaliana jasmonate-zim-domain protein 13 (JAZ13) protein sequence is shown below (SEQ ID NO:19).

| | |
|---|---|
| 1 | MKGCSLDLHL SPMASTLQSC HQDSTVNDRS STIRSKEINA |
| 41 | FYSGRLSEYD LVEIQMRAII EMASKDREVT ALELVPVRLE |
| 81 | SPLGCSVKRS VKRFLEKRKK RSKSFTLTPN YISSTSSSSS |
| 121 | SLHNF |

The Arabidopsis thaliana Jaz13 gene encoding the JAZ13 protein with SEQ ID NO:19 is located on chromosome 3, and a cDNA encoding the SEQ ID NO:19 is shown below as SEQ ID NO:20.

| | |
|---|---|
| 1 | TTGATTACTT TGATACGAAA ATCGACCAAA GTAAGAATAT |
| 41 | TTACCTAGAG AGGATCATGA AGGGTTGCAG CTTAGATCTT |
| 81 | CACCTATCTC CAATGGCCTC TACGCTTCAA TCTTGTCATC |
| 121 | AAGATTCTAC AGTTAATGAT CGTTCTTCAA CCATAAGATC |
| 161 | TAAGGAAATC AATGCATTTT ATAGTGGGAG ATTAAGTGAG |
| 201 | TACGATCTTG TAGAGATCCA GATGAGAGCA ATAATAGAGA |
| 241 | TGGCGAGCAA GGATCGTGAA GTAACAGCGT TAGAGTTAGT |
| 281 | GCCGGTGAGA CTGGAATCAC CGTTAGGATG TTCGGTGAAG |
| 321 | AGATCTGTGA AAAGGTTCTT GGAGAAGAGG AAGAAGAGAA |
| 361 | GCAAATCTTT TACACTTACA CCTAATTACA CCTCAAGTAC |
| 401 | TTCCTCATCA TCCTCCTCTC TTCATAATTT CTAATCATAA |
| 441 | TTTTATTATG TTTTCCTTCT AGTTATCAAT CAAAACAAAA |
| 481 | AAATCTTTGT TTCTTTCTTT TTTCTTTTTT CCATTATGGG |
| 521 | TTTCTATAGC TCTCATTTAT CTCTTGTAAT TTTTCCCGAT |
| 561 | ACTCGACGAT GAATTTCGAG TTTTTTTTTT TGATCTGTTT |
| 601 | TAAATCAAGA CATTCTAGTA CCATTGGAGT CTGTATAAAA |
| 641 | TTCAGATCAT TTGGATCGTT ATTTTTTTCC TAATTCATGT |
| 681 | ATGAAGTGTC ACACTTCTCC TACAATGAAT TATGAGGTTG |
| 721 | TCCGTTTATT CCAAGTTAGC TCTATGTACT TTGACGTAAG |
| 761 | CTAATGCAAC TTGTAAAATG TTGGGAACTC TTCTATTACT |
| 801 | TTTTTTCCTT TACAAAATAA GAAAATGCAC GCAT |

Chromosomal sequences that encode repressors of jasmonic acid responses from many plant types and species can be modified to reduce or eliminate the expression and/or function of the encoded protein. For example, chromosomal sequences encoding jasmonic acid repressor genes from agriculturally important plants such as alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and/or wheat can be modified reduce or eliminate the expression and/or function of one or more encoded jasmonic acid regulatory proteins.

In some cases, more than one genetic or chromosomal segment encoding a jasmonic acid regulatory protein can be modified to reduce or eliminate the expression and/or function of the encoded protein(s). In some cases, more than two genes or chromosomal segments encoding jasmonic acid regulatory proteins can be modified to reduce or eliminate the expression and/or function of the encoded proteins. In some cases, more than three genes or chromosomal segments encoding jasmonic acid regulatory proteins can be modified to reduce or eliminate the expression and/or function of the encoded proteins. In some cases, more than four genes or chromosomal segments encoding jasmonic acid regulatory proteins can be modified to reduce or eliminate the expression and/or function of the encoded proteins.

The following are examples of “JAZ-related” proteins and nucleic acids that can be modified to reduce or eliminate the expression and/or function thereof, and thereby generate plants with improved resistance to insects.

One example of a Brassica rapa protein called TIFY 10A-like (NCBI accession no. XP_009117562.1; GI:685367109; SEQ ID NO:21) has significant sequence identity to the Arabidopsis thaliana JAZ1 protein with SEQ ID NO:1, as illustrated by the sequence comparison shown below. Domains of sequence homology are identified by asterisks below the sequence comparison. 73.0% identity in 211 residues overlap; Score: 634.0; Gap frequency: 11.4%

| 73.0% identity in 211 residues overlap; Score: 634.0; Gap frequency: | | |
|---|---|---|
| 11.4% | | |
| Seq:1 | 1 | MSLFPCEASNMDSMVQDVKPTNFFPRQPSFSSSSSSLPKEDVEKMTQ---TTRSVKPESQ |
| Seq21 | 63 | MSLFPCEASNMEPIGQDVKPKNLFPRQPSFSSSSSSLPKEDILKMTQATSSTRSVKPEPQ |
| | | *********** ***** ******************** ***** ******* * |
| Seq:1 | 58 | TAPLTIFYAGQVIVFNDFSAEKAKEVINLASKGTANS-------------------LAKN |
| Seq21 | 123 | TAPLTIFYGGQVIVFNDFSAEKAKEVMDLASKGTANTFTGFTSNVNNNIQSVYTTNLANN |
| | | ******** ***************** ******** ** * |
| Seq:1 | 99 | QTDIRSNIATIANQVPHPRKTTTQEPIQSSPTPLT-ELPIARRASLHRFLEKRKDRVTSK |
| Seq21 | 183 | QTEMRSNIAPIPNQLPHLMKTTTQNPVQSSSTAMACELPIARRASLHRFLAKRKDKVTSK |
| | | ** ***** * ** ** ***** * *** * ************** ********* |
| Seq:1 | 158 | APYQLCDPANASSNPQTTGNM-SWLGLAAEI |
| Seq21 | 243 | APYQLNDPAKASSKPQTGDNTTSWLGLAAEM |
| | | ***** ******* *** * ******** |

This JAZ-related Brassica rapa protein, called TIFY 10A-like (NCBI accession no. XP_009117562.1; GI:685367109), has the following sequence (SEQ ID NO:21).

| | |
|---|---|
| 1 | MSSPMESSDF AATRRFSRKP SFSQTCSRLS QYLKENGSFG |
| 41 | DLSLGMACKP EVNGISRQPT TTMSLFPCEA SNMEPIGQDV |
| 81 | KPKNLFPRQP SFSSSSSSLP KEDILKMTQA TSSTRSVKPE |
| 121 | PQTAPLTIFY GGQVIVFNDF SAEKAKEVMD LASKGTANTF |
| 161 | TGFTSNVNNN IQSVYTTNLA NNQTEMRSNI APIPNQLPHL |
| 201 | MKTTTQNPVQ SSSTAMACEL PIARRASLHR FLAKRKDRVT |
| 241 | SKAPYQLNDP AKASSKPQTG DNTTSWLGLA AEM |

A cDNA encoding the SEQ ID NO:21 protein is available as NCBI accession number XM_009119314.1 (GI:685367108), and a chromosomal segment encoding the SEQ ID NO:21 protein is available as NCBI accession number AENI01008623.1 (GI:339949964).

One example of a Brassica oleracea protein, also referred to as protein TIFY 10A-like (NCBI accession no. XP_013583936.1; GI:922487335; SEQ ID NO:22), has significant sequence identity to the Arabidopsis thaliana JAZ1 protein with SEQ ID NO:1, as illustrated by the sequence comparison shown below. Domains of sequence homology are identified by asterisks below the sequence comparison. 72.9% identity in 192 residues overlap; Score: 633.0; Gap frequency: 2.6%

| 72.9% identity in 192 residues overlap; Score: 633.0; Gap frequency: | | |
|---|---|---|
| Seq:1 | 1 | MSLFPCEASNMDSMV--QDVKPTNLFPRQPSFSSSSSSLPKEDVLKMTQTT-RSVKPESQ |
| Seq22 | 61 | MSLFPCEASNVGSMAALQDVKPKNLFPRQPSFSSSSSSIPKEDVPKMTQTTTRSLKPEPQ |
| | | ********** ** ***** *************** ***** ****** ** *** * |
| Seq:1 | 58 | TAPLTIFYAGQVIVFNDFSAEKAKEVINLASKGTANSLAKNQTDIRSNIATIANQVPHPR |
| Seq22 | 121 | TAPLTIFYGGQVIVFNDFSAEKAKEVMNLANKGTANTFTGFTSTLNNNIAPTPNQVPHLM |
| | | ******** ***************** *** ***** *** ***** |
| Seq:1 | 118 | KTTTQEPIQSSPTPLT-ELPIARRASLHRFLEKRKDRVTSKAPYQLCDPAKASSNPQTTG |
| Seq22 | 181 | KAATQDPKQTSSAAMACELPIARRASLHRFLAKRKDRVTSKAPYQLNDPAKAYSKPQTGN |
| | | * ** * * * ************** ************** ***** * *** |
| Seq:1 | 111 | NM-SWLGLAAEI |
| Seq22 | 241 | TTTSWLGLAADM |
| | | ******* |

This JAZ-related Brassica oleracea protein referred to as protein TIFY 10A-like (NCBI accession no. XP_013583936.1; GI:922487335) has the following sequence (SEQ ID NO:22).

| | |
|---|---|
| 1 | MSSSMECSTT RRSSSGKPSF SLTCSRLSQY LKENGSFGDL |
| 41 | SLGMSCKPDT NGMSPKPTTT MSLFPCEASN VGSMAAAQDV |
| 81 | KPKNLFPRQP SFSSSSSSIP KEDVPKMTQT TIRSLKPEPQ |
| 121 | TAPLTIFYGG QVIVFNDFSA EKAKEVMNLA NKGTANTFTG |
| 161 | FTSTLNNNTA PTPNQVPHLM KAATQDPKQT SSAAMACELP |
| 201 | IARRASLHRF LAKRKDRVTS KAPYQLNDPA KAYSKPQTGN |
| 241 | ITTSWLGLAA DM |

A cDNA encoding the SEQ ID NO:22 protein is available as NCBI accession number XM_013728482.1 (GI:922487334), and a chromosomal segment encoding the SEQ ID NO:22 protein is available as NCBI accession number NC_027752.1 (GI:919506312).

An uncharacterized Zea mays protein referred to as LOC100276383 (NCBI accession no. NP_001308779.1 (GI:1013071036) has significant sequence identity to the Arabidopsis thaliana JAZ1 protein with SEQ ID NO:1, as illustrated by the sequence comparison shown below. Domains of sequence homology are identified by asterisks below the sequence comparison.

| 39.0% identity in 123 residues overlap; Score: 201.0; Gap frequency: | | |
|---|---|---|
| 0.8% | | |
| Seq:1 | 61 | LTIFYAGQVIVFNDFSAEKAYEVINLASKGTANSLAKNQTDIRSNIATIANQVPHPRKTT |
| Seq23 | 100 | LTIFYGGKVLVFDDFPADKAKDLMQLASKGSPVVQNVALPQPSAAAAVTTDKAVLDPVIS |
| | | ***** * * ** ** * *** ***** * |
| Seq:1 | 121 | TQEPIQSSPTPLTELPIARRASLHRFLEKRKDRVTSKAPYQLCDPAKASSNPQTTGNMSW |
| Seq23 | 160 | LAAAKKPARTNASDMPIMRKASLHRFLEKRKDRLNAKTPYQTA-PSDAAPVKKEPESQPW |
| | | * ** * ************* * *** * * * |
| Seq:1 | 181 | LGL |
| Seq23 | 219 | LGL |
| | | *** |

This JAZ-related uncharacterized Zea mays protein referred to as LOC100276383 (NCBI accession no. NP_001308779.1 (GI:1013071036) has the following sequence (SEQ ID NO:23).

| | |
|---|---|
| 1 | MAASARPGER ATSFAVACSL LSRFVRQNGV AAADLGLRIK |
| 41 | GEVEQQRTPA TTNSLPGAEG EEVERRKETM ELFPQSVGFS |
| 81 | IKDAAAPREE QGDKEKPKQL TIFYGGKVLV FDDFPADKAK |
| 121 | DLMQLASKGS PVVQNVALPQ PSAAAAVTTD KAVLDPVISL |
| 161 | AAAKKPARTN ASDMPIMRKA SLHRFLEKRK DRLNAKTPYQ |
| 201 | TAPSDAAPVK KEPESQPWLG LGPNAVDSSL NLS |

A cDNA encoding the SEQ ID NO:23 protein is available as NCBI accession number NM_001321850.1 (GI:1013071035), and a chromosomal segment encoding the SEQ ID NO:23 protein is on Zea mays chromosome 7 at NC_024465.1 (165496371 . . . 165497455), sequence available as NCBI accession number NC_024465.1 (GI:662248746).

A Glycine max protein referred to as protein TIFY 10A-like (NCBI accession no. NP_001276307.1 (GI:574584782)) has significant sequence identity to the Arabidopsis thaliana JAZ1 protein with SEQ ID NO:1, as illustrated by the sequence comparison shown below. Domains of sequence homology are identified by asterisks below the sequence comparison.

| 45.5% identity in 145 residues overlap; Score: 271.0; Gap frequency: | | |
|---|---|---|
| 4.8% | | |
| Seq:1 | 42 | VLKMTQTTRSVKPESQTAPLTIFYAGQVIVFNDFSAEKAKEVINLASKGTANSLAKNQTD |
| Seq24 | 101 | IMVKSSAFKSMEKEPKAAQLTIFYAGQVVVFDDFPAEKLEEITSLAGKGISQS-----QN |
| | | * * * ********* ** ** *** * ** ** * |
| Seq:1 | 102 | IRSNIATIANQVPHPRKTTTQEPIQSSPTPLTELPIARRASLHRFLEKRKDRVTSKAPYQ |
| Seq24 | 156 | TSAYAHTHNQQVNHPSFVPNISPQAPSRPLVCDLPIARKASLHRFLSKRYDRIAAKAPYQ |
| | | * ** ** * * ***** ******* ***** ***** |
| Seq:1 | 162 | LCDPAKASSNPQTTGNMSWLGLAAE |
| Seq24 | 216 | INNPNSASSKPAE--SMSWLGLGAQ |
| | | * *** * ****** * |

This JAZ-related Glycine max protein referred to as protein TIFY 10A-like (NCBI accession no. NP_001276307.1 (GI:574584782) has the following sequence (SEQ ID NO:24).

| | |
|---|---|
| 1 | MSSSSEYLVF SSHHPANSPA EKSTFSQTCS LLSQYIKEKG |
| 41 | TFGDLTLGMT CTAETNGSPE TSCHSATTME LFPTIITQRN |
| 61 | PTTVDFLSPQ TAYPHHSEVP IMVKSSAFKS MEKEPKAAQL |
| 121 | TIFYAGQVVV FDDFPAEKLE EITSLAGKGI SQSQNTSAYA |
| 161 | HTHNQQVNHP SFVPNISPQA PSRPLVCDLP IARKASLHRF |
| 201 | LSKRKDRIAA KAPYQINNPN SASSKPAESM SWLGLGAQST |

A cDNA encoding the SEQ ID NO:24 protein is available as NCBI accession number NM_001289378.1 (GI:574584781), and a chromosomal segment encoding the SEQ ID NO:24 protein is on Glycine max chromosome 13 at NC_016100.2 (22541885 . . . 22544240), sequence available as NCBI accession number NC_016100.2 (GI:952545303).

An Oryza sativa protein referred to as protein TIFY 10b (Japonica Group; NCBI accession no. XP_015647536.1 (GI:1002286463) has significant sequence identity to the Arabidopsis thaliana JAZ1 protein with SEQ ID NO:1, as illustrated by the sequence comparison shown below. Domains of sequence homology are identified by asterisks below the sequence comparison.

| 38.5% identity in 156 residues overlap; Score: 213.0; Gap frequency: | | |
|---|---|---|
| 4.5% | | |
| Seq:1 | 34 | SSSLPKEDVLKMTQTTRSVKPESQTAPLTIFYAGQVIVFNDFSAEKAKEVINLASKGTA- |
| Seq25 | 77 | SAGFGQQDAITADSAADAREQEPEKRQLTIFYGGKVLVFNDFPADKAKGLMQLASKGSPV |
| | | * * * ***** * * ***** * *** ***** |
| Seq:1 | 93 | ---NSLAKNQTDIRSNI-ATIANQVPHPRKTTTQEPIQS-SPTPLTELPIARRASLHRFL |
| Seq25 | 137 | APQNAAAPAPAAVTDNTKAPMAVPAPVSSLPTAQADAQKPARANASDMPIARKASLHRFL |
| | | * * * * * * * * * **** ******* |
| Seq:1 | 148 | EKRKDRVTSKAPYQLCDPAKASSNPQTTGNMSWLGL |
| Seq25 | 197 | EKRKDRLNAKTPYQ-ASPSDATPVKKEPESQPWLGL |
| | | ****** * *** * * **** |

This JAZ-related Oryza sativa protein referred to as protein TIFY 10b (Japonica Group; NCBI accession no. XP_015647536.1 (GI:1002286463) that has significant sequence identity to the Arabidopsis thaliana JAZ1 protein, has the following sequence (SEQ ID NO:25).

| | |
|---|---|
| 1 | MAASARPVGV GGERATSFAM ACSLLSRYVR QNGAAAAELG |
| 41 | LGIRGEGEAP RAAPATMSLL PGEAERKKET MELFPQSAGF |
| 81 | GQQDAITADS AADAREQEPE KRQLTIFYGG KVLVFNDFPA |
| 121 | DKAKGLMQLA SKGSPVAPQN AAAPAPAAVT DNTKAPMAVP |
| 161 | APVSSLPTAQ ADAQKPARAN ASDMPIARKA SLHRFLEKRK |
| 201 | DRLNAKTPYQ ASPSDATPVK KEPESQPWLG LGPNAVVKPI |
| 241 | ERGQ |

A cDNA encoding the SEQ ID NO:25 protein is available as NCBI accession number XM_015792050.1 (GI:1002286462), and a chromosomal segment encoding the SEQ ID NO:25 protein is on Oryza sativa chromosome 7 at NC_029262.1 (25347990 . . . 25350243), sequence available as NCBI accession number NC_029262.1 (GI:996703426).

An uncharacterized Zea mays protein with NCBI accession no. ACF88234.1 (SEQ ID NO:26) has significant sequence identity to the Arabidopsis thaliana JAZ2 protein with SEQ ID NO:3, as illustrated by the sequence comparison shown below. Domains of sequence homology are identified by asterisks below the sequence comparison.

| 35.1% identity in 235 residues overlap; Score: 221.0; Gap frequency: | | |
|---|---|---|
| 9.8% | | |
| Seq:3 | 14 | RKPSFSQTCTRLSRYLKEKGSFGDLSLGMTCKPDVNGGSRQPTMMNLFPCEASGMDSSAG |
| Seq26 | 10 | RATSFAVACSLLSRFVRQNGA-APAQLGLGIKGEVEQ-QRTPATINLLP----GADGEET |
| | | * ** * *** * ** * * * * ** * * * |
| Seq:3 | 74 | QEDIKPKTMFPRQSSFSSSSSSGTKEDVQMIKETTKSVKPESQSAPLTIFYGGRVMVFDD |
| Seq26 | 64 | ERRKETMELFPQSAGF------GVKDAAAAPREQENKEKPKQ----LTIFYGGKVLVFDD |
| | | ** * * * * ** ******* * **** |
| Seq:3 | 134 | FSAEKAKEVIDLANKGSAKSFTCFTAEVNNNHSAYSQKEIASSPNPVCSPAKTAAQEPIQ |
| Seq26 | 114 | FPADKAKDLMQLASKGGPVVQNVVLPQPSAPAAAVTDKAV---PVPVIS--LPAAQADAK |
| | | * * *** ** ** * * * ** * *** |
| Seq:3 | 194 | PNPASLACELPIARRASLHRFLEKRKDRITSKAPYQIDGS--AEASSKPTNPAWL |
| Seq26 | 169 | KPTRTNASDMPIMRKASLHRFLEKRKDRLNANAPYQTSPSDAAPVKKEPESQAWL |
| | | * ** * ************* **** * * * *** |

This JAZ-related Zea mays protein with NCBI accession no. ACF88234.1 that has significant sequence identity to the Arabidopsis thaliana JAZ2 protein, has the following sequence (SEQ ID NO:26).

| | |
|---|---|
| 1 | MAASAPPGER ATSFAVACSL LSRFVRQNGA APAQLGLGIK |
| 41 | GEVEQQRTPA TINLLPGADG EETERRKETM ELFPQSAGFG |
| 81 | VKDAAAAPRE QENKEKPKQL TIFYGGKVLV FDDFPADKAK |
| 121 | DLMQLASKGG PVVQNVVLPQ PSAPAAAVTD KAVPVPVISL |
| 161 | PAAQADAKKP TRTNASDMPI MRKASLHRFL EKRKDRLNAN |
| 201 | APYQTSPSDA APVKKEPESQ AWLGLGPNAV KSNLNLS |

This JAZ-related Zea mays protein with NCBI accession no. ACF88234.1 is encoded by a gene on chromosome 2 at NC_024460.2 (218018545 . . . 218021029) of the Zea mays genome.

An uncharacterized Triticum aestivum (wheat) protein with NCBI accession no. SPT16989.1 (SEQ ID NO:27) has significant sequence identity to the Arabidopsis thaliana JAZ2 protein with SEQ ID NO:3, as illustrated by the sequence comparison shown below. Domains of sequence homology are identified by asterisks below the sequence comparison.

| 44.8% identity in 116 residues overlap; Score: 201.0; Gap frequency: | | |
|---|---|---|
| 5.2% | | |
| Seq3 | 114 | ESQSAPLTIFYGGRVMVFDDFSAEKAKEVIDLANKGSAKSFTCFTAEVNNNHSAYSQKEI |
| Seq21 | 91 | EEDKSQLTIFYGGKVLVFNDFPADKAKGLMQLAGKGSPVVQNVSATTTAADTDKVQTAVL |
| | | * ******* * ** ** * *** ** *** |
| Seq3 | 174 | ASSPNPVCSPAKTAAQEPIQPNPASLACELPIARRASLHRFLEKRKDRITSKAPYQ |
| Seq27 | 151 | APASSLPTGPVD--APKPARPN----ASDLPIARKASLHRFLEKRKDRLHAKAPYQ |
| | | * * * * ** * ***** ************* ***** |

This JAZ-related Triticum aestivum (wheat) with NCBI accession no. SPT16989.1 that has significant sequence identity to the Arabidopsis thaliana JAZ2 protein, has the following sequence (SEQ ID NO:27).

| | |
|---|---|
| 1 | MAASARQGER ATSFAMACSL LSRYVRQNGA AAAELGLGIN |
| 41 | KGEAEAQRAA DTKSPLPGAE GEEAGRKKET MELFPQSAGL |
| 81 | QDAAAPDATR EEDKSQLTIF YGGKVLVFND FPADKAKGLM |
| 121 | QLAGKGSPVV QNVSATTTAA DTDKVQTAVL APASSLPTGP |
| 161 | VDAPKPARPN ASDLPIARKA SLHRFLEKRK DRLHAKAPYQ |
| 201 | APPSDATPVK KEFENQPWLG LGPNAALKRN Q |

An uncharacterized Glycine max (soybean) protein with NCBI accession no. XP_003542368.1 (SEQ ID NO:28) has significant sequence identity to the Arabidopsis thaliana JAZ2 protein with SEQ ID NO:3, as illustrated by the sequence comparison shown below. Domains of sequence homology are identified by asterisks below the sequence comparison.

| | |
|---|---|
| 42.6% | identity in 230 residues overlap; Score: 314.0; Gap frequency: |
| 12.6% | |
| Seq:3 | 15 KPSFSQTCTRLSRYLKEKGSFGDLSLGMTCKPDVNGGSR----QPTMMNLFPCEASGMDS |
| Seq28 | 22 KSTFSQTCSLLSQYIKEKGTFGDLTLGMTCTAETNGSPETSCHSATTMELFPTIITQRNP |
| | * ***** ** * **** **** ***** ** * * *** |
| Seq:3 | 71 SAGQEDIKPKTMFPRQSSFSSSSSSGTKEDVQMIKETTKSVKPESQSAPLTIFYGGRVMV |
| Seq28 | 82 TT-VDFLSPQTAYPHHS----------EVPIMVKSSAFKSMEKEPKAAQLTIFYAGQVVV |
| | * * * * ** * * ***** * * * |
| Seq:3 | 131 FDDFSAEKAKEVIDLANKGSAKSFTCFTAEVNNNHSAYSQKEIASSPNPVCSPAKTAAQE |
| Seq28 | 131 FDDFPAEKLEEITSLAGKGISQS---------QNTSAYAHTHNQQVNHPSFVP-NISPQA |
| | **** *** * ** ** * * *** * * * |
| Seq:3 | 191 PIQPNPASLACELPIARRASLHRFLEKRKDRITSKAPYQIDGSAEASSKP |
| Seq28 | 181 PSRP----LVCDLPIARKASLHRFLSKRKDRIAAKAPYQINNPNSASSKP |
| | * * * * ***** ******* ****** ****** ***** |

This JAZ-related Glycine max protein with NCBI accession no. XP_003542368.1 that has significant sequence identity to the Arabidopsis thaliana JAZ2 protein, has the following sequence (SEQ ID NO:28).

| | |
|---|---|
| 1 | MSSSSEYLVF SGHHPANSPA EKSTFSQTCS LLSQYIKEKG |
| 41 | TFGDLTLGMT CTAETNGSPE TSCHSATTME LFPTIITQRN |
| 81 | PTTVDFLSPQ TAYPHHSEVP IMVKSSAFKS MEKEPKAAQL |
| 121 | TIFYAGQVVV FDDFPAEKLE EITSLAGKGI SQSQNTSAYA |
| 181 | HTHNQQVNHP SFVPNISPQA PSRPLVCDLP IARKASLHRF |
| 201 | LSKRKDRIAA KAPYQINNPN SASSKPAESM SWLGLGAQST |
| 241 | QV |

This JAZ-related Glycine max protein with NCBI accession no. XP_003542368.1 is encoded by a gene at NC_038249.1 (22541885 . . . 22544240) on chromosome 13 of the Glycine max genome.

An uncharacterized Zea mays protein referred to as LOC103647411 (NCBI accession no. NP_001288506.1; SEQ ID NO:29) has significant sequence identity to the Arabidopsis thaliana JAZ3 protein with SEQ ID NO:5, as illustrated by the sequence comparison shown below. Domains of sequence homology are identified by asterisks below the sequence comparison.

| | |
|---|---|
| 36.6% | identity in 161 residues overlap; Score: 165.0; Gap frequency: |
| 6.8% | |
| Seq5: | 177 AQLTIFYAGSVCVYDDISPEKAKAIMLLAGNGSSMPQVFSPPQTHQQVVHHTRASVDSSA |
| Seq29 | 167 AQLTIFYAGSVNVFNNVSAEKAQELMFLASRGSSAPVACKPEAPPTLAPAKVTAPEVLLP |
| | *********** * * *** * ** *** * * * |
| Seq5: | 237 MPPSFMPTISYLSPEAGSSTNGLGATKATRGLTSTYH-NNQANGSNINCPVP-------- |
| Seq29 | 227 AKQMLFQKPQHLSPPPSSVPGILQSAALPRSASSSSNLDSPAPKSSVPLAVPPVSQAPPA |
| | *** * * * * * * ** |
| Seq5: | 288 --VSCSTNVMAPTVALPLARKASLARFLEKRKERVTSVSPY |
| Seq29 | 287 TLIATTTAAAIMPRAVPQARKASLARFLEKRKERVTTAAPY |
| | * * * ****************** ** |

This JAZ-related Zea mays protein referred to as LOC103647411 (NCBI accession no. NP_001288506.1) has the following sequence (SEQ ID NO:29).

| | |
|---|---|
| 1 | MERDFLAAIG KEQQHPRKEK AGGGAEESAY FGAAAVPAMD |
| 41 | WSFASKPCAA PALMSFRSAA REEPSFPQFS ALDGTKNTAP |
| 81 | RMLTHQRSFG PDSTQYAALH RAQNGARVVP VSSPFSQSNP |
| 121 | MFRVQSSPSL PNSTAFKQPP FAISNAVASS TVGSYGGTRD |
| 161 | AVRPRTAQLT IFYAGSVNVF NNVSAEKAQE LMFLASRGSS |
| 201 | APVACKPEAP PTLAPAKVTA PEVLLPAKQM LFQKPQHLSP |
| 241 | PPSSVPGILQ SAALPRSASS SSNLDSPAPK SSVPLAVPPV |
| 281 | SQAPPATLIA TTTAAAIMPR AVPQARKASL ARFLEKRKER |
| 321 | VTTAAPYPSA KSPLESSDTF GSGSASANAN DKSSCTDIAL |
| 361 | SSNHEESLCL GGQPRSIISF SEESPSTKLQ I |

A cDNA encoding the SEQ ID NO:29 protein is available as NCBI accession number NM_001301577.1 and a chromosomal segment encoding the SEQ ID NO:29 protein is on chromosome 2 at NC_024460.2 (184842608 . . . 184845336, complement) of the Zea mays genome, sequence available as NCBI accession number NC_024460.1 (GI:662249846).

A Triticum aestivum jasmonate ZIM-domain transcriptional repressor protein with (NCBI accession no. QBQ83004.1; SEQ ID NO:30) has significant sequence identity to the Arabidopsis thaliana JAZ3 protein with SEQ ID NO:5, as illustrated by the sequence comparison shown below, where the two sequences have about 30% sequence identity. Domains of sequence homology are identified by asterisks below the sequence comparison.

| | |
|---|---|
| Seq5 | 1 MERDFLG-LGSKNSPITVKEETSESS--------RDSAPNRG-----MNWSFSNKVSASS |
| Seq30 | 1 MERDFLGTIGHEQLQQQQQQQQRQRAAAEDAAARKESAYFGGGGVPPMDWSFAGRAGAAP |
| | ******* * ** * * *** * |
| Seq5 | 47 SQFLSFR--PTQEDRH-----RKSGNYHLPHSGSFMPSSVADV-YDSTRKAPYSSVQGVR |
| Seq30 | 61 A-VMSFRSAPREEQRGELAYPKQQASRVLTPQRSFGAESHGSVQYAAAARAAYGGQP--- |
| | *** * * * * * * * * * * |
| Seq5 | 99 MFPNSNQHEETNAVSMSMPGFQSHHYAPGGRSFMNNNNNSQP---LVGVPIMAPPISIL- |
| Seq30 | 117 --PQQHQHAPNGARVIPM----SSPFNPNNPMFRVQSSPNLPNGVAAGSPFKQPPFVMNN |
| | * ** * * * * * * * * ** |
| Seq5 | 155 PPPGSIVGTTDIRSSSKPIGSPAQLTIFYAGSVCVYDDISPEKAKAIMLLAGNGSS---- |
| Seq30 | 171 AVAASTVGVYKSRDMPKP--KTAQLTIFYAGSVNVFNNVSAEKAQELMFLASRGSLPTAP |
| | * ** * ** *********** * * *** * ** ** |
| Seq5 | 211 ------------MPQVFSPPQTH--QQVVHHTRASVD----SSAMPPSFMPTISYLSPEA |
| Seq30 | 229 TTVTRSPDATFFTPAKLAAPEASPAKQMLAHIPQRVSPPLPAISKPMSIMSQAACL-PKS |
| | * * * * * * * |
| Seq5 | 253 GSSTNGLGATKATRGLTSTYHNNQANGSNINCPVPVSCSTNVMAPTVALPLARKASLARF |
| Seq30 | 288 TSSSNTDSAVPKSSGQLVVPPTSQTSSST--HPVTLSSTTAASIMPRAVPQARKASLARF |
| | ** * * * * * ** * * * ********* |
| Seq5 | 313 LEKRKERVTSVSPY 326 |
| Seq30 | 346 LEKRKERVTTTAPY 359 |
| | ********* ** |

This Triticum aestivum jasmonate ZIM-domain transcriptional repressor protein with significant sequence identity to the Arabidopsis thaliana JAZ3 protein with SEQ ID NO:5 and NCBI accession no. QBQ83004.1 has the following sequence (SEQ ID NO:30).

| | |
|---|---|
| 1 | MERDFLGTIG HEQLQQQQQQ QQRQRAAAED AAARKESAYF |
| 41 | GGGGVPPMDW SFAGRAGAAP AVMSFRSAPR EEQRGELAYP |
| 81 | KQQASRVLTP QRSFGAESHG SVQYAAAARA AYGGQPPQQH |
| 121 | QHAPNGARVI PMSSPFNPNN PMFRVQSSPN LPNGVAAGSP |
| 161 | FKQPPFVMNN AVAASTVGVY KSRDMPKPKT AQLTIFYAGS |
| 201 | VNVFNNVSAE KAQELMFLAS RGSLPTAPTT VTRSPDATFF |
| 241 | TPAKLAAPEA SPAKQMLAHI PQRVSPPLPA ISKPMSIMSQ |
| 281 | AACLPKSTSS SNTDSAVPKS SGQLVVPPTS QTSSSTHPVT |
| 321 | LSSTTAASIM PRAVPQARKA SLARFLEKRK ERVTTTAPYP |
| 361 | SAKSPMESSD TVGSANDNNS KSSSCTEIAF SSNHEESLRL |
| 401 | GRPRNISFSG ESPSTKLHI |

A cDNA encoding the SEQ ID NO:30 Triticum aestivum jasmonate protein has the sequence provided as NCBI accession number MH063273.1.

A Glycine max protein referred to as protein TIFY 6B-like isoform X1 (NCBI accession no. XP_003534135.1 (GI:356531138; SEQ ID NO:31) has significant sequence identity to the Arabidopsis thaliana JAZ3 protein with SEQ ID NO:5, as illustrated by the sequence comparison shown below. Domains of sequence homology are identified b asterisks below the sequence comparison.

| | |
|---|---|
| 38.9% | identity in 378 residues overlap; Score: 417.0; Gap frequency: |
| 8.5% | |
| Seq5: | 1 MERDFLGLGSKNSP-ITVKEETSESSRDSAPNRGMNWSFSNKVSASSSQFLSFRPTQEDR |
| Seq31 | 1 MEREFFGLSSKNGAWTTMKDDAVNKSRDQVRSSGMQWSFPNKVSALP-QFLSFKTNQEDK |
| | *** * ** *** * * *** ** *** ***** ***** *** |
| Seq5: | 60 HRKSGNYHLPHSGSFMPSSVADVYDSTRKA--------------PYSSVQGVRMFPNS-- |
| Seq31 | 60 PRKTILEPLASSG-YMAMSTQYAFDSNQKSFLGLTNRNLSISKHAAGNKQGMTVYPLQCC |
| | ** * ** * * ** * ** * |
| Seq5: | 104 -NQHEETNAVSMSMPGFQ-----SHHYAPGGRSFMNNNNNSQPLVGVPIMAPPISILPPP |
| Seq31 | 119 DAQSEEARIFSVSNQSNQVSPVLQSNLASTGLNMVNSVIKPQPF-GSKSSGTPLSILPSI |
| | * ** * * * * * * ** * * **** |
| Seq5: | 158 GSIVGTTDIRSSSKPIGSPAQLTIFYAGSVCVDDDISPEKAKAIMLLAGNGSSMPQVFSP |
| Seq31 | 178 GSIVGSTDLRNNSKSSTMPTQLTIFYAGSVCVYDDISPEKAKAIMLMAGNGYTPTEKMEL |
| | ***** ** * ** * ************************** **** |
| Seq5: | 218 PQTHQQVVHHTRASVD----SSAMPPSFMPTISYLSPEAGSSTNGLGATKATRGLTSTYH |
| Seq31 | 238 PTVKLQPAISIPSKDDGFMISQSYPPSTFPTPLPLTSHVNSQPGGGSSSNKEISIIRQVG |
| | * * * * *** ** * * * |
| Seq5: | 274 NNQANGSNINCPV--PVSCSTNVMAPTVALPLARKASLARFLEKRKERVTSVSPYCLDKK |
| Seq31 | 298 PSTAPTNHLESPIIGSIGSASKEKAQPVCLPQARKASLARFLEKRKGRMMRTSPYLYMSK |
| | * * * * ** ************** * *** * |
| Seq5: | 332 SSIDCRRSMSECISSSLS |
| Seq31 | 358 KSPECSSSGSDSVSFSLN |
| | * * * * * ** |

This JAZ-related Glycine max protein referred to as protein TIFY 6B-like isoform X1 (NCBI accession no. XP_003534135.1 (GI:356531138) has the following sequence (SEQ ID NO:31).

| | |
|---|---|
| 1 | MEREFFGLSS KNGAWTTMKD DAVNKSRDQV RSSGMQWSFP |
| 41 | NKVSALPQFL SFKTNQEDKP RKTILEPLAS SGYMAMSTQY |
| 81 | AFDSNQKSFL GLTNRNLSIS KHAAGNKQGM TVYPLQCCDA |
| 121 | QSEEARIFSV SNQSNQVSPV LQSNLASTGL NMVNSVIKPQ |
| 161 | PFGSKSSGTP LSILPSIGSI VGSTDLRNNS KSSTMPTQLT |
| 201 | IFYAGSVCVY DDISPEKAKA IMLMAGNGYT PTEKMELPTV |
| 241 | KLQPAISIPS KDDGFMISQS YPPSTFPTPL PLTSHVNSQP |
| 281 | GGGSSSNKEI SIIRQVGPST APTNHLESPI IGSIGSASKE |
| 321 | KAQPVCLPQA RKASLARFLE KRKGRMMRTS PYLYMSKKSP |
| 361 | ECSSSGSDSV SFSLNFSGSC SLPATN |

A cDNA encoding the SEQ ID NO:31 protein is available as NCBI accession number XM_003534087.3 (GI:955341633), and a chromosomal segment encoding the SEQ ID NO:31 protein is on Glycine max chromosome 9 at NC_016096.2 (39883473 . . . 39889992), sequence available as NCBI accession number NC_016096.2 (GI:952545307).

An Oryza sativa protein referred to as protein TIFY 6b (NCBI accession no. XP_015612402.1 (GI:1002297967), SEQ ID NO:32) has significant sequence identity to the Arabidopsis thaliana JAZ3 protein with SEQ ID NO:5, as illustrated by the sequence comparison shown below. Domains of sequence homology are identified by asterisks below the sequence comparison.

| | |
|---|---|
| 37.3% | identity in 177 residues overlap; Score: 142.0; Gap frequency: |
| 10.2% | |
| Seq5: | 172 PIGSPAQLTIFYAGSVCVYDDISPEKAKAIMLLAGNGS---------SMFQ--VFSPPQT |
| Seq32 | 187 PKAKAAQLTIFYAGSVNVFNNVSPEKAQELMFLASRGSLPSAPTTVARMPEAHVFPPAKV |
| | * *********** * ***** * ** ** ** ** * |
| Seq5: | 221 HQQVVHHTRASV-DSSAMPPSFMPTISY---LSPEAGSSTNGLGATKATRGLTSTYHNNQ |
| Seq32 | 247 TVPEVSPTKPMMLQKPQLVSSPVPAISKPISVVSQATSLPRSASSSNVDSNVTKSSGPLV |
| | * * * * ** * * * |
| Seq5: | 277 ANGSNINCPV-PVSCSTNVMAPTV--ALPLARKASLARFLEKRKERVTSVSPYCLDK |
| Seq32 | 307 VPPTSLPPPAQPETLATTTAAAIMPRAVPQARKASLARFLEKRKERVTTVAPYPLAK |
| | * * * * * * ****************** * ** * * |

This JAZ-related Oryza sativa protein, referred to as protein TIFY 6b (NCBI accession no. XP_015612402.1 (GI:1002297967), has the following sequence (SEQ ID NO:32).

| | |
|---|---|
| 1 | MERDFLGAIG KDEEQRRHAE ERKESDYFGA GGGAAAAAMD |
| 41 | WSFASRAALM SFRSSSSAAA AAAREETREL AFPHFSALDG |
| 81 | AKMQQASHVL ARQKSFGAES HGIPQYAAAA AVHGAHRGQP |
| 121 | PHVLNGARVI PASSPFNPNN PMFRVQSSPN LPNAVGAGGG |
| 161 | AFKQPPFAMG NAVAGSTVGV YGTRDMPKAK AAQLTIFYAG |
| 201 | SVNVFNNVSP FKAQELMFLA SRGSLPSAPT TVARMPEAHV |
| 241 | FPPAKVTVPE VSPTKPMMLQ KPQLVSSPVP AISKPISVVS |
| 281 | QATSLPRSAS SSNVDSNVTK SSGPLVVPPT SLPPPAQPET |
| 321 | LATTTAAAIM PRAVPQARKA SLARFLEKRK ERVTTVAPYP |
| 361 | LAKSPLESSD TMGSANDNKS SCTDIALSSN RDESLSLGQP |
| 401 | RTISFCEESP STKLQI |

A cDNA encoding the SEQ ID NO:32 protein is available as NCBI accession number XM_015756916.1 (GI:1002297966), and a chromosomal segment encoding the SEQ ID NO:32 protein is on Oryza sativa chromosome 9 at NC_029264.1 (14056084 . . . 14060320, complement), sequence available as NCBI accession number NC_029264.1 (GI:996703424).

An uncharacterized Zea mays protein referred to as LOC100273108 (NCBI accession no. NP_001141029.1 (GI:226500626), SEQ ID NO:33) has significant sequence identity to the Arabidopsis thaliana JAZ4 protein with SEQ ID NO:7. For example, the Zea mays SEQ ID NO:33 protein has domains of 40 residues having 55% sequence identity from positions 138-178, and 26 residues having 77% sequence identity from positions 258-284 homology with the Arabidopsis thaliana JAZ4 protein. This JAZ-related uncharacterized Zea mays protein, referred to as LOC100273108 (NCBI accession no. NP_001141029.1 (GI:226500626), has the following sequence (SEQ ID NO:33).

| | |
|---|---|
| 1 | MAKSGASFPE SSWMERDFLA AIGKEQQHPH KEEAGAEESA |
| 41 | YFGGAGAAAA APAMDWSFAS KPGAAPALMS FRSASFPQFS |
| 81 | SFDGAKNPAP RILTHQRSFG PDSTHYAAAH RTQHALNGAR |
| 121 | VTPVSSPFNQ NSPMFRVQSS PSLPNGTAFK QPPFAINNNA |
| 161 | AASSTVGFYG TRDVVRPKTA QLTIFYAGSV NVFDNVSAEK |
| 201 | AQELMLLASR GSLPSSAPVA RKPEAPILAP AKVTAPEVLH |
| 241 | ATQMLFQKPQ HVSPPSSAIS KPIPGILQAA SLPRSASSSN |
| 281 | LDSPFPKSSV PFPVSPVSQA PRAQPATIAA TTAAAIMPRA |
| 321 | VPQARKASLA RFLEKRKERV TTAAPYPSAK SPMESSDTFG |
| 361 | SGSANDKSSC TDIALSSNHE ESLCLGQPRN ISFIQESPST |
| 401 | KLQI |

A cDNA encoding the SEQ ID NO:33 protein is available as NCBI accession number NM_001147557.1 (GI:226500625), and a chromosomal segment encoding the SEQ ID NO:33 protein is on Zea mays chromosome 7 at NC_024465.1 (108871356 . . . 108874213, complement), sequence available as NCBI accession number NC_024465.1 (GI:662248746).

A Glycine max protein, referred to as protein TIFY 6B isoform X5 (NCBI accession number XP_006580448.1 (GI:571456655; SEQ ID NO:34), has significant sequence identity to the Arabidopsis thaliana JAZ4 protein with SEQ ID NO:7, as illustrated by the sequence comparison shown below. Domains of sequence homology are identified with asterisks below the sequence comparison.

| | |
|---|---|
| 37.0% | identity in 322 residues overlap; Score: 273.0; Gap frequency: |
| 8.7% | |
| Seq7: | 1 MERDFLGLGSKLSPITVKEETNEDSAPSRG-----MMDWSFSSKVGSGPQFLSFGTSQQE |
| Seq34 | 1 MERDFMGLNLKEPLAVVKFFMNNDGCKNSGFKKGRIAQWPFSNKVSALPHLMSFKASQDD |
| | ***** ** * **** * * * * ** ** * ** ** |
| Seq7: | 56 TRVNTVNDHLLSSAAMD-QNQRTYFSSLQEDRVFPGSSQQDQTTITVSMSEPNYINSFI- |
| Seq34 | 61 KTKNTVSDTLSSSGFMSILSQEAFDTSQKRSAGEPQMFSVPNQAISVSLGNPFLKNHFAA |
| | *** * * ** * * * * * ** * * * |
| Seq7: | 114 --NHQHLGGSPIMAP----PVSVFPAPTTIRSSSKPLPPQLTIFYAGSVLVYQDIAPEKA |
| Seq34 | 121 AGQKPLLGGIPVTTSHSVLPSAVAVAGMTESCNSVKPSAQLTIFYAGTVNIFDDISAEKA |
| | *** * * * * * ******** * ** *** |
| Seq7: | 168 QAIMLLAGNG-PHAKPVSQPKPQKLVHHSLPTTDPPTMPPSFLPSISYIVSETRSSGSNG |
| Seq34 | 181 QAIMLLAGNSLSAASNMAQPNVQVPISKLGAGAGVPVSQPANTSPGSGLSSPLSVSSHTG |
| | ********* * ** * * * * * * * |
| Seq7: | 227 V-TGLGPTKTKASLASTRNN--QTAAFSMAP----------TVGLPQTRKASLARFLEKR |
| Seq34 | 241 VQSGSGLTSTDEFLAAKTTGVPNTPICNVEPPKVVSATTMLTSAVPQARKASLARFLEKR |
| | * * * * * ** * * * ** ************ |
| Seq7: | 274 KERVINVSPYYVDNKSSIDCRT |
| Seq34 | 301 KERVMSAAPYNL-NEESEECAT |
| | **** ** ** * * * |

This JAZ-related Glycine max protein, referred to as protein TIFY 6B isoform X5 (NCBI accession number XP_006580448.1 (GI:571456655), has the following sequence (SEQ ID NO:34).

| | |
|---|---|
| 1 | MERDFMGLNL KEPLAVVKEE MNNDGCKNSG FKKGRIAQWP |
| 41 | FSNKVSALPH LMSFKASQDD KTKNTVSDTL SSSGFMSILS |
| 61 | QEAFDTSQKR SAGEPQMFSV PNQAISVSLG NPFLKNHFAA |
| 121 | AGQKPLLGGI PVTTSHSVLP SAVAVAGMTE SCNSVKPSAQ |
| 161 | LTIFYAGTVN IFDDISAEKA QAIMLLAGNS LSAASNMAQP |
| 201 | NVQVPISKLG AGAGVPVSQP ANTSPGSGLS SPLSVSSHTG |
| 241 | VQSGSGLTST DEFLAAKTTG VPNTPICNVE PPKVVSATTM |
| 281 | LTSAVPQARK ASLARFLEKR KERVMSAAPY NLNKKSEECA |
| 321 | TAEYAGVNFS ATNTVLAKQG |

A cDNA encoding the SEQ ID NO:34 protein is available as NCBI accession number XM_006580385.2 (GI:955322108), and a chromosomal segment encoding the SEQ ID NO:34 protein is on Glycine max chromosome 5 at NC_016092.2 (41222014 . . . 41225906), sequence available as NCBI accession number NC_016092.2 (GI:952545311).

An Oryza sativa protein, referred to as protein TIFY 6a isoform X2 (NCBI accession number XP_015651050.1 (GI:1002293416; SEQ ID NO:35), has significant sequence identity to the Arabidopsis thaliana JAZ4 protein with SEQ ID NO:7. For example, the Oryza sativa SEQ ID NO:35 protein has domains of 26 residues having 81% sequence identity from positions 258-284 of the Arabidopsis thaliana JAZ4 protein with SEQ ID NO:7, and 47 residues having 45% sequence identity from positions 138-185 of the Arabidopsis thaliana JAZ4 protein with SEQ ID NO:7. This JAZ-related Oryza sativa protein, referred to as protein TIFY 6a isoform X2 (NCBI accession number XP_015651050.1 (GI:1002293416), has the following sequence (SEQ ID NO:35).

| | |
|---|---|
| 1 | MERDFLGAIW RKEEAAGKPE EHSDYRGGGG GASAAMQWQF |
| 41 | PATKVGAASS AFMSFRSSAA AAREEDPKEA AVFDRFSLSG |
| 81 | FRPPPRPSPG DAFDGAAAMK QRQFGFNGRQ QYAAAAQHGH |
| 121 | REQGVDSYGV AAPHHFPSPS PSPRHPVPFG HANPMLRVHS |
| 161 | LPNVAGGSPY RNQSFSVGNS VAGSTVGVYG GPRDLQNPKV |
| 201 | TQMTIFYDGL VNVFDNIPVE KAQELMLLAS RASIPSPPSA |
| 241 | ARKSDSPISA AAKLTVPEAL PARQIVVQKP EASVPLVSGV |
| 281 | SNPITIVSQA VTLPKSFSSS NDSAGPKSGG LPLAVTPLSQ |
| 321 | ASPSQPIPVA TTNASAIMPR AVPQARKASL ARFLEKPKER |
| 361 | VSSVAPYPSS KSPLESSDTI GSPSTPSKSS CTDITPSTNN |
| 401 | CEDSLCLGQP RNISFSSQEP PSTKLQI |

A cDNA encoding the SEQ ID NO:35 protein is available as NCBI accession number XM_015795564.1 (GI:1002293415), and a chromosomal segment encoding the SEQ ID NO:35 protein is on Oryza sativa chromosome 8 at NC_029263.1 (20624989 . . . 20627964, complement), sequence available as NCBI accession number NC_029263.1 (GI:996703425).

A Triticum aestivum jasmonate ZIM-domain transcriptional repressor protein with NCBI accession no. ABK63978.1 (SEQ ID NO:36) has significant sequence identity to the Arabidopsis thaliana JAZ4 protein with SEQ ID NO:7. For example, the Triticum aestivum SEQ ID NO:36 protein has domains of 36 residues having 67% sequence identity from positions 139-175 of the Arabidopsis thaliana JAZ4 protein with SEQ ID NO:7 and 26 residues having 58% sequence identity from positions 258-284 of the Arabidopsis thaliana JAZ4 protein with SEQ ID NO:7. This Triticum aestivum jasmonate ZIM-domain transcriptional repressor protein with NCBI accession no. ABK63978.1, has the following sequence (SEQ ID NO:36).

| | |
|---|---|
| 1 | LANGRSGMLP MSSPPANPGQ LTIFYGGSVC VYDSVPPEKA |
| 41 | QAIMLIAAAA AAASKSNGTA AVKPPAMSAT NAIQAMLTRS |
| 81 | LSLQSTSVAX GQPQAVADPG SICKLQADLP IAPRHSLQRF |
| 121 | LEKRRDRVVS KAPYGARKPF EGMGASSGME SVAEGRP |

A Zea mays protein referred to as hypothetical protein Zm00014a_023069 protein with NCBI accession no. PWZ14661.1 (SEQ ID NO:37) has significant sequence identity to the Arabidopsis thaliana JAZ5 protein with SEQ ID NO:9, as illustrated by the sequence comparison shown below. Domains of sequence homology are identified by asterisks below each sequence comparison.

| 31.4% identity in 207 residues overlap; Score: 131.0; Gap frequency: | | |
|---|---|---|
| 11.6% | | |
| Seq9 | 7 | NAKAQAPEKSDFTRRCSLLSRYLKEKGSFGNIDLGLYRKPDSSLALPGKFDPPGKQNAMH |
| Seq37 | 4 | HAPARDKTTSGFAATCSLLSQFLKEKKG-GLQGLGGLAMAPAPAAGAGAFRPPTTMNLLS |
| | | * * * * ***** **** * ** * * * ** * |
| Seq9 | 67 | KAGHSKGEPSTSSGGKVKDVADLSESQ-PGSSQLTIFFGGKVLVYNEFPVDKAKEIMEVA |
| Seq37 | 63 | ALDAAKATVGEPEGHGQRTGGNPREAAGEEAQQLTIFYGGKVVVFDRFPSAKVKDLLQIV |
| | | * * * ***** **** * ** * * |
| Seq9 | 126 | KQAKPVTEINIQTPINDENNNNKSSMVLPDLNEPTDNNHLTKEQQQQQEQNQIVERIARR |
| Seq37 | 123 | ------------SPPGADAVVDGAGAAVPTQNLPRPPHDSLSADLP----------IARR |
| | | * * * * **** |
| Seq9 | 186 | ASLHRFFAKRKDRAVARAPYQVNQNAG |
| Seq37 | 761 | NSLHRFLEKRKDRITAKAPYQVNSSVG |
| | | ***** ***** * ****** * |

This Zea mays protein referred to as hypothetical protein Zm00014a_023069 with NCBI accession no. PWZ14661.1, has the following sequence (SEQ ID NO:37).

| | |
|---|---|
| 1 | MAGHAPARDK TTSGFAATCS LLSQFLKEKK GGLQGLGGLA |
| 41 | MAPAPAAGAG AFRPPTTMNL LSALDAAKAT VGEPEGHGQR |
| 81 | TGGNPREAAG EEAQQLTIFY GGKVVVFDRF PSAKVKDLLQ |
| 121 | IVSPPGADAV VDGAGAAVPT QNLPRPPHDS LSADLPIARR |
| 161 | NSLHRFLEKR KDRITAKAPY QVNSSVGAEA SKAEKPWLGL |
| 201 | GQEGSDGRQA GDVIDE |

A Glycine max protein referred to as a TIFY 10A protein with NCBI accession no. XP_003546514.1 (SEQ ID NO:38) has significant sequence identity to the Arabidopsis thaliana JAZ5 protein with SEQ ID NO:9, as illustrated by the sequence comparison shown below. Domains of sequence homology are identified by asterisks below each sequence comparison.

| 35.6% identity in 219 residues overlap; Score: 206.0; Gap frequency: | | |
|---|---|---|
| 9.6% | | |
| Seq9: | 10 | AQAPEKSDFTRRCSLLSRYLKEKGSFGNIDLGLYR--KPDSSL--ALPGKFDPPGKQNAM |
| Seq38 | 16 | ARSPEKSSFSQTCSLLSQYIKEKGSFGDLTLGMTSCGSPETSCQSATTMNLFPPKENNVA |
| | | * **** * ***** * ******* ** * * * ** * |
| Seq9: | 66 | HK--AGHSKGEPSTSSGGKVKDVADLSESQPQSS--------QLTIFFGGKVLVYNEFPV |
| Seq38 | 76 | PKNLTAMDLLSPQASSYGPSEEIPTLVNSSAIKSVSKGAKTAQMTIFYGGQVVVFDDFPA |
| | | * * ** * * * * * *** ** * * ** |
| Seq9: | 116 | DKAKEIMEVA-KQAKPVTEINIQTPINDENNNNKSSMVLPDLNE-----PTDN-NHLTKE |
| Seq38 | 136 | DKASEIMSYATKGGIPQSQNNSVYTYTQSQPSFPPTLIRTSADSSAPIIPSVNITNSIRE |
| | | *** *** * * * * * * * |
| Seq9: | 169 | QQQQQEQNQIVERIARRASLHRFFAKRKDRAVARAPYQV |
| Seq38 | 196 | HPQASSRPVVYLPIARKASLHRFLEKRKDRIASKAPYQV |
| | | * *** ****** ***** ***** |

This Glycine max protein referred to as a TIFY 10A protein with NCBI accession no. XP_003546514.1, has the following sequence (SEQ ID NO:38).

| | |
|---|---|
| 1 | MSSSSEYSEF SGQKPARSPE KSSFSQTCSL LSQYIKEKGS |
| 41 | FGDLTLGMTS CGSPETSCQS ATTMNLFPPK ENNVAPKNLT |
| 81 | AMDLLSPQAS SYGPSEEIPT LVNSSAIKSV SKGAKTAQMT |
| 121 | IFYGGQVVVF DDFPADKASE IMSYATKGGI PQSQNNSVYT |
| 161 | YTQSQPSFPP TLIRTSADSS APIIPSVNIT NSIREHPQAS |
| 201 | SRPVVYLPIA RKASLHRFLE KRKDRIASKA PYQVANGPSN |
| 241 | KAAESMPWLG LSASSPQI |

A cDNA encoding the SEQ ID NO:38 protein is available as NCBI accession no. XM_003546466.4 and a chromosomal segment encoding the SEQ ID NO:38 protein is on Glycine max chromosome 15 atNC_038251.1 (17292772 . . . 17295396).

An unnamed Triticum aestivum protein with NCBI accession no. SPT20417.1 (SEQ ID NO:39) has significant sequence identity to the Arabidopsis thaliana JAZ5 protein with SEQ ID NO:9, as illustrated by the sequence comparison shown below. Domains of sequence homology are identified by asterisks below each sequence comparison.

| 31.5% identity in 124 residues overlap; Score: 109.0; Gap frequency: | | |
|---|---|---|
| 5.6% | | |
| Seq9: | 89 | LSESQPGSSQLTIFFGGKVLVYNEFPVDKAKEIMEVAKQAKPVTEINIQTPINDE--NNN |
| Seq39 | 57 | MSSPPANPGQLTIFYGGSVCVYDSVPPEKAQAIMLIAAAAAAASKSNGTAAVKPPAMSAT |
| | | * ***** ** * ** * ** ** * * * |
| Seq9: | 147 | NKSSMVLPDLNEPTDNNHLTKEQQQQQEQNQIVER-----IARRASLHRFFAKRKDRAVA |
| Seq39 | 117 | NAIQAMLTRSLSLQSTSVANGQPQAVADPGSICKLQADLPIARRHSLQRFLEKRRDRVVS |
| | | * * * * **** ** ** ** ** * |
| Seq9: | 202 | RAPY |
| Seq39 | 177 | KAPY |
| | | *** |

This unnamed Triticum aestivum protein with NCBI accession no. SPT20417.1, has the following sequence (SEQ ID NO:39).

| | |
|---|---|
| 1 | MDLLERSAAT IKAEAGEAQR KEAERKEQEL EKEQETQQPG |
| 41 | LTGRPPLANG RSGMLPMSSP PANPGQLTIF YGGSVCVYDS |
| 81 | VPPEKAQAIM LIAAAAAAAS KSNGTAAVKP PAMSATNAIQ |
| 121 | AMLTRSLSLQ STSVANGQPQ AVADPGSICK LQADLPIARR |
| 161 | HSLQRFLEKR RDRVVSKAPY GAGKPSEGMG ASSGMEAVAE |
| 201 | GKAQ |

A Zea mays protein referred to as TIFY 10b with NCBI accession no. PWZ12604.1 (SEQ ID NO:40) has significant sequence identity to the Arabidopsis thaliana JAZ6 protein with SEQ ID NO:11, as illustrated by the sequence comparison shown below. Domains of sequence homology are identified by asterisks below each sequence comparison.

| 38.1% identity in 105 residues overlap; Score: 156.0; Gap frequency: | | |
|---|---|---|
| 1.9% | | |
| Seq11 | 106 | QLTIFFGGKVMVFNEFPEDKAKEIMEVAKEANHVAVDSKNSQSHMNLDKSNVVIPDLNEP |
| Seq40 | 100 | QLTIFYGGKVLVFDDFPADKAYDLMQLASKGSPVVQNVVLPQP--SAAAAVTTDKAVLDP |
| | | ***** **** ** ** **** * * * * * |
| Seq11 | 166 | TSSGNNEDQETGQQHQVVERIARRASLHRFFAKRKDRAVARAPYQ |
| Seq40 | 158 | VISLAAAAKKPARTNASDMPIMRKASLHRFLEKRKDRLNAKTPYQ |
| | | * * * ****** ***** * *** |

This TIFY 10b Zea mays protein with NCBI accession no. PWZ12604.1 has the following sequence (SEQ ID NO:40).

| | |
|---|---|
| 1 | MAASARPGER ATSFAVACSL LSRFVRQNGV AAALLGLRIK |
| 41 | GEVEQQRTPA TTSLLPGAEG EEVERRKETM ELFPQSVGFS |
| 81 | IKDAAAPPRE EQGDKEKPKQ LTIFYGGKVL VFDDFPADKA |
| 121 | KDLMQLASKG SPVVQNVVLP QPSAAAAVTT DKAVLDPVIS |
| 161 | LAAAAKKPAR TNASDMPIMR KASLHRFLEK RKDRLNAKTP |
| 201 | YQTAPSDAAP VKKEPESQPW LGLGPNAVDS SLNLS |

A Glycine max protein referred to as TIFY 10a-like isoform X1 with NCBI accession no. XP_006587054.1 (SEQ ID NO:41) has significant sequence identity to the Arabidopsis thaliana JAZ6 protein with SEQ ID NO:11, as illustrated by the sequence comparison shown below. Domains of sequence homology are identified by asterisks below each sequence comparison.

| 33.0% identity in. 227 residues overlap; Score: 233,0; Gap frequency: | | |
|---|---|---|
| 6.2% | | |
| Seq11 | 5 | QAPEKSNFSQRCSLLSRYLKEKGSFGNINMGLARKSDLELAGKFDLKGQQNVIKKVETSE |
| Seq41 | 17 | RSPEKSSFSQTCSLLSQYIKEKGSFGDLTLGMTSCGSPETSCQSATTMNLFPTKENNVTP |
| | | **** *** ***** * ******* * * * |
| Seq11 | 65 | TRPFKLIQKFSIGEASTSTEDKAIYIDLSEPAKVAPESGNSQLTIFFGGKVMVFNEFPED |
| Seq41 | 77 | KDLTAMDLFSPQASSYRPSEEIPTLINSSAIKSVSKSAKTAQMTIFYGGQVVVFDDFPAD |
| | | * * * * * *** ** * ** ** * |
| Seq11 | 125 | KAKEIMEVAKEA------NHVAVDSKNSQS------HMNLDKSNVVIPDLNEPTSSGNNE |
| Seq41 | 137 | KASEIMSYATKGIPQSQNNSVFTYTPSQPSFPANLVRTSADSSAPIIPSVN--ITNSIHE |
| | | ** *** * * * * * * ** * * |
| Seq11 | 173 | DQETGQQHQVVERIARRASLHRFFAKRKDRAVAPAPYQVNQHGSHLP |
| Seq41 | 195 | HPQASSRPVVYLPIARKASLHRFLEKRKDRIASKAPYQLANGSSNQP |
| | | * *** ****** ***** **** * * |

This Glycine max protein (TIFY 10a-like isoform X1) with NCBI accession no. XP_006587054.1 has the following sequence (SEQ ID NO:41).

| | |
|---|---|
| 1 | MSSSSEYSQF SGQKPARSPE KSSFSQTCSL LSQYIKEKGS |
| 41 | FGDLTLGMTS CGSPETSCQS ATTMNLFPTK ENNVTPKDLT |
| 81 | AMDLFSPQAS SYRPSEEIPT LINSSAIKSV SKSAKTAQMT |
| 121 | IFYGGQVVVF DDEPADKASE IMSYATKGIP QSQNNSVFTY |
| 161 | TPSQPSFPAN LVRTSADSSA PIIPSVNITN SIHEHPQASS |
| 201 | RPVVYLPIAR KASLHRFLEK RKDRIASKAP YQLANGSSNQ |
| 241 | PAESMPWLGL SASSPRI |

A chromosomal segment encoding the SEQ ID NO:41 protein is on Glycine max chromosome 9 atNC_038245.1 (7366501 . . . 7369207).

An Oryza sativa protein referred to as TIFY 10b with NCBI accession no. A2YNP2.1 (SEQ ID NO:42) has significant sequence identity to the Arabidopsis thaliana JAZ6 protein with SEQ ID NO:11, as illustrated by the sequence comparison shown below. Domains of sequence homology are identified by asterisks below each sequence comparison.

| 31.6% identity in 206 residues overlap; Score: 182.0; Gap frequency: | | |
|---|---|---|
| 5.8% | | |
| Seq11 | 10 | SNFSQRCSLLSRYLKEKGSFGNINMGLARKSDLELAGKFDLKGQQNVIKKVETSETRPFK |
| Seq42 | 16 | TSFAMACSLLSRYVRQNGAAA-AELGLGIRGEGE-APRAAPGTMSLLPGEAERKKETMEL |
| | | * ******* * ** * * * |
| Seq11 | 70 | LIQKFSIGEASTSTEDKAIYIDLSEPAKVAPESGNSQLTIFFGGKVMVFNEFPEDKAKEI |
| Seq42 | 74 | FPQSAGFGQQDAITADSAADAREQEPEK-------RQLTIFYGGKVLVFNDFPADKAKGL |
| | | * * * * * ** * ***** **** *** ** **** |
| Seq11 | 130 | MEVAKEANHVAVDSKNSQSHMNLD---KSNVVIPDLNEPTSSGNNEDQETGQQHQVVERI |
| Seq42 | 127 | MQLASKGSTVAPQNAVAPAPAAVTDNTKAPMAVPAPVSSLPTAQADAQKPARANASDMPI |
| | | * * ** * * * * |
| Seq11 | 187 | ARRASLHRFFAKRKDRAVARAPYQVN |
| Seq42 | 187 | ARKASLHRFLEKRKDRLNAKTPYQAS |
| | | ** ****** ***** * *** |

This Oryza sativa protein (TIFY 10b) with NCBI accession no. A2YNP2.1 has the following sequence (SEQ ID NO:42).

| | |
|---|---|
| 1 | MAASARPVGV GGERATSFAM ACSLLSRYVR QNGAAAAELG |
| 41 | LGIRGEGEAP RAAPGTMSLL PGEAERKKET MELFPQSAGF |
| 81 | GQQDAITADS AADAREQEPE KRQLTIFYGG KVLVFNDFPA |
| 121 | DKAKGLMQLA SKGSTVAPQN AVAPAPAAVT DNTKAPMAVP |
| 161 | APVSSLPTAQ ADAQKPARAN ASDMPIARKA SLHRFLEKRK |
| 201 | DRLNAKTPYQ ASPSDATPVK KEPESQPWLG LGPNAVVKPI |
| 241 | ERGQ |

A Zea mays protein referred to as protein TIFY5 with NCBI accession no. PWZ15752.1 (SEQ ID NO:43) has significant sequence identity to the Arabidopsis thaliana JAZ7 protein with SEQ ID NO:13. For example, the Zea mays SEQ ID NO:43 protein has domains of 65 residues having 32% sequence identity from positions 26-91 of the Arabidopsis thaliana JAZ7 protein with SEQ ID NO:13 and 21 residues having 62% sequence identity from positions 122-143 of the Arabidopsis thaliana JAZ7 protein with SEQ ID NO:13. This Zea mays protein referred to as protein TIFY 5 with NCBI accession no. PWZ15752.1 has the following sequence (SEQ ID NO:43).

| | |
|---|---|
| 1 | MDGGRDVDEG GVTGAVAAAA AQERRWRGGG GDDEESSGLS |
| 41 | NGGGGVELSL RLRTGADDGA ATAAALSPLP LPPPAEARRN |
| 81 | MTIFYNGRVC AADVTEIQAR AIISMASEET LADHRGRRRR |
| 121 | QQQQQLTRGD GGDGRQODGD SSSSTTTSAV ALARRCARGR |
| 161 | GLVGPAVEID QAADAGLSMK RSLQLFLQKR KARTAAAAAP |
| 201 | PYAGGRQAQA VRR |

A Glycine max protein referred to as protein TIFY5A with NCBI accession no. XP_003546080.1 (SEQ ID NO:44) has significant sequence identity to the Arabidopsis thaliana JAZ7 protein with SEQ ID NO:13, as illustrated by the sequence comparison shown below. Domains of sequence homology are identified by asterisks below each sequence comparison.

| 41.8% identity in 91 residues overlap; Score: 157.0; Gap frequency: | | |
|---|---|---|
| 2.2% | | |
| Seq13 | 55 | KQESQILTIFYNGHMCVSSDLTHLEANAILSLASRDVEEKSLSLRSSDGSDPPTIPNNST |
| Seq44 | 42 | QEQQQPLTIFYDGKICVA-DVTELQAKSILMLANRKLEERVRTPTGSEPSSPTVMQSNNQ |
| | | * ***** * ** * * * * ** ** * ** * * * * |
| Seq13 | 115 | RFHYQKA-SMKRSLHSFLQKRSLRIQATSPY |
| Seq44 | 101 | LYSPGTGPSMRKSLQRFLQKRRNRVQEASPY |
| | | ** ** ***** * * *** |

This Glycine max protein referred to as protein TIFY5A with NCBI accession no. XP_003546080.1 has the following sequence (SEQ ID NO:44).

| | |
|---|---|
| 1 | MRRNCNLELA LFPPSDSGPP MVDNVEEEAS EISPMQNLFH |
| 41 | RQEQQQPLTI FYDGKICVAD VTELQAKSIL MLANRKLEER |
| 81 | VRTPTGSEPS SPTVMQSNNQ LYSPGTGPSM RKSLQRFLQK |
| 121 | RRNPVQEASP YRH |

An unnamed Triticum aestivum protein with NCBI accession no. SPT17867.1 (SEQ ID NO:45) has significant sequence identity to the Arabidopsis thaliana JAZ7 protein with SEQ ID NO:13. For example, the Triticum aestivum SEQ ID NO:45 protein has domains of 31 residues having 45% sequence identity from positions 61-92 of the Arabidopsis thaliana JAZ7 protein with SEQ ID NO:13 and 24 residues having 67% sequence identity from positions 122-146 of the Arabidopsis thaliana JAZ7 protein with SEQ ID NO:13. This unnamed Triticum aestivum protein with NCBI accession no. SPT17867.1 has the following sequence (SEQ ID NO:45).

| | |
|---|---|
| 1 | MAAASRSAPE WWRDGGSVDD GGAFVELSLR LRTGSSSTAR |
| 41 | RSMTIFYNGR VVAVDVTELQ AREIITMASQ QILTEQQDSG |
| 81 | GGGGGTAVAQ YGAHENPSQP APQRWAPLLA SRSLRQGAGA |
| 121 | AAPVTSQAAA AGLSMKRSLQ RFLQKRKTRV AAMGSPYAGG |
| 161 | RRAMPS |

A Zea mays protein referred to as putative tify domain/CCT motif transcription factor family protein (NCBI accession no. DAA40037.1 (GI:414589466); SEQ ID NO:46) has significant sequence identity to the Arabidopsis thaliana JAZ9 protein with SEQ ID NO:15. For example, the Zea mays SEQ ID NO:46 protein has domains of 48 residues having 52% sequence identity from positions 218-266 of the Arabidopsis thaliana JAZ9 protein with SEQ ID NO:15, and 31 residues having 55% sequence identity from positions 119-150 of the Arabidopsis thaliana JAZ9 protein with SEQ ID NO:15. This JAZ-related uncharacterized Zea mays protein, referred to as putative tify domain/CCT motif transcription factor family protein (NCBI accession no. DAA40037.1 (GI:414589466)), has the following sequence (SEQ ID NO:46).

| | |
|---|---|
| 1 | MDWSFASKPC AAPALMSFRS AAREEPSFPQ FSALDGTKNT |
| 41 | APRMLTHQRS FGPDSTQYAA LHRAQNGARV VPVSSPFSQS |
| 81 | NPMFRVQSSP SLPNSTAFKQ PPFAISNAVA SSTVGSYGGT |
| 121 | RDAVRPRTAQ LTIFYAGSVN VFNNVSAEKA QELMFLASRG |
| 161 | SSAPVACKPE APPTLAPAKV TAPEVLLPAK QMLFQKPQHL |
| 201 | SPPPSSVPGI LQSAALPRSA SSSSNLDSPA PKSSVPLAVP |
| 241 | PVSQAPPATL IATTTAAAIM PRAVPQARKA SLARFLEKRK |
| 281 | ERVTTAAPYP SAKSPLESSD TFGSGSASAN ANDKSSCTDI |
| 321 | ALSSNHEESL CLGGQPRSII SFSEESPSTK LQI |

A chromosomal segment encoding the SEQ ID NO:46 protein is on Zea mays chromosome 2 at NC_024460.1 (180086924 . . . 180089758, complement), sequence available as NCBI accession number NC_024460.1 (GI:662249846).

A Glycine max protein referred to as protein TIFY 6A isoform X6 (NCBI accession no XP_006580449.1 (GI:571456657; SEQ ID NO:47) has significant sequence identity to the Arabidopsis thaliana JAZ9 protein with SEQ ID NO:15, as illustrated by the sequence comparison shown below. Domains of sequence homology are identified by asterisks below each sequence comparison.

| 39.8% identity in 176 residues overlap | | |
|---|---|---|
| Seq15 | 117 | SPQLTIFYGGTISVFNDISPDKAQAIMLCAGNGLKGETGDSKP----------------- |
| Seq47 | 156 | SAQLTIFYAGTVNIFDDISAEKAQAIMLLAGNSLSAASNMAQPNVQVPISKLGAGAGVPV |
| | | * ****** ** * *** ******* *** * * |
| Seq15 | 160 | VREAERMYGKQIHN-------TAATSSSSATHTDNFSRCRDTPVAATNAMSMIESFNAAP |
| Seq47 | 216 | SQPANTSPGSGLSSPLSVSSHTGVQSGSGLTSTDEFLAAKTTGVPNPTICNVEPPKVVSA |
| | | * * * * * * ** * * * * |
| Seq15 | 213 | RNMIPS-VPQARKASLARFLEKRKERLMSAMPYK--KMLLDLSTGESSGMNYSSTS |
| Seq47 | 276 | TTMLTSAVPQARKASLARFLEKRKERVMSAAPYNLNKKSEECATAEYAGVNFSATN |
| | | * * ******************* *** ** * * * * * * * |

This JAZ-related Glycine max protein, referred to as protein TIFY 6A isoform X6 (NCBI accession no. XP_006580449.1 (GI:571456657)) has the following sequence (SEQ ID NO:47).

| | |
|---|---|
| 1 | MERDFMGLNL KEPLAVVKEE MNNDGCKNSG FKKGRIAQWP |
| 41 | FSNKVSALPH LMSFKASQDD KTKNTVSDTL SSSGFMSILS |
| 81 | QEAFDTSQKR SAGEPQMFSV PNQAISVSLG NPFLKNHFAA |
| 121 | AGQKPLLGGI PVTTSHSVLP SAVAVAGMTE SCVKPSAQLT |
| 161 | IFYAGTVNIF DDISAEKAQA IMLLAGNSLS AASNMAQPNV |
| 201 | QVPISKLGAG AGVPVSQPAN TSPGSGLSSP LSVSSHTGVQ |
| 241 | SGSGLTSTDE FLAAKTTGVP NTPICNVEPP KVVSATTMLT |
| 281 | SAVPQARKAS LARFLEKRKE RVMSAAPYNL NKKSEECATA |
| 321 | EYAGVNFSAT NTVLAKQG |

A cDNA encoding the SEQ ID NO:47 protein is available as NCBI accession number XM_006580386.2 (GI:955322109), and a chromosomal segment encoding the SEQ ID NO:47 protein is on Glycine max chromosome 5 at NC_016092.2 (41222014 . . . 41225906), sequence available as NCBI accession number NC_016092.2 (GI:952545311).

An unknown Oryza sativa protein with NCBI accession no. BAD28520.1 (GI:50251455; SEQ ID NO:48) has significant sequence identity to the Arabidopsis thaliana JAZ9 protein with SEQ ID NO:15. For example, the Oryza sativa SEQ ID NO:48 protein has domains of 66 residues having 41% sequence identity from positions 84-150 of the Arabidopsis thaliana JAZ9 protein with SEQ ID NO:15, and 41 residues having 56% sequence identity from positions 218-259 of the Arabidopsis thaliana JAZ9 protein with SEQ ID NO:15. This JAZ-related Oryza sativa protein with NCBI accession no. BAD28520.1 (GI:50251455) has the following sequence (SEQ ID NO:48).

| | |
|---|---|
| 1 | MQQASHVLAR QPPHVLNGAR VIPASSPFNP NNPMFRVQSS |
| 41 | PNLPNAVGAG GGAFKQPPFA MGNAVAGSTV GVYGTRDMPK |
| 81 | AKAAQLTIFY AGSVNVFNNV SPEKAQELMF LASRGSLPSA |
| 121 | PTTVARMPEA HVFPPAKVTV PEVSPTKPMM LQKPQLVSSP |
| 161 | VPAISKPISV VSQATSLPRS ASSSNVDSNV TKSSGPLVVP |
| 201 | PTSLPPPAQP ETLATTTAAA IMPRAVPQAR KASLARFLEK |
| 241 | RKERVTTVAP YPLAKSPLES SDTMGSANDN KSSCTDIALS |
| 281 | SNRDESLSLG QPRTISFCEE SPSTKLQI |

A chromosomal segment encoding the SEQ ID NO:48 protein is on Oryza sativa chromosome 9 at NC_029264.1 (14056084 . . . 14060320, complement), sequence available as NCBI accession number NC_029264.1 (GI:996703424).

An uncharacterized Zea mays protein referred to as LOC100384222 (NCBI accession no. NP_001182812.1 (GI:308044557); SEQ ID NO:49) has significant sequence identity to the Arabidopsis thaliana JAZ10 protein with SEQ ID NO:17, as illustrated by the sequence comparison shown below. Domains of sequence homology are identified by asterisks below each sequence comparison.

| 36.2% identity in 94 residues overlap; Score: 126.0; Gap frequency: | | |
|---|---|---|
| 3.2% | | |
| Seq17 | 105 | MTIFYNGSVSVF-QVSRNKAGEIMKVANEAASKKDESSMETDLSVILPTTLRPKLFGQNL |
| Seq49 | 96 | LTIFYGGKVVVFDRFPSAKVKDLLQIVSPPGA--DAVVDGAGAGAAVPTQNLPRPSHDSL |
| | | **** * * ** * * ** * * |
| Seq17 | 164 | EGDLPIARRKSLQRFLEKRKERLVSTSPYYPTSA |
| Seq49 | 154 | SADLPIARRNSLHRFLEKRKDRITAKAPYQVNSS |
| | | ******* ** ******* * ** * |

This JAZ-related uncharacterized Zea mays protein referred to as LOC100384222 (NCBI accession no. NP_001182812.1 (GI:308044557)) has the following sequence (SEQ ID NO:49).

| | |
|---|---|
| 1 | MAGHAPARDK TTTGFAATCS LLSQFLKEKK GGLQGLGGLA |
| 41 | MAPAPAAGAG AFRPPTTMNL LSALDAAKAT VGEPEGHGQR |
| 81 | TGGNPREAAG EEAQQLTIFY GGKVVVFDRF PSAKVKDLLQ |
| 121 | IVSPPGADAV VDGAGAGAAV PTQNLPRPSH DSLSADLPIA |
| 161 | RRNSLHRFLE KRKDRITAKA PYQVNSSVGA EASKAEKPWL |
| 201 | GLGQEQEGSD GRQAGEEM |

A cDNA encoding the SEQ ID NO:49 protein is available as NCBI accession number NM_001195883.1 (GI:308044556), and a chromosomal segment encoding the SEQ ID NO:49 protein is on Zea mays chromosome 7 at NC_024465.1 (121257106 . . . 121259180, complement), sequence available as NCBI accession number NC_024465.1 (GI:662248746).

An uncharacterized Glycine max protein referred to as LOC100306524 (NCBI accession number NP_001236269.1 (GI:351723837; SEQ ID NO:50) has significant sequence identity to the Arabidopsis thaliana JAZ10 protein with SEQ ID NO:17, as illustrated by the sequence comparison shown below. Domains of sequence homology are identified by asterisks below each sequence comparison.

| 36.6% identity in 123 residues overlap; Score: 114.0; Gap frequency: | | |
|---|---|---|
| 12.2% | | |
| Seq17 | 85 | SPVHASLARSSTELVSGTVPMTIFYNGSVSVFQ-VSRNKAGEIMKVANEAASKKDESSME |
| Seq50 | 38 | SPNKSVPASGLDAVIPSANQLTIFYNGSVCVYDGIPAEKVHEIMLIAAAAAKSTEMKKIG |
| | | ** * ******** * * *** * ** |
| Seq17 | 144 | TDLSVILPTTLRP---------------KLFGQLEGDLPIARRKSLQRFLEKRNERLVST |
| Seq50 | 98 | TQTTLISPAPSRPSSPHGITNNIGSSQKSSICRLQAEFPIARRHSLQRFLEKRRDRLGSK |
| | | * * * ** * * ***** ********* ** * |
| Seq17 | 190 | SPY |
| Seq50 | 158 | TPY |
| | | ** |

This JAZ-related uncharacterized Glycine max protein referred to as LOC100306524 (NCBI accession number NP_001236269.1 (GI:351723837) has the following sequence (SEQ ID NO:50).

| | |
|---|---|
| 1 | MAAGVTVKSE VLESSPPEGV CSNTVENHLV QTNLSDGSPN |
| 41 | KSVPASGLDA VIPSANQLTI FYNGSVCVYD GIPAEKVHEI |
| 81 | MLIAAAAAKS TEMKKIGTQT TLISPAPSRP SSPHGITNNI |
| 121 | GSSQKSSICR LQAEFPIARR HSLQRFLEKR RDRLGSKTPY |
| 161 | PSSPTTKVAD NIENNFCADN APELISLNRS EEEFQPTVSA |
| 201 | S |

A cDNA encoding the SEQ ID NO:50 protein is available as NCBI accession number NM_001249340.2 (GI:402766138), and a chromosomal segment encoding the SEQ ID NO:50 protein is on Glycine max chromosome 15 at NC_016102.2 (18552881 . . . 18556339), sequence available as NCBI accession number NC_016102.2 (GI:952545301).

An Oryza sativa protein referred to as protein TIFY 9 with NCBI accession no. XP_015634258.1 (GI:1002259863) has significant sequence identity to the Arabidopsis thaliana JAZ10 protein with SEQ ID NO:17, as illustrated by the sequence comparison shown below. Domains of sequence homology are identified by asterisks below each sequence comparison.

| 40.0% identity in 110 residues overlap; Score: 119.0; Gap frequency: | | |
|---|---|---|
| 13.6% | | |
| Seq17 | 83 | PISPVHASLARSSTELVSGTVPMTIFYNGSVSVFQVSRNKAGEIMKVANEAASKKDESSM |
| Seq51 | 65 | PPPPSTAPVPEEMPGAAAAAAPMTLFYNGSVAVFDVSHDKAEAIMRMATEATKAKGLA-- |
| | | * * * *** ****** ** ** ** * ** * |
| Seq17 | 143 | ETDLSVILPTTLRPKLFGQNLEGDLPIARRKSLQRFLEKRKERLVSTSPY |
| Seq51 | 123 | ------------RGNAIVGNFAKE-PLTRTKSLQRFLSKRKERLTSLGPY |
| | | * * * * ******* ****** * ** |
| 66.7% identity in 12 residues overlap; Score: 44.0; Gap frequency: | | |
| 0.0% | | |
| Seq17 | 2 | SKATIELDFLGL |
| Seg51 | 3 | TRAPVELDFLGL |
| | | * ******* |

This JAZ-related Oryza sativa protein referred to as protein TIFY 9 with NCBI accession no. XP_015634258.1 (GI:1002259863) has the following sequence (SEQ ID NO:51).

| | |
|---|---|
| 1 | MSTRAPVELD FLGLRAAAAD ADDRHAKSGG SSASSSSSIR |
| 41 | GMETSAIARI GPHLLRRVIA AAGPPPPPST APVPEEMPGA |
| 81 | AAAAAPMTLF YNGSVAVFDV SHDKAEAIMR MATEATKAKG |
| 121 | LARGNAIVGN FAKEPLTRTK SLQRFLSKRK ERLTSLGPYQ |
| 161 | VGGPAAVGAT TSTTTKSFLA KEEEHTAS |

A cDNA encoding the SEQ ID NO:51 protein is available as NCBI accession number XM_015778772.1 (GI:1002259862), and a chromosomal segment encoding the SEQ ID NO:51 protein is on Oryza sativa chromosome 4 at NC_029259.1 (19492605 . . . 19497181), sequence available as NCBI accession number NC_029259.1 (GI:996703429).

An uncharacterized Zea mays protein referred to as LOC100217316 isoform X2 with NCBI accession no. XP_008667401.1 (SEQ ID NO:52) has significant sequence identity to the Arabidopsis thaliana JAZ13 protein with SEQ ID NO:19, as illustrated by the sequence comparison shown below. Domains of sequence homology are identified by asterisks below each sequence comparison.

| 28.0% identity in 50 residues overlap; Score: 54.0; Gap | | |
|---|---|---|
| frequency: 6.0% | | |
| Seq 19 | 16 | TLQSCHDQSTVNDRSSTIRSKEINAFYSGRLS---EYDLVEIQMRAIIEM |
| Seq 52 | 235 | TIRTCYPQTPNGTGFATNRSAYIDMLFANKLHAFVEYDTIEDAARAIVEL |
| | | * * * ** * *** * *** * |
| 42.9% identity in 14 residues overlap; Score: 33.0; Gap | | |
| frequency: 0.0% | | |
| Seq 19 | 47 | SEYDLVEIQMRAII |
| Seq 52 | 197 | TESDLEELQARIVV |
| | | * ** * * * |
| 50.0% identity in 14 residues overlap; Score: 31.0; Gap | | |
| frequency: 0.0% | | |
| Seq19 | 101 | RSKSFTLTPNYTSS |
| Seq52 | 451 | RGKPQTLTPKVSES |
| | | * * **** * |

This uncharacterized Zea mays protein referred to as LOC100217316 isoform X2 with NCBI accession no. XP_008667401.1 has the following sequence (SEQ ID NO:52).

| | |
|---|---|
| 1 | MSQQEAVDPP SATDERLGGL PRSGSTSRLN AQAPEFVPRA |
| 41 | AAVPPPPPQQ KVVRLFAPPP HAAFFVAAPR PPPPPFEYYA |
| 81 | AVATGGGGRF GPPAAAAEQE AEAEQPPRDG SFDDPVPKIR |
| 121 | KQVEYYFSDI NLATTEHLMR FISKDPEGYV PISVVAGFKK |
| 161 | IKALVQSNSM LASALRTSSK LVVSDDGARV KREQPFTESD |
| 201 | LEELQARIVV AENLPDDHCY QNLMRLFSVV GSVRTIRTCY |
| 241 | PQTPNGTGPA TNRSAKLDML FANKLHAFVE YDTIEDAARA |
| 281 | IVELNDERNW RSGLRVRLLS TCMGGKGKKG GHESDGYGDE |
| 321 | ENVSTSDQPY DKYLEETPQM SDVPGEHMTE DSAGDMGRGR |
| 361 | VRGRGRGGRG RGRGYHQQNN NQHHQHYQNS SHHSNSSSTR |
| 401 | PVGTPPPSGH PVMIEQQQQQ QAAQPQPLTA ANKQPPGPRM |
| 441 | PDGSRGFSMG RGKPQTLIPK VSESEPEQ |

A cDNA encoding the SEQ ID NO:52 protein is available as NCBI accession number XM_008669179.2, and a chromosomal segment encoding the SEQ ID NO:52 protein is on Zea mays chromosome 2 at NC_024460.2 (226688215 . . . 226698574).

Chromosomal sites encoding any of the conserved amino acids and conserved domains illustrated by the sequence comparisons shown above can be deleted or mutated to reduce the activity of the proteins described herein.

For example, a wild type plant can express JAZ polypeptides or JAZ-related polypeptides with at least 70%, or at least 75%, or at least 80%, or at least 85%, or at least 90%, or at least 95%, or at least 97%, or at least 98%, or at least 99% sequence identity to any of SEQ ID NOs:1, 3, 5, 7, 9, 11, 13, 17, 19, 21-51 or 52.

However, the mutant jazD plant cells, plants, and/or seeds with improved insect and biotic stress resistance can express some JAZ and/or JAZ-related polypeptides such as the JAZ8, JAZ11, and JAZ12 proteins. In other words, endogenous JAZ8, JAZ11, and JAZ12 genes are not modified or mutated in the jazD plant cells, plants, and seeds described herein.

However, such jazD plant cells, plants, and/or seeds having reduced activity of JAZ1, JAZ2, JAZ3, JAZ4, JAZ5, JAZ6, JAZ7, JAZ9, JAZ10, and JAZ13 can have less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs: 1, 3, 5, 7, 9, 11, 13, 17, 19, 21-51 or 52.

The mutant JAZ and/or JAZ-related polypeptides can, for example, have mutations in at least one conserved amino acid position, or at least two conserved amino acid positions, or at least three conserved amino acid positions, or at least five conserved amino acid positions, or at least seven conserved amino acid positions, or at least eight conserved amino acid positions, or at least ten conserved amino acid positions, or at least fifteen amino acid positions, or at least twenty conserved amino acid positions, or at least twenty-five amino acid positions. In some cases, an entire conserved JAZ and/or JAZ-related domain or the entire endogenous JAZ and/or JAZ-related gene or chromosomal segment is deleted or mutated.

The conserved amino acids and/or domains are in some cases mutated by deletion or replacement with amino acids that have dissimilar physical and/or chemical properties. Examples of amino acids with different physical and/or chemical properties that can be employed are shown in Tables 1 and 2.

### Cdk8 Mutations

As described herein, loss-of-function mutations of cdk8 can improve the pest resistance, poor growth, and poor reproduction of jazD mutant plants. The Cdk8 gene is also named the CdkE1 or Hen3 gene in some species.

One example of a wild type Arabidopsis thaliana CDK8 protein sequence is provided by accession no. AT5G63610.1, shown below as SEQ ID NO:53.

| | |
|---|---|
| 1 | MGDGSSSRSN SSNSTSEKPE WLQQYNLVGK IGEGTYGLVF |
| 41 | LARTKIPPKR PIAIKKEKQS KDGDGVSPTA IREIMLLREI |
| 81 | SHENVVKLVN VHINFADMSL YLAFDYAEYD LYEIIRHHRD |
| 121 | KVGHSLNIYI VKSLLWQLLN GLNYLHSNWI IHRDLKPSNI |
| 161 | LVMGDAEEHG IVKIADEGLA RIYQAPLKPL SDNGVVVTIW |
| 201 | YRAPELLLGS KHYTSAVDMW AVGCIFAELT TLKPLFQGAE |
| 241 | AKSSQNPFQL DQLDKIFKIL GHPIMDKWPT LVNLPHWQND |
| 281 | VQHIQAHKYD SVGLHNVVHL NQKSPAYDLL SKMLEYDPLK |
| 321 | RITASQALEH EYFRMDPLPG RNAFVASQPM EKNVNYPTRP |
| 361 | VDTNTDFEGT TSINPPQAVA AGNVAGNMAG AHGMGSRSMP |
| 401 | RPMVAHNMQR MQQSQGMMAY NFPAQAGLNP SVPLQQQRGM |
| 441 | AQPHQQQQLR RKDPGMGMSG YAPPNKSRRL |

The wild type Arabidopsis thaliana CDK8 protein with SEQ ID NO:53 is encoded by a cDNA (At5G63610) with the following sequence (SEQ ID NO:54).

| | |
|---|---|
| 1 | GCAAGTGGCT AAAAAAATTA CAAATCTAGT TTCCATTCTC |
| 41 | AGCGTCGGCT GCTTGGAACG TCACCGTTTT CTGGAAAACG |
| 81 | CAATCTTCTC CCTTCCGTGA CGTCTCACCG GAATTTTCTC |
| 121 | GCTTTTGTCT ACTCTCCTCC ATCTCCGAGG TTCTCCAAGC |
| 161 | TCAGCTCCTC TTCCCATCAT TCATCCGACC GCCTTATCCG |
| 201 | GTCAGATCCT TTACGTATTT CTATTTTCCT GATCGTCGAT |
| 241 | TTTTGAGAAA TGTAAAAACA GATCGTATAA GGCCTCGAAG |
| 281 | TTTTTAATTT GAAAGTGGTA TCGAAATTTT TTGGTCTTTG |
| 321 | ATTAGGTTAG GGCACCGTAG CTCTGGGTAT TGAATTTGTA |
| 361 | GGGTTTTCCT CTGGTTATTG GTCTTTGGAG CTTGGTAATT |
| 401 | TCTGCTGAAT TGATTGATCC CTTTTCCATC TTTTGAAGTA |
| 441 | AAGTCTCGAG CTTTCGTGTC TCGATGTAGA TGAATTCTAT |
| 481 | TTTGAATATG AGATTTGATA AGACGTCAAT TGCTGATAAT |
| 521 | TTGGAGTCTT TGTGTCTGAA TTTGTTCATA TGAAGTTTTC |
| 561 | TGAGGGATGT GAATTTTATT GTCTGCTAAT TTTGAAACGT |
| 601 | TCCTTTTGGA ATTTGGTTTG TGAGGAGTCC TAGATCTTTT |
| 641 | TCTGTGAAGT TTCTTGCTTG TAAGTTTTCT GGATCACTTG |
| 681 | ATTGAGTCTA GAATCTAGAT AGATTACATG TACGGTTTGA |
| 721 | TTCCTTTGGC TGATTTTCCA AAGTTTTGTT CAAATTTCAG |
| 761 | GAGAACTACA AAGAGGAAAC CAAGATTGTT TTGTTTTGTT |
| 801 | AGACTCTACC CCTTTTCCGA TTCACATGGT AAGGACATTG |
| 841 | AGGTAGAGAA TAATACTAAA AAGCAATGGG AGATGGGAGT |
| 881 | TCCAGTAGAT CCAACAGCTC AAACAGCACT AGTGAGAAAC |
| 921 | CAGAGTGGCT GCAACAGTAC AATCTCGTTG GTAAGATTGG |
| 961 | TGAAGGCACT TATGGTCTTG TTTTCTTGGC TAGAACCAAG |
| 1001 | ACTCCGCCTA AAAGACCTAT TGCTATCAAG AAGTTTAAGC |
| 1041 | AGTCCAAAGA TGGAGATGGA GTTTCCCCGA CTGCTATCCG |
| 1081 | CGAGATCATG TTGCTTAGAG AGATTTCCCA TGAGAACGTC |
| 1121 | GTGAAGCTTG TGAATGTCCA CATCAATTTT GCAGACATGT |
| 1161 | CTCTGTATCT TGCCTTTGAT TATGCCGAGT ACGATCTCTA |
| 1201 | TGAAATCATC AGGCACCACA GAGACAAAGT CGGCCATTCG |
| 1241 | TTAAACACAT ACACAGTTAA GTCTTTGCTC TGGCAGCTTC |
| 1281 | TCAACGGATT GAACTATCTT CACAGTAATT GGATTATACA |
| 1321 | CAGAGATTTG AAACCGTCGA ATATCTTGGT TATGGGTGAT |
| 1361 | GCAGAAGAGC ACGGAATAGT GAAAATAGCT GATTTCGGGC |
| 1401 | TCGCAAGGAT ATATCAAGCT CCGTTGAAAC CACTATCGGA |
| 1441 | TAACGGAGTT GTGGTCACAA TCTGGTACCG AGCACCAGAG |
| 1481 | CTGCTTCTTG GTTCGAAGCA CTACACGAGC GCTGTTGATA |
| 1521 | TGTGGGCAGT TGGGTGTATA TTCGCGGAGT TACTAACTCT |
| 1561 | TAAACCGTTG TTTCAAGGAG CAGAAGCGAA ATCGTCTCAA |
| 1601 | AACCCTTTCC AGTTAGATCA ACTTGACAAG ATATTCAAGA |
| 1641 | TCTTAGGCCA CCCGACGATG GATAAATGGC CAACACTAGT |
| 1681 | TAACCTTCCA CACTGGCAAA ATGATGTTCA ACACATTCAA |
| 1721 | GCTCACAAAT ACGACAGTGT GGGTCTCCAC AACGTGGTTC |
| 1761 | ACCTGAATCA GAAAAGTCCT GCGTATGATC TGTTATCCAA |
| 1801 | AATGCTGGAA TATGATCCTC TAAAGCGGAT CACGGCTTCA |
| 1841 | CAAGCACTAG AACACGAGTA TTTCCGAATG GATCCTCTCC |
| 1881 | CAGGACGGAA CGCATTTGTA GCCAGCCAAC CGATGGAGAA |
| 1921 | GAATGTCAAT TACCCAACTC GTCCAGTAGA TACAAACACC |
| 1961 | GATTTCGAAG GCACGACAAG CATCAATCCG CCTCAAGCAG |
| 2001 | TAGCAGCAGG AAACGTAGCA GGGAACATGG CAGGAGCTCA |
| 2041 | TGGAATGGGC AGTAGATCGA TGCCAAGACC AATGGTTGCA |
| 2081 | CATAACATGC AGAGGATGCA GCAATCTCAA GGCATGATGG |
| 2121 | CTTATAATTT CCCGGCACAG GCAGGGCTTA ACCCGAGTGT |
| 2161 | TCCGCTGCAG CAGCAGCGCG GGATGGCTCA ACCGCACCAG |
| 2201 | CAGCAACAGC TAAGAAGGAA AGATCCCGGA ATGGGTATGT |
| 2241 | CAGGTTACGC ACCTCCTAAC AAATCCAGAC GCCTCTAAAG |
| 2281 | GTAAAATCGA GATCATCAGT CTCGGGTTAG AATCTGTGTG |
| 2321 | TTTGCCGCAG AAGAAAGCGT TGCGATTTGC TTTATAGAGT |
| 2361 | AGAGTTAGAT TGTAATGCAG CATGTGGAAT GTTGCTATTC |
| 2401 | ATATGGATGG ATTGGATTCT CTGTAGTTTT TGTATAAACA |
| 2441 | TCCTCTCAAG TATTTGTTAA TTATATTAGA TCATCATTTC |
| 2481 | TCTTAACATC ATTTCTCAAA ACGTAGTAAA TAGGAGATTT |
| 2521 | GCCAAGTGAA AAATATATAT AATGAGACAG TTATTATGAA |
| 2561 | C |

In Arabidopsis thaliana, the CDK8 gene resides on chromosome 5 at 25463362-25465922 bp.

Chromosomal sequences that encode CDK8 proteins from many plant types and species can be modified to reduce or eliminate the expression and/or function of the encoded protein. For example, the Arabidopsis thaliana CDK8 gene can be mutated to generate a null allele such as the sjd56 mutant CDK8 allele, which has a C1684T mutation altering a glutamine reside to a stop codon in the encoded protein. For example, the sjd56 mutation is shown in the CDK8 SEQ ID NO:54 nucleic acid sequence below, now referred to as SEQ ID NO:55 and illustrating that the position of this mutation can vary b 20-30 nucleotides.

| | |
|---|---|
| 1 | GCAAGTGGCT AAAAAAATTA CAAATCTAGT TTCCATTCTC |
| 41 | AGCGTCGGCT GCTTGGAACG TCACCGTTTT CTGGAAAACG |
| 81 | CAATCTTCTC CCTTCCGTGA CGTCTCACCG GAATTTTCTC |
| 121 | GCTTTTGTCT ACTCTCCTCC ATCTCCGAGG TTCTCCAAGC |
| 161 | TCAGCTCCTC TTCCCATCAT TCATCCGACC GCCTTATCCG |
| 201 | GTCAGATCCT TTACGTATTT CTATTTTCCT GATCGTCGAT |
| 241 | TTTTGAGAAA TGTAAAAACA GATCGTATAA GGCCTCGAAG |
| 281 | TTTTTAATTT GAAAGTGGTA TCGAAATTTT TTGGTCTTIG |
| 321 | ATTAGGTTAG GGCACCGTAG CTCTGGGTAT TGAATTTGTA |
| 361 | GGGTTTTCCT CTGGTTATTG GTCTTTGGAG CTTGGTAATT |
| 401 | TCTGCTGAAT TGATTGATCC CTTTTCCATC TTTTGAAGTA |
| 441 | AAGTCTCGAG CTTTCGTGTC TCGATGTAGA TGAATTCTAT |
| 481 | TTTGAATATG AGATTTGATA AGACGTCAAT TGCTGATAAT |
| 521 | TTGGAGTCTT TGTGTCTCAA TTTGTTCATA TGAAGTTTTC |
| 561 | TGAGGGATGT GAATTTTATT GTCTGCTAAT TTTGAAACGT |
| 601 | TCCTTTTGGA ATTTGGTTTG TGAGGAGTCC TAGATCTTTT |
| 641 | TCTGTGAAGT TTCTTGCTTG TAAGTTTTCT GGATCACTTG |
| 681 | ATTGAGTCTA GAATCTAGAT AGATTACATG TACGGTTTGA |
| 721 | TTCCTTTGGC TGATTTTCCA AAGTTTTGTT CAAATTTCAG |
| 761 | GAGAACTACA AAGAGCAAAC CAAGATTGTT TTGTTTTGTT |
| 801 | AGACTCTACC CCTTTTCCGA TTCACATGGT AAGCACATTG |
| 841 | AGGTAGAGAA TAATACTAAA AAGCAATGGG AGATGGGAGT |
| 881 | TCCAGTAGAT CCAACAGCTC AAACAGCACT AGTGAGAAAC |
| 921 | CAGAGTGGCT GCAACAGTAC AATCTCGTTG GTAAGATTGG |
| 961 | TGAAGGCACT TATGGTCTTG TTTTCTTGGC TAGAACCAAG |
| 1001 | ACTCCGCCTA AAAGACCTAT TGCTATCAAG AAGTTTAAGC |
| 1041 | AGTCCAAAGA TGGAGATCGA GTTTCCCCGA CTGCTATCCG |
| 1081 | CGAGATCATG TTGCTTAGAG AGATTTCCCA TGAGAACGTC |
| 1121 | GTGAAGCTTG TGAATGTCCA CATCAATTTT GCAGACATGT |
| 1161 | CTCTGTATCT TGCCTTTGAT TATGCCGAGT ACGATCTCTA |
| 1201 | TGAAATCATC AGGCACCACA GAGACAAAGT CGGCCATTCG |
| 1241 | TTAAACACAT ACACAGTTAA GTCTTTGCTC TGGCAGCTTC |
| 1281 | TCAACGGATT GAACTATCTT CACAGTAATT GGATTATACA |
| 1321 | CAGAGATTTG AAACCGTCGA ATATCTTGGT TATGGGTGAT |
| 1361 | GCAGAAGAGC ACGGAATAGT GAAAATAGCT GATTTCGGGC |
| 1401 | TCGCAAGGAT ATATCAAGCT CCGTTGAAAC CACTATCGGA |
| 1441 | TAACGGAGTT GTGGTCACAA TCTGGTACCG AGCACCAGAG |
| 1481 | CTGCTTCTTG GTTCGAAGCA CTACACGAGC GCTGTTGATA |
| 1521 | TGTGGGCAGT TGGGTGTATA TTCGCGGAGT TACTAACTCT |
| 1561 | TAAACCGTTG TTTCAAGGAG CAGAAGCGAA ATCGTCTCAA |
| 1601 | AACCCTTTCC AGTTAGATCA ACTTGACAAG ATATTCAAGA |
| 1641 | TCTTAGGCCA CCCGACGATG GATAAATGGC CAACACTAGT |
| 1681 | TAACCTTCCA CACTGGCAAA ATGATGTTCA ACACATTCAA |
| 1721 | GCTCACAAAT ACGACAGTGT GGGTCTCCAC AACGTGGTTC |
| 1761 | ACCTGAATCA GAAAAGTCCT GCGTATGATC TGTTATCCAA |
| 1801 | AATGCTGGAA TATGATCCTC TAAAGCGGAT CACGGCTTCA |
| 1841 | CAAGCACTAG AACACGAGTA TTTCCGAATG GATCCTCTCC |
| 1881 | CAGGACGGAA CGCATTTGTA GCCAGCCAAC CGATGGAGAA |
| 1921 | GAATGTGAAT TACCCAACTC GTCCAGTAGA TACAAACACC |
| 1961 | GATTTCGAAG GCACGACAAG CATCAATCCG CCTCAAGCAG |
| 2001 | TAGCAGCAGG AAACGTAGCA GGGAACATGG CAGGAGCTCA |
| 2041 | TGGAATGGGC AGTAGATCGA TGCCAAGACC AATGGTTGCA |
| 2081 | CATAACATGC AGAGGATGCA GCAATCTCAA GGCATGATGG |
| 2121 | CTTATAATTT CCCGGCACAG GCAGGGCTTA ACCCGAGTGT |
| 2161 | TCCGCTGCAG CAGCAGCGCG GGATGGCTCA ACCGCACCAG |
| 2201 | CAGCAACAGC TAAGAAGGAA AGATCCCGGA ATGGGTATGT |
| 2241 | CAGGTTACGC ACCTCCTAAC AAATCCAGAC GCCTCTAAAG |
| 2281 | GTAAAATCGA GATCATCAGT CTCGGGTTAG AATCTGTGTG |
| 2321 | TTTGCCGCAG AAGAAAGCGT TGCGATTTGC TTTATAGAGT |
| 2361 | AGAGTTAGAT TGTAATGCAG CATGTGGAAT GTTGCTATTC |
| 2401 | ATATGGATGG ATTGGATTCT CTGTAGTTTT TGTATAAACA |
| 2441 | TCCTCTCAAG TATTTGTTAA TTATATTAGA TCATCATTTC |
| 2481 | TCTTAACATC ATTTCTCAAA ACGTAGTAAA TAGGAGATTT |
| 2521 | GCCAAGTGAA AAATATATAT AATGAGACAG TTATTATGAA |
| 2561 | C |

As shown in the Examples, such sjd56 mutations of the CDK8 gene can improve plant pest resistance, growth, and seed production.

CDK8 genes from a variety of species can be modified (mutated) to improve their pest resistance, growth, and seed production. For example, chromosomal sequences encoding CDK8 genes from agriculturally important plants such as alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and/or wheat can be modified reduce or eliminate the expression and/or function of CDK8 proteins.

For example, a wild type Zea mays CDK8 protein has NCBI accession number AQK66278.1, and the sequence shown below as SEQ ID NO:56.

| | |
|---|---|
| 1 | MGDGRTGGAN RPAWLQQYEL IGKIGEGTYG LVFLARLKPP |
| 41 | HPAPGRKGPP IAIKKFKQSK EGDGVSPTAI REIMLLREIN |
| 81 | HENVVKLVNV HINHADMSLY LAFDYAEHDL YEIIRHHREK |
| 121 | LSSSINPYTV KSLLWQLLNG LNYLHSNWII HRDLKPSNIL |
| 161 | VMGEGDEHGI IKIADFGLAR IYQAPLKPLC DNGVVVTIWY |
| 201 | RAPELLLGGK HYTSAVDMWA VGCIFAELLT LKPLFQGVEA |
| 241 | KNPPNPFQLD QLDKIFKVLG HPTVEKWPTL ANLPWWQNDH |
| 281 | QHIQGHKYEN PGFHNIVHLP PKSPAFDLLS KMLEYDPRKR |
| 321 | ITAAQALEHE YFRMDPLPGR NALLPSQPGE KIVQYPIRPV |
| 361 | DTTTDFEGTT SLQPTOPPSG NAPPGGQSVA RPMPRQMPQQ |
| 401 | PMVGGIPRVA GGVTMAAFNA ASQAGMAGLN PGNMPMQRGA |
| 441 | GGQSHPHQLR RKADQGMGMQ NPGYPQQKRR F |

The Zea mays CDK8 protein with SEQ ID NO:56 is encoded by the LOC100284562 gene on chromosome 5 at NC_024463.2 (46913511 . . . 46918664, complement). A cDNA that encodes the SE ID NO:55 CDK8 protein is shown below as SE ID NO:57.

| | |
|---|---|
| 1 | GATATGTTAG CACTTAGCAG CATTCTTTGG TCCAACAAGT |
| 41 | CGAGAGAAGC GGGCCGTACG CCACCACGGC AACGGAGAAG |
| 81 | AGGACTTTCA GCTGCGGCGG CTGGCCGGCG CGGCGACGGG |
| 121 | GATGGGGGAT GGGCGCACAG GCGGCGCCAA CCGTCCGGCG |
| 161 | TGGCTGCAGC AGTACGAACT GATTGGCAAG ATTGGGGAGG |
| 201 | GGACCTATGG CCTCGTCTTC CTCGCGCGCC TTAAGCCGCC |
| 241 | CCACCCGGCA CCTGGCCGAC GCGGCCCCCC TATCGCCATA |
| 281 | AAGAAGTTTA AGCAGTCAAA GGAGGGGGAC GGAGTATCAC |
| 321 | CCACCGCAAT TAGAGAGATC ATGCTCCTGC GCGAGATCAA |
| 361 | CCACGAGAAT GTCGTCAAGC TCGTCAATGT GCACATCAAC |
| 401 | CACGCTGACA TGTCCCTATA CCTCGCATTC GATTACGCAG |
| 441 | AGCACGACCT CTATGAGATT ATCAGGCATC ACAGGGAGAA |
| 481 | GCTGAGTTCC TCCATTAACC CATACACTGT CAAATCCTTG |
| 521 | CTGTGGCAAC TGCTCAACGG CCTCAACTAT CTTCACAGTA |
| 561 | ACTGGATTAT ACATCGAGAT CTAAAGCCTT CCAACATACT |
| 601 | GGTCATGGGA GAAGGAGATG AACATGGAAT TATAAAGATA |
| 641 | GCCGATTTTG GACTTGCTAG GATATATCAA GCTCCACTGA |
| 681 | AACCATTATG TGATAATGGG GTTGTTGTAA CTATCTGGTA |
| 721 | TCGTGCTCCT GAGCTGTTAC TTGGGGGGAA ACACTACACC |
| 761 | AGTGCTGTCG ATATGTGGGC AGTTGGTTGC ATTTTTGCTG |
| 801 | AACTGCTTAC ACTGAAACCT CTATTCCAAG GTGTGGAAGC |
| 841 | AAAAAATCCT CCGAACCCAT TCCAGCTTGA TCAACTCGAC |
| 881 | AAGATTTTTA AGGTCTTAGG CCACCCTACA GTTGAAAAGT |
| 921 | GGCCTACCCT TGCCAATCTT CCATGGTGGC AAAACGACCA |
| 961 | CCAACACATT CAAGGACATA AGTATGAGAA CCCAGGTTTC |
| 1001 | CATAACATTG TTCATTTACC ACCAAAGAGT CCTGCATTTG |
| 1041 | ATCTTCTCTC AAAAATGCTT GAGTATGATC CCCGAAAGCG |
| 1081 | TATAACAGCT GCACAAGCTT TGGAGCATGA GACCTTAGTA |
| 1121 | ACCAGGTTCC CGGATCGATG GGATCGAGGA ACGGGAACGT |
| 1161 | GGTACGCGAT ACTTTCGGAT GGACCCACTA CCTGGACGAA |
| 1201 | ACGCGCTTTT ACCATCCCAG CCAGGGGAGA AAATTGTACA |
| 1241 | GTATCCTATT CGTCCAGTAG ATACTACAAC ACATTTTGAA |
| 1281 | GGAACAACAA GCCTTCAACC AACTCAACCG CCATCAGGGA |
| 1321 | ACGCTCCTCC TGGAGGTCAA TCTGTAGCAA GACCCATGCC |
| 1361 | ACGACAAATG CCGCAGCAAC CTATGGTTGG GGGGATTCCA |
| 1401 | AGAGTGGCAG GTGGAGTAAC CATGGCTGCC TTCAACGCTG |
| 1441 | CCTCACAGGC TGGCATGGCT GGGCTAAATC CTGGTAACAT |
| 1481 | GCCTATGCAG AGAGGCGCAG GTGGTCAGTC TCATCCGCAC |
| 1521 | CAGTTGAGAA GGAAGCCGGA TCAAGGCATG GGGATGCAGA |
| 1561 | ACCCTGGGTA TCCTCAGCAG AAGAGACGAT TCTGACGCTA |
| 1601 | TCAAGATGGA GCCATCTGCT GTATATCAGG TGTTTGAAAC |
| 1641 | ACGTTGCCTG TGTAAGCTGC TGTAGTTTTG TTATCAGCAT |
| 1681 | CCGAATGCCA ATGCTGGCAC CTGTAAAACA CATTAATCAG |
| 1721 | TCGAGAGTCC AGATACCAGT TGTCCTTATG GGTTATGATC |
| 1761 | TAAGCTGCTC GAATTTGGCT GATTTGGTTT GCAACAGAAA |
| 1801 | GGTCTTGCTT TTGCTCATGG CCCAGTGGAA TTATCCACAT |
| 1841 | GCGTAGGAAA TTTAGCATCT ATTTGGCTTG AGAAAAGATT |
| 1881 | TTCATTAAAT TCTAGTGGCA GTAAATATTT TTATGGCCAC |
| 1921 | AAACTACACA GAATTGAGCA GTTGAGCT |

Another wild type Zea mays CDK8 protein has NCBI accession number PWZ24329.1, and the sequence shown below as SEQ ID NO:58.

| | |
|---|---|
| 1 | MGDGRTGGAN RPAWLQQYEL IGKIGEGTYG LVFLARLKPP |
| 41 | HPAPGRRGPP IAIKKFKQSK EGDGVSPTAI REIMLLPEIN |
| 81 | HENVVKLVNV HINHADMSLY LAFDYAEHDL YEIIRHHREK |
| 121 | LSSSINPYTV KSLLWQLLNG LNYLHSNWII HRDLKPSNIL |
| 161 | VWCHQLYRNI IAQFLQTCPL ADTYFICATK VMGEGDEHGI |
| 201 | IKIADFGLAR IYQAPLKPLC DNGVVVTIWY RAPELLLGGK |
| 241 | HYTSAVDMWA VGCIFAELLT LKPLFQGVEA KNPPNPFQLD |
| 281 | QLDKIFKVLG HPTVEKWPTL ANLPWWQNDH QHIQGHKYEN |
| 321 | PGFHNIVHLP PKSPAFDLLS KMLEYDPRKR ITAAQALEHE |
| 361 | YFRMDPLPGR NALLPSQPGE KIVQYPIRPV DTTTDFEGTT |
| 401 | SLQPTQPPSG NAPPGGQSVA RPMPRQMPQQ PMVGGIPRVA |
| 441 | GGVTMAAFNA ASQAGMAGLN PGNMPMQRGA GGQSHPHQLR |
| 481 | RKADQGMGMQ NPGYPQQKPR F |

A wild type Glycine max CDK8 protein has NCBI accession number XP 003532085.1, and the sequence shown below as SEQ ID NO:59.

| | |
|---|---|
| 1 | MGDGSGNRWS RAEWVQQYDL LGKIGEGTYG LVFLARTKGT |
| 41 | PSKSIAIKKF KQSKDGDGVS PTAIREIMLL PEITHENVVK |
| 81 | LVNVHINHAD MSLYLAFDYA EHDLYEIIRH HRDKLNHSIN |
| 121 | QYTVKSLLWQ LLNGLSYLHS NWMIHRDLKP SNILVMGEGE |
| 161 | EHGVVKIADF GLARIYQAPL KPLSDNGVVV TIWYRAPELL |
| 201 | LGAKHYTSAV DMWAVGCIFA ELLTLKPLFQ GAEVKATSNP |
| 241 | FQLDQLDKIF KVLGHPTLEK WPSLASLPHW QQDVQHIQGH |
| 281 | KYDNAGLYNV VHLSPKSPAY DLLSKMLEYD PRKRLTAAQA |
| 321 | LEHEYFRIEP LPGRNALVPC QLCERIVNYP TRPVDTTTDL |
| 361 | EGTTNLPPSQ TVNAVSGSMP GPHGSNRSVP RPVNVVGMQR |
| 401 | MPPQAMAAYN LSSQAAMGDG MNPGGISKQR GVPQAEQPQQ |
| 441 | LBRKEQMGMP GYPAQQKSRR I |

The Glycine max CDK8 protein with SEQ ID NO:59 is encoded by the LOC100807993 gene on chromosome 8 at NC_038244.1 (211278 . . . 221643, complement). A cDNA that encodes the SEQ ID NO:58 CDK8 protein is shown below as SEQ ID NO:60.

| | |
|---|---|
| 1 | CATTTCAATT TTAGGACACG GCTGCCTATC CCCTTGCGAT |
| 41 | CGAAGAGAGA TGGGGGACGG AAGTGGGAAC CGGTGGAGCA |
| 81 | GGGCGGAGTG GGTGCAGCAG TACGATCTCT TAGGCAAAAT |
| 121 | CGGAGAAGGC ACTTACGGCC TCGTCTTCCT GGCCCGAACC |
| 161 | AAAGGCACTC CCTCCAAATC CATCGCCATC AAAAAGTTCA |
| 201 | AGCAATCCAA GGACGGCGAC GGCGTCTCCC CCACCGCCAT |
| 241 | CCGCGAAATC ATGCTGCTCA GGGAGATTAC GCACGAGAAC |
| 281 | GTCGTCAAGC TCGTCAATGT CCACATCAAC CACGCCGACA |
| 321 | TGTCGCTCTA CCTCGCCTTT GATTACGCCG AGCACGATCT |
| 361 | CTATGAAATT ATTAGGCATC ACAGGGATAA ACTCAACCAT |
| 401 | TCCATTAACC AATACACTGT TAAGTCTTTG CTCTGGCAGT |
| 441 | TGCTCAATGG ACTAAGCTAT CTGCACAGTA ATTGGATGAT |
| 481 | ACATCGAGAT TTGAAGCCAT CGAATATATT GGTTATGGGA |
| 521 | GAAGGAGAGG AACATGGAGT TGTTAAGATT GCTGACTTTG |
| 561 | GACTTGCGAG GATATATCAA GCTCCTCTGA AGCCGTTATC |
| 601 | TGATAATGGG GTTGTTGTAA CCATTTGGTA TCGTGCACCC |
| 641 | GAGTTGCTTC TTGGAGCAAA ACATTATACT AGTGCTGTTG |
| 681 | ATATGTGGGC TGTGGGATGC ATTTTTGCTG AGTTGTTGAC |
| 721 | CTTGAAGCCG CTATTTCAAG GGGCAGAAGT CAAAGCTACA |
| 761 | TCAAATCCCT TTCAGCTCGA CCAACTTGAC AAGATATTTA |
| 801 | AGGTTTTAGG CCATCCCACA TTAGAAAAGT GGCCTTCCTT |
| 841 | AGCAAGTCTT CCACATTGGC AACAAGATGT GCAACATATA |
| 881 | CAAGGACACA AATATGATAA TGCTGGTCTC TATAATGTTG |
| 921 | TACACCTGTC TCCAAAAAGC CCCGCATATG ACCTCTTGTC |
| 961 | AAAGATGCTT GAATATGATC CTCGTAAGCG TTTAACAGCA |
| 1001 | GCACAAGCTT TGGAGCATGA GTATTTCAAA ATTGAACCAT |
| 1041 | TACCTGGACG GAATGCACTT GTACCCTGCC AACTTGGAGA |
| 1081 | GAAAATTGTA AATTATCCCA CTCGTCCAGT GGACACCACT |
| 1121 | ACTGATCTTG AAGGAACAAC CAATCTGCCA CCTTCACAAA |
| 1161 | CGGTAAATGC AGTTTCTGGC AGCATGCCTG GTCCTCATGG |
| 1201 | GTCAAATAGA TCTGTTCCTC GGCCAGTGAA TGTTGTTGGA |
| 1241 | ATGCAAAGAA TGCCCCCTCA AGCAATGGCA GCTTATAATC |
| 1281 | TCTCATCTCA GGCAGCCATG GGAGACGGAA TGAATCCTGG |
| 1321 | GGGTATCTCA AAGCAACGAG GTGTTCCACA GGCCCATCAG |
| 1361 | CCGCAACAGT TGAGAAGGAA GGAGCAAATG GGGATGCCGG |
| 1401 | GATACCCTGC ACAACAGAAG TCAAGACGAA TATAAGGTTT |
| 1441 | CTGCTGGAAG AGAGACTACG TGAAGATAAA TTTGGGGTCA |
| 1481 | ATACTTCAGT GCCTGAACTC ATGCAGGACA TTTCTGGACA |
| 1521 | GGGTTTGTCT CAATACTTGC AAACCTCTCA CTTTATTGCA |
| 1561 | ATCAAAGATT GGGTGCATTC TTCTCTGGAA TTTTGATGCT |
| 1601 | AAAATGCCAA ATGTATGCTG GAACACCAAT GAAGCCATAA |
| 1641 | AAGGGTATAA ACGTATGAAA GGGTTAAGCT ACTGTAAGCA |
| 1681 | CATGTATATC ATGATTATAA CAATGCAATT CTATTGTATT |
| 1721 | TCTCAGCTTT TGGGCAAGAT CAATGTCAGT GAAACCAAAT |
| 1761 | GTTAATCATC CATTGGGTTT TCATAATGAA ACTTTTCACG |
| 1801 | ATTAAATTTA TAATATGCTA CTTTGTATTC GTCGAATATT |
| 1841 | TTGCCTCACA TGATTGAAGA TAGTTCAAAT ATCA |

Another wild type Glycine max CDK8 protein has NCBI accession number XP 003525137.1, and the sequence shown below as SEQ ID NO:61.

| | |
|---|---|
| 1 | MGDGSGSRWS RAEWVQQYDL LGKIGEGTYG LVFLARTKSP |
| 41 | VGTPSKSIAI KKFKQSKDGD GVSPTAIREI MLLREITHEN |
| 81 | VVKLVNVHIN HADMSLYLAF DYAEHDLYEI IRHHRDKLNH |
| 121 | SINQYTVKSL LWQLLNGLSY LHSNNMIHRD LKPSNILVMG |
| 161 | EGEEHGVVKI ADFGLARIYQ APLKPLSDNG VVVTIWYRAP |
| 201 | ELLLGAKHYT SAVDMWAMGC IFAELLTLKP LFQGAEVKAT |
| 241 | SNPFQLDQLD KIFKVLGHPT LEKWPSLASL PHWQQDVQHI |
| 281 | QGHKYDNAGL YNVVHLSPKS PAYDLLSKML EYDPRKRLTA |
| 321 | AQALEHEYFK IEPLPGRNAL VPCQLGEKIV NYPTRPVDTT |
| 361 | TDLEGTTNLP PSQTVNAVSG SMPGPHGSNR SVPRPMNVVG |
| 401 | MQRLPPQAMA AYNLSSQAAM GDGMNPGDIS KHRGVPQAHQ |
| 441 | PQQLRRKEQM GMPGYPAQQK SRRL |

The wild type Glycine max CDK8 protein with SEQ ID NO:61 is encoded by the LOC100794990 gene on chromosome 5 at NC_038241.1 (37955973 . . . 37967547, complement). A cDNA that encodes the SEQ ID NO:61 CDK8 protein is shown below as SEQ ID NO:62.

| | |
|---|---|
| 1 | TTTCAATTTT CAGACGCTGC TGCCTATCCC CTTGCGATCG |
| 41 | AACAGAACAG AAGAGAGATG GGGGACGGAA GTGGGAGCCG |
| 81 | GTGGAGCAGG GCGGAGTGGG TGCAGCAGTA CGATCTCTTA |
| 121 | GGAAAAATCG GCGAAGGCAC TTACGGCCTC GTCTTCCTCG |
| 161 | CCCGAACCAA ATCCCCCGTT GGCACTCCCT CCAAATCCAT |
| 201 | TGCCATAAAA AAGTTCAAGC AATCCAAGGA CGGCGACGGC |
| 241 | GTCTCCCCCA CCGCCATCCG CGAAATCATG TTGCTGAGGG |
| 281 | AGATTACGCA CGAGAACGTC GTCAAGCTCG TCAACGTACA |
| 321 | CATCAACCAC GCCGACATGT CTCTCTACCT CGCCTTCGAT |
| 361 | TACGCCGAGC ACGATCTCTA TGAAATTATT AGGCATCACA |
| 401 | GGGACAAACT CAACCATTCC ATTAATCAGT ACACTGTTAA |
| 441 | GTCTTTGCTC TGGCAGTTGC TCAATGGACT AAGCTATCTG |
| 481 | CACAGTAATT GGATGATACA TCGTGATTTG AAGCCATCGA |
| 521 | ATATATTGGT TATGGGTGAA GGAGAGGAAC ATGGAGTTGT |
| 561 | TAAGATTGCT GACTTTGGAC TTGCGAGGAT ATATCAAGCT |
| 601 | CCTCTGAAGC CGTTATCTGA CAATGGGGTT GTTGTAACCA |
| 641 | TTTGGTATCG TGCACCTGAG TTGCTTCTTG GAGCAAAACA |
| 681 | TTATACCAGT GCTGTTGATA TGTGGGCTAT GGGATGCATT |
| 721 | TTTGCTGAGT TGTTGACCTT GAAGCCACTA TTTCAACCGG |
| 761 | CAGAAGTCAA AGCTACATCA AATCCCTTTC AGCTTGACCA |
| 801 | ACTTGACAAG ATATTTAAGG TTTTAGGCCA TCCCACATTA |
| 841 | GAAAAGTGGC CTTCCTTAGC AAGTCTTCCA CATTGGCAAC |
| 881 | AAGATGTGCA ACATATACAA GGACACAAAT ATGACAATGC |
| 921 | CGGTCTCTAT AATGTTGTAC ACCTGTCTCC AAAAAGCCCT |
| 961 | GCATATGACC TCTTGTCAAA GATGCTTCAA TATGATCCTC |
| 1001 | GTAAGCGTTT AACAGCAGCA CAAGCTTTGG AGCATGAGTA |
| 1041 | TTTCAAAATT GAACCATTAC CTGGACGAAA TGCACTTGTA |
| 1081 | CCCTGCCAAC TTGGAGAGAA AATTGTAAAT TATCCCACTC |
| 1121 | GTCCAGTGGA CACTACAACT GATCTTGAAG GGACAACCAA |
| 1161 | TCTGCCACCT TCACAAACGG TAAATGCAGT TTCTGGTAGC |
| 1201 | ATGCCTGGTC CTCATGGGTC AAATAGATCT GTGCCTCGGC |
| 1241 | CAATGAATGT TGTTCCAATG CAAAGACTGC CCCCTCAAGC |
| 1281 | AATGGCAGCT TATAATCTCT CATCTCAGGC AGCCATGGGA |
| 1321 | GATGGAATGA ATCCTGGGGA TATCTCAAAG CATCGAGGTG |
| 1361 | TTCCACAGGC CCATCAGCCA CAACAGTTGA GAAGGAAGGA |
| 1401 | GCAAATGGGG ATGCCGGGAT ACCCTGCACA ACAGAAGTCA |
| 1441 | AGACGATTAT AAGGTTTCTG CTGGAAGAGA GACTAAGTGA |
| 1481 | AGATAGATTT GGGGTCAATA CTTCAGTACC TGAACTCATG |
| 1521 | CAGGACATTT CTGGACAGTG TTTGCCTTCA ATACTTGCAA |
| 1561 | GCCTCACTTT ATTGCAATCA AAGATTGGGT GCATTCTTCT |
| 1601 | CTGGAATTTT GATGCTAAAA TGCCAAATGT ATGCTGGAAC |
| 1641 | ACCAATGAAG CCATAAAAGG GAATAAACGT ATGAAAGGGT |
| 1681 | TAAGCTACTG TAAGCACATG TATATCATGA TTATAACAAT |
| 1721 | GCAATTCTAT TGTATTTCTT AGCTTTTGGG CAAGATCAAT |
| 1761 | GTCAGTAAAC CAAATGTTGA TCATCCATTA GGTTTTCATA |
| 1801 | ATGGAACTTT TCTTGATTAA ATCTATAACA TGCTACTTTG |
| 1841 | TATTTGTAGA ATATTTTGCC TCACATGATT GAAGATAGTT |
| 1881 | CAAATATCAC TTGCCTTTGG TATTTCCGTT TTGAATTTTT |
| 1921 | CTGTGATCAC TGGAATCACA GACTTTTCAC TCCCAAGGAG |
| 1961 | ATTATTGAAG CTTTCTGTGA GTATGATGTA AACTTTGTTC |
| 2001 | GGAGACGTAG TAGTATGAAG ATATCAAAAG CAGCAATTGG |
| 2041 | GAGAA |

A wild type Triticum aestivum CDK8 protein has NCBI accession number AAD10483.1, and the sequence shown below as SEQ ID NO:63.

| | |
|---|---|
| 1 | MEQYEKVEKI GEGTYGVVYK ARDRTTNETI ALKKIRLEQE |
| 41 | DEGVPSTAIR EISLLKEMQH GNIVKLHDVV HSEKRIWLVF |
| 81 | EYLDLDLKKF MDSCPEFAKS PALIKSYLYQ ILPGVAYCHS |
| 121 | HRVLHRDLKP QNLLIDRRTN ALKLADFGLA RAFGIPVRTF |
| 161 | THEVVTLWYR APEILLGARQ YSTPVDVWSV GCIFAEMVNQ |
| 201 | KPLFPGDSEI DELFKIFRVL GTPNEQTWPG VSSLPDYKSA |
| 241 | FPRWQAEDLA TVVPNLEPVG LDLLSKMLRF EPNKRITARQ |
| 281 | ALEHFYFKDM EMVQ |

The Triticum aestivum CDK8 protein with SEQ ID NO:63 is encoded by the cdc2TaA gene. A cDNA that encodes the SEQ ID NO:62 CDK8 protein is shown below as SEQ ID NO:64.

| | |
|---|---|
| 1 | GCCCCCCTCT CCCCCTCCCC CCCACCCCCC CAATGGCGGC |
| 41 | AGCAGCAGCA GCAGCAGCAG CAGCTTCGCC CGCCGCAGCC |
| 81 | GCTCTCCCCC GCCCCTCCTC CCCGTGATCC CCTTCCCCTT |
| 121 | CCCCTCCCCC GCTTCCTCCT CTCCCCCCTC CCGCCTCCTC |
| 161 | ACCCATTTCC CACGCCCGCG CCGCCGCCGC CGCCGTAGCA |
| 201 | TTGGACGCCG ACCCGATGGA GCAGTACGAG AAGGTGGAGA |
| 241 | AGATCGGGGA GGGCACGTAC GGGGTGGTGT ACAAGGCCCG |
| 281 | GGACAGGACC ACCAACGAGA CCATCGCGCT CAAGAAGATC |
| 321 | CGCCTGGAGC AGGAGGACGA GGGCGTCCCC TCCACCGCCA |
| 361 | TCCGCGAGAT CTCGCTCCTC AAGGAGATGC AGCACGGCAA |
| 401 | CATCGTCAAG CTGCACGATG TTGTCCACAG CGAGAAGCGC |
| 441 | ATATGGCTCG TCTTTGAGTA CCTGGATCTG GACCTGAAGA |
| 481 | AGTTCATGGA CTCCTGTCCA GAGTTTGCCA AGAGCCCCGC |
| 521 | CTTGATCAAG TCATATCTCT ATCAGATACT CCGCGGCGTT |
| 561 | GCTTACTGTC ATTCTCATAG AGTTCTTCAT CGAGATTTGA |
| 601 | AACCTCAGAA TTTATTGATA GACCGGCGTA CTAATGCACT |
| 641 | GAAGCTTGCA GACTTTGGTT TAGCAAGGGC ATTTGGAATT |
| 681 | CCTGTCCGTA CATTTACTCA TGAGGTAGTA ACATTATGGT |
| 721 | ACAGAGCTCC TGAAATCCTT CTTGGAGCAA GGCAGTATTC |
| 761 | CACACCAGTT GACGTGTGGT CAGTGGGCTG TATCTTTGCA |
| 801 | GAAATGGTGA ACCAGAAACC ACTGTTCCCT GGCGATTCTG |
| 841 | AGATTGATGA GCTATTTAAG ATATTCAGGG TACTCGGCAC |
| 881 | TCCAAATGAA CAAACTTGGC CAGGCGTGAG TTCCTTGCCT |
| 921 | GACTACAAGT CCGCCTTCCC CAGGTGGCAG GCAGAGGACC |
| 961 | TTGCAACCGT TGTCCCCAAT CTTGAACCTG TTGGCCTGGA |
| 1001 | CCTTCTCTCG AAAATGCTTC GGTTCGAGCC AAACAAGAGG |
| 1041 | ATCACGGCTA GGCAGGCTCT TGAGCATGAG TACTTCAAGG |
| 1081 | ACATGGAGAT GGTACAGTGA GCTGGCTATG TGGTAGTGAC |
| 1121 | TGGCATATGT ATGAGCTGAG CTGCTCGTTT CATTCCTTTT |
| 1161 | GTGAACGCTC |

A wild type Oryza sativa Japonica Group CDK8 protein has NCBI accession number XP_015614383.1, and the sequence shown below as SEQ ID NO:65.

| | |
|---|---|
| 1 | MGDGRVGGGT NRPAWLQQYE LVGKIGEGTY GLVFLARLKQ |
| 41 | SHPHAAAGVG RRGSPIAIKK FKQSKEGDGV SPTAIREIML |
| 81 | LREINHENVV KLVNVHINHA DMSLYLAFDY AEHDLYEIIR |
| 121 | HHREKLNLPI NPYTVKSLLW QLLNGLNYLH SNWIIHRDLK |
| 161 | PSNILVMGEG EEHGIIKIAD FGLARIYQAP LKPLSDNGVV |
| 201 | VTIWYRAPEL LLGAKHYTSA VDMWAVGCIF AELLTLKPLF |
| 241 | QGVEAKATPN PFQLDQLDKI FKVLGHPTVE KWPTLANLPC |
| 281 | WQNDQQHIQG HKYENTGLHN IVHLPQKSPA FDLLSKMLEY |
| 321 | DPRKRITAAQ ALEHEYFRMD PLPGRNALLP SQAGEKIVQY |
| 361 | PVRPVDTTTD FEGTTSLQPT QAPSGNAAPG NQSVVPRPIP |
| 401 | RQMQQPMVGM SRMGGTNMAA FGAAPQGGIA GMNPGNIPMQ |
| 441 | RGAGAQSHPH QLRRKADQGM GMQNPGYPTQ QKRRF |

The Oryza sativa CDK8 protein with SEQ ID NO:65 is encoded by the LOC4349519 gene on chromosome 10 at NC_029265.1 (23148732 . . . 23153285, complement). A cDNA that encodes the SEQ ID NO:65 CDK8 protein is shown below as SEQ ID NO:66.

| | |
|---|---|
| 1 | GAGCGTATTT TGGCTTTACG CCTTCGTGTG GAGTAAACGC |
| 41 | CCTTTCTGTT GGGCGGGTTC GGCTGGATCT TTTGTTCCCC |
| 81 | CTTTTCCTTT CTTCTCCGGC AGCGGCGGCG GCGATGGGGG |
| 121 | ACGGCCGCGT CGGAGGTGGA ACGAATCGGC CGGCATGGCT |
| 161 | GCAGCAATAC GAACTAGTGG GCAAGATTGG CGAGGGGACC |
| 201 | TACGGCCTCG TCTTCCTCGC TCGCCTCAAA CAATCGCATC |
| 241 | CCCACGCTGC CGCTGGCGTT GGCCGCCGTG GCTCTCCCAT |
| 281 | CGCCATCAAG AAGTTCAAGC AGTCCAAGGA GGGCGACGGT |
| 321 | GTCTCGCCCA CCGCCATCAG AGAGATCATG CTTCTGCGTG |
| 361 | AGATCAACCA CGAGAATGTT GTCAAGCTCG TCAATGTTCA |
| 401 | CATCAACCAC GCCGACATGT CCCTCTACCT CGCCTTCGAT |
| 441 | TACGCCGAGC ACGATCTCTA TGAGATTATC AGGCATCACA |
| 481 | GAGAGAAGCT TAACCTCCCC ATAAATCCCT ACACAGTCAA |
| 521 | ATCTTTGCTC TGGCAACTGC TCAATGGTCT CAACTATCTC |
| 561 | CATAGTAACT GGATTATCCA TCGAGATCTC AAGCCTTCTA |
| 601 | ATATACTGGT CATGGGAGAA GGAGAAGAAC ATGGAATTAT |
| 641 | AAAGATTGCT GATTTTGGAC TCGCTAGGAT ATATCAAGCT |
| 681 | CCATTAAAGC CATTAAGTGA TAACGGGGTT GTTGTTACCA |
| 721 | TCTGGTATCG GGCTCCAGAG TTGTTACTTG GGGCAAAGCA |
| 761 | CTACACAAGT GCTGTTGATA TGTGGGCAGT TGGTTGCATT |
| 801 | TTTGCTGAAT TGCTTACACT CAAACCACTG TTCCAAGGTG |
| 841 | TTGAAGCCAA AGCTACTCCA AACCCGTTTC AACTTGATCA |
| 881 | ACTAGACAAG ATTTTTAAGG TCTTAGGTCA TCCTACCGTT |
| 921 | GAGAAATGGC CTACCCTCGC TAATCTTCCA TGCTGGCAAA |
| 961 | ACGATCAACA ACACATTCAA GGGCATAAGT ATGAGAACAC |
| 1001 | AGGACTTCAT AATATTGTTC ACTTGCCTCA GAAGAGTCCT |
| 1041 | GCGTTTGATC TTCTCTCAAA AATGCTCGAG TATGATCCTC |
| 1081 | GAAAGCGTAT AACAGCTGCG CAAGCTTTGG AACATGAGTA |
| 1121 | CTTTCGAATG GATCCTCTGC CTGGACGGAA TGCACTTTTA |
| 1161 | CCATCGCAGG CTGGAGAGAA AATTGTGCAA TATCCTGTGC |
| 1201 | GTCCAGTTGA TACCACAACT GATTTTGAAG GAACAACAAG |
| 1241 | CCTTCAACCA ACTCAAGCGC CATCAGGGAA CGCAGCTCCT |
| 1281 | GGCAACCAGT CTGTGGTACC GAGACCCATT CCGAGGCAAA |
| 1321 | TGCAACAACC CATGGTCGGT ATGTCGAGAA TGGGTGGTAC |
| 1361 | AAACATGGCG GCCTTTGGTG CAGCTCCGCA AGGAGGCATA |
| 1401 | GCTGGGATGA ATCCTGGTAA TATTCCAATG CAGAGGGGCG |
| 1441 | CTGGAGGCCA ATCTCATCCG CATCAGTTGA GAAGGAAAGG |
| 1481 | TGATCAAGGG ATGGGGATGC AGAACCCCGG TTATCCTACT |
| 1521 | CAACAGAAGA GGCGGTTCTG ACCGACTGAA TTTGTAATTG |
| 1561 | TATATCTATT TGGTGTGTTA CTTGTGAGCA CGCTTAGCTT |
| 1601 | TTGCGGTGGT TGCTCCTAGT CGTACAGTGA GAATTGTATC |
| 1641 | TGTTCTGTTG TAATTGAACG CCATCACAAC CAACACCTCT |
| 1681 | ACTAGTTAGT TACTAGAGTG ACTACGGAGA CAGGGCCAGG |
| 1721 | TTGCCGATGA TGCCATCACC AATGGAGACA GGCATACCCA |
| 1761 | GCCAGAGTTT CGCCAATACT CTGCCCCCTG AACCCAACCA |
| 1801 | ATGAATGAAT TGGCATCGTA CGATCTATTT CA |

For example, a wild type plant can have cdk8 nucleic acids or express CDK8 polypeptides or CDK8-related polypeptides with at least 70%, or at least 75%, or at least 80%, or at least 85%, or at least 90%, or at least 95%, or at least 97%, or at least 98%, or at least 99% sequence identity to any of SEQ ID NOs:53-66. Plant cells from such wild type plants can be mutated, and mutant plants can be generated therefrom as described herein to provide modified jazD cdk8 plants and plant seed with improved plant growth and seed yields.

The mutant cdk8 plant cells, plants, and/or seeds with increased jasmonic acid responses and improved insect resistance can express mutant CDK8 and/or CDK8-related polypeptides that have reduced activity. In some cases, detectable levels of CDK8 proteins are not expressed Such cdk8 mutant plant cells and plant tissues have reduced CDK8 activity can cdk8 nucleic acids or cdk8 polypeptides that have less than 99%, or less than 98%, or less than 95%, or less than 90%, or less than 85%, or less than 75%, or less than 60%, or less than 50%, or less than 40%, or less than 30%, or less than 20% sequence identity to any of SEQ ID NOs: 53-66.

The mutant CDK8 and/or CDK8-related polypeptides can, for example, have mutations in at least one conserved amino acid position, or at least two conserved amino acid positions, or at least three conserved amino acid positions, or at least five conserved amino acid positions, or at least seven conserved amino acid positions, or at least eight conserved amino acid positions, or at least ten conserved amino acid positions, or at least fifteen amino acid positions, or at least twenty conserved amino acid positions, or at least twenty-five amino acid positions. In some cases, an entire conserved CDK8 and/or JAZ-related domain or the entire endogenous Cdk8 and/or Cdk8-related gene or chromosomal segment is deleted or mutated.

The conserved amino acids and/or domains are in some cases mutated by deletion or replacement with amino acids that have dissimilar physical and/or chemical properties. Examples of amino acids with different physical and/or chemical properties that can be employed are shown in Tables 1 and 2.

### Plant Modification

Mutations can be introduced into any of the wild type JAZ, JAZ-related, CDK8 or CDK8-related plant genomes by introducing targeting vectors, T-DNA, transposons, nucleic acids encoding TALENS, CRISPR, or ZFN nucleases, and combinations thereof into a recipient plant cell to create a transformed cell. Cells from virtually any dicot or monocot species can be stably modified or transformed, and these cells can be regenerated into transgenic plants, through the application of the techniques disclosed herein. The plant cells, plants, and seeds can therefore be monocotyledons or dicotyledons.

The cell(s) that undergo transformation may be in a suspension cell culture or may be in an intact plant part, such as an immature embryo, or in a specialized plant tissue, such as callus, such as Type I or Type II callus.

Transformation of the cells of the plant tissue source can be conducted by any one of a number of methods available to those of skill in the art. Examples include: Transformation by direct DNA transfer into plant cells by electroporation (U.S. Pat. Nos. 5,384,253, 5,472,869, Dekeyser et al., The Plant Cell. 2:591 602 (1990)); direct DNA transfer to plant cells by PEG precipitation (Hayashimoto et al., Plant Physiol. 93:857 863 (1990)); direct DNA transfer to plant cells by microprojectile bombardment (McCabe et al., Bio/Technology. 6:923 926 (1988); Gordon Kamm et al., The Plant Cell. 2:603 618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf disk protocol (Horsch et al., Science 227:1229 1231 (1985). Monocots such as Zea mays can be transformed via microprojectile bombardment of embryogenic callus tissue or immature embryos, or by electroporation following partial enzymatic degradation of the cell wall with a pectinase containing enzyme (U.S. Pat. Nos. 5,384,253, 5,472,869). For example, embryogenic cell lines derived from immature Zea mays embryos can be transformed by accelerated particle treatment as described by Gordon Kamm et al. (The Plant Cell. 2:603 618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation can be carried out with “naked” DNA where the expression cassette may be simply carried, for example, on any E. coli derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are exemplary Zea mays tissue sources. Selection of tissue sources for transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the targeting vector and/or other nucleic acids for an effective period of time. This may range from a less than one second pulse of electricity for electroporation to a 2-3-day co-cultivation in the presence of plasmid bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Where one wishes to introduce DNA by means of electroporation, it is contemplated that the method of Krzyzek et al. (U.S. Pat. No. 5,384,253) may be advantageous. In this method, certain cell wall degrading enzymes, such as pectin degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic cells were bombarded with intact cells of the bacteria E. coli or Agrobacterium tumefaciens containing plasmids with either the β-glucouronidase or bar gene engineered for expression in maize. Bacteria were inactivated by ethanol dehydration prior to bombardment. A low level of transient expression of the β-glucouronidase gene was observed 24-48 hours following DNA delivery. In addition, stable transformants containing the bar gene can be recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, is that the isolation of protoplasts (Christou et al., PNAS. 84:3962 3966 (1987)), the formation of partially degraded cells, or the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon Kamm et al., The Plant Cell. 2:603 618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth here in one may obtain up to 1000 or more foci of cells transiently expressing a marker gene. The number of cells in a focus which express the exogenous gene product 48 hours post bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macroprojectiles or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Examples of plants and/or plant cells that can be modified as described herein include alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and wheat. In some embodiments, the plant is a Brassicaceae or other Solanaceae species. In some embodiments, the plant or cell can be a maize plant or cell. In some embodiments, the plant is not a species of Arabidopsis, for example, in some embodiments, the plant is not Arabidopsis thaliana.

An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. In an illustrative embodiment embryogenic Type II callus of Zea mays L. can be selected with sub-lethal levels of bialaphos. Slowly growing tissue was subsequently screened for expression of the luciferase gene and transformants can be identified.

### Regeneration and Seed Production

Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to have the mutations. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants in order to introgress the mutations into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced, Jaz or Cdk8 mutations, the plant is self-pollinated at least once in order to produce a homozygous backcross converted inbred containing the mutations. Progeny of these plants are true breeding.

Alternatively, seed from transformed mutant plant lines regenerated from transformed tissue cultures is grown in the field and self-pollinated to generate true breeding plants.

Seed from the fertile transgenic plants can then be evaluated for the presence of the desired Jaz or Cdk8 mutation, and/or the expression of the desired mutant protein. Transgenic plant and/or seed tissue can be analyzed using standard methods such as SDS polyacrylamide gel electrophoresis, liquid chromatography (e.g., HPLC) or other means of detecting a mutation.

Once a transgenic plant with a mutant sequence and having improved growth and insect resistance is identified, seeds from such plants can be used to develop true breeding plants. The true breeding plants are used to develop a line of plants with an increase insect resistance relative to wild type, and acceptable growth characteristics while still maintaining other desirable functional agronomic traits. Adding the mutation to other plants can be accomplished by back-crossing with this trait and with plants that do not exhibit this trait and studying the pattern of inheritance in segregating generations. Those plants expressing the target trait (insect resistance, good growth) in a dominant fashion are preferably selected. Back-crossing is carried out by crossing the original fertile transgenic plants with a plant from an inbred line exhibiting desirable functional agronomic characteristics while not necessarily expressing the trait of an increased insect resistance and good plant growth. The resulting progeny are then crossed back to the parent that expresses the increased insect resistance and good plant growth. The progeny from this cross will also segregate so that some of the progeny carry the trait and some do not. This back-crossing is repeated until an inbred line with the desirable functional agronomic traits, and with expression of the trait involving an increase in insect resistance and good plant growth. Such insect resistance and good plant growth can be expressed in a dominant fashion.

The new transgenic plants can also be evaluated for a battery of functional agronomic characteristics such as growth, lodging, kernel hardness, yield, resistance to disease and insect pests, drought resistance, and/or herbicide resistance.

Plants that may be improved by these methods include but are not limited to agricultural plants of all types, oil and/or starch plants (canola, potatoes, lupins, sunflower and cottonseed), forage plants (alfalfa, clover and fescue), grains (maize, wheat, barley, oats, rice, sorghum, millet and rye), grasses (switchgrass, prairie grass, wheat grass, sudangrass, sorghum, straw-producing plants), softwood, hardwood and other woody plants (e.g., those used for paper production such as poplar species, pine species, and eucalyptus). In some embodiments the plant is a gymnosperm. Examples of plants useful for pulp and paper production include most pine species such as loblolly pine, Jack pine, Southern pine, Radiata pine, spruce, Douglas fir and others. Hardwoods that can be modified as described herein include aspen, poplar, eucalyptus, and others. Plants useful for making biofuels and ethanol include corn, grasses (e.g., miscanthus, switchgrass, and the like), as well as trees such as poplar, aspen, willow, and the like. Plants useful for generating dairy forage include legumes such as alfalfa, as well as forage grasses such as bromegrass, and bluestem.

### Determination of Stably Transformed Plant Tissues

To confirm the presence of Jaz, and/or Cdk8 mutations in the regenerating plants, seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from the introduced Jaz or Cdk8 mutants. For example, PCR also be used to reverse transcribe RNA into DNA, using enzymes such as reverse transcriptase, and then this DNA can be amplified through the use of conventional PCR techniques.

Information about mutations can also be obtained by primer extension or single nucleotide polymorphism (SNP) analysis.

Further information about the nature of the RNA product may be obtained by Northern blotting. This technique will demonstrate the presence of an RNA species and give information about the integrity of that RNA. The presence of some mutations can be detected by Northern blotting. The presence or absence of an RNA species (e.g., a Jaz or cdk8 RNA) can also be determined using dot or slot blot Northern hybridizations. These techniques are modifications of Northern blotting and also demonstrate the presence or absence of an RNA species.

While Southern blotting and PCR may be used to detect the presence of Jaz, and/or cdk8 mutations or the presence of a PIF4 expression cassette, they do not provide information as to whether the preselected DNA segment is being expressed.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products, or the absence thereof, that have been separated by electrophoretic techniques. Additional techniques may be employed to absolutely confirm the identity of a mutation such as evaluation by screening for reduced transcription (or no transcription) of Jaz, and/or cdk8 mRNAs, or by amino acid sequencing following purification. The Examples of this application also provide assay procedures for detecting and quantifying insect resistance and plant growth. Other procedures may be additionally used.

The expression of a gene product can also be determined by evaluating the phenotypic results of its expression. These assays also may take many forms including but not limited to analyzing changes in the insect resistance, growth characteristics, or other physiological properties of the plant. Expression of selected DNA segments encoding different amino acids or having different sequences and may be detected by amino acid analysis or sequencing.

The jazD cdk8 plants and seeds described herein can also be identified and characterized phenotypically. For example, the jazD cdk8 plant's vegetative weight or vegetative weight of a jazD cdk8 plant grown from jazD cdk8 plant seeds is within at least about 40%, or at least about 50%, or within at least 60%, or at least about 70% of the average vegetative weight of a wild type plant grown for the same time and under the same conditions as a wild type plant. Similarly, jazD cdk8 plants or plants grown from jazD cdk8 plant seeds have a seed yield that is at least 10%, or at least 20%, or at least 30%, or at least 40% greater than the average seed yield of wild type plants.

The jazD cdk8 plants or plants grown from jazD cdk8 plant seeds have at least 5% less, 10% less, 20% less, 30% less, 40% less, 50% less, 60% less, 70% less, 80% less, 90% less, or 100% less leaf damage from insect feeding than average insect feeding of a wild type plant of the same species grown for the same time under the same conditions.

The jazD cdk8 plants or plants grown from jazD cdk8 plant seeds have at least about 10%, or at least about 20%, or at least about 30%, or at least about 40%, or at least about 50%, or at least about 60%, or at least about 70%, or at least about 80%, or at least about 90% fewer insects or insect larvae than an average number of insects or insect larvae of wild type plants of the same species grown for the same time under the same conditions.

### Defense Against Pests

As illustrated herein, loss of function mutations of Jaz and cdk8 genes, such as those provided by loss of function of the JazD cdk8 genes, can improve plant resistance to insects. Plants with such mutations can produce a variety of compounds that can repel, metabolically undermine, or otherwise discourage insects and/or insect larvae from infesting plant tissues. Such compounds are referred to as defense compounds. In some cases, the defense compounds are aliphatic glucosinolates. Examples of defense compounds include:
3MSOP: 3-methylsulphinylpropyl glucosinolate, glucoiberin;
4MSOB: 4-methylsulphinylbutyl glucosinolate, glucoraphanin;
5MSOP: 5-methylsulphinylpentyl glucosinolate, glucoalyssin;
6MSOH: 6-methylsulphinylhexyl glucosinolate, glucohesperin;
7MSOH: 7-methylsulphinylheptyl glucosinolate, glucoibarin;
3MTP: 3-methylthiopropyl glucosinolate, glucoiberverin;
8MSOO: 8-methylsulphinyloctyl glucosinolate, glucohirsutin;
4MTB: 4-methylthiobutyl glucosinolate, glucoerucin;
5MTP: 5-methylthiopentyl glucosinolate, glucoberteroin;
7MTH: 7-methylthioheptyl glucosinolate;
Or a combination thereof.

Mutation of jaz and/or cdk8 genes in plants can lead to increased synthesis of at least one defense compound, at least two defense compounds, at least three defense compounds, at least four defense compound, at least five defense compounds, at least six defense compounds, at least seven defense compound, at least eight defense compounds, or at least nine defense compounds.

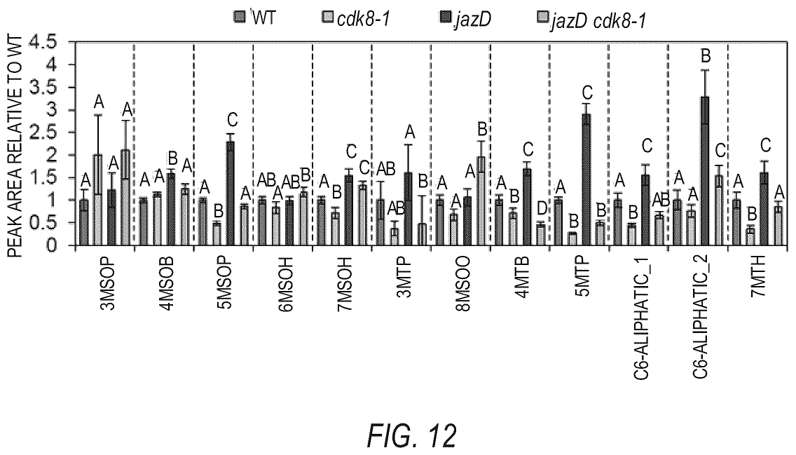

The defense compounds can be produced by a variety of plant tissues. Examples of plant tissues where the defense compounds can be made include leaves, stems, seeds, or a combination thereof. For example, plant leaves can have increased content of a variety of defense compounds in plants with loss of function JazD cdk8 genes, as illustrated in FIG. 12.

The defense compounds can be at least 2%, at least 3%, at least 4%, at least 5%, at least 7%, at least 10%, at least 13%, at least 15%, at least 17%, at least 20%, at least 25%, at least 30%, at least 40%, at least 50%, at least 70%, at least 80%, at least 90%, or at least 100% greater levels in plants with loss of function Jaz mutations, loss of function cdk8 mutations, or a combination thereof, than in unmodified parental or wild type plants.

### Definitions

As used herein, the singular forms “a,” “an,” and “the” are intended to include the plural forms as well, unless the context clearly indicates otherwise. Also, as used herein, “and/or” refers to, and encompasses, any and all possible combinations of one or more of the associated listed items. Unless otherwise defined, all terms, including technical and scientific terms used in the description, have the same meaning as commonly understood by one of ordinary skill in the art to which this invention pertains.

The term “about”, as used herein, can allow for a degree of variability in a value or range, for example, within 10%, within 5%, or within 1% of a stated value or of a stated limit of a range.

The term “heterologous” when used in reference to a nucleic acid or protein refers to a nucleic acid or protein that has been manipulated in some way. For example, a heterologous nucleic acid includes a nucleic acid from one species introduced into another species. A heterologous nucleic acid also includes a nucleic acid that is native to an organism that has been altered in some way (e.g., mutated, added in multiple copies, present in a locus within the genome, expressed from an autonomously replicating vector, linked to a non-native promoter, linked to a mutated promoter, or linked to an enhancer sequence, etc.). Heterologous nucleic acids may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). In some cases, heterologous nucleic acids are distinguished from endogenous plant genes in that the heterologous nucleic acids can be joined to nucleotide sequences comprising regulatory elements such as promoters that are not found naturally associated with the nucleic acid. In another example, the heterologous nucleic acids are associated with portions of the chromosome not found in nature (e.g., genes expressed in loci where the gene is not normally expressed).

The terms “identical” or percent “identity”, as used herein, in the context of two or more nucleic acids, or two or more polypeptide sequences, refer to two or more sequences or subsequences that are the same or have a specified percentage of nucleotides or amino acid residues that are the same (e.g., 75% identity, 80% identity, 85% identity, 90% identity, 95% identity, 97% identity, 98% identity, 99% identity, or 100% identity in pairwise comparison). Sequence identity can be determined by comparison and/or alignment of sequences for maximum correspondence over a comparison window, or over a designated region as measured using a sequence comparison algorithm, or by manual alignment and visual inspection. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. A “reference sequence” is a defined sequence used as a basis for a sequence comparison; a reference sequence may be a subset of a larger sequence.

The term “nucleic acid,” “nucleic acid segment” or “nucleic acid of interest” refers to any RNA or DNA, where the manipulation of which may be deemed desirable for any reason (e.g., treat or reduce the incidence of disease, confer improved qualities, etc.), by one of ordinary skill in the art. Such nucleic acids include, but are not limited to, coding sequences of structural genes (e.g., disease resistance genes, reporter genes, selection marker genes, oncogenes, drug resistance genes, growth factors, etc.), and noncoding regulatory sequences which do not encode an mRNA or protein product (e.g., promoter sequence, polyadenylation sequence, termination sequence, enhancer sequence, etc.).

As used herein, the term “plant” is used in its broadest sense. It includes, but is not limited to, any species of grass (fodder, ornamental or decorative), crop or cereal, fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

The term “seed” refers to a ripened ovule, consisting of the embryo and a casing.

Vegetative tissues or vegetative plant parts do not include plant seeds, and instead include non-seed tissues or parts of a plant. The vegetative tissues can include reproductive tissues of a plant, but not the mature seeds.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

The following Example illustrate some of the experiments performed and experimental results obtained during the development of the invention. Appendix A may provide further information.

### Example 1: Materials and Methods

This Example illustrates some of the materials and methods that were used in the development of the invention.

### Plant Material and Growth Conditions

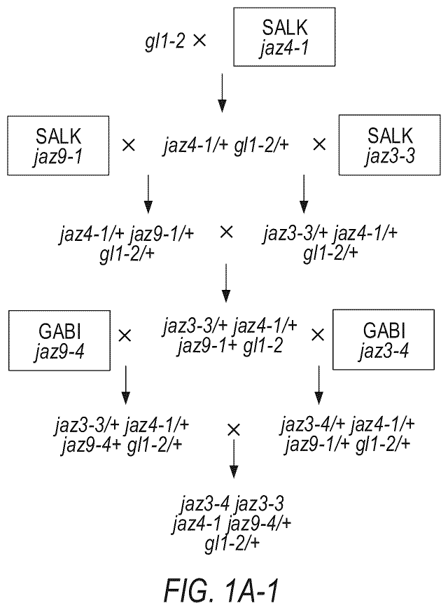

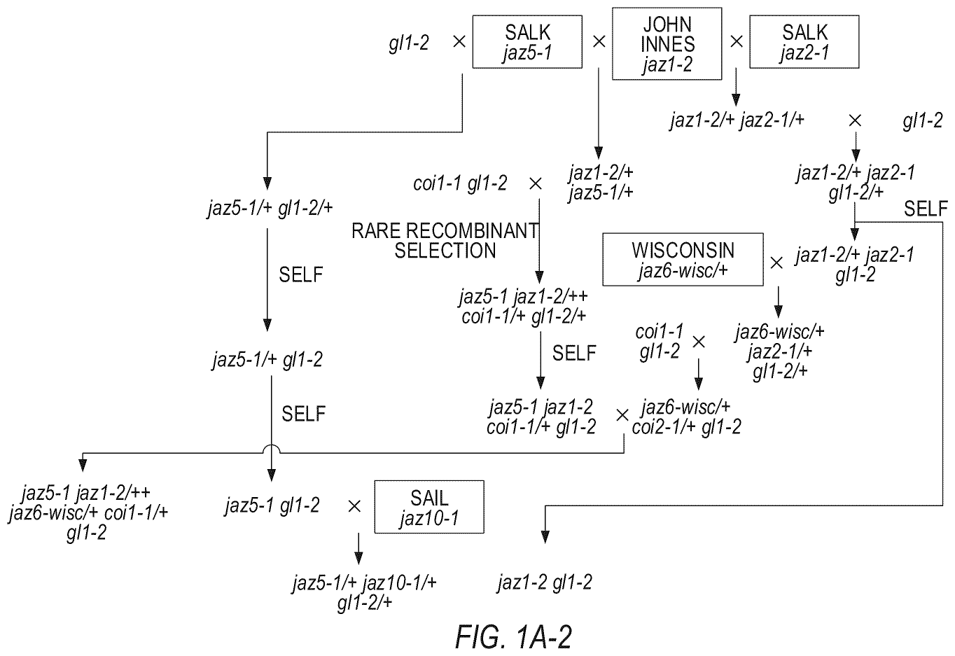

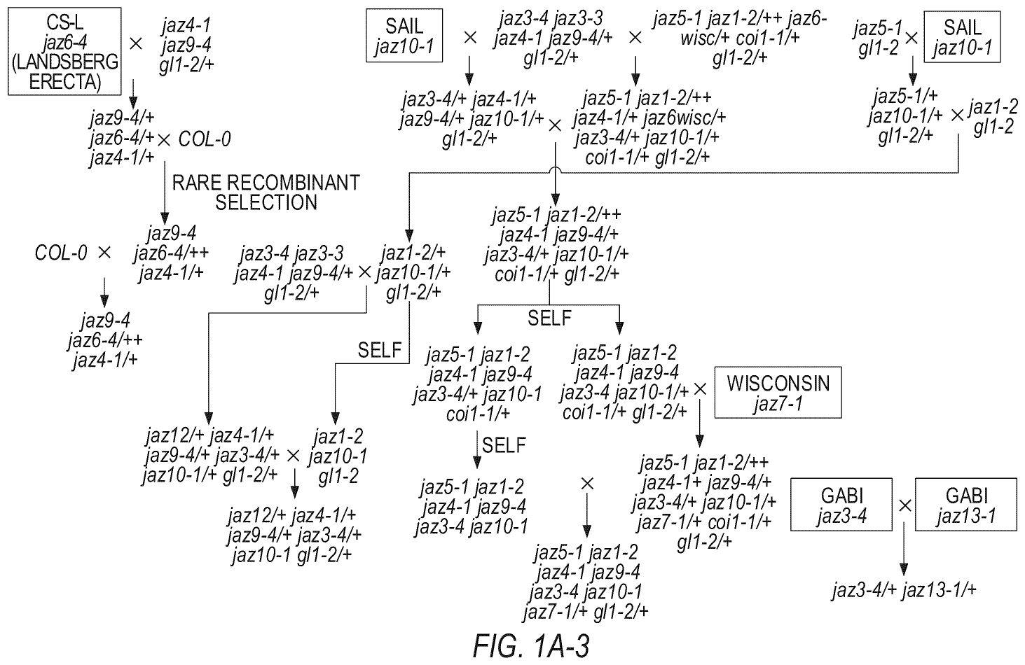

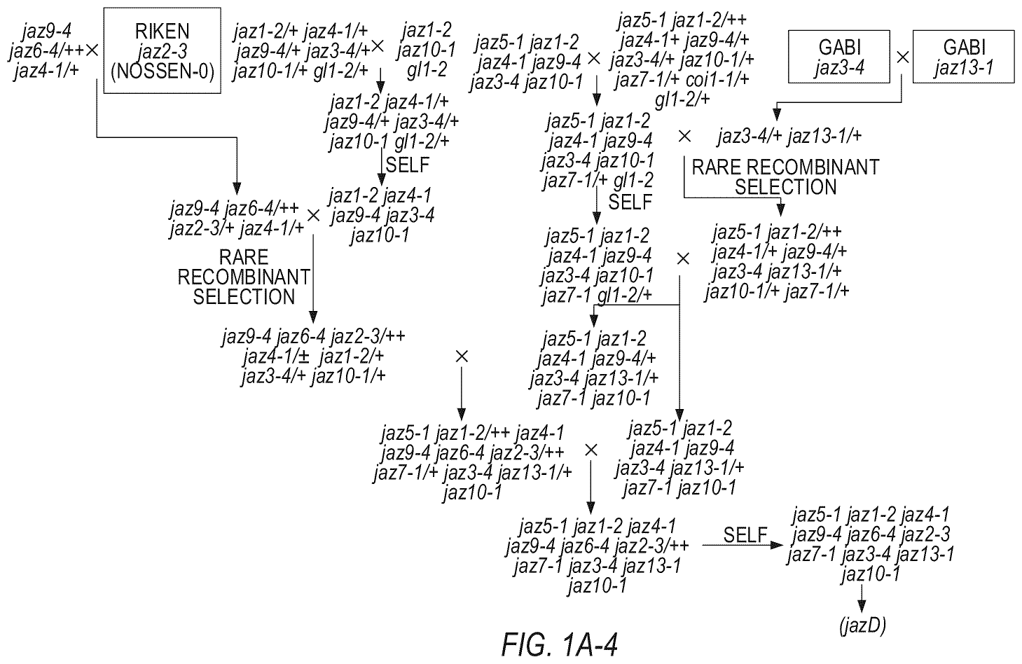

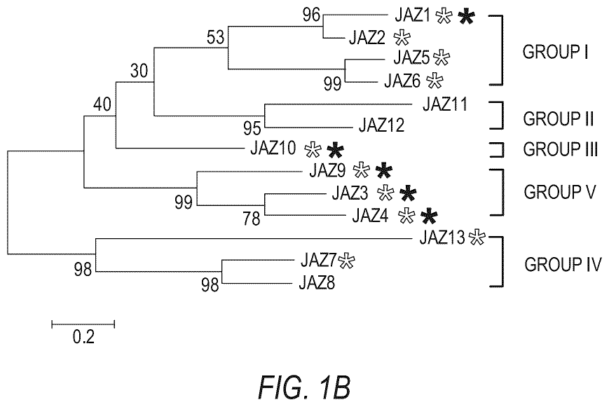

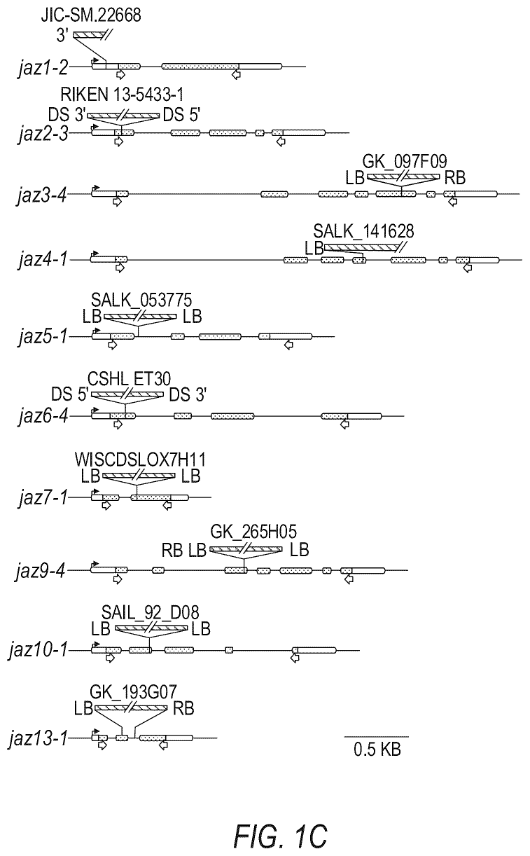

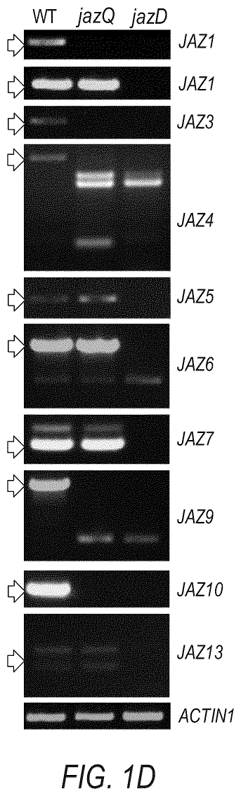

The Columbia accession (Col-0) of A. thaliana was used as wild type for all experiments. Plants with jazD were constructed by crossing jazQ (Campos et al., Nat. Commun. 7: 12570 (2016)) to other transfer DNA (T-DNA) or transposon insertion mutants obtained from the Arabidopsis Biological Research Center (ABRC; Ohio State University). The following jaz-single mutants were combined with jazQ as described in FIG. 1A-1D, and were named as follows: jaz2-3(RIKEN_13-5433-1) (Gimenez-Ibanez et al. New Phytol 213:1378-1392 (2017)), jaz5-1 (SALK_053775) (Thines et al. Nature 448:661-665 (2007)), jaz6-4 (CSHL_ET30) (described herein), jaz7-1 (WiscDsLox7H11) (Thines et al. Nature 448:661-665 (2007)), jaz8-V (Thireault et al., PlantJ82:669-679 (2015)), and jaz13-1 (GK_193G07) (Thireault et al., PlantJ82:669-679 (2015)). As illustrated in FIG. 1D, these jazD mutations eliminate transcription from Jaz1, Jaz2, Jaz3, Jaz4, Jaz5, Jaz6, Jaz7, Jaz9, Jaz10 and Jaz13 genes. Although an amplicon appears in the Jaz4 gel, this amplicon is unrelated to Jaz4 and does not indicate that a Jaz4 transcript was expressed.

Additional details onjaz-single mutants and the breeding scheme used to obtain jazD are provided in Table 3 and FIG. 1, respectively.

TABLE 3
Mutants used for construction of jazD and jazU.

| Mutant | Original name | Source | Accession | Mutagen | Resistance¹ |
|---|---|---|---|---|---|
| jaz1-2 | SM _3.22668 | JIC SM | Col-0 | dSpm transposon | Basta (confirmed) |
| jaz2-3 | RIKEN_13-5433-1 | RIKEN | No-0 | Ds transposon | Hygromycin |
| | | | | | (confirmed) |
| jaz3-4 | GK-097F09 | GABI Kat | Col-0 | T-DNA (pAC161) | Sulfadiazine |
| | | | | | (confirmed) |
| jaz4-1 | SALK_141628 | SALK | Col-0 | T-DNA | Kanamycin |
| | | | | (pROK2) | (silenced) |
| jaz5-1 | SALK_053775 | SALK | Col-0 | T-DNA | Kanamycin |
| | | | | (pROK2) | (confirmed) |
| jaz6-4 | CSHL_ET30 | CSHL | Ler | Ds transposon | Kanamycin |
| | | | | (Enhancer trap GUS) | (confirmed) |
| jaz7-1 | WiscDsLox7H11 | Wisconsin | Col-0 | T-DNA | Basta |
| | | | | (pWiscDsLox) | (not tested) |
| jaz8-V² | N/A | ABRC | Vash-1 | SNP | N/A |
| jaz9-4 | GK_265H05 | GABI kat | Col-0 | T-DNA | Sulfadiazine |
| | | | | (pAC161) | (confirmed) |
| jaz10-1 | SAIL_92_D08 | SAIL | Col-0 | T-DNA | Basta |
| | | | | (pCSA110) | (confirmed) |
| | | | | | GUS |
| jaz13-1 | GK_193G07 | GABI kat | Col-0 | T-DNA | Sulfadiazine |
| | | | | (pAC161) | (not tested) |

¹Resistance of the mutant line to the indicated selectable marker was tested and confirmed.
²The C-to-A nonsense mutation present in JAZ8 from accession Vash-1 was backcrossed four times to Col-0 to generate a line (#28-6-30) that was used for subsequent genetic crosses (Thireault et al., Plant J 82: 669-679 (2015)).
N/A, not applicable.

Efforts were made to reduce chromosomal contributions from other accessions by testing multiple SSLP polymorphic markers over many generations, so that the majority of jazD genome is derived from Col-0 (FIG. 1). Following sowing of seeds in soil, potted plants were covered with a transparent plastic dome for 10 days. Soil-grown plants were maintained under a 16-hour light (100 μE m⁻²s⁻¹) and 8-hour dark photoperiod at 20° C. unless otherwise noted. Immediately after seed harvest, small seeds were eliminated by passing bulk seed through a brass sieve with a 250 m pore size. Seeds retained after sieving (referred to as “sieved seeds”) were dried for two weeks in 1.5 mL Eppendorf tubes containing Drierite desiccant. PCR analysis PCR-based genotyping of jazD and lower-order mutants was performed using primer sets flanking DNA insertion sites and a third primer recognizing the T-DNA border (Table 4).

TABLE 4
Primers used for genotyping

| Gene | Locus | Primer | Sequence (5′-3′) |
|---|---|---|---|
| JAZ1 | AT1G19180 | JAZ1_F | ACCGAGACACATTCCCGATT |
| | | | (SEQ ID NO: 67) |
| | | JAZ1_R | CATCAGGCTTGCATGCCATT |
| | | | (SEQ ID NO: 68) |
| | | JAZ1_border | ACGAATAAGAGCGTCCATTTTAGAG |
| | | | (SEQ ID NO: 69) |
| JAZ2 | AT1G74950 | JAZ2_F | TCTTCCTCGTGACAAAACGCA |
| | | | (SEQ ID NO: 70) |
| | | JAZ2_R | CCAAACACAGAACCATCTCCACA |
| | | | (SEQ ID NO: 71) |
| | | JAZ2_border | CCGGATCGTATCGGTTTTCG |
| | | | (SEQ ID NO: 72) |
| JAZ3 | AT3G17860 | JAZ3_F | ACGGTTCCTCTATGCCTCAAGTC |
| | | | (SEQ ID NO: 73) |
| | | JAZ3_R | GTGGAGTGGTCTAAAGCAACCTTC |
| | | | (SEQ ID NO: 74) |
| | | JAZ3_border | ATAACGCTGCGGACATCTACATT |
| | | | (SEQ ID NO: 75) |
| JAZ4 | AT1G48500 | JAZ4_F | TCAGGAAGACAGAGTGTTCCC |
| | | | (SEQ ID NO: 76) |
| | | JAZ4_R | TGCGTTTCTCTAAGAACCGAG |
| | | | (SEQ ID NO: 77) |
| | | JAZ4_border | TTGGGTGATGGTTCACGTAG |
| | | | (SEQ ID NO: 78) |
| JAZ5 | AT1G17380 | JAZ5_F | GCTTATACCGAAACCCGATTCCAG |
| | | | (SEQ ID NO: 79) |
| | | JAZ5_R | GGCTCATTGAGATCAGGAAACCA |
| | | | (SEQ ID NO: 80) |
| | | JAZ5_border | TTGGGTGATGGTTCACGTAG |
| | | | (SEQ ID NO: 81) |
| JAZ6 | AT1G72450 | JAZ6_F | GACACACATCACTGTCACTTC |
| | | | (SEQ ID NO: 82) |
| | | JAZ6_R | AGTTTCTGAGGTCTCTACCTTC |
| | | | (SEQ ID NO: 83) |
| | | JAZ6_border | CCGTTTTGTATATCCCGTTTCCGT |
| | | | (SEQ ID NO: 84) |
| JAZ7 | AT2G34600 | JAZ7_F | ATGCGACTTGGAACTTCGCC |
| | | | (SEQ ID NO: 85) |
| | | JAZ7_R | GGAGGATCCGAACCGTCTG |
| | | | (SEQ ID NO: 86) |
| | | JAZ7_border | ACGTCCGCAATGTGTTATTA |
| | | | (SEQ ID NO: 87) |
| JAZ8 | AT1G30135 | JAZ8_F | TGTCCTAAGAGTCCGCCGTTGT |
| | | | (SEQ ID NO: 88) |
| | | JAZ8_R | TTTGGAGGATCCGACCCGTTTG |
| | | | (SEQ ID NO: 89) |
| JAZ9 | AT1G70700 | JAZ9_F | TACCGCATAATCATGGTCGTC |
| | | | (SEQ ID NO: 90) |
| | | JAZ9_R | TCATGCTCATTGCATTAGTCG |
| | | | (SEQ ID NO: 91) |
| | | JAZ9_border | CTTTGAAGACGTGGTTGGAACG |
| | | | (SEQ ID NO: 92) |
| JAZ10 | AT15G13220 | JAZ10_F | ATTTCTCGATCGCCGTCGTAGT-3 |
| | | | (SEQ ID NO: 93) |
| | | JAZ10_R | GCCAAAGAGCTTTGGTCTTAGAGTG |
| | | | (SEQ ID NO: 94) |
| | | JAZ10_border | GTCTAAGCGTCAATTTGTTTACACC |
| | | | (SEQ ID NO: 95) |
| JAZ13 | AT3G22275 | JAZ13_F | GCACGTGACCAAATTTGCAGA |
| | | | (SEQ ID NO: 96) |
| | | JAZ13_R | TGAAGAGAGGAGGATGATGAGGA |
| | | | (SEQ ID NO: 97) |
| | | JAZ13_border | AAACCTCCTCGGATTCCATTGC |
| | | | (SEQ ID NO: 98) |

PCR reactions were performed with the following condition: 95° C. for 5 min, followed by 35 cycles of denaturation (30 s at 95° C.), annealing (30 s at 56° C.) and elongation (1.5 min at 72° C.). Final elongation step was performed at 72° C. for 10 min and completed reactions were maintained at 12° C. The jaz8-V mutant was distinguished from wild-type JAZ8 amplicons by digestion with AflII (New England Biolabs). The presence or absence of full-length JAZ transcripts in Col-0, jazQ, and jazD plants was determined by reverse transcription (RT) PCR. RNA was extracted from rosette leaves of soil-grown plants using a RNeasy kit (Qiagen). cDNA was reverse transcribed with a High-Capacity cDNA Reverse Transcription kit (Applied Biosystems, ABI). RT-PCR reactions were performed with primer sets designed to amplify target JAZ genes and the internal control ACTIN1 (At2g37620) by GoTaq Green Master Mix (Promega). Primer sets and additional details of the RT-PCR procedures are provided in Table 5.

TABLE 5
Primers used for RT-PCR

| | | | | Annealing | PCR |
|---|---|---|---|---|---|
| Gene | Locus | Primer | Sequence (5′-3′) | (° C.) | cycles |
| JAZ1 | AT1G19180 | JAZl_RT_F | ATGTCGAGTTCTAT | 52 | 30 |
| | | | GGAATG (SEQ ID NO: 99) | | |
| | | JAZ1_RT_R | TCATATTTCAGCTGC | | |
| | | | TAAAC (SEQ ID NO: 100) | | |
| JAZ2 | AT1G74950 | JAZ2_RT_F | ATGTCGAGTTTTTCT | 52 | 30 |
| | | | GCCGA (SEQ ID NO: 101) | | |
| | | JAZ2_RT_R | TTACCGTGAACTGA | | |
| | | | GCCAAG (SEQ ID NO: 102) | | |
| JAZ3 | AT3G17860 | JAZ3_RT_F | ATGGAGAGAGATTT | 52 | 30 |
| | | | TCTCGGG (SEQ ID NO: 103) | | |
| | | JAZ3_RT_R | TTAGGTTGCAGAGC | | |
| | | | TGAGAGAAG (SEQ ID NO: 104) | | |
| JAZ4 | AT1G48500 | JAZ4_RT_F | ATGGAGAGAGATTT | 64.7 | 40 |
| | | | TCTCGGGCTGG (SEQ ID NO: 105) | | |
| | | JAZ4_RT_R | TTAGTGCAGATGAT | | |
| | | | GAGCTGGAGGA (SEQ ID NO: 106) | | |
| JAZ5 | AT1G17380 | JAZ5_RT_F | ATGTCGTCGAGCAA | 54 | 35 |
| | | | TGAAAA (SEQ ID NO: 107) | | |
| | | JAZ5_RT_R | CTATAGCCTTAGAT | | |
| | | | CGAGAT (SEQ ID NO: 108) | | |
| JAZ6 | ATIG72450 | JAZ6RT_F | ATGTCAACGGGACA | 54 | 35 |
| | | | AGCGC (SEQ ID NO: 109) | | |
| | | JAZ6_RT_R | CTAAAGCTTGAGTT | | |
| | | | CAAGGT (SEQ ID NO: 110) | | |
| JAZ7 | AT2G34600 | JAZ7_RT_F | ATGATCATCATCAT | 58 | 40 |
| | | | CAAAAACTG (SEQ ID NO: 111) | | |
| | | JAZ7_RT_R | CTATCGGTAACGGT | | |
| | | | GGTAAG (SEQ ID NO: 112) | | |
| JAZ9 | AT1G70700 | JAZ9_RT_F | ATGGAAAGAGATTT | 52 | 40 |
| | | | TCTGGG (SEQ ID NO: 113) | | |
| | | JAZ9_RT_R | TTATGTAGGAGAAG | | |
| | | | TAGAAG (SEQ ID NO: 114) | | |
| JAZ10 | AT5G13220 | JAZ10_RT_F | ATGTCGAAAGCTAC | 57 | 40 |
| | | | CATAGAAC (SEQ ID NO: 115) | | |
| | | JAZ10_RT_R | GATAGTAAGGAGAT | | |
| | | | GTTGATACTAATCTCT | | |
| | | | (SEQ ID NO: 116) | | |
| JAZ13 | AT3G22275 | JAZ13_RT_F | ATGAAGGGTTGCAG | 56 | 35 |
| | | | CTTAGA (SEQ ID NO: 117) | | |
| | | JAZ13_RT_R | TTAGAAATTATGAA | | |
| | | | GAGAGGAGG (SEQ ID NO: 118) | | |
| ACTIN1 | AT2G37620 | Actin1_F | ATGGCTGATGGTGA | 67.2 | 40 |
| | | | AGACATTCAA (SEQ ID NO: 119) | | |
| | | Actinl_R | TCAGAAGCACTTCC | | |
| | | | TGTGAACAAT (SEQ ID NO: 120) | | |

### Growth Measurements

For relative growth rate (RGR) analysis, five plants per genotype were harvested every two days beginning and ending 11 and 29 days, respectively, after seed sowing. Excised shoots were lyophilized for determination of dry weight. Relative growth rate (RGR) was calculated from the slope of the log(dry weight) over the duration of the time course. Leaf area of 23-day-old plants was determined by photographing rosettes from the top with a Nikon D80 camera. The resulting images were used to measure projected leaf area with GIMP software (see website at gimp.org).

### Root Elongation Assays

Seeds were surface sterilized with 50% (v/v) bleach for three min, washed 10 times with sterile water and stratified in dark at 4° C. for two days. Seedlings were grown on 0.7% (w/v) agar media containing half-strength Linsmaier and Skoog (LS; Caisson Labs) salts supplemented with 0.8% (w/v) sucrose and the indicated concentration of MeJA (Sigma-Aldrich). Each square Petri plate (Fisher; 100×100×15 mm) contained five seedlings per genotype. Plates were incubated vertically in a growth chamber maintained at 21° C. for eight days under 16-hour-light (80 μE m⁻²s⁻¹)/8-hour-dark conditions. The length of primary roots was measured using ImageJ software (see website at imagej.nih.gov/ij/).

### Coronatine Treatment

The eighth true leaf of 40-day-old plants grown under 12-hour-light/12-hour-dark conditions were spotted with 5 μL of sterile water (mock) or a solution containing 50 μM coronatine (Sigma-Aldrich, C8115) prepared in sterile water. Photographs were taken two and four days after treatment.

### Insect and Pathogen Assays

Insect feeding assays were performed at 20° C. under a short-day photoperiod of 8-hour light and 16-hour dark. Neonate Trichoplusia ni larvae (Benzon Research) were transferred to fully expanded rosette leaves of 9-week-old plants. Four larvae were reared on each of 12 plants for approximately 12 days, after which larval weights were measured (Herde et al. Methods Mol Biol 1011:51-61 (2013)). Botrytis cinerea bioassays were performed as described previously (Rowe et al. Mol Plant Microbe Interact 20:1126-1137 (2007)), with minor modifications. Detached leaves from 10-week-old short-day-grown (8-hour light/16-hour dark) plants were placed in Petri dishes containing filter paper moistened with 10 mL sterile water, with petioles submerged in the water. Each leaflet was inoculated with a single 4 μL droplet of Botrytis cinereal spore suspension (5,000 spores/mL in 50% organic grape juice). Petri dishes were sealed with Micropore surgical tape (3M Health Care) and kept under the same conditions used for plant growth. Photographs were taken after five days and lesion area was measured using the ImageJ software (see website at imagej.nih.gov/ij/).

### Seed Yield Measurements

Individual plants were grown in 6.5-cm square pots. An inverted plastic cone and plastic tube (Arasystem 360 kit; Arasystem) were fitted to each plant 23 days after seed sowing to collect all seeds from dehiscing siliques. Seeds collected from individual plants were harvested and dried with Drierite desiccant for two weeks, after which total seed mass per plant was measured. Average seed mass was determined by weighing dry seeds in batches of 200 (Jofuku et al., Proc Natl Acad Sci USA 102:3117-3122 (2005)). For each plant, the weights of three sample batches were measured and averaged. The silique length and number of seeds per silique were measured by sampling the fully-elongated seventh, ninth and eleventh siliques on the main stem (Roux et al., Genetics 166:449-460 (2004)).

### Germination Assays

Germination assays were performed on half-strength LS agar plates without sucrose. Unsieved seeds were surface sterilized and stratified in dark at 4° C. for two days. Plates were incubated vertically under continuous light at 21° C. and germination was scored daily for seven days by radicle emergence from the seed coat (Dekkers et al., Planta 218:579-588 (2004)).

### RNA-Seq Analysis

Global gene expression profiling was performed on the Illumina HiSeq 2000 platform at the Michigan State University Research Technologies Service Facility (see website at rtsf.natsci.msu.edu/). Rosettes of 23-day-old soil-grown Col-0, jazQ, and jazD plants were harvested for RNA extraction 6 h after the beginning of the light period. Three independent RNA samples (biological replicates) were used for each genotype, with each replicate derived from pooling rosette leaves from 20 plants. Raw sequencing reads were filtered with Illumina quality control tool FASTX-Toolkit (see website at hannonlab.cshl.edu/fastx_toolkit/) and then mapped to TAIR10 gene models by RSEM (version 1.2.25) (Li et al., BMC Bioinformatics 12:323 (2011)). mRNA abundances for all Arabidopsis genes were expressed as transcripts per million (TPM). The average TPM ±s.e.m for all genes is shown in Dataset S1, sheet a. DESeq2 (version 3.3) (Anders, Genome Biol 11:R106 (2010)) was used to normalize expected counts from RSEM and to determine differential gene expression by comparing normalized counts in Col-0 to those in mutants. DAVID (version 6.8) (Huang et al., Nat Protoc 4:44-57 (2009)) and MapMan (version 3.6.0) (Thimm et al., The Plant 37:914-939 (2004)) was used to perform gene ontology (GO) analysis of enriched functional categories. Over-represented and under-represented GO categories among differentially expressed genes were assessed by hypergeometric test with Benjamini & Hochberg's false discovery rate (FDR) correction at P<0.05. Analysis of the induction or repression of metabolic pathways was performed by Kyoto Encyclopedia of Genes and Genomes (KEGG) Mapper (see website at genome.jp/kegg/pathway.html) (Kanehisa & Goto, Nucleic Acids Res 28:27-30 (2000)). Data deposition: RNA sequencing data from this study have been deposited in the Gene Expression Omnibus (CEO) database, see website at ncbi.nlm.nih.gov/geo (accession no. GSE 1 16681).

### Quantitative Proteomic Analysis

Quantitative proteomic analysis was performed with proteins extracted from leaf tissue of 23-day-old soil-grown Col-0 and jazD plants. Proteins from three biological replicates (20 plants/replicate) of each genotype were extracted with the following extraction buffer: 100 mM Tris-HCl (pH 6.8), 150 mM NaCl, 10% glycerol (v/v), 4% SDS (w/v), 200 mM DTT, and protease inhibitor (Sigma-Aldrich, 1 tablet/10 mL buffer). Protein concentrations were determined by Bradford assay. Trypsin-digested peptides derived from these proteins were derivatized with a tandem mass tag (TMT) labeling kit (ThermoFisher) for quantification by mass spectrometry (MS) performed at the Michigan State University Proteomics Core Facility (see website at rtsf.natsci.msu.edu/proteomics/). Briefly, protein samples were digested with trypsin using the Filter-Aided Sample Preparation (FASP) protocol according to Wisniewski et al. (Nat Methods 6:359-362 (2009)). Samples were then labeled with TMTsixple Isobaric Label Reagents (ThermoFisher) according to manufacturer's protocol. After labeling, all six samples were combined and dried by vacuum centrifugation. The combined peptide samples were separated over a pH gradient (pH 3-10) into six fractions using an Agilent OffGel 3100 fractionator (www.agilent.com) according to manufacturer's protocol. Dried fractions were washed and eluted. Eluted peptides were sprayed into a ThermoFisher Q-Exactive mass spectrometer (www.thermo.com) using a FlexSpray nano-spray ion source. Survey scans were taken in the Orbitrap (70,000 resolution, determined at m/z 200) and the top ten ions in each survey scan were then subjected to automatic higher energy collision induced dissociation (HCD) with fragment spectra acquired at 35,000 resolution. Conversion of MS/MS spectra to peak lists and quantitation of TMT reporter ions was done using Proteome Discover, v1.4.1.14. Peptide-to-spectrum matching was performed with the Sequest HT and Mascot search algorithms against the TAIR10 protein sequence database appended with common laboratory contaminants (downloaded from the website arabidopsis.org and thegpm.org, respectively). The output from both search algorithms was then combined and analyzed using Scaffold Q+S (version 4.5.3) to probabilistically validate protein identifications and quantification. Assignments validated using the Scaffold 1% FDR confidence filter were considered true.

### Gas Exchange Measurements and ¹³C Discrimination Analysis

Plants grown under short-day photoperiod (8 h light/16 h dark) in ‘Cone-tainers’ (Steuwe and Sons, Tangent, OR, USA) were used for gas exchange analysis. The measurements were performed on LI-6400XT and LI-6800 systems (LI-COR Biosciences, Lincoln, NE, USA) as described by Campos et al. (Nat Commun 7:12570 (2016)). Daytime respiration was determined from slope-intercept regression analysis of the common intersection of five CO₂ response curves (using intercellular CO₂ below 10 Pa) measured at decreasing, sub-saturating irradiances (Walker et al., Plant Cell Environ 38:2462-2474 (2015)). Leaf tissue was freeze-dried and used for the measurement of the ratio of ¹³CO₂ to ¹²CO₂ by mass spectrometry at the Stable Isotope Ratio Facility for Environmental Research, University of Utah (Salt Lake City, UT). Isotopic ratios and CO₂ partial pressure at Rubisco were calculated as described (Weraduwage et al. Front Plant Sci 6:167 (2015); Farquhar et al. Funct Plant Biol 9:121-137 (1982); Farquhar et al. Annu Rev Plant Biol 40:503-537 (1989)).

### Protein, Lipid and Cell Wall Measurements

For protein, lipid and cell wall measurements, leaf tissue was harvested from 23-day-old plants grown under our standard long-day conditions. Excised shoots were lyophilized to determine the dry weight. Total protein was extracted using a Plant Total Protein Extraction Kit (PE0230, Sigma-Aldrich) and quantified by Bradford assay. Lipid extraction, thin-layer chromatography (TLC) of polar and neutral lipids, transesterification, and gas chromatography were performed as described previously (Wang & Benning, J Vis Exp 49:2518 (2011); Wang et al. Plant Cell (2018)). For polar lipids, lipid separation was performed by activated ammonium sulfate-impregnated silica gel TLC plates (TLC Silica gel 60, EMD Chemical) with a solvent consisting of acetone, toluene, and water (91:30:7.5 by volume). Lipids were visualized by brief exposure to iodine vapor on TLC plates. Acyl groups of the isolated lipids were then converted to methyl esters, which were subsequently quantified by a gas chromatography. Cell wall was extracted with a solution containing 70% ethanol, chloroform/methanol solution (1:1 v/v) and acetone as described (Foster et al. J Vis Exp 37:1837 (2010)). Starch was removed from the extracts using amylase and pullulanase (Sigma-Aldrich). Protein, lipid and cell wall content was normalized to leaf dry weight.

### Glucosinolate Measurements

Plants were grown under long-day conditions (16-hour day and 8-hour night) for 23 days. Rosette leaves were harvested and frozen in liquid nitrogen immediately. Two plants were pooled for each sample, with three biological replicates collected per sample. Frozen tissue was homogenized with a TissueLyser II (Qiagen) and glucosinolates were extracted following published procedures (Glauser et al. Phytochem Anal 23:520-528 (2012)), with minor modifications. Briefly, 80% methanol (v/v) was added to homogenized tissues and the mixture was vortexed for 5 min. Extracts were then centrifuged at 16,000×g for 5 min and the supernatant was transferred to a 2-mL glass vial (RESTEK). Samples were analyzed in the MSU Mass Spectrometry Facility by ultrahigh pressure liquid chromatography (UPLC) coupled to quadrupole time-of-flight mass spectrometry (QTOFMS) using Waters Xevo G2-XS. Data analysis and processing were performed as described previously (Glauser et al. Phytochem Anal 23:520-528 (2012)).

### Sucrose Rescue Assays

The effect of exogenous sucrose on leaf biomass and root growth was determined by growing seedlings on square Petri plates (Greiner Bio-One; 120×120×17 mm). In order to control for variation in seed quality, seeds were sieved after drying with desiccant for two weeks (see above). After sterilization and washing, seeds were sown without stratification on 0.7% (w/v) agar media containing half-strength LS salts supplemented with sucrose or sorbitol. Each plate contained ten (for biomass) or five (for root growth) seeds of Col-0 and mutant lines. Plates were placed in the dark at 4° C. for four days and then incubated horizontally (for leaf biomass) or vertically (for root growth) in growth chambers maintained at 21° C. under 16 h at a light intensity of 80 μE m⁻² s⁻¹ and 8-hour dark. ImageJ was used to measure root length after 11 days. Plant biomass and projected leaf area were measured after 16 days.

### Example 2: Reduced Growth and Fertility of a jazD Mutant Is Associated with Extreme Sensitivity to JA

This Example describes the growth and fertility of the jazD mutant plants.

The insertion mutations used to construct a series of higher-order jaz mutants are shown in FIG. 1 with which to interrogate the biological consequences of chronic JAZ deficiency in Arabidopsis. The 13-member JAZ family in Arabidopsis is comprised of five phylogenetic groups (I-V) that are common to angiosperms (FIG. 1). The jazQ mutant harbors mutations in the sole member (JAZ10) of group III, all three members of group V (JAZ3, JAZ4, JAZ9), and one member (JAZ1) of the largest group I clade. Building on the jazQ chassis, the inventors used genetic crosses to introduce five additional mutations that target the remaining group I members (JAZ2, JAZ5, JAZ6) and two genes (JAZ7 and JAZ13) within group IV (FIG. 1B-1D). The resulting homozygous jaz1-jaz7, jaz9, jaz10, jaz13 decuple mutant, referred to hereafter as jazD, thus targets all JAZs except for JAZ8 and the two group II genes (JAZ11 and JAZ12).

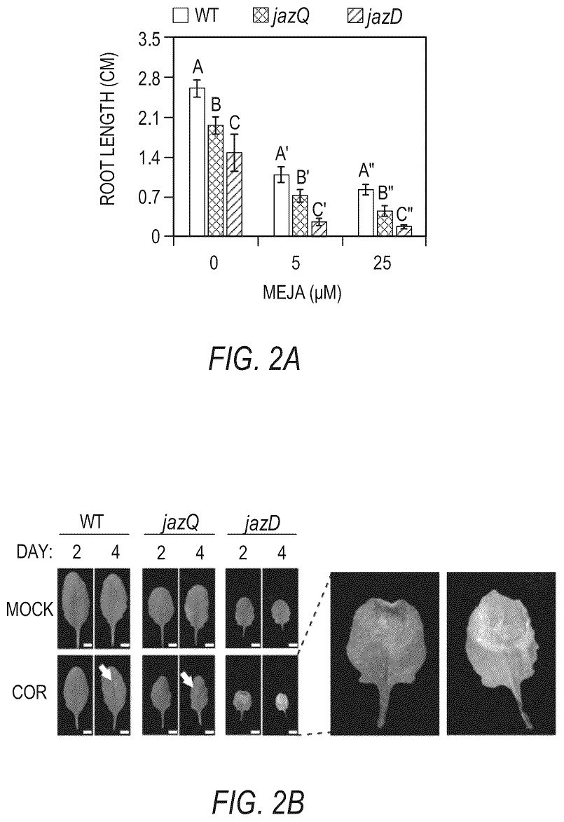

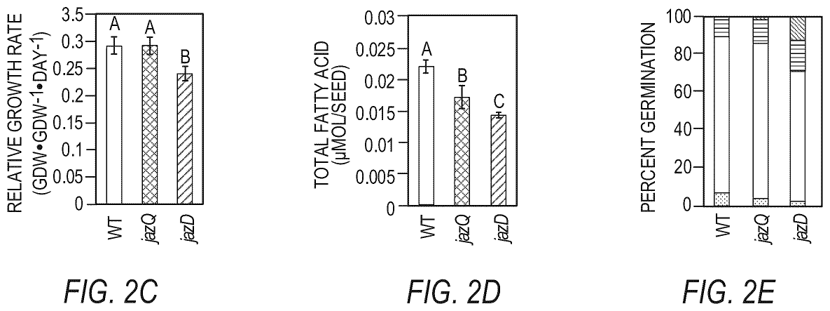

Cultivation of plants in the absence of exogenous jasmonic acid showed that, whereas jazQ roots and leaves grow more slowly than wild type (WT) Col-0, growth of jazD plants was even slower than jazQ (FIG. 2A-2B). Soil-grown jazD plants displayed less leaf area and shorter petioles than jazQ, and also accumulated more anthocyanins (FIG. 2B). Leaf biomass measurements taken over a 20-day time course confirmed that the relative growth rate (RGR) of jazD rosettes during this developmental stage was significantly less than wild type (FIG. 2C).

The relative growth rate (RGR) of jazQ was comparable to wild type, despite the reduced biomass of jazQ rosettes at later times in development, which may reflect growth changes occurring before the first time point of sampling (11 days after sowing) or the lack of statistical power needed to resolve small differences in RGR that are compounded over time into larger differences in rosette size. Although bulk protein, lipid, and cell wall content of rosette leaves were similar between all three genotypes under the growth conditions employed, the ratio of leaf dry weight (DW) to fresh weight was increased in jazD relative to wild type and jazQ.

The restricted growth of jazD roots and leaves was associated with changes in flowering time under long-day growth conditions. The jazD plants were delayed in their time-to-flowering compared with jazQ but contained a comparable number of leaves at the time of bolting.

The response of jazQ and jazD mutants was next compared to exogenous jasmonic acid. Root growth assays showed that the extent of JAZ deficiency, where jazD has more than jazQ and JazQ has more than wild type, was inversely correlated with root length under a range of MeJA concentrations (FIG. 2A). The growth of jazD roots effectively arrested in the presence of 5 μM MeJA (FIG. 2A).

Shoot responsiveness to the hormone was assessed by treating intact leaves with coronatine (COR), which is a potent agonist of the JA-Ile receptor. Wild type and jazQ leaves exhibited visible accumulation of anthocyanin pigments at the site of COR application (i.e., midvein) within 4 days of the treatment, with no apparent signs of chlorosis (FIG. 2B). In contrast, jazD leaves exhibited visible chlorosis at the site of COR application within 2 days of treatment and, strikingly, near complete loss of chlorophyll and spreading of necrosis-like symptoms throughout the leaf 4 days after treatment, leading to tissue death (FIG. 2B).

These data indicate that progressive loss of JAZ genes in jazQ and jazD results in both quantitative (e.g., root growth inhibition) and qualitative (e.g., COR-induced tissue necrosis) differences in jasmonate responsiveness. These results also indicate that the hypersensitivity of jazD results, at least in part, from loss of JAZ-mediated negative-feedback control of JA responses.

Measurements of reproductive output showed that, whereas the total seed yield of jazQ was only marginally affected, seed production by jazD plummeted to about one-third of wild type levels (Table 6).

TABLE 6
Seed and fruit production in higher-order jaz mutants

| | Seed yield | Average | Silique | No. seed | |
|---|---|---|---|---|---|
| | per plant^{†} | seed mass^{‡} | length^{§} | per | No. silique |
| Genotype | (mg) | (μg) | (cm) | silique^{§} | per plant^{§} |
| WT | 608.3 ± 103.8 | 21.6 ± 1.3 | 1.59 ± 0.07 | 63 ± 11 | 451 ± 77 |
| jazQ | 524.3 ± 98.5 | 17.3 ± 0.9* | 1.70 ± 0.06 | 58 ± 6 | 533 ± 100 |
| jazD | 192.7 ± 70.0* | 16.6 ± 0.7* | 1.45 ± 0.08* | 37 ± 4* | 329 ± 119* |

Data show the mean ± SD of at least 10 plants per genotype.
Asterisks denote significant difference compared with WT plants according to Tukey's HSD test (*P < 0.05).
^{†}Seed yield was determined by collecting all seeds from individual WT Col-0 and jaz mutant plants.
^{‡}Average seed mass was determined by weighing batches of 200 seeds.
^{§}Fully elongated 7th, 9th, and 11th siliques were collected for measurements of silique traits. These traits were used to calculate the estimated number of siliques per plant.

The reduced fecundity of jazD resulted from a combination of decreased average mass per seed and lower total seed number per plant. Mutant plants produced fewer seeds per silique, and the size and number of siliques per plant were reduced as well (Table 4). The reduced size of jazD seeds correlated with a reduction in total fatty acid per seed (FIG. 2D). Analysis of seed fatty acid profiles showed that jazQ and jazD seeds contain less oleic acid (18:1) and more linoleic acid (18:2), indicating that alterations in fatty acid metabolism occur in these jaz mutants during seed development.

The effect of jazD on seed size and lipid abundance was associated with reduced rates of seed germination (FIG. 2E). These findings indicate that constitutive jasmonic acid responses resulting from JAZ depletion are associated with poor reproductive performance.

### Example 3: Constitutive Activation of JA-Mediated and Ethylene-Mediated Defense Pathways in jazD Plants

Having established the effects of jazQ and jazD on growth and reproduction, in this Example the inventors assessed how these mutations impact JA-mediated signaling pathways for defense.

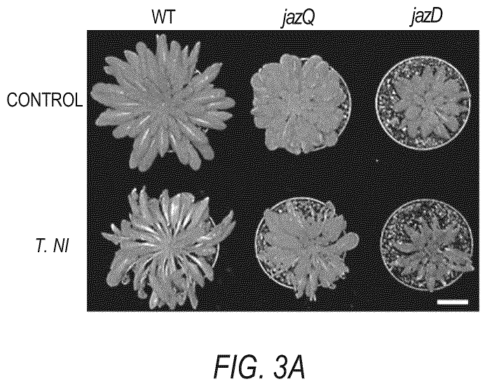

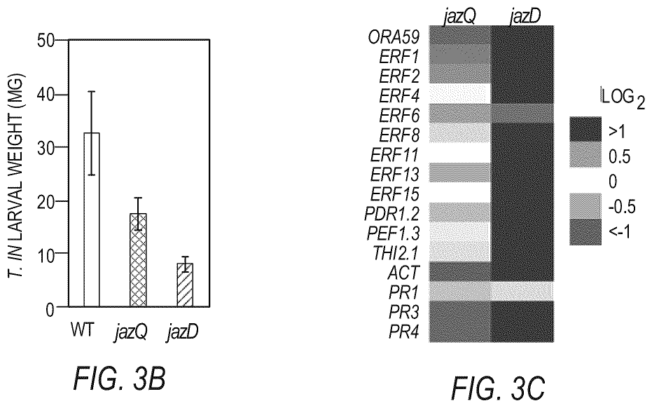

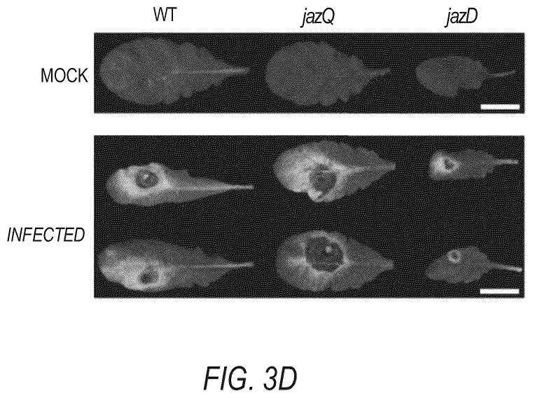

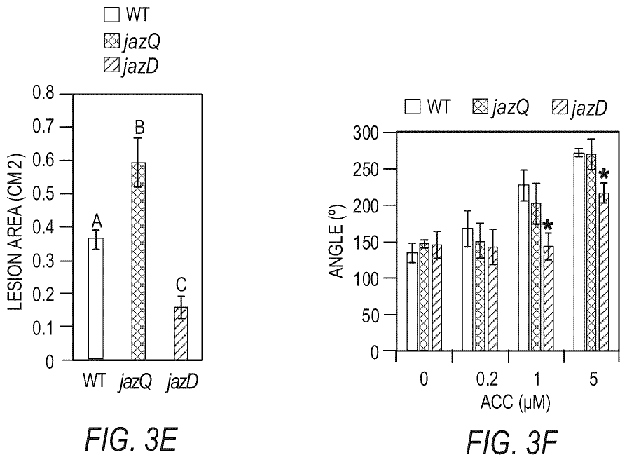

Short-day conditions were used to promote leaf biomass and delay flowering in plants used for insect bioassays. Under such short-day conditions jaz-mediated leaf growth restriction was observed (FIG. 3A). Insect bioassays were performed with the generalist herbivore Trichoplusia ni.

As shown in FIGS. 3A-3B, the strength of host resistance to insect feeding positively correlated with the severity of jaz mutation, where the insect resistance of jazD plants was greater than jazQ plants, and the insect resistance of jazQ plants was greater than that of wild type plants. These results are consistent with a role for JAZ proteins in the negative regulation of defense (FIG. 3A-3B).

Messenger RNA sequencing (RNA-seq) was used to investigate the molecular basis of the enhanced anti-insect resistance. Global transcript profiles revealed that the total number of differentially expressed genes in jazD leaves (relative to wild type) was more than 10-fold greater than that in jazQ (2,107 for jazD and 186 for jazQ). Among the 186 genes whose expression was statistically different in the jazQ vs. wild type comparison, the majority (59%) of these were also differentially expressed in jazD. Gene Ontology (GO) analysis of 1,290 genes expressed to higher levels in jazD than WT showed that “response to JA/wounding,” as well as “defense response,” were among the biological processes most statistically over-represented in this comparison. These results, together with analysis of metabolic pathways that are differentially activated in jaz mutants (see below), indicate that the strength of anti-insect resistance correlates with the extent of JAZ deficiency and concomitant reprogramming of gene expression.

Analysis of the RNA-seq data also revealed that ethylene-response genes were highly expressed in jazD but not jazQ. For example, antifungal defense genes controlled by the synergistic action of JA and ethylene were modestly repressed in jazQ but induced in jazD (FIG. 3C). Among these were genes encoding the AP2/ERFs ERF1 and ORA59, which integrate JA and ethylene signals to promote the expression of antimicrobial compounds, including various defensins (PDFs), pathogenesis-related (PR) proteins, and hydroxycinnamic acid amides (HCAAs) (FIG. 3C). Strikingly, several PDF transcripts (e.g., PDF1.2) were among the most abundant of all mRNAs in jazD leaves, with expression levels comparable to that of the most highly expressed photosynthesis transcripts.

In agreement with the RNA-seq data, jazQ plants were slightly more susceptible than wild type to the necrotrophic pathogen Botrytis cinerea, whereas jazD leaves were more resistant to the spread of disease lesions (FIG. 3D-3D). To determine whether jazQ and jazD differentially affect other ethylene responses, the inventors assessed apical hook formation in ethylene-elicited seedlings. Consistent with studies showing that apical hook formation is attenuated by JA signaling (Song et al. Plant Cell 26:263-279 (2014)), FIG. 3F shows that stimulation of hook curvature in response to treatment with the ethylene precursor 1-aminocyclopropane-1-carboxylic acid (ACC) was reduced in jazD but not jazQ seedlings. These data indicate that whereas jazQ moderately activates JA responses and increases resistance to insect feeding, jazD strongly induces both the JA and ethylene branches of immunity to confer robust resistance to insect feeding and infection by B. cinerea.

To validate the RNA-seq results and gain additional insight how jazD promotes leaf defense, the inventors used quantitative tandem mass spectrometry to quantify global changes in protein abundance in jazD leaves vs. wild type leaves. Among a total of 4,850 unique proteins identified in both genotypes, 149 accumulated to higher in jazD leaves while 120 proteins accumulated to lower levels in jazD leaves (threshold fold-change >1.2, P<0.05). GO analysis of the 120 down-regulated proteins revealed enrichment of functional categories related to cytokinin response, cold response, and various functional domains of photosynthesis (Table 5A-5B).

Table 7A-7B list biological processes in which proteins whose abundance in jazD leaves was increased or decreased in comparison to wild-type Col-0 based on gene ontology (GO) analysis. Enriched functional categories were determined with DAVID (version 6.8) using the hypergeometric test with Benjamini & Hochberg's false discovery rate (FDR) correction.

TABLE 7A
Upregulated in jazD

| GO ID | GO description | P value |
|---|---|---|
| 0009695 | jasmonic acid biosynthetic process | <0.0001 |
| 0055114 | oxidation-reduction process | <0.0001 |
| 0009611 | response to wounding | <0.0001 |
| 0009651 | response to salt stress | <0.0001 |
| 0009753 | response to jasmonic acid | <0.0001 |
| 0008652 | cellular amino acid biosynthetic | <0.0001 |
| | process | |
| 0000162 | tryptophan biosynthetic process | <0.0001 |
| 0050832 | defense response to fungus | <0.0001 |
| 0006952 | defense response | 0.0002 |
| 0019762 | glucosinolate catabolic process | 0.0010 |
| 0006564 | serine biosynthetic process | 0.0113 |
| 0080027 | response to herbivore | 0.0226 |
| 0009414 | response to water deprivation | 0.0336 |

TABLE 7B
Downregulated jazD

| GO ID | GO description | P value |
|---|---|---|
| 0009735 | response to cytokinin | <0.0001 |
| 0015979 | photosynthesis | <0.0001 |
| 0009409 | response to cold | <0.0001 |
| 0010207 | photosystem II assembly | 0.0001 |
| 0019684 | photosynthesis, light reaction | 0.0079 |
| 0042549 | photosystem II stabilization | 0.0239 |
| 0042742 | defense response to bacterium | 0.0257 |

Analysis of proteins that were more abundant in jazD showed there was good agreement with the corresponding mRNA levels determined by RNA-seq; transcripts encoding 78% of these 149 proteins were also induced in jazD plants. As expected, there was strong enrichment in this protein set of GO categories associated with response to JA, herbivore, and fungal attack, among other defense-related processes (Table 7A-7B). For example, the proteomic analysis revealed that jazD coordinately up-regulated the abundance of most JA biosynthetic enzymes, as well as canonical JA marker proteins, such as VSP1 and VSP2.

Leaves from jazD plants exhibited high expression levels of an agmatine coumaroyl transferase (At5g61160) and an associated transporter (At3g23550) involved in the production of antifungal HCAAs. Transcripts encoding the acyl-CoA N-acyltransferase NATA1 (At2g39030), which catalyzes the formation of the defense compound N(6)-acetylornithine, were 50-fold higher in jazD leaves compared with leaves from wild type and jazQ plants. Such expression was accompanied by increased NATA1 protein abundance. Perhaps most striking was the up-regulation in jazD leaves, at both the mRNA and protein levels, of most known structural and enzymatic components of the endoplasmic reticulum (ER)-derived ER body, which is implicated in induced immunity (Nakano et al. Plant J 89: 204-220 (2017); Yamada et al. Plant Cell Physiol 52:2039-2049 (2011)). These findings establish a central role for JAZ proteins as negative regulators of diverse leaf defense traits.

### Example 4: Reprogramming of Primary and Specialized Metabolism in jazD Plants

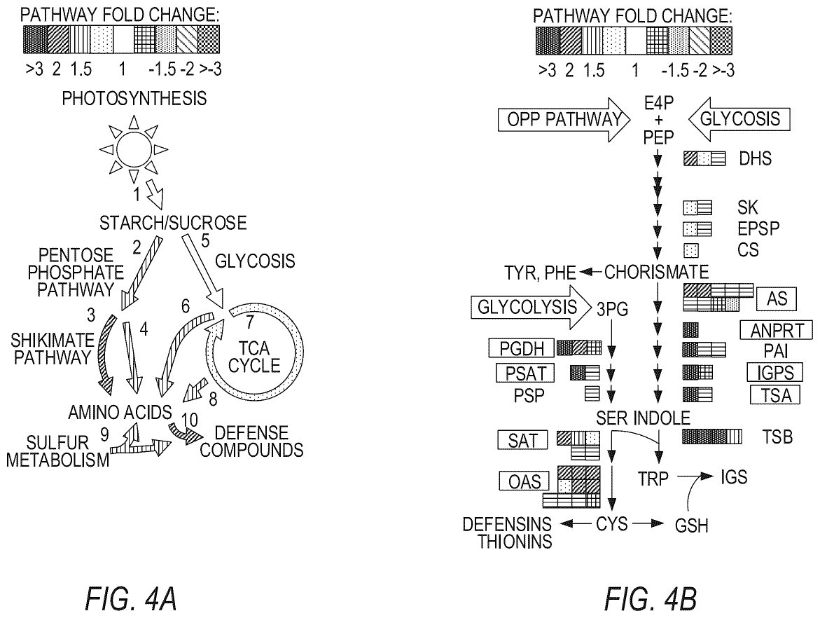

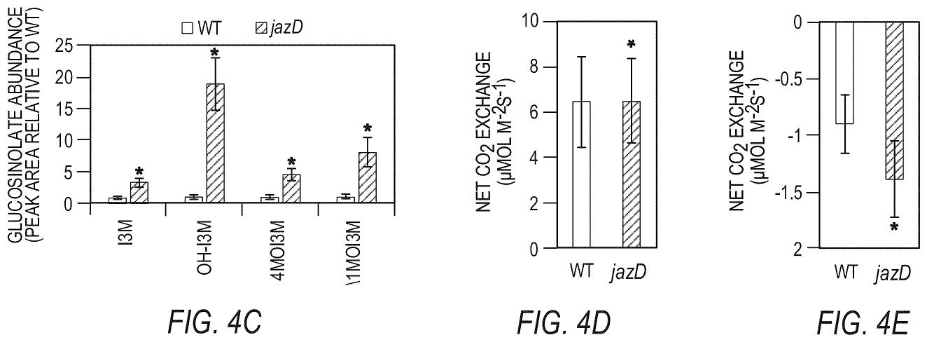

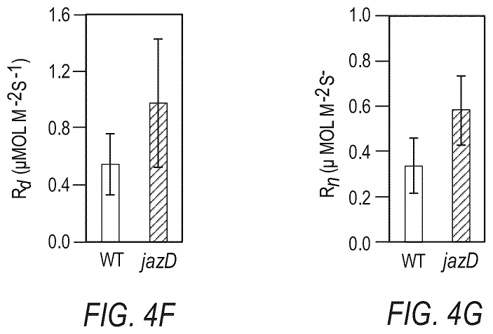

To investigate how the activation of multiple defense pathways influences primary metabolism, RNA-seq and proteomics data were used to infer metabolic pathways that are altered in jazD leaves. Mapping of differentially expressed genes to Kyoto Encyclopedia of Genes and Genomes pathway databases showed that the tricarboxylic acid (TCA) cycle, oxidative pentose phosphate pathway, sulfur assimilation and metabolism, and various amino acid biosynthetic pathways were among the processes most highly induced in jazD, whereas photosynthesis components were generally down-regulated (FIG. 4A).

One prominent example of a metabolic pathway that was upregulated in jazD was the shikimate pathway for the biosynthesis of aromatic amino acids. Trp biosynthetic enzymes involved in the production of indole glucosinolates (IGs) showed particularly high expression at the mRNA and protein levels (FIG. 4B). Consistent with this finding, genes encoding enzymes in the phosphoserine pathway that supplies Ser for the biosynthesis of Trp and Cys were highly up-regulated in jazD, as was the abundance of the corresponding enzymes as determined from proteomics data (FIG. 4B).

LC-MS analysis of leaf extracts showed that several indole glucosinolates accumulate to high levels in jazD (FIG. 4C), thereby validating the omics data. The inventors also found that pathways involved in sulfur assimilation and cysteine biosynthesis, as well as ascorbate and glutathione metabolic pathways that protect against oxidative stress, were strongly up-regulated in jazD (FIG. 4B). These data indicate that genetic depletion of JAZ proteins recapitulates the transcriptional effects of exogenous JA and demonstrate that JAZ proteins exert control over pathways that operate at the interface of primary and specialized metabolism.

The inventors then addressed the question of whether jazD modulates net carbon assimilation. Despite the down-regulation of photosynthetic mRNAs and proteins in jazD, modeling of photosynthetic parameters derived from gas-exchange data indicated that the leaf area-based photosynthetic rate of jazD plants was comparable to wild type (FIG. 4D). This finding was confirmed by ¹³C isotope discrimination measurements, which showed that the degree of CO₂ resistance through mesophyll cells was similar in WT, jazQ, and jazD leaves. In contrast to photosynthesis, the net loss of CO₂ from jazD leaves in the dark exceeded that of wild type by about 50% (FIG. 4E). Increased cellular respiration in jazD was confirmed by experiments showing that the mutant had increased respiration in both the day and night portions of the photoperiod (FIG. 4F-4G). These findings indicate that increased cellular respiration is associated with high-level production of defense compounds.

GO analysis of the 817 down-regulated genes in jazD leaves showed enrichment for growth-related processes, including “response to light stimulus,” “cell wall organization,” “response to abiotic stimulus,” “carbohydrate biosynthetic process,” and “lipid biosynthetic process.”

### Example 4: jazD Plants Exhibit Symptoms of Carbon Starvation

Increased respiration and partitioning of carbon to metabolic defense pathways, in the absence of compensatory changes in photosynthesis, raised the possibility that jazD plants have a carbon deficit.

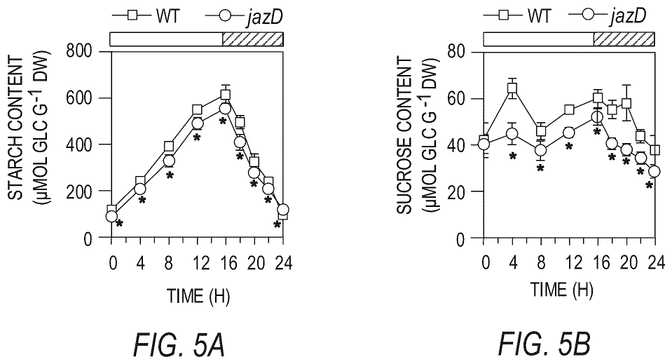

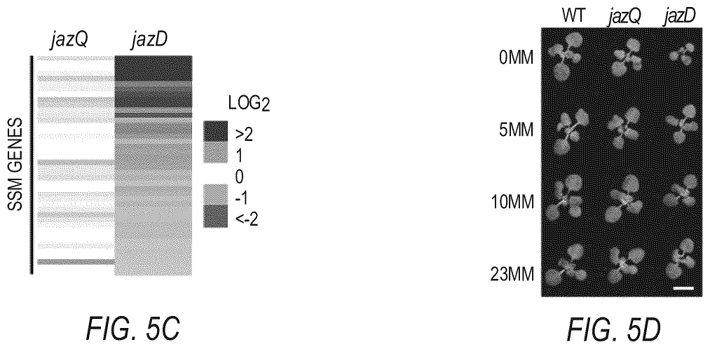

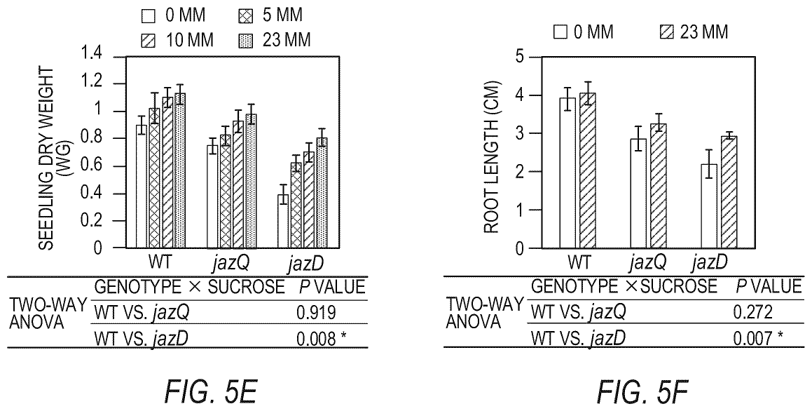

Time-course studies showed that the rates of starch accumulation (wild type: 0.103 μmol Glc g⁻¹ dry weight h⁻¹; jazD: 0.113 μmol Glc g⁻¹ dry weight h⁻¹) and degradation (WT: −0.220 g⁻¹ dry weight h⁻¹; jazD: −0.186 μmol Glc g⁻¹ dry weight h⁻¹) were comparable between wild type and jazD (FIG. 5A). However, starch levels in jazD leaves were slightly lower than wild type at all times of the diel cycle except at the end of the night, when starch was mostly depleted but modestly elevated in jazD relative to wild type. jazD leaves also had consistently lower sucrose levels (FIG. 5B). The inventors also found that genes involved in starch and sucrose metabolism were generally down-regulated in jazD, including the mRNA and protein abundance of the plastidic starch biosynthetic enzyme phosphoglucomutase (PGM1, At5g51820).

To test whether these changes in central metabolism are associated with carbon deficit, the RNA-seq data was used to query the expression of genes that are induced by conditions (e.g., prolonged darkness) leading to carbon starvation. The inventors found that 42 of 278 (15%) sugar starvation marker (SSM) genes defined by Baena-González et al. (Nature 448:938-942 (2007)), including several DARK INDUCIBLE (DIN) genes that respond to reduced energy status, were expressed to much higher levels in jazD than WT and jazQ (FIG. 5C).

The inventors also examined the expression of EIN3-regulated glutamate dehydrogenases (GDH) that replenish 2-oxoglutarate for the TCA cycle and are considered metabolic markers of carbon deficiency. Both the transcript and protein abundance of GDH1 (At5g18170) and GDH2 (At5g07440) were statistically increased in jazD in comparison with WT, consistent with a carbon deficit in this mutant.

To test the hypothesis that carbon limitation contributes to the slow growth of jaz mutants, the inventors compared the growth of WT, jazQ, and jazD seedlings on agar medium supplemented with sucrose. FIG. 5D-5E show that although exogenous sucrose promotes increased biomass in all genotypes tested, the stimulatory effect on the growth of jazD shoots was statistically greater than that of wild type and jazQ. Exogenous sucrose also enhanced the root growth of jazD in comparison with wild type and jazQ (FIG. 5F). Control experiments with sorbitol showed that the growth-promoting effect of sucrose was not attributed to changes in osmotic strength of the growth medium. These data provide evidence that the reduced growth of jazD but not jazQ results in part from a limitation in carbon supply.

### Example 5: A jaz1-jaz10 and jaz13 Undecuple Mutant Produces Few Viable Seeds

The ability of jazD plants to perceive and respond to exogenous jasmonate (JA) suggested that the remaining JAZ proteins in the mutant can actively repress JA-responsive genes. The inventors hypothesized that mutation of these remaining JAZ loci (i.e., JAZ8, JAZ11, and JAZ12) in the jazD background may further enhance the level of growth-defense antagonism. To test this, the inventors focused on JAZ8 because of its established role in repressing JA responses and the availability of a naturally occurring jaz8-null allele (Thireault et al. Plant J 82:669-679 (2015)). The increased expression of JAZ8 in jazD leaves (>15-fold relative to WT) was also consistent with a role in negative-feedback control of JA responses.

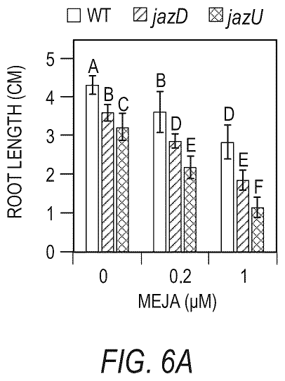

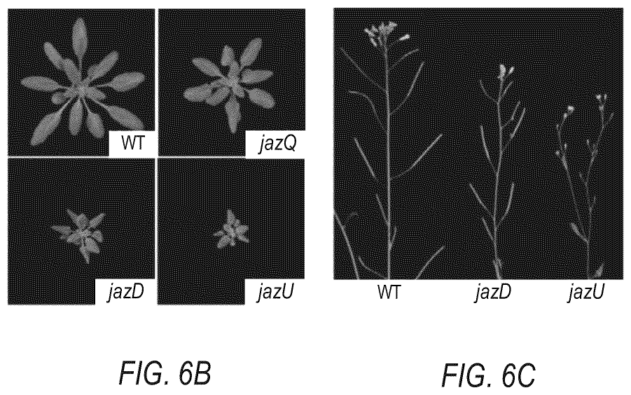

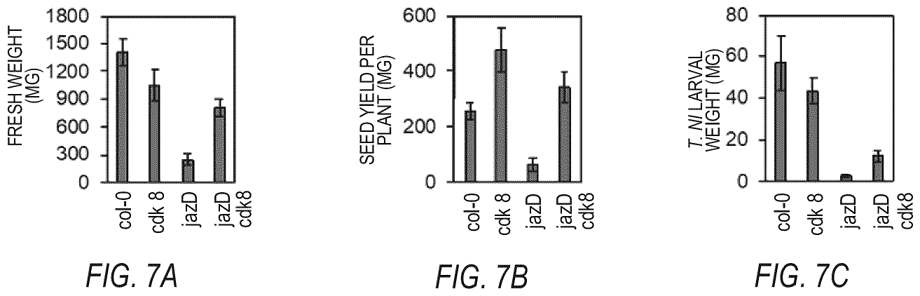

Screening of progeny derived from genetic crosses between jazD and jaz8 resulted in the identification of an undecuple mutant (jazU) homozygous for mutations in JAZ1-JAZ10 and JAZ13. Root growth assays showed that jazU roots were even shorter than jazD in the presence of very low concentrations (e.g., 1 μM) of MeJA (FIG. 7A). When grown on JA-free medium, jazU showed an even stronger constitutive short-root phenotype than jazD (FIG. 6A). Similarly, the rosette morphology of jazU confirmed the progressive effect of JAZ depletion on restriction of rosette growth, including reduced biomass, leaf area, and petiole length (FIG. 6B). Most strikingly, jazU plants exhibited near complete loss of viable seed production (FIG. 6C). Less than 3% of jazU flowers set fruit; although jazU pollen was viable in crosses, among flowers that produced fruit, most senesced and aborted during silique filling. Among the few jazU flowers that did produce seeds, seed set per silique was severely reduced, with recovery of only a few viable seeds per plant. The collective seed-yield phenotype of jazQ, jazD, and jazU supports a key role for JAZ proteins in promoting reproductive vigor.

### Example 6: CDK8 Mutation Restores Growth and Seed Yields of jaz8 Plants

This Example illustrates that cdk8 loss-of-function mutations improve the growth and seed yields of jazD plants.

The inventors used jazD in a genetic suppressor screen to identify 11 independent sjd (suppressor of jazD) mutants in which rosette growth was partially restored while maintaining enhanced production of defense compounds.

Genome sequencing revealed that one suppressor line (sjd56) carries a null mutation in CYLIN-DEPENDENT KINASE 8 (CDK8, also known as CDKE1 and At5G63610)), which encodes a component of the Mediator complex.

The cdk8 mutation not only partially restores vegetative growth but also fully recovers the low seed yield of jazD, while maintaining robust defense against insect herbivores (FIG. 7A-7C).

Fifteen sjd56-like F2 plants were generated from a cross between sjd56 and jazD parental lines. Sanger sequencing was performed on the genomes of the F2 progeny, demonstrating that each of the fifteen sjd56-like F2 plants had the C1684T mutation, shown in the nucleic acid segment provided below (SEQ ID NO:121).

CCTTCCACAC TGGCAAAATG ATGTTCAACA CATTCAAGCT
CACAAATACG ACAGTGTGGG TCTC

The sjd56 C1684T mutation truncates the CDK8 protein by altering a glutamine residue to a stop a codon.

To generate additional jazD plant lines that include the sjd56 CDK8 mutation, jazD (jaz1-SM, jaz2-RK, jaz3-GK, jaz4-1, jaz5-1, jaz6-DT, jaz7-1, jaz9-4, jaz10-1, jaz13-1) plants were crossed with T-DNA insertion CDK8 mutant lines, cdk8-1 or cdk8-2. The progeny of this screen were screened by PCR-genotyping using primer sets flanking DNA insertion sites and a third primer flanking the T-DNA border.

### Example 7: Null CDK8 Mutant Exhibits Increased Growth and Similar Defenses as jazD

This Example illustrates that jazD plants with a null CDK8 mutation (e.g., sjd56 plants) exhibit increased growth and improved resistance to insects compared to jazD and wild type plants.

Wild type Col-0 (WT), jazD and sjd56 plants were grown under different conditions.

In one experiment, the different plant types were grown under short-day (8-h-light/16-h-dark) conditions, and at 58 days of growth, the rosette fresh weight and projected leaf area of the different plant types was measured.

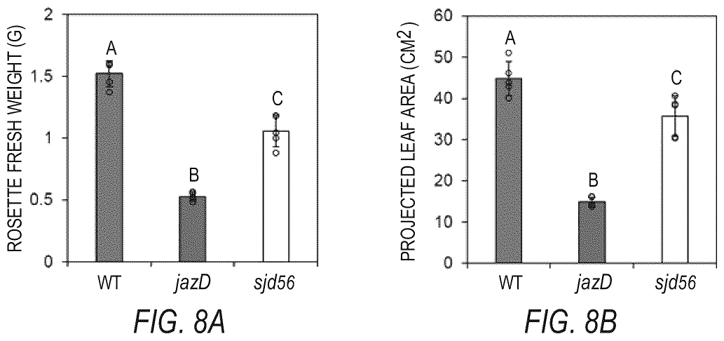

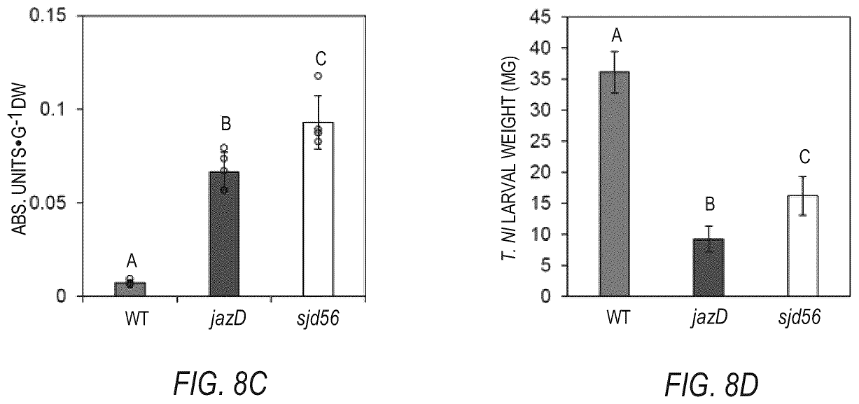

As shown in FIG. 8A-8B, the sjd56 plants exhibit greater rosette fresh weight and greater projected leaf area than the jazD plants, but somewhat less rosette fresh weight and less projected leaf area than wild type plants.

In another experiment, wild type Col-0 (WT), jazD and sjd56 plants were grown under long-day (16-h-light/8-h-dark) conditions, and at 23 days of growth anthocyanin levels were measured in the leaves of the different plant types.

FIG. 8C shows that the anthocyanin levels in leaves of sjd56 plants are significantly greater than in leaves of wild type Col-0 (WT) and jazD plants.

In a third experiment, wild type Col-0 (WT), jazD and sjd56 plants were grown under photoperiods of 16-h-light/8-h-dark for 67 days, and Trichoplusia ni (T. ni) were allowed to feed on the plants during the last ten days of growth. FIG. 8D shows the weight of Trichoplusia ni (T ni) after feeding for ten days. As illustrated, substantially more Trichoplusia ni (T ni) were present on wild type Col-0 (WT) and even on jazD plants than on the sjd56 plants.

### Example 8: Cdk8 Mutations Restore Growth and Reproduction while Delaying Vegetative and Reproductive Transitions of jazD

This Example illustrates that combining cdk8 null mutations overcomes the reduced growth observed in plants with the jazD genetic background.

The growth flowering and seed production of plants with jazD cdk8-1 and jazD cdk8-2 genotypes (generated as described in Example 6) were evaluated.

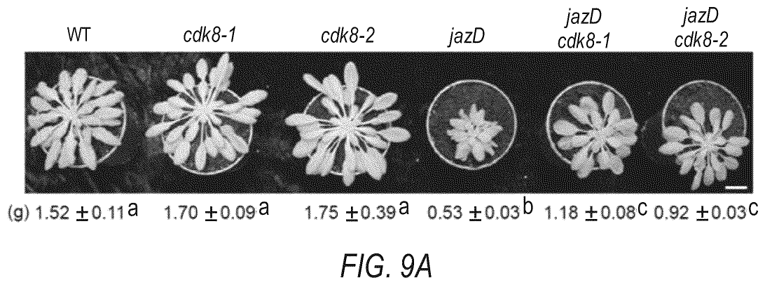

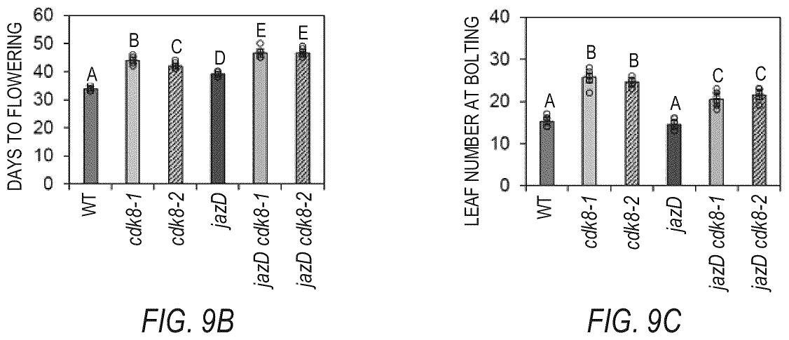

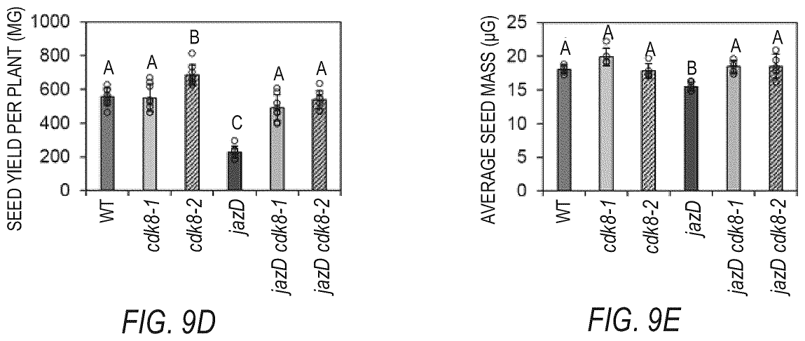

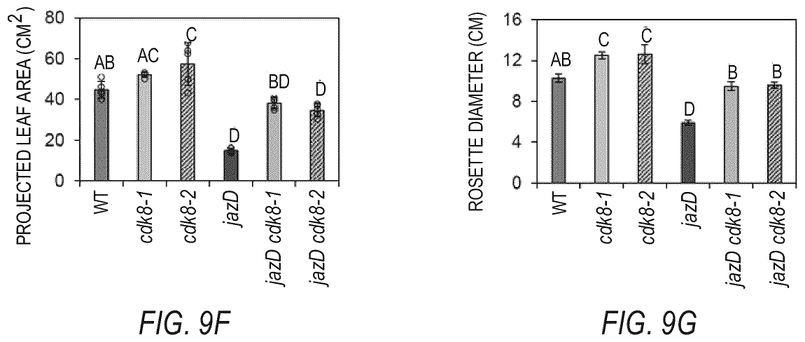

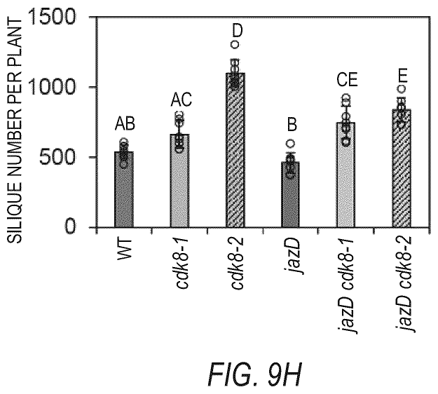

Col-0 (WT), cdk8-1, cdk8-2, jazD, jazD cdk8-1 and jazD cdk8-2 plants were grown under short-day conditions (8-h-ligh/16-h-dark) for 58 days, and the rosette fresh weights and leaf diameters were then measured. As illustrated in FIGS. 9A and 9G, the rosette fresh weights of jazD cdk8-1 and jazD cdk8-2 plants after 58 days of growth were significantly greater than the rosette fresh weights of jazD plants, approaching the rosette fresh weights of wild type plants. FIG. 9F graphically illustrates that loss of cdk8 increases leaf diameter in jazD plants.

In another experiment, plants were grown under long-day (16-h-light/8-h-dark) conditions in soil. The number of days to flowering and the bolting leaf numbers were then measured. FIG. 9B graphically illustrates that as compared to wild type or jazD plants, the time until the first flowers appear was slightly longer for plants with cdk8 null mutations, including the jazD cdk8-1 and jazD cdk8-2 plants. FIG. 9C shows the number of rosette leaves at the time of bolting is greater for cdk8-1, cdk8-2, jazD cdk8-1 and jazD cdk8-2 plants compared to wild type and jazD plants.

Seed yield and seed mass of WT, cdk8-1, cdk8-2, jazD, jazD cdk8-1 and jazD cdk8-2 plants were also measured. Seed numbers were evaluated by collecting all seeds from individual plants. Average seed mass was determined by weighing batches of 100 seeds.

As shown in FIG. 9D-9E, seed yield and seed mass for plants with cdk8 null mutations, including the jazD cdk8-1 and jazD cdk8-2 plants was greater than determined for jazD plants, and was similar to that observed for wild type plants.

Further studies indicate that although silique length and seeds per silique are about the same for jazD and jazD plants with null cdk8 mutations, the number of siliques per plant is greater for jazD cdk8-1 and jazD cdk8-2 plants than in wild type and jazD plants (FIG. 9H). Hence, loss of cdk8 can positively impact the reproduction of jazD plants.

### Example 9: Cdk8 Mutations Partially Recover the Defense Phenotypes of jazD

This Example illustrates the pest resistance provided by combining cdk8 null alleles into jazD plants.

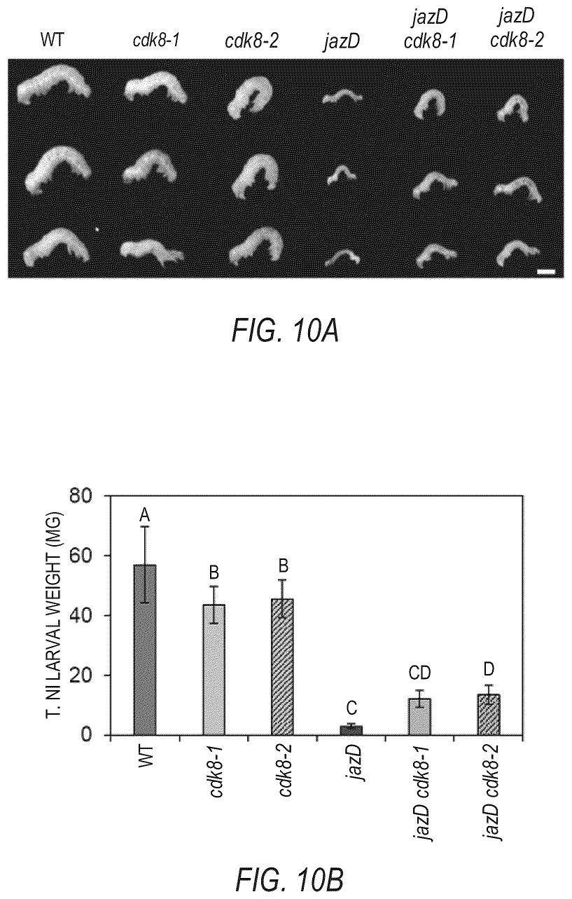

Trichoplusia ni (T. ni) larvae were allowed to feed on short-day-grown (8-h-light/16-h-dark) WT Col-0 (WT), cdk8-1, cdk8-2, jazD, jazD cdk8-1 and jazD cdk8-2 plants for nine days. FIG. 10A provides images of larvae isolated from the different plant types. As illustrated, larval sizes are significantly smaller when maintained on jazD, jazD cdk8-1 and jazD cdk8-2 plants than larvae maintained on wild type plants. FIG. 10B graphically illustrates the weights of larvae isolated from the different plant types. The data show the mean±SD of at least 18 larvae per genotype. As shown in FIG. 10B, larval weights are significantly less when the larvae feed on jazD, jazD cdk8-1 and jazD cdk8-2 plants.

### Example 10: The Increased Production of Defense Compounds in jazD is Partially Regulated by CDK8

This Example illustrates production of various plant defense compounds by jazD and jazD cdk8 plants.

Col-0 (WT), cdk8-1, jazD, and jazD cdk8-1 plants were grown under long-day conditions (16-h-light/8-h-dark) in soil. Defense compounds were extracted from leaves of 23-day-old plants grown under long-day conditions (16-h-light/8-h-dark).

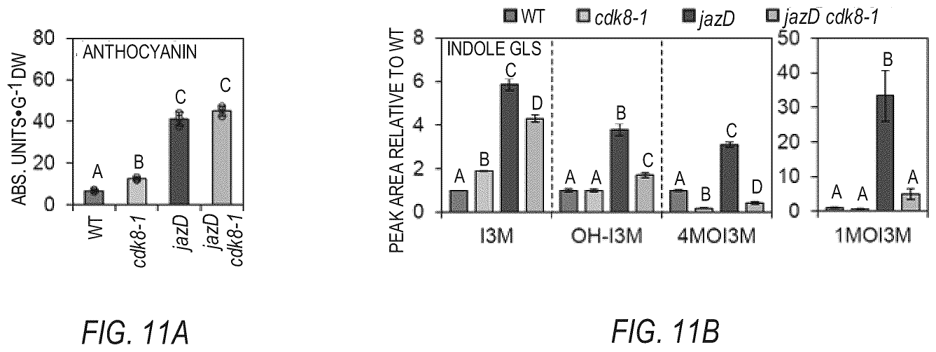

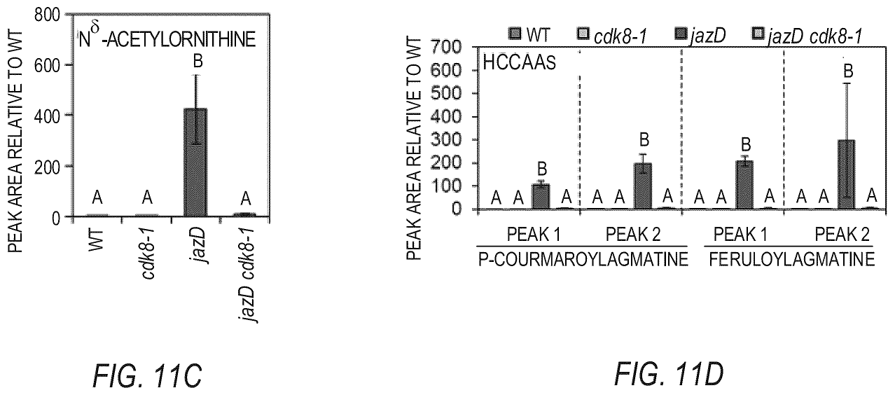

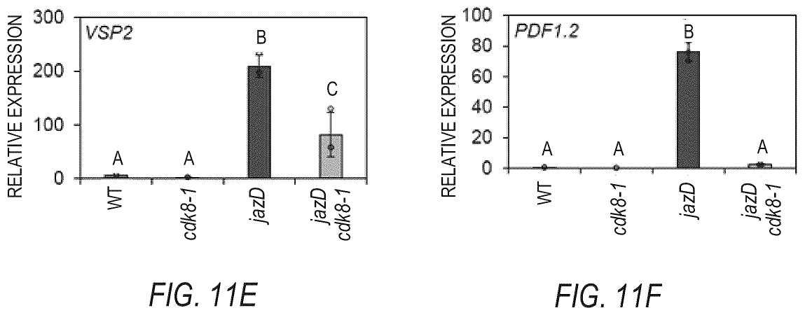

FIG. 11A graphically illustrates anthocyanin levels in leaves of 25-day-old wild type Col-0 (WT), cdk8, jazD and jazD cdk8 plants. FIG. 11B-11D graphically illustrate indole glucosinolates, Nδ-acetylornithine, and hydroxycinnamic acid amides (HCAAs) levels in WT, cdk8, jazD and jazD cdk8 leaves. Comparison of Peak area for the indicated compound in the WT sample was set to “1” and the peak area of the same compound in other genotypes was normalized to the WT sample. Abbreviations: I3M: indol-3-ylmethyl, glucobrassicin; OH-13M: 4-hydroxyindol-3-ylmethyl, hydroxyglucobrassicin; 4MOI3M: 4-methoxyindol-3-ylmethyl, methoxyglucobrassicin; 1MOI3M: 1-methoxyindol-3-ylmethyl, neoglucobrassicin. Data show the mean±SD of three biological replicates per genotype. Letters denote significant differences according to Tukey's HSD test (P<0.05).

In a second experiment, Col-0 (WT), cdk8-1, jazD, and jazD cdk8-1 plants were grown under long-day conditions (16-h-light/8-h-dark) in soil and leaves of 25-day-old were collected for quantitative PCR analysis.

FIG. 11E graphically illustrates relative expression levels of VEGETATIVE STORAGE PROTEIN 2 (VSP2, AT5G24770) while FIG. 11F graphically illustrates relative expression levels of PLANT DEFENSIN 1.2 (PDF1.2, AT5G44420). PP2A (AT1g13320) was used for qPCR normalization. Data show the mean±SD of three biological replicates per genotype. Letters denote significant differences according to Tukey's HSD test (P<0.05).

### Example 11: Cdk8 Mutations Promotes Production of Aliphatic Glucosinolates in jazD

This Example illustrates some of the compounds generated by leaves of plants of various genotypes, including the from leaves of jazD, cdk8, jazD and jazD cdk8 plants.

Aliphatic glucosinolates were extracted from leaves of 23-day-old plants grown under long-day conditions (16-h-light/8-h-dark). Peak area for the compound in the wild type (WT) sample was set to “1” and the peak area of the same compound in other genotypes was normalized to the WT sample.

FIG. 12 graphically illustrates aliphatic glucosinolate levels in WT, cdk8, jazD and jazD cdk8 leaves. The compounds detected included:
3MSOP: 3-methylsulphinylpropyl glucosinolate, glucoiberin;
4MSOB: 4-methylsulphinylbutyl glucosinolate, glucoraphanin;
5MSOP: 5-methylsulphinylpentyl glucosinolate, glucoalyssin;
6MSOH: 6-methylsulphinylhexyl glucosinolate, glucohesperin;
7MSOH: 7-methylsulphinylheptyl glucosinolate, glucoibarin;
3MTP: 3-methylthiopropyl glucosinolate, glucoiberverin;
8MSOO: 8-methylsulphinyloctyl glucosinolate, glucohirsutin;
4MTB: 4-methylthiobutyl glucosinolate, glucoerucin;
5MTP: 5-methylthiopentyl glucosinolate, glucoberteroin;
7MTH: 7-methylthioheptyl glucosinolate.
   The data shown in FIG. 12 are the mean±SD of three biological replicates per genotype, and the letters denote significant differences according to Tukey's HSD test (P<0.05).

### Example 12: Increased Resistance of jazD to 5-Methyl-Tryptophan (5-MT) is Partially Dependent on CDK8

This Example illustrates that loss of cdk8 further reduces jazD root lengths.

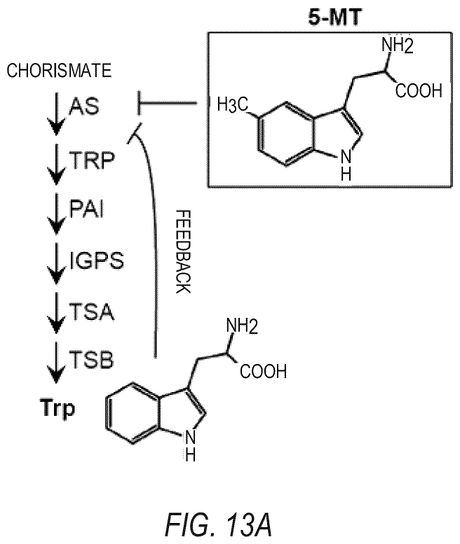

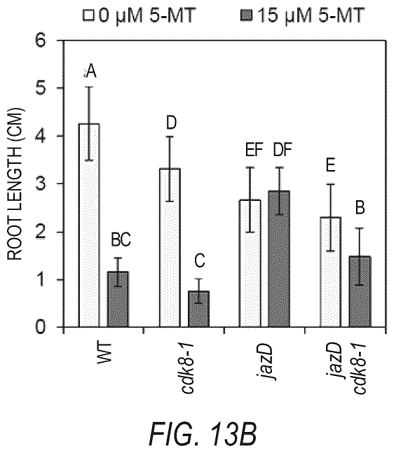

FIG. 13A is a schematic of tryptophan biosynthesis from chorismate. Tryptophan feedback inhibits the activity of anthranilate synthase (AS). Although 5-methyl-tryptophan (5-MT) inhibits anthranilate synthase activity, it cannot be used for the production of proteins. The abbreviations used in FIG. 13A are: TRP, anthranilate phosphoribosyltransferase; PAI, phosphoribosylanthranilate isomerase; IGPS, indole-3-glycerol-phosphate synthase; TSA, tryptophan synthase alpha subunit; TSB, tryptophan synthase beta subunit.

FIG. 13B graphically illustrates root length of WT, cdk8-1, jazD, and jazD cdk8-1 10-day-old seedlings grown on medium supplemented with 0 or 15 μM of 5-methyl-tryptophan (5-MT). The data shown in FIG. 13B are the mean±SD of at least 24 seedlings per genotype at each 5-MT concentration, while the letters denote significant differences according to Tukey's HSD test (P<0.05).

### REFERENCES

1. Pieterse CMJ, Leon-Reyes A, Van der Ent S, Van Wees SCM (2009) Networking by small-molecule hormones in plant immunity. Nat Chem Biol 5:308-316.
2. Santner A, Calderon-Villalobos LIA, Estelle M (2009) Plant hormones are versatile chemical regulators of plant growth. Nat Chem Biol 5:301-307.
3. Howe G A, Major I T, Koo A J (2018) Modularity in jasmonate signaling for multistress resilience. Annu Rev Plant Biol 69:387-415.
4. Wasternack C, Hause B (2013) Jasmonates: Biosynthesis, perception, signal transduction and action in plant stress response, growth and development. An update to the 2007 review in Annals of Botany. Ann Bot 111:1021-1058.
5. Campos M L, Kang J-H, Howe GA (2014) Jasmonate-triggered plant immunity. J Chem Ecol 40:657-675.
6. Howe G A, Jander G (2008) Plant immunity to insect herbivores. Annu Rev Plant Biol 59:41-66.
7. Wu J, Baldwin I T (2010) New insights into plant responses to the attack from insect herbivores. Annu Rev Genet 44:1-24.
8. Chini A, Gimenez-Ibanez S, Goossens A, Solano R (2016) Redundancy and specificity in jasmonate signalling. Curr Opin Plant Biol 33:147-156.
9. Yan Y, et al. (2007) A downstream mediator in the growth repression limb of the jasmonate pathway. Plant Cell 19:2470-2483.
10. Zhang Y, Turner J G (2008) Wound-induced endogenous jasmonates stunt plant growth by inhibiting mitosis. PLoS One 3:e3699.
11. Havko N E, et al. (2016) Control of carbon assimilation and partitioning by jasmonate: An accounting of growth-defense tradeoffs. Plants (Basel) 5:E7.
12. Major I T, et al. (2017) Regulation of growth-defense balance by the JASMONATE ZIM DOMAIN (JAZ)-MYC transcriptional module. New Phytol 215:1533-1547.
13. Attaran E, et al. (2014) Temporal dynamics of growth and photosynthesis suppression in response to jasmonate signaling. Plant Physiol 165:1302-1314.
14. Bömer M, et al. (Jun. 19, 2018) COI1-dependent jasmonate signalling affects growth, metabolite production and cell wall protein composition in Arabidopsis. Ann Bot, 10.1093/aob/mcy109.
15. Zust T, Agrawal A A (2017) Trade-offs between plant growth and defense against insect herbivory: An emerging mechanistic synthesis. Annu Rev Plant Biol 68:513-534.
16. Karasov T L, Chae E, Herman J J, Bergelson J (2017) Mechanisms to mitigate the tradeoff between growth and defense. Plant Cell 29:666-680.
17. Guo Q, Major I T, Howe G A (2018) Resolution of growth-defense conflict: Mechanistic insights from jasmonate signaling. Curr Opin Plant Biol 44:72-81.
18. Thines B, et al. (2007) JAZ repressor proteins are targets of the SCF(COI1) complex during jasmonate signalling. Nature 448:661-665.
19. Chini A, et al. (2007) The JAZ family of repressors is the missing link in jasmonate signalling. Nature 448:666-671.
20. Kazan K, Manners J M (2013) MYC2: The master in action. Mol Plant 6:686-703.
21. Fernández-Calvo P, et al. (2011) The Arabidopsis bHLH transcription factors MYC3 and MYC4 are targets of JAZ repressors and act additively with MYC2 in the activation of jasmonate responses. Plant Cell 23:701-715.
22. Qi T, Huang H, Song S, Xie D (2015) Regulation of jasmonate-mediated stamen development and seed production by a bHLH-MYB complex in Arabidopsis. Plant Cell 27:1620-1633.
23. Figueroa P, Browse J (2015) Male sterility in Arabidopsis induced by overexpression of a MYC5-SRDX chimeric repressor. Plant J 81:849-860.
24. Pauwels L, et al. (2010) NINJA connects the co-repressor TOPLESS to jasmonate signalling. Nature 464:788-791.
25. Shyu C, et al. (2012) JAZ8 lacks a canonical degron and has an EAR motif that mediates transcriptional repression of jasmonate responses in Arabidopsis. Plant Cell 24: 536-550.
26. Zhang F, et al. (2015) Structural basis of JAZ repression of MYC transcription factors in jasmonate signalling. Nature 525:269-273.
27. Qevik V, et al. (2012) MEDIATOR25 acts as an integrative hub for the regulation of jasmonate-responsive gene expression in Arabidopsis. Plant Physiol 160:541-555.
28. An C, et al. (2017) Mediator subunit MED25 links the jasmonate receptor to transcriptionally active chromatin. Proc Natl Acad Sci USA 114:E8930-E8939.
29. Katsir L, Schilmiller AL, Staswick PE, He SY, Howe GA (2008) COI1 is a critical component of a receptor for jasmonate and the bacterial virulence factor coronatine. Proc Natl Acad Sci USA 105:7100-7105.
30. Yan J, et al. (Aug. 7, 2018) Dynamic perception of jasmonates by the F-box protein COI1. Mol Plant, 10.1016/j.molp.2018.07.007.
31. Browse J (2009) Jasmonate passes muster: A receptor and targets for the defense hormone. Annu Rev Plant Biol 60:183-205.
32. Thireault C, et al. (2015) Repression of jasmonate signaling by a non-TIFY JAZ protein in Arabidopsis. Plant J 82:669-679.
33. Gimenez-Ibanez S, et al. (2017) JAZ2 controls stomata dynamics during bacterial invasion. New Phytol 213:1378-1392.
34. Li R, et al. (2017) Flower-specific jasmonate signaling regulates constitutive floral defenses in wild tobacco. Proc Natl Acad Sci USA 114:E7205-E7214.
35. Campos M L, et al. (2016) Rewiring of jasmonate and phytochrome B signalling uncouples plant growth-defense tradeoffs. Nat Commun 7:12570.
36. Berrocal-Lobo M, Molina A, Solano R (2002) Constitutive expression of ETHYLENERESPONSE-FACTORI in Arabidopsis confers resistance to several necrotrophic fungi. Plant J 29:23-32.
37. Pre M, et al. (2008) The AP2/ERF domain transcription factor ORA59 integrates jasmonic acid and ethylene signals in plant defense. Plant Physiol 147:1347-1357.
38. Li J, et al. (2018) Jasmonic acid/ethylene signaling coordinates hydroxycinnamic acid amides biosynthesis through ORA59 transcription factor. Plant J 95:444-457.
39. Song S, et al. (2014) Interaction between MYC2 and ETHYLENE INSENSITIVE3 modulates antagonism between jasmonate and ethylene signaling in Arabidopsis. Plant Cell 26:263-279.
40. Muroi A, et al. (2009) Accumulation of hydroxycinnamic acid amides induced by pathogen infection and identification of agmatine coumaroyl transferase in Arabidopsis thaliana. Planta 230:517-527.
41. Dobritzsch M, et al. (2016) MATE transporter-dependent export of hydroxycinnamic acid amides. Plant Cell 28:583-596.
42. Adio A M, et al. (2011) Biosynthesis and defensive function of Nδ-acetylornithine, a jasmonate-induced Arabidopsis metabolite. Plant Cell 23:3303-3318.
43. Nakano R T, et al. (2017) PYK10 myrosinase reveals a functional coordination between endoplasmic reticulum bodies and glucosinolates in Arabidopsis thaliana. Plant J 89: 204-220.
44. Yamada K, Hara-Nishimura I, Nishimura M (2011) Unique defense strategy by the endoplasmic reticulum body in plants. Plant Cell Physiol 52:2039-2049.
45. Benstein R M, et al. (2013) Arabidopsis phosphoglycerate dehydrogenasel of the phosphoserine pathway is essential for development and required for ammonium assimilation and tryptophan biosynthesis. Plant Cell 25:5011-5029.
46. Kruse C, et al. (2007) Sulfur-enhanced defense: Effects of sulfur metabolism, nitrogen supply, and pathogen lifestyle. Plant Biol (Stuttg) 9:608-619.
47. Sasaki-Sekimoto Y, et al. (2005) Coordinated activation of metabolic pathways for antioxidants and defense compounds by jasmonates and their roles in stress tolerance in Arabidopsis. Plant J 44:653-668.
48. Yatusevich R, et al. (2010) Genes of primary sulfate assimilation are part of the glucosinolates biosynthetic network in Arabidopsis thaliana. Plant J 62:1-11.
49. Bolton M D (2009) Primary metabolism and plant defense-Fuel for the fire. Mol Plant Microbe Interact 22:487-497.
50. Baena-Gonzilez E, Rolland F, Thevelein J M, Sheen J (2007) A central integrator of transcription networks in plant stress and energy signalling. Nature 448:938-942.
51. Fujiki Y, et al. (2001) Dark-inducible genes from Arabidopsis thaliana are associated with leaf senescence and repressed by sugars. Physiol Plant 111:345-352.
52. Gibon Y, et al. (2009) Adjustment of growth, starch turnover, protein content and central metabolism to a decrease of the carbon supply when Arabidopsis is grown in very short photoperiods. Plant Cell Environ 32:859-874.
53. Tsai K-J, Lin C-Y, Ting C-Y, Shih M-C(2016) Ethylene-regulated glutamate dehydrogenase fine-tunes metabolism during anoxia reoxygenation. Plant Physiol 172: 1548-1562.
54. Miyashita Y, Good AG (2008) NAD(H)-dependent glutamate dehydrogenase is essential for the survival of Arabidopsis thaliana during dark-induced carbon starvation. J Exp Bot 59:667-680.
55. Jin J, et al. (2017) Plant TFDB 4.0: Toward a central hub for transcription factors and regulatory interactions in plants. Nucleic Acids Res 45:D1040-D1045.
56. Zhu Z, et al. (2011) Derepression of ethylene-stabilized transcription factors (EIN3/EIL1) mediates jasmonate and ethylene signaling synergy in Arabidopsis. Proc Natl Acad Sci USA 108:12539-12544.
57. Solano R, Stepanova A, Chao Q, Ecker JR (1998) Nuclear events in ethylene signaling: A transcriptional cascade mediated by ETHYLENE-INSENSITIVE3 and ETHYLENE-RESPONSEFACTORI. Genes Dev 12:3703-3714.
58. Müller M, Munné-Bosch S (2015) Ethylene response factors: A key regulatory hub in hormone and stress signaling. Plant Physiol 169:32-41.
59. Lorenzo O, Chico JM, Sánchez-Serrano JJ, Solano R (2004) JASMONATE-INSENSITIVE1 encodes a MYC transcription factor essential to discriminate between different jasmonate-regulated defense responses in Arabidopsis. Plant Cell 16:1938-1950.
60. Caarls L, et al. (2017) Arabidopsis JASMONATE-INDUCED OXYGENASES downregulate plant immunity by hydroxylation and inactivation of the hormone jasmonic acid. Proc Natl Acad Sci USA 114:6388-6393.
61. Smirnova E, et al. (2017) Jasmonic acid oxidase 2 hydroxylates jasmonic acid and represses basal defense and resistance responses against Botrytis cinerea infection. Mol Plant 10:1159-1173.
62. Schweizer F, et al. (2013) Arabidopsis basic helix-loop-helix transcription factors MYC2, MYC3, and MYC4 regulate glucosinolate biosynthesis, insect performance, and feeding behavior. Plant Cell 25:3117-3132.
63. Gigolashvili T, et al. (2007) The transcription factor HIG1/MYB51 regulates indolic glucosinolate biosynthesis in Arabidopsis thaliana. Plant J 50:886-901.
64. Yang D-L, et al. (2012) Plant hormone jasmonate prioritizes defense overgrowth by interfering with gibberellin signaling cascade. Proc Natl Acad Sci USA 109: E1192-E1200.
65. Hou X, Lee L Y C, Xia K, Yan Y, Yu H (2010) DELLAs modulate jasmonate signaling via competitive binding to JAZs. Dev Cell 19:884-894.
66. Machado R A R, Baldwin I T, Erb M (2017) Herbivory-induced jasmonates constrain plant sugar accumulation and growth by antagonizing gibberellin signaling and not by promoting secondary metabolite production. New Phytol 215:803-812.
67. Agrawal A A (1998) Induced responses to herbivory and increased plant performance. Science 279:1201-1202.
68. Baldwin I T (1998) Jasmonate-induced responses are costly but benefit plants under attack in native populations. Proc Natl Acad Sci USA 95:8113-8118.
69. Farmer E E, Dubugnon L (2009) Detritivorous crustaceans become herbivores on jasmonate-deficient plants. Proc Natl Acad Sci USA 106:935-940.
70. Smith A M, Stitt M (2007) Coordination of carbon supply and plant growth. Plant Cell Environ 30:1126-1149.
71. Bomblies K, Weigel D (2007) Hybrid necrosis: Autoimmunity as a potential gene-flow barrier in plant species. Nat Rev Genet 8:382-393.
72. Qi T, et al. (2015) Regulation of jasmonate-induced leaf senescence by antagonism between bHLH subgroup IIIe and IIId factors in Arabidopsis. Plant Cell 27:1634-1649.
73. Shan X, et al. (2011) The role of Arabidopsis Rubisco activase in jasmonate-induced leaf senescence. Plant Physiol 155:751-764.
74. Ueda J, Kato J (1980) Isolation and identification of a senescence-promoting substance from wormwood (Artemisia absinthium L.). Plant Physiol 66:246-249.
75. Orozco-Cirdenas ML, Narviez-Visquez J, Ryan CA (2001) Hydrogen peroxide acts as a second messenger for the induction of defense genes in tomato plants in response to wounding, systemin, and methyl jasmonate. Plant Cell 13:179-191.
76. Oh Y, Baldwin I T, Gilis I (2012) NaJAZh regulates a subset of defense responses against herbivores and spontaneous leaf necrosis in Nicotiana attenuata plants. Plant Physiol 159:769-788.
77. Chen Y, et al. (2017) Salt and methyl jasmonate aggravate growth inhibition and senescence in Arabidopsis seedlings via the JA signaling pathway. Plant Sci 261:1-9.
78. Machado R A R, et al. (2013) Leaf-herbivore attack reduces carbon reserves and regrowth from the roots via jasmonate and auxin signaling. New Phytol 200: 1234-1246.
79. Sulpice R, et al. (2014) Arabidopsis coordinates the diurnal regulation of carbon allocation and growth across a wide range of photoperiods. Mol Plant 7:137-155.
80. Wang K, et al. (2018) Two abscisic acid-responsive plastid lipase genes involved in jasmonic acid biosynthesis in Arabidopsis thaliana. Plant Cell 30:1006-1022.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements

1. A plant, plant cell, or plant seed comprising at least one endogenous cdk8 loss-of-function mutation in one or more endogenous CDK8 genes and at least one endogenous loss-of-function jaz mutation in one or more endogenous JAZ genes.
2. The plant, plant cell, or plant seed of statement 1, wherein the one or more endogenous JAZ genes is a JAZ1, JAZ2, JAZ3, JAZ4, JAZ5, JAZ6, JAZ7, JAZ9, JAZ10, or JAZ13 gene; or wherein the one or more endogenous JAZ genes comprise a combination of two or more JAZ1, JAZ2, JAZ3, JAZ4, JAZ5, JAZ6, JAZ7, JAZ9, JAZ10, or JAZ13 genes.
3. The plant, plant cell, or plant seed of statement 1 or 2, wherein the one or more endogenous JAZ genes is a least one endogenous loss-of-function mutation in each of JAZ1, JAZ2, JAZ3, JAZ4, JAZ5, JAZ6, JAZ7, JAZ9, JAZ10, and JAZ13.
4. The plant, plant cell, or plant seed of statement 1, 2 or 3, wherein endogenous expression of the one or more endogenous JAZ gene, the cdk8 gene, or a combination thereof is reduced by at least 20%, or at least 30%, or at least 40%, or at least 50%, or at least 60%, or at least 70%, or at least 80%, or at least 90%, or at least 95%, or at least 97%, or at least 99% compared to wild type plant cells, plants, and seeds of the same species (that do not have the jaz or cdk8 mutation(s)).
5. The plant, plant cell, or plant seed of statement 1, 2 or 3, wherein endogenous expression of the one or more endogenous JAZ genes or the cdk8 gene, or a combination thereof is undetectable.
6. The plant, plant cell, or plant seed of statement 1-4 or 5, wherein at least one of the plant's, the plant cell's, or the plant seed's endogenous JAZ8, JAZM1, or JAZ12 genes are not modified or mutated.
7. The plant, plant cell, or plant seed of statement 1-5 or 6, wherein the plant's vegetative weight, vegetative weight of a plant generated from the plant cell, or vegetative weight of a plant grown from the plant seed is within at least about 40%, or at least about 50%, or within at least 60%, or at least about 70% of the average vegetative weight of a wild type plant grown for the same time and under the same conditions as a wild type plant.
8. The plant, plant cell, or plant seed of statement 1-6 or 7, wherein the plant, a plant generated from the plant cell, or a plant grown from the plant seed has a rosette weight of about 40% to about 120%, or about 50% to about 110% of the rosette weight of wild type plants grown for the same time and under the same conditions.
9. The plant, plant cell, or plant seed of statement 1-7 or 8, wherein the plant, a plant generated from the plant cell, or a plant grown from the plant seed has a seed yield of at least 10%, or at least 20%, or at least 30%, or at least 40% greater than the average seed yield of wild type plants.
10. The plant, plant cell, or plant seed of statement 1-8 or 9, wherein the plant, a plant generated from the plant cell, or a plant grown from the plant seed has at least 5% less, 10% less, 20% less, 30% less, 40% less, 50% less, 60% less, 70% less, 80% less, 90% less, or 100% less leaf damage from insect feeding than average insect feeding of a wild type plant of the same species grown for the same time under the same conditions.
11. The plant, plant cell, or plant seed of statement 1-9 or 10, wherein the plant, a plant generated from the plant cell, or a plant grown from the plant seed has at least about 10%, or at least about 20%, or at least about 30%, or at least about 40%, or at least about 50%, or at least about 60%, or at least about 70%, or at least about 80%, or at least about 90% fewer insects or insect larvae than an average number of insects or insect larvae of wild type plants of the same species grown for the same time under the same conditions.
12. The plant, plant cell, or plant seed of statement 1-10 or 11, wherein compared to wild type or an unmodified parental plant line, the plant, a plant generated from the plant cell, or a plant grown from the plant seed has higher levels of defense compounds that reduce the incidence or number of insect or insect larvae on the plant.
13. The plant, plant cell, or plant seed of statement 1-11 or 12, wherein compared to wild type or an unmodified parental plant line, leaves of the plant, a plant generated from the plant cell, or a plant grown from the plant seed has higher levels of aliphatic glucosinolates that reduce the incidence or number of insect or insect larvae on the plant.
14. The plant, plant cell, or plant seed of statement 1-12 or 13, wherein compared to wild type or an unmodified parental plant line, the plant, a plant generated from the plant cell, or a plant grown from the plant seed has higher levels of one or more of 3-methylsulphinylpropyl glucosinolate (glucoiberin); 4-methylsulphinylbutyl glucosinolate (glucoraphanin); 5-methylsulphinylpentyl glucosinolate (glucoalyssin); 6-methylsulphinylhexyl glucosinolate (glucohesperin); 7-methylsulphinylheptyl glucosinolate (glucoibarin); 3-methylthiopropyl glucosinolate (glucoiberverin); 8-methylsulphinyloctyl glucosinolate (glucohirsutin); 4-methylthiobutyl glucosinolate (glucoerucin); 5-methylthiopentyl glucosinolate (glucoberteroin); or 7-methylthioheptyl glucosinolate.
15. The plant, plant cell, or plant seed of statement 1-13 or 14, wherein the plant, a plant generated from the plant cell, or a plant grown from the plant seed exhibits resistance to environmental stress compared to a wild type plant of the same species grown for the same time and under the same environmental conditions.
16. The plant, plant cell, or plant seed of statement 4-14, or 15, wherein the wild type plant, wild type plant cell, or wild type plant seed expresses JAZ polypeptides or JAZ-related polypeptides with at least 70%, or at least 75%, or at least 80%, or at least 85%, or at least 90%, or at least 95%, or at least 97%, or at least 98%, or at least 99% sequence identity to any of SEQ ID NO:1, 3, 5, 7, 9, 11, 13, 17, 19, 21-51, or 52.
17. The plant, plant cell, or plant seed of statement 4-15, or 16, wherein the wild type plant, wild type plant cell, or wild type plant seed expresses CDK8 polypeptides or CDK8-related polypeptides with at least 70%, or at least 75%, or at least 80%, or at least 85%, or at least 90%, or at least 95%, or at least 97%, or at least 98%, or at least 99% sequence identity to any of SEQ ID NO: 53, 56, 58, 59, 61, 63, or 65.
18. The plant, plant cell, or plant seed of statement 1-16 or 17, wherein the endogenous loss-of-function mutation of the JAZ1, JAZ2, JAZ3, JAZ4, JAZ5, JAZ6, JAZ7, JAZ9, JAZ10, JAZM3 or cdk8 gene comprises substitution(s) or deletion(s) at chromosomal loci of the JAZ1, JAZ2, JAZ3, JAZ4, JAZ5, JAZ6, JAZ7, JAZ9, JAZ10, JAZM3 or cdk8 gene.
19. The plant, plant cell, or plant seed of statement 1-17 or 18, wherein the endogenous loss-of-function mutation of the JAZ1, JAZ2, JAZ3, JAZ4, JAZ5, JAZ6, JAZ7, JAZ9, JAZ10, JAZM3 or cdk8 gene comprises insertion(s) at chromosomal loci of the JAZ1, JAZ2, JAZ3, JAZ4, JAZ5, JAZ6, JAZ7, JAZ9, JAZ10, JAZM3 or cdk8 gene.
20. The plant, plant cell, or plant seed of statement 1-18 or 19, which is a food crop species (e.g., sugar beets, beets, tomatoes, lettuce, spinach, carrots, peppers, peas, broccoli, beans, asparagus), a legume species (e.g., peas, beans, lentils, peanuts), a fiber-containing plant species, a tree species, flax, a grain species (e.g., maize, wheat, barley, oats, rice, sorghum, millet, and rye), a grass species (e.g., switchgrass, prairie grass, wheat grass, sudangrass, sorghum, straw-producing plants), a woody plant species (e.g., a poplar species, pine species, or eucalyptus species), a softwood, a hardwood, an oil and/or starch producing plant species (e.g., canola, potatoes, lupins, sunflower and cottonseed), a forage plant species (e.g., alfalfa, clover, or fescue).
21. The plant, plant cell, or plant seed of statement 1-19 or 20, wherein the one or more endogenous JAZ genes is a combination of two or more, or three or more, or four or more, or five or more, or six or more, or seven or more, or eight or more, or nine or more JAZ1, JAZ2, JAZ3, JAZ4, JAZ5, JAZ6, JAZ7, JAZ9, JAZ10, or JAZM3 genes.
22. A method comprising cultivating the plant, plant cell or plant seed of statement 1- or 21 to produce a mature plant.
23. The method of statement 22, further comprising harvesting the mature plant or harvesting seeds, grain, fruit, vegetables, or biomass of the mature plant.
24. The method of statement 22 or 23, wherein the mature plant has less average insect damage or less insect larval and/or less adult insect feeding than a wild plant cultivated for the same time and under similar growing conditions.
25. The method of statements 22, 23 or 24, wherein the mature plant has greater seed yield than a wild plant cultivated for the same time and under similar growing conditions.
26. A method comprising (a) introducing into one or more plant cell(s) at least one chromosomal loss-of-function mutation into one or more endogenous JAZ genes and introducing into the one or more plant cell(s) at least one chromosomal loss-of-function mutation into at least one endogenous cdk8 gene; and (b) generating a plant from the one or more plant cell(s).
27. The method of statement 26, wherein the one or more endogenous JAZ genes is a JAZ1, JAZ2, JAZ3, JAZ4, JAZ5, JAZ6, JAZ7, JAZ9, JAZ10, or JAZM3 gene; or wherein the one or more endogenous JAZ genes comprise a combination of two or more JAZ1, JAZ2, JAZ3, JAZ4, JAZ5, JAZ6, JAZ7, JAZ9, JAZ10, or JAZ13 genes.
28. The method of statement 26 or 27, wherein the one or more endogenous JAZ genes has at least 70%, or at least 75%, or at least 80%, or at least 85%, or at least 90%, or at least 95%, or at least 97%, or at least 98%, or at least 99% sequence identity to any of SEQ ID NO:1, 3, 5, 7, 9, 11, 13, 17, 19, 21-51 or 52.
29. The method of statement 26, 27 or 28, wherein the endogenous cdk8 gene has at least 70%, or at least 75%, or at least 80%, or at least 85%, or at least 90%, or at least 95%, or at least 97%, or at least 98%, or at least 99% sequence identity to any of SEQ ID NO: 53, 56, 58, 59, 61, 63, or 65.
30. The method of statement 26-28 or 29, wherein the plant generated from the one or more plant cell(s) comprises a deletion of at least one chromosomal JAZ1, JAZ2, JAZ3, JAZ4, JAZ5, JAZ6, JAZ7, JAZ9, JAZ10, or JAZM3 site, a substitution within at least one chromosomal JAZ1, JAZ2, JAZ3, JAZ4, JAZ5, JAZ6, JAZ7, JAZ9, JAZ10, or JAZ13 site, or an insertion into at least one chromosomal JAZ1, JAZ2, JAZ3, JAZ4, JAZ5, JAZ6, JAZ7, JAZ9, JAZ10, or JAZM3 site.
31. The method of statement 26-29 or 30, wherein the plant generated from the one or more plant cell(s) comprises a deletion of a chromosomal cdk8 site, a substitution within a chromosomal cdk8 site, or an insertion into a chromosomal cdk8 site.
32. The method of statement 26-30 or 31, wherein the endogenous expression of the JAZ or cdk8 gene in the plant generated from the one or more plant cell(s) is reduced by at least 20%, or at least 30%, or at least 40%, or at least 50%, or at least 60%, or at least 70%, or at least 80%, or at least 90%, or at least 95%, or at least 97%, or at least 99% compared to wild type or parental plants of the same species (that do not have the jazD or cdk8 mutation(s)).
33. The method of statement 26-31 or 32, wherein endogenous expression of the one or more JAZ or cdk8 gene in the plant generated from the one or more plant cell(s) is undetectable.
34. The method of statement 26-32 or 33, wherein endogenous JAZ8, JAZ11, and JAZ12 genes in the plant generated from the one or more plant cell(s) or the progeny thereof are not modified or mutated.
35. The method of statement 26-33 or 34, wherein vegetative weight of plant generated from the one or more plant cell(s) or the progeny thereof is within at least about 40%, or at least about 50%, or within at least 60%, or at least about 70% of the average vegetative weight of a wild type plant grown for the same time and under the same conditions as a wild type plant.
36. The method of statement 26-34 or 35, wherein the plant generated from the one or more plant cell(s) or the progeny thereof has a rosette weight of about 40% to about 120%, or about 50% to about 110% of the rosette weight of wild type plants grown for the same time and under the same conditions.
37. The method of statement 26-35 or 36, wherein the plant generated from the one or more plant cell(s) or the progeny thereof has a seed yield of at least 10%, or at least 20%, or at least 30%, or at least 40% greater than the average seed yield of wild type plants.
38. The method of statement 26-36 or 37, wherein the plant generated from the one or more plant cell(s) or the progeny thereof has at least 5% less, 10% less, 20% less, 30% less, 40% less, 50% less, 60% less, 70% less, 80% less, 90% less, or 100% less leaf damage from insect feeding than average insect feeding of a wild type plant of the same species grown for the same time under the same conditions.
39. The method of statement 26-37 or 38, wherein the plant generated from the one or more plant cell(s) or the progeny thereof has at least about 10%, or at least about 20%, or at least about 30%, or at least about 40%, or at least about 50%, or at least about 60%, or at least about 70%, or at least about 80%, or at least about 90% fewer insects or insect larvae than an average number of insects or insect larvae of wild type plants of the same species grown for the same time under the same conditions.
40. The method of statement 26-38 or 39, wherein the plant generated from the one or more plant cell(s) or the progeny thereof exhibits resistance to environmental stress compared to a wild type plant of the same species under the same environmental conditions.
41. The method of statement 26-39 or 40, wherein the one or more endogenous JAZ genes with the mutation is two or more JAZ genes, or three or more JAZ genes, or four or more JAZ genes, or five or more JAZ genes, or six or more JAZ genes, or seven or more JAZ genes, or eight or more JAZ genes, or nine or more JAZ genes.

The specific plants, seeds, compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a plant” or “a seed” or “a cell” includes a plurality of such plants, seeds or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed:

1. A plant, plant cell, or plant seed comprising at least one endogenous cdk8 loss-of-function mutation in one or more endogenous CDK8 genes and at least one endogenous loss-of-function jaz mutation in one or more endogenous JAZ genes.


SEQUENCE LISTING (121 sequences)

SEQ ID NO: 1 - protein, 187 aa, Arabidopsis thaliana
MSLFPCEASNMDSMVQDVKPTNLFPRQPSFSSSSSSLPKEDVLKMTQTTRSVKPESQTAPLTIFYAGQVIVFNDFSAEKAKEVINLASKGTANSLAKNQTDIRSNIATIANQVPHPRKTTTQEPIQSSPTPLTELPIARRASLHRFLEKRKDRVTSKAPYQLCDPAKASSNPQTTGNMSWLGLAAEI

SEQ ID NO: 2 - DNA, 1527 bp, Arabidopsis thaliana
  source 1..1527: mol_type = genomic DNA
atattggaggtaggaagaagaactctgcaaccaaaccaaccaaccccaaagccaaacaaagttttatagagaccttccatttctccctctcgtgacaaacgcaatttgcagagaagcaacagcaacaacaagaagaagaagaaaaagatttgagattactttgtatcgatttagctattcgagaaactcttgccgtttgaaagttttaattgttaaagatgtcgagttctatggaatgttctgagttcgtcggtagccggagatttactgggaagaagcctagcttctcacagacgtgtagtcgattgagtcagtatctaaaagagaacggtagctttggagatctgagcttaggaatggcatgcaagcctgatgtcaatggtaagaaaccttctctttctcctagatccacttcttttttcgttttctctgttttttatttcttgaatcttgatcttgaaaacttttcaagaaaattttgaatcgatttcaaagaaattagggagagttagtttgctaaattttgacatagaaaatgattggagagagttctaacttttggatcatatatatttgcaggaactttaggcaactcacgtcagccgacaacaaccatgagtttattcccttgtgaagcttctaacatggattccatggttcaagatgttaaaccgacgaatctgtttcctaggcaaccaagcttttcttcctcatcttcctctcttccaaaggaagatgttttgaaaatgacacagactaccagatctgtgaaaccagagtctcaaactgcaccattgactatattctacgccgggcaagtgattgtattcaatgacttttctgctgagaaagccaaagaagtgatcaacttggcgagcaaaggcaccgctaatagcttagccaagaatcaaaccgatatcagaagcaacatcgctactatcgcaaaccaagttcctcatccaagaaaaaccacaacacaagagccaatccaatcctccccaacaccattgacagaacttcctattgctagaagagcttcacttcaccggttcttggagaagagaaaggacagagttacgtcaaaggcaccataccaattatgcgatccagccaaagcgtcttcaaaccctcaaaccacaggcaacatgtcgtggctcggtttagcagctgaaatatgaatgctaaccaccctcaagccgtaccaagaaattcttttgacgacgttgcttcaagacaagatataaaagctcctatcttcatgctttttgatttaagatacaaactactcaatgattaggaaacttcatatatttgtatgtattgattagtgatcaattattgttagtattcgttatagtctgtttttctactagttattgtcgcctgtctaaatccccttgctatgggttatctcaaaattagtttcgtatgtaactaattttgtaagaacaataatttttgttgacgaaccatactatcaaatactctaaattatatcttgataaatctacctatcaggtaagtagg

SEQ ID NO: 3 - protein, 249 aa, Arabidopsis thaliana
MSSFSAECWDFSGRKPSFSQTCTRLSRYLKEKGSFGDLSLGMTCKPDVNGGSRQPTMMNLFPCEASGMDSSAGQEDIKPKTMFPRQSSFSSSSSSGTKEDVQMIKETTKSVKPESQSAPLTIFYGGRVMVFDDFSAEKAKEVIDLANKGSAKSFTCFTAEVNNNHSAYSQKEIASSPNPVCSPAKTAAQEPIQPNPASLACELPIARRASLHRFLEKRKDRITSKAPYQIDGSAEASSKPTNPAWLSSR

SEQ ID NO: 4 - DNA, 1282 bp, Arabidopsis thaliana
  source 1..1282: mol_type = genomic DNA
gcaaccagcgaaaaaaaagtaataaagaggtcctccatttcttcctcgtgacaaaacgcacttggcagagaaagataaacaagaaccctaagtttttttataagattcgagaaaattcaacaactcaggaaggaagatccttttgctccaatttctcaatcgaaacgatttcaatttcggtttcaacgatgtcgagtttttctgccgagtgttgggacttctctggtcgtaaaccgagcttttcacaaacatgtactcgattgagtcgttacctgaaggagaagggtagttttggagatctgagcttagggatgacatgcaagcccgacgttaatggaggttcacgtcagcctacaatgatgaatctgttcccttgtgaagcttcaggaatggattcttctgctggtcaagaagacattaaaccgaagactatgtttccgagacaatcaagcttttcttcttcctcttcctctgggaccaaagaagatgtacagatgatcaaagagactactaaatctgtgaagccagagtctcaatctgctccgttgactatattctacggtggtcgagttatggtgtttgatgatttttctgctgagaaagctaaagaagtcattgatttggctaacaaaggaagtgccaaaagcttcacatgtttcacagctgaagtaaacaataaccatagtgcttattctcaaaaagagattgcttctagcccaaatcctgtttgtagtcctgcaaaaaccgcagcacaagagccaattcagcctaacccggcctctttagcctgcgaactcccgattgcaagaagagcttcacttcatcggttccttgagaagaggaaggataggatcacatcaaaggcaccataccaaatagacggttcagctgaagcgtcttccaagcctactaacccagcttggctcagttcacggtaaacttcgagcctgtccgacccagaaggcacaacttgagagaccttcttgtaagattcttctgatgctccatcgttacaaatatcaagctgctcctctgttcattttttctatagattaatttcacccctagtagttttgtttgtttaactcccccgaaaactcattatatttgtatgaaatcaatatcaatagtgttcaatgtttgcttctggggtttaagttttagccagtgtgtataaccctttcctctgccgatctcaacattagcttgcaacttttgtaagaaacatcacttgtgtttttgtgttgatggccattaatataatccaagtttatttaatccg

SEQ ID NO: 5 - protein, 352 aa, Arabidopsis thaliana
MERDFLGLGSKNSPITVKEETSESSRDSAPNRGMNWSFSNKVSASSSQFLSFRPTQEDRHRKSGNYHLPHSGSFMPSSVADVYDSTRKAPYSSVQGVRMFPNSNQHEETNAVSMSMPGFQSHHYAPGGRSFMNNNNNSQPLVGVPIMAPPISILPPPGSIVGTTDIRSSSKPIGSPAQLTIFYAGSVCVYDDISPEKAKAIMLLAGNGSSMPQVFSPPQTHQQVVHHTRASVDSSAMPPSFMPTISYLSPEAGSSTNGLGATKATRGLTSTYHNNQANGSNINCPVPVSCSTNVMAPTVALPLARKASLARFLEKRKERVTSVSPYCLDKKSSTDCRRSMSECISSSLSSAT

SEQ ID NO: 6 - DNA, 3253 bp, Arabidopsis thaliana
  source 1..3253: mol_type = genomic DNA
gcgatttgttaataaaactagaaattgcggtgaattaacttcattccacgttttttcattttctccctcaaaagtctctgttttttttcctttttccggcgaagctctatttagcttgattccggcgtttaacacgcgttttaatcgaaacagacatttgagatcgaattaattttgtagcgggctgtgtctttattatagatggagagagattttctcgggttgggttcgaaaaattctccgatcactgtcaaggaggaaaccagcgaaagctctagagattcaggttatttattactcttctcaatttttctgattctgattgtttttaaatcgtagatttgtttgattgattaggagttattaggactacttgtagtatggaatttgtttttggatagctgattttatggcttgctcgggaactggaattgtcagtttgttgcttggagcagaacattgtcctttgcttttctcgggagatgtagaatttggatttggaaaaactagtgttcttttccaaagccttgtcttaaacatgctttcggtcggagaaattaacgagaactaatctcaagcttctaacataattaaactcggtaaacttttttttactagagtaaatttttttgttttgtttgaagagtcttataattgagaaatactttattagtttatactaaaaaaaaaacgaatacgtaaaatgttggaaaagaggggatgtatagagactgatacaaaaatgataaaatagagacggttggtagtaggtagaaagattaaatatactcaaaagagtgagttggattagtttataagatgattaacttcttgattgtgtgagttggattagtttatgagattattaaaatattgattgtgtatttgtgttgtgtgttgattaagcggaacttgcgttagaatattgttcaaggtacaatgtggaaataatagttttctcaccacgaggaatataattatttcaactttgttttcttatcagccaaaacgtgccacaccataaaagtagtgcatcaacatgtggtgtggtgtggtggggttaaagtttgaatctctctttaatttaaactattaaaacaaacttaaattattggagtttcgtacaatgactttcaatcaaatgttttagaattagacacggttttcgaaagtggttttccctcgttgaatttgtcaacagtatcagattctacattgttggttactaatcttttccttgaagtaggtgttgaattaatcctctgttgtttatgtaaggagatctcgagacatttatggttaacagttaacactacatgtttgactttaaactgattatcttttattctttttcttttgtagctcccaacagaggaatgaactggtctttctcaaacaaagtatcagcttcttcttctcagtttctatccttcaggccaactcaagaagatagacatagaaagtctggaaattatcatcttcctcactctggttccttcatgccatcatcagtagctgatgtttatgattcaacccgcaaagctccttacagttctgtacaggtatttgtcatcaaaacctatgttaaccaagacccttgtgttttttttatccttcgcaagatagctttaaaagtgagccctgttttatgagcatatagtaattggttttgagtctagtttagcacaagttcatggcaattagtttgtggatctaatcttggtttaatactgattcattttaagtgtaagctaagcttctcatttttgataagttagttcatacaatgcctcacacctactttatggcttgttactctcagggagtgaggatgttccctaattccaatcaacacgaagaaactaacgcagtttccatgtcgatgccgggtttccagtctcatcattatgcaccaggaggaagaagcttcatgaacaataacaataactcacaacctttggtaggagttcctatcatggcacctccaatttcaatccttcctcctccaggttccattgtagggactactgatattaggtacccactagtcatcatatcatacagaaactctttctacattttcatagttgactaaagacttatttttgtcagatcttcttccaagccaataggttcacctgcgcagttgacgatcttttatgccggttcagtttgtgtttacgatgacatatctcctgaaaaggtatctcaatcattttcttccatatatgcatctcttttactcgtaaggtatggtactcatttgctttctttcatttctcaggcaaaggcgataatgttgctagctgggaacggttcctctatgcctcaagtcttttcgccgcctcaaactcatcaacaagtggtccatcatactcgtgcctctgtcgattcttcagctatgcctcctagcttcatgcctacaatatcttatcttagccctgaagctggaagtagcacaaacggactcggagccacaaaagcgacaagaggcttgacgtcaacatatcacaacaaccaagctaatggatccaatattaactgcccagtaccagtttcttgttctaccaatgtaatggctccaacaggtaaaaaacaaagtcagagacctgatactacattcgccatctaacttactagtattttcatggatgtaacttcattctcgttctgtttcttatgcagtggcattacctctggctcgcaaagcatccctggctaggtttttagagaaacgcaaagaaaggtacgcaacacttctttagaatacaccattcaatagtttcttgggctaactctctttctcgctgtgggtttctcagggtcacgagcgtatccccatattgcttagacaagaagtcatcgacagattgtcgcagatcaatgtctgaatgcattagttcttctctcagctctgcaacctaatttcatctacagtaagaaggttgctttagaccactccacatccatatttgcatttcaatggcggtcttttcaatgtctcagttaatttttcctcactcgccacactgagtttctccttagctttatatatacgatagtgtatactttgtttacatgttttttggtggaatggaacttatgagagcatatcagatatgtacttgggaaaattagtagaaactgtttgtttctttttttttaactctgttcttttgtatatatcactgaagctcgcatatgtataattcatgtaatggaattgcatcgcttctgtttccctaagttattt

SEQ ID NO: 7 - protein, 310 aa, Arabidopsis thaliana
MERDFLGLGSKLSPITVKEETNEDSAPSRGMMDWSFSSKVGSGPQFLSFGTSQQETRVNTVNDHLLSSAAMDQNQRTYFSSLQEDRVFPGSSQQDQTTITVSMSEPNYINSFINHQHLGGSPIMAPPVSVFPAPTTIRSSSKPLPPQLTIFYAGSVLVYQDIAPEKAQAIMLLAGNGPHAKPVSQPKPQKLVHHSLPTTDPPTMPPSFLPSISYIVSETRSSGSNGVTGLGPTKTKASLASTRNNQTAAFSMAPTVGLPQTRKASLARFLEKRKERVINVSPYYVDNKSSIDCRTLMSECVSCPPAHHLH

SEQ ID NO: 8 - DNA, 3187 bp, Arabidopsis thaliana
  source 1..3187: mol_type = genomic DNA
attagaggaatcataaatcggcggtgtgtgtaacttcaactcacgtttttcatttctctccaaagtccttcaattgttactaattctctctgatctctcatttcttctcttctccggtgacattttttttctcccccgcgaaagctaaaccgtttttgtattctcaacgattgataagcctgatggagagagattttctcgggctgggatcaaagttatctccgataactgtgaaggaggaaactaacgaagattcaggtaattcatcttcaacatcttccattatgatctgatgattgtgtttttcatctcacttttttttgtttctatttttgtaatctctttttttgtttattgttcaagtacatatatattgtttttctagcttgattgggagtcctactgtctggttttttcttgaacaagaaattttttcttcgttttctcgggaagagaaaaaataaattagggtttcttttttcttgatatatatttaagaaattaggttttagtactatagacagaaatttagctactcgaatttgtttgacgtagccgatgaaaaaacacgttttgggactcgatagttagaaaattcatacgttcacgatctacttttgaagtttttttcattaaatattttttgcaaactacaaatgtacaagtatacaactatacaagcaaacaccaaacttgttgacgttagtaatttaacaagtgttagtattatctttgaaaaataatattcagagaacaaacttgattttctaggtgactaggtgatgcatgtttctaaagctgttggtaatgttgagtgttttcaaaataatttcgtttttttcttcaaacagccgacaccgacagaacaaaaatgctatattttttttgttgcttacaaaattgatcaattggtttcaatacaatagtatcttctttagaaaagattgtttttttcaaagccggattgaatattgagaattagaacattggctggttattctttttgaaaagtttatgccattttttaaggtttattaagcaacttgaattctatcagtattatttaaaaacgaagacgtgaaatgttgggaaaagaatgcgttatatagcgaccggctgacgattagagatttaacaacaaatgcaagttgaattatataaaagcaagattgattgtgacttgattaagttttatttctatccaagtagactcattgattaagttaggatcatgttgggtattaaatttagatcaagttacaatttggatgaataatttacttacccacgaggaatttaatagttagttcttgtctttttatattccgaaacgtgccatttcttgaaagtatttgtatgatcactattttccccagtgtgtttggctttatgcagatttgttcattgttgatgaatctaatgttaagagtcgtccactttagcatagctagatctgagtgtttcctagtttgataaaatctaaagacatttgctcatgtttcagccccaagtagaggtatgatggattggtcattctcaagcaaagtcggttctggtcctcagtttctttcttttgggacatcccaacaagaaacgcgtgtaaacacagtcaatgatcatttgctttcttctgctgcaatggatcaaaaccagagaacttacttcagctcactacaggttaggctatttcttgaaaagaaaaaaagtagtgataaagtgtgatttagtgaccttgtaagaaagcttggcaattggtttagtttcttctggtctcaaaattgatacaaaatgatctcaggaagacagagtgttcccaggttccagtcagcaagaccaaacaaccatcacagtctccatgtccgaaccaaactacatcaacagtttcataaaccaccaacatttaggaggatctcctatcatggcacctccagtttcagtatttcctgctccaaccactattaggcatgcactgcattctatcttcttctgtttaacatcagatacagaacctctttacttctatagttgactcgagctcctttatgttcatctccagatcttcttcaaaaccacttccccctcagttgacaatcttttatgccggttcagtattagtttaccaagacatagctcctgaaaaggtaaccaaatttccttcaatatgtgttacattacagtccaagctatccactgactaagtattcaatcaaagaaataagtttcacgtatagacatgctgaagttatagaaagttactaacctggtttcaacatacagtatgttaatgattcatagatatgataaatctttgtccttacttcttcatttattttgtattcataggcccaagctatcatgttgctagccggaaatggacctcatgctaaaccggtttcacaacctaaacctcaaaaactggttcatcactctcttccaaccactgatcctccaactatgcctcctagtttcctgccttccatctcttacattgtctctgaaaccagaagtagtggatccaacggggttactggacttggaccaacaaaaacaaaggcgagtttagcatccacgcgcaacaaccaaactgctgccttctctatggctccaacaggttataaatgaagtcttaactcctattaatgttttgtcatcaaacttctatcttaggtttagtttgttataaccaaaaaatcttgctatgatttaatacagtgggtttaccacaaacacgcaaagcatccttggctcggttcttagagaaacgcaaagaaaggtactgagctacaagattattcacttattcacaatatcaaaacacaggtttgctgtatattggcttcgttttcttgcagggtcattaacgtatcaccttattacgtagacaacaagtcatcaatagactgtagaacactgatgtctgaatgtgtaagctgtcctccagctcatcatctgcactaaaaccaatttagacccctcattgttctaaaggctttttcttttttctctggctctgtatcctatagactatagtatagttgttatagcttttgtttattcagattttagtacactgggcttgtaaaagcaagttatttatatatatcctataaatttaatttggatactgtatgttttgtctttactcttgcatgtgtataaaaaacataaaagtaagactattcaagct

SEQ ID NO: 9 - protein, 274 aa, Arabidopsis thaliana
MSSSNENAKAQAPEKSDFTRRCSLLSRYLKEKGSFGNIDLGLYRKPDSSLALPGKFDPPGKQNAMHKAGHSKGEPSTSSGGKVKDVADLSESQPGSSQLTIFFGGKVLVYNEFPVDKAKEIMEVAKQAKPVTEINIQTPINDENNNNKSSMVLPDLNEPTDNNHLTKEQQQQQEQNQIVERIARRASLHRFFAKRKDRAVARAPYQVNQNAGHHRYPPKPEIVTGQPLEAGQSSQRPPDNAIGQTMAHIKSDGDKDDIMKIEEGQSSKDLDLRL

SEQ ID NO: 10 - DNA, 1765 bp, Arabidopsis thaliana
  source 1..1765: mol_type = genomic DNA
taatcatggatgaaaattcctttcttcacactagatatagttctttaactagttaaaaatgcatgcgatggaatattactaaatatgatataatctcatggctttatgtaagatttgttttttggtttttttggttgttgttaataaatttattattgagaagtttaattctattttggtcacaatatattgaaatatttttaagaaactaaaaagttcctatttatttttgttttcattaatttatgagaggctattaaagtcacagaaacttattgggtgaatgagtttataaacacatgagctattgagctagtagcctcttgtactcttccattttacgcgcaatccacgcaccaacaaaaagaaaagaaaagaagagataaagaatatctttaaaaagtaagtgtggagaattctttcttctcaataaacaacaacatgtcgtcgagcaatgaaaatgctaaggcacaagcgccggagaaatctgactttacccggagatgtagtttgctcagccgttacttgaaggagaagggtagtttcggaaacattgatcttggcttataccgaaaacccgattccagtctcgcgttgcccggaaaattcgatccaccagggaaacaaaatgcgatgcataaggcagggcattccaaaggcgaaccctctacctcatcaggaggcaaagtcaaagatgttgctgacctcagtgaatcacagccaggaagttcgcagctgaccatattcttcggagggaaagttttagtatataatgagttccccgtagacaaagctaaagagattatggaagtagcaaaacaagccaagcctgtgactgagattaacattcagacaccaatcaatgacgaaaacaacaacaacaagagcagcatggttcttcctgatctcaatgagcctactgataataatcacctaacaaaggaacaacaacagcaacaagaacaaaatcagatcgtggaacgtatagcacgtagagcttccctccatcgattctttgctaaacggaaagacagagctgtggctagggctccgtaccaagttaaccaaaacgcaggtcatcatcgttatcctcccaagccagagattgtaaccggtcaaccactagaggcaggacagtcgtcacaaagaccgccggataacgccattggtcaaaccatggcccatatcaaatcagacggtgataaagatgatattatgaagattgaagaaggccaaagttcgaaagatctcgatctaaggctatagtaatatttgctaaatttcttgtaggaactgagtttttagattaacgtttcgatttttctgacttatctaagtgattttattttgctttgtactacagtatgtaatcttattctaacttgaatattcattcataaacacaatagacgatagtaaagttatattataattagttaactacgtacaacacttgggagttaaattacataacgttaagcgagaaatagcaaattagacaagaggaagaatatttaggagttgtgaattgatctgactgcaataacatgaagaggaatctgactgcaatcgtaatgcgggtaaagatggttgaaagtgatcagagctcctttctaatttatttagggtgtaatttatgaaaatgattattattggagtgtatatcaagttttcactaaactcaggggtgtttattgtaattagttgtcaggttcaagttcattgaaggcgtgtctgatttggacagtgattgggcctgagccat

SEQ ID NO: 11 - protein, 269 aa, Arabidopsis thaliana
MSTGQAPEKSNFSQRCSLLSRYLKEKGSFGNINMGLARKSDLELAGKFDLKGQQNVIKKVETSETRPFKLIQKFSIGEASTSTEDKAIYIDLSEPAKVAPESGNSQLTIFFGGKVMVFNEFPEDKAKEIMEVAKEANHVAVDSKNSQSHMNLDKSNVVIPDLNEPTSSGNNEDQETGQQHQVVERIARRASLHRFFAKRKDRAVARAPYQVNQHGSHLPPKPEMVAPSIKSGQSSQHIATPPKPKAHNHMPMEVDKKEGQSSKNLELKL

SEQ ID NO: 12 - DNA, 1604 bp, Arabidopsis thaliana
  source 1..1604: mol_type = genomic DNA
aaattaatagcctataatatgtttgaccataaaaagaatttcttcttcttgaaccatcataagaaaaatgtgtgtttagtctattgatcagttttgtgttcaaaaaaaaaaaaaaaatgtctatcgatcagttaggtgtaaaaaaaaaagttacaaaactcgtgacaaaaacattctatattggacacacatcactgtcacttcagactaaataaaaaaaaagaacacgttatttcgttttctttatttattcgggagaggttaaaagccacagaaacttattggctagaattggttatttataacaacaacacatgagcaaaaagctcaaaacatctacatactctttggaatcctcgattttttgtacgtgtaaagaagtcacacaagaaaatcttgggttgttgtaatcttcatcacactagtatgtcaacgggacaagcgccggagaagtccaatttttctcagagatgtagtctgctcagccggtacttgaaggagaagggaagttttgggaatattaatatggggttggctcgaaaatccgatcttgaactcgccggaaaattcgatctcaaaggacaacaaaatgtgattaagaaggtagagacctcagaaactagaccgttcaagttgattcagaagttttctattggtgaggcctctacttctaccgaagacaaagccatatatattgatctcagtgaaccggcaaaagtagcaccggagtctggaaattcacagttgaccatattctttggaggaaaagttatggttttcaacgagtttcctgaagacaaagctaaggagataatggaagtagctaaagaagcgaatcatgttgctgttgattctaagaacagtcagagtcacatgaatcttgacaaaagcaacgtggtgattcccgatcttaacgagccaacgagttccgggaacaatgaagatcaagaaactgggcagcaacatcaggttgtggaacgcattgcaagaagagcttctcttcatcgattctttgctaaacgaaaagacagggctgtggctagagctccatatcaagtgaaccaacacggtagtcatcttcctcccaagccagagatggttgctccatcgataaagtcaggccaatcgtcgcaacacattgcaactcctccaaaaccaaaggcccataaccatatgccgatggaggtggacaagaaagaaggacaatcttccaaaaaccttgaactcaagctttagggcgtataaaatgcacgatcgagttcacgtttctagttttcacttatttaggatttgaacccaaatacccttttatattttcttccattacttttgaccaatttaagttatttatagtactgtattacgtagctagtatttatatttcaaaacatacatattttgatacttgttttttagattctttaattaaaattgtcatctggattaccctttatcgaaattttttaatcacctgatataatctcaccagtgatggatttgcgttgttagtaatttttctaagtggcaaaagtatattaacctataataggtttcaaagatatacatataatgtttctatcaaagatattagtataatattttac

SEQ ID NO: 13 - protein, 148 aa, Arabidopsis thaliana
MIIIIKNCDKPLLNFKEMEMQTKCDLELRLLTSSYDSDFHSSLDESSSSEISQPKQESQILTIFYNGHMCVSSDLTHLEANAILSLASRDVEEKSLSLRSSDGSDPPTIPNNSTRFHYQKASMKRSLHSFLQKRSLRIQATSPYHRYR

SEQ ID NO: 14 - DNA, 823 bp, Arabidopsis thaliana
  source 1..823: mol_type = genomic DNA
gttgtgttctgtccaaactctgttctaatgccagcttttgtctcgtcttttctctatccttatcttccctctatcttcgatcccaacacatacacaacacgcacacacacatatataaatcaactgactgacacatacaatcatgatcatcatcatcaaaaactgcgacaagcctttactcaatttcaaagagatggagatgcaaacaaaatgcgacttggaacttcgccttcttacttcttcttatgattctgatttccatagctcgttggacgaatcaagcagctctgaaatttcacaaccaaagcaagaatctcagatattaaccattttctacaacgggcacatgtgtgtttcttcagatcttacccatcttgaggctaacgctatactatcgctagcgagtagagatgtggaagagaaatctttatccttgagaagttcagacggttcggatcctccaacaatcccaaacaattcgactcgatttcattatcaaaaggcctctatgaagagatctcttcacagttttcttcagaaacgaagtcttcggattcaagcaacttccccttaccaccgttaccgatagcactatctatttgatttcatttttgtgattctcttcaattttttttttactgtaacataataatccaattgtcttgaattctttttctgtgtgtttggatggattacagaccttaattaggtagagtattaaagtttcataatttccagtaacttgtgtttagagttcaagaggttgacaaaatttatcaacggtctcctaaaatgggtaaaccgagaaacttttatacgaaaa

SEQ ID NO: 15 - protein, 267 aa, Arabidopsis thaliana
MERDFLGLSDKQYLSNNVKHEVNDDAVEERGLSTKAAREWGKSKVFATSSFMPSSDFQEAKAFPGAYQWGSVSAANVFRRCQFGGAFQNATPLLLGGSVPLPTHPSLVPRVASSGSSPQLTIFYGGTISVFNDISPDKAQAIMLCAGNGLKGETGDSKPVREAERMYGKQIHNTAATSSSSATHTDNFSRCRDTPVAATNAMSMIESFNAAPRNMIPSVPQARKASLARFLEKRKERLMSAMPYKKMLLDLSTGESSGMNYSSTSPT

SEQ ID NO: 16 - DNA, 2297 bp, Arabidopsis thaliana
  source 1..2297: mol_type = genomic DNA
gcaaagagttaaataagcctctccaaaagtgtgtctgtaacattaccaaaacgaaaccttccttgtggattcccacttctttcttctgttttcttcttcctcttctttaaattggatgttttgggcaagaaacagagagaaacacgttaatttgagagtttgtcattgaatatttggtttgcaatggaaagagattttctgggtttgagcgacaagcagtatctaagtaataacgttaagcatgaggttaacgatgatgctgtcgaagaacgaggtttgtgttcttgtctcgagaatcttttattttaatgtttcaagaagagatcagttttcacttttaacatagccgtataaagttgtttatttaaatataatttttcagattccaaaacttgaaaaaaaaaagattccattaaatcttttataaaaatgagattggatagattagtcaaattgacgaccataaaaaatgatacttatagggttaagtacgaaggcagctagagaatgggggaagtcaaaggtttttgctacttcaagtttcatgccttcttcagatttccaggttggttcatcttaaaatttaacttactctgtatcagtttcagatgttatggctaatctaatggttctataagctaccgcataatcatggtcgtcttttagcatgtgcaagaggagtactcaattatggtcttgattaaaaagaagaatttactttcaaattatgttaaacacatcaatcacatatttatgagaaaagttgttttcgtaagagatagccaccggaaaatggtcggataaatggccgaactttatcatttttgtgtatgtggccaatcattaaccagggaaaaaaaattgttggataagtgctagttaagagctggtagggtcggtcgtctgccagccgcaaagttagggaaaaaataatttaatattttgtggcgtttggtgtttggcgtttggatcacgtttatttcttggcatttttctaaatttagaatgtacaaaaaatttaaagacgttgacgattaaaatttgaatttaacaaattaggaggctaaggcgtttccgggtgcataccagtggggatcagtttctgcggccaatgttttccgcagatgccaatttggtggtgcgtttcaaaacgcgacgccgcttttactaggcggttcagttcctttaccaactcatccttctcttgttccacggtaatttccatattatgatgcaaaaacattcaacaatttttttgctcttttcatattttgatttggttatgtgggtttgtggaaacagagtggcttcctccggatcatctcctcagctcacaatcttttatggcggaactataagcgtctttaatgacatatctcccgataaggtatatataatcaagattcatacaaataacatttacataacatttacatgttctaaaacggactattcatgatatgtgagtaggctcaagccatcatgttatgcgccgggaacggtttgaaaggtgaaactggagatagcaaaccggttcgagaagctgaaagaatgtatggaaaacaaatccataacactgctgctacctcatcaagctctgccactcacactgataatttctcaaggtgtagggacacacccgttgctgcgactaatgcaatgagcatgatcgaatcattcaatgcagctcctcgtaacatgattccttcaggtatgtgtgtctaatatcaacatcaaaacaaaatataatcaagatttttgcttcctcaaatcatatgtctaaactcgaaaattgcttttttccagtccctcaagctcggaaagcatccttggctcggttcttggagaagcgcaaagagaggtttgattttgtattttttttctttatagaaaattttgaggtttttcaattgaatctaaaagaattgatgttgttggtgcaggcttatgagtgcaatgccatacaagaagatgcttcttgatttgtcgaccggagaatccagtggaatgaattactcttctacttctcctacataaaacctacactttttttttttttttttacaatggtaatttgtaattgtaatcattagattatgattatatagttaccatttatattcttacgagcaggagaagacgttagggcgtctctgtatttgatcattgtttgtaatgctttggtctgtttattgtaggattacattataactttaagaactaacagatatatgtttgtcatggactcatgtctgtcaagaatttaatatcaaataaaattcactataatttttttt

SEQ ID NO: 17 - protein, 197 aa, Arabidopsis thaliana
MSKATIELDFLGLEKKQTNNAPKPKFQKFLDRRRSFRDIQGAISKIDPEIIKSLLASTGNNSDSSAKSRSVPSTPREDQPQIPISPVHASLARSSTELVSGTVPMTIFYNGSVSVFQVSRNKAGEIMKVANEAASKKDESSMETDLSVILPTTLRPKLFGQNLEGDLPIARRKSLQRFLEKRKERLVSTSPYYPTSA

SEQ ID NO: 18 - DNA, 1927 bp, Arabidopsis thaliana
  source 1..1927: mol_type = genomic DNA
aaaaactctcacatgagaaatcagaatccgttattattcctccatttattcatctcaaaacccatatctctctgtcttgatctctctctcactttctaataagatcaaagaagatgtcgaaagctaccatagaactcgatttcctcggacttgagaagaaacaaaccaacaacgctcctaagcctaagttccagaaatttctcgatcgccgtcgtagtttccgaggttcgtttggtttttagtcgctctctcttttttttttcttgcgataaatcgaatttattcatatggaactcctgcagatattcaaggtgcgatttcgaaaatcgatccggagattatcaaatcgctgttagcttccactggaaacaattccgattcatcggctaaatctcgttcggttccgtctactccgagggaagatcagcctcagatcccgatttctccggtccacgcgtctctcgccaggtatttttgtctttccggtaaagttttttttttctttctaacttttttggcgctaccagaaaagacgaaaaaatttgaaattcaaattttcaaaacattcattttcctcaggtctagtaccgaactcgtttcgggaactgttcctatgacgattttctacaatggaagtgtttcagttttccaagtgtctcgtaacaaagctggtgaaattatgaaggtcgctaatgaagcagcatctaagaaagacgagtcgtcgatggagacagatctttcggtaattcttccgaccactctaagaccaaagctctttggccagaatctagaaggaggttagtataataaaaataaaaatcacttagtgctggattcttctagaattttagttacatattattgcatgtagagatctaagaagagtttgttgttagagaggaattggttgctaattagtttggaattagatatcaaagagttaaagactatagtttatgtctatacgtattaatatacgttattaataaaagtataaacatgttgtttaatttctgataagaaactggtttatgcgtgtgtatgcagatcttcccatcgcaaggagaaagtcactgcaacgttttctcgagaagcgcaaggagaggtaatgattcttcaacaatccaaggatttttacccccaaataattaaagaaaggtttttatttttctctctctcgaccttttttttactataagttatttaagatagtaattatgggtcctgcctcttttactctcacatacaacttaagattcaactagttttgttcaacaacgcacatgcttatacgtagatagataatggagatcagtagtaatatcggtatacgtaggttactattgtaatggaacttttaaaaagcgcgttgactttgagtctttgactctagttctgtttgctacaccgacaagttatatttttcaaaatgatgagaaaacgaggagaaacaccggaaaaaaatttgaacttttacttttatcagaccatacggccaaagaaagatctgtatattatataagttatcacaaaacgcggtttcacattttctttttcgtcttgttgtgtttgcagattagtatcaacatctccttactatccgacatcggcctaaacgatctctttttagattgggacatggaccaaatttgtctttttcaatcggaagacatccatgttcgtttttggatttggcttatttccaatcttcttttgaagccttcttcgtcgttgctaaatcgtatactattcacgacaaacgtttttaggagattacgttacctactaagattatatatattggtttgtttttaaaaatgtctattatctttattgtcattgatagcttgatttaagaagctctctcttatcccgtgaccttctacttttgttttattttttagtatatggtaaagaaaattataac

SEQ ID NO: 19 - protein, 125 aa, Arabidopsis thaliana
MKGCSLDLHLSPMASTLQSCHQDSTVNDRSSTIRSKEINAFYSGRLSEYDLVEIQMRAIIEMASKDREVTALELVPVRLESPLGCSVKRSVKRFLEKRKKRSKSFTLTPNYTSSTSSSSSSLHNF

SEQ ID NO: 20 - DNA, 834 bp, Arabidopsis thaliana
  source 1..834: mol_type = genomic DNA
ttgattactttgatacgaaaatcgaccaaagtaagaatatttacctagagaggatcatgaagggttgcagcttagatcttcacctatctccaatggcctctacgcttcaatcttgtcatcaagattctacagttaatgatcgttcttcaaccataagatctaaggaaatcaatgcattttatagtgggagattaagtgagtacgatcttgtagagatccagatgagagcaataatagagatggcgagcaaggatcgtgaagtaacagcgttagagttagtgccggtgagactggaatcaccgttaggatgttcggtgaagagatctgtgaaaaggttcttggagaagaggaagaagagaagcaaatcttttacacttacacctaattacacctcaagtacttcctcatcatcctcctctcttcataatttctaatcataattttattatgttttccttctagttatcaatcaaaacaaaaaaatctttgtttctttcttttttcttttttccattatgggtttctatagctctcatttatctcttgtaatttttcccgatactcgacgatgaatttcgagtttttttttttgatctgttttaaatcaagacattctagtaccattggagtctgtataaaattcagatcatttggatcgttatttttttcctaattcatgtatgaagtgtcacacttctcctacaatgaattatgaggttgtccgtttattccaagttagctctatgtactttgacgtaagctaatgcaacttgtaaaatgttgggaactcttctattactttttttcctttacaaaataagaaaatgcacgcat

SEQ ID NO: 21 - protein, 273 aa, Brassica rapa
MSSPMESSDFAATRRFSRKPSFSQTCSRLSQYLKENGSFGDLSLGMACKPEVNGISRQPTTTMSLFPCEASNMEPIGQDVKPKNLFPRQPSFSSSSSSLPKEDILKMTQATSSTRSVKPEPQTAPLTIFYGGQVIVFNDFSAEKAKEVMDLASKGTANTFTGFTSNVNNNIQSVYTTNLANNQTEMRSNIAPIPNQLPHLMKTTTQNPVQSSSTAMACELPIARRASLHRFLAKRKDRVTSKAPYQLNDPAKASSKPQTGDNTTSWLGLAAEM

SEQ ID NO: 22 - protein, 252 aa, Brassica oleracea
MSSSMECSTTRRSSSGKPSFSLTCSRLSQYLKENGSFGDLSLGMSCKPDTNGMSRKPTTTMSLFPCEASNVGSMAAAQDVKPKNLFPRQPSFSSSSSSIPKEDVPKMTQTTTRSLKPEPQTAPLTIFYGGQVIVFNDFSAEKAKEVMNLANKGTANTFTGFTSTLNNNIAPTPNQVPHLMKAATQDPKQTSSAAMACELPIARRASLHRFLAKRKDRVTSKAPYQLNDPAKAYSKPQTGNTTTSWLGLAADM

SEQ ID NO: 23 - protein, 233 aa, Zea mays
MAASARPGERATSFAVACSLLSRFVRQNGVAAADLGLRIKGEVEQQRTPATTNSLPGAEGEEVERRKETMELFPQSVGFSIKDAAAPREEQGDKEKPKQLTIFYGGKVLVFDDFPADKAKDLMQLASKGSPVVQNVALPQPSAAAAVTTDKAVLDPVISLAAAKKPARTNASDMPIMRKASLHRFLEKRKDRLNAKTPYQTAPSDAAPVKKEPESQPWLGLGPNAVDSSLNLS

SEQ ID NO: 24 - protein, 240 aa, Glycine max
MSSSSEYLVFSSHHPANSPAEKSTFSQTCSLLSQYIKEKGTFGDLTLGMTCTAETNGSPETSCHSATTMELFPTIITQRNPTTVDFLSPQTAYPHHSEVPIMVKSSAFKSMEKEPKAAQLTIFYAGQVVVFDDFPAEKLEEITSLAGKGISQSQNTSAYAHTHNQQVNHPSFVPNISPQAPSRPLVCDLPIARKASLHRFLSKRKDRIAAKAPYQINNPNSASSKPAESMSWLGLGAQST

SEQ ID NO: 25 - protein, 244 aa, Oryza sativa
MAASARPVGVGGERATSFAMACSLLSRYVRQNGAAAAELGLGIRGEGEAPRAAPATMSLLPGEAERKKETMELFPQSAGFGQQDAITADSAADAREQEPEKRQLTIFYGGKVLVFNDFPADKAKGLMQLASKGSPVAPQNAAAPAPAAVTDNTKAPMAVPAPVSSLPTAQADAQKPARANASDMPIARKASLHRFLEKRKDRLNAKTPYQASPSDATPVKKEPESQPWLGLGPNAVVKPIERGQ

SEQ ID NO: 26 - protein, 237 aa, Zea mays
MAASARPGERATSFAVACSLLSRFVRQNGAAPAQLGLGIKGEVEQQRTPATINLLPGADGEETERRKETMELFPQSAGFGVKDAAAAPREQENKEKPKQLTIFYGGKVLVFDDFPADKAKDLMQLASKGGPVVQNVVLPQPSAPAAAVTDKAVPVPVISLPAAQADAKKPTRTNASDMPIMRKASLHRFLEKRKDRLNANAPYQTSPSDAAPVKKEPESQAWLGLGPNAVKSNLNLS

SEQ ID NO: 27 - protein, 231 aa, Triticum aestivum
MAASARQGERATSFAMACSLLSRYVRQNGAAAAELGLGINKGEAEAQRAADTKSPLPGAEGEEAGRKKETMELFPQSAGLQDAAAPDATREEDKSQLTIFYGGKVLVFNDFPADKAKGLMQLAGKGSPVVQNVSATTTAADTDKVQTAVLAPASSLPTGPVDAPKPARPNASDLPIARKASLHRFLEKRKDRLHAKAPYQAPPSDATPVKKEFENQPWLGLGPNAALKPNQ

SEQ ID NO: 28 - protein, 242 aa, Glycine max
MSSSSEYLVFSGHHPANSPAEKSTFSQTCSLLSQYIKEKGTFGDLTLGMTCTAETNGSPETSCHSATTMELFPTIITQRNPTTVDFLSPQTAYPHHSEVPIMVKSSAFKSMEKEPKAAQLTIFYAGQVVVFDDFPAEKLEEITSLAGKGISQSQNTSAYAHTHNQQVNHPSFVPNISPQAPSRPLVCDLPIARKASLHRFLSKRKDRIAAKAPYQINNPNSASSKPAESMSWLGLGAQSTQV

SEQ ID NO: 29 - protein, 391 aa, Zea mays
MERDFLAAIGKEQQHPRKEKAGGGAEESAYFGAAAVPAMDWSFASKPCAAPALMSFRSAAREEPSFPQFSALDGTKNTAPRMLTHQRSFGPDSTQYAALHRAQNGARVVPVSSPFSQSNPMFRVQSSPSLPNSTAFKQPPFAISNAVASSTVGSYGGTRDAVRPRTAQLTIFYAGSVNVFNNVSAEKAQELMFLASRGSSAPVACKPEAPPTLAPAKVTAPEVLLPAKQMLFQKPQHLSPPPSSVPGILQSAALPRSASSSSNLDSPAPKSSVPLAVPPVSQAPPATLIATTTAAAIMPRAVPQARKASLARFLEKRKERVTTAAPYPSAKSPLESSDTFGSGSASANANDKSSCTDIALSSNHEESLCLGGQPRSIISFSEESPSTKLQI

SEQ ID NO: 30 - protein, 419 aa, Triticum aestivum
MERDFLGTIGHEQLQQQQQQQQRQRAAAEDAAARKESAYFGGGGVPPMDWSFAGRAGAAPAVMSFRSAPREEQRGELAYPKQQASRVLTPQRSFGAESHGSVQYAAAARAAYGGQPPQQHQHAPNGARVIPMSSPFNPNNPMFRVQSSPNLPNGVAAGSPFKQPPFVMNNAVAASTVGVYKSRDMPKPKTAQLTIFYAGSVNVFNNVSAEKAQELMFLASRGSLPTAPTTVTRSPDATFFTPAKLAAPEASPAKQMLAHIPQRVSPPLPAISKPMSIMSQAACLPKSTSSSNTDSAVPKSSGQLVVPPTSQTSSSTHPVTLSSTTAASIMPRAVPQARKASLARFLEKRKERVTTTAPYPSAKSPMESSDTVGSANDNNSKSSSCTEIAFSSNHEESLRLGRPRNISFSGESPSTKLHI

SEQ ID NO: 31 - protein, 386 aa, Glycine max
MEREFFGLSSKNGAWTTMKDDAVNKSRDQVRSSGMQWSFPNKVSALPQFLSFKTNQEDKPRKTILEPLASSGYMAMSTQYAFDSNQKSFLGLTNRNLSISKHAAGNKQGMTVYPLQCCDAQSEEARIFSVSNQSNQVSPVLQSNLASTGLNMVNSVIKPQPFGSKSSGTPLSILPSIGSIVGSTDLRNNSKSSTMPTQLTIFYAGSVCVYDDISPEKAKAIMLMAGNGYTPTEKMELPTVKLQPAISIPSKDDGFMISQSYPPSTFPTPLPLTSHVNSQPGGGSSSNKEISIIRQVGPSTAPTNHLESPIIGSIGSASKEKAQPVCLPQARKASLARFLEKRKGRMMRTSPYLYMSKKSPECSSSGSDSVSFSLNFSGSCSLPATN

SEQ ID NO: 32 - protein, 416 aa, Oryza sativa
MERDFLGAIGKDEEQRRHAEERKESDYFGAGGGAAAAAMDWSFASRAALMSFRSSSSAAAAAAREETRELAFPHFSALDGAKMQQASHVLARQKSFGAESHGIPQYAAAAAVHGAHRGQPPHVLNGARVIPASSPFNPNNPMFRVQSSPNLPNAVGAGGGAFKQPPFAMGNAVAGSTVGVYGTRDMPKAKAAQLTIFYAGSVNVFNNVSPEKAQELMFLASRGSLPSAPTTVARMPEAHVFPPAKVTVPEVSPTKPMMLQKPQLVSSPVPAISKPISVVSQATSLPRSASSSNVDSNVTKSSGPLVVPPTSLPPPAQPETLATTTAAAIMPRAVPQARKASLARFLEKRKERVTTVAPYPLAKSPLESSDTMGSANDNKSSCTDIALSSNRDESLSLGQPRTISFCEESPSTKLQI

SEQ ID NO: 33 - protein, 404 aa, Zea mays
MAKSGASFPESSWMERDFLAAIGKEQQHPHKEEAGAEESAYFGGAGAAAAAPAMDWSFASKPGAAPALMSFRSASFPQFSSFDGAKNPAPRILTHQRSFGPDSTHYAAAHRTQHALNGARVTPVSSPFNQNSPMFRVQSSPSLPNGTAFKQPPFAINNNAAASSTVGFYGTRDVVRPKTAQLTIFYAGSVNVFDNVSAEKAQELMLLASRGSLPSSAPVARKPEAPILAPAKVTAPEVLHATQMLFQKPQHVSPPSSAISKPIPGILQAASLPRSASSSNLDSPFPKSSVPFPVSPVSQAPRAQPATIAATTAAAIMPRAVPQARKASLARFLEKRKERVTTAAPYPSAKSPMESSDTFGSGSANDKSSCTDIALSSNHEESLCLGQPRNISFIQESPSTKLQI

SEQ ID NO: 34 - protein, 340 aa, Glycine max
MERDFMGLNLKEPLAVVKEEMNNDGCKNSGFKKGRIAQWPFSNKVSALPHLMSFKASQDDKTKNTVSDTLSSSGFMSILSQEAFDTSQKRSAGEPQMFSVPNQAISVSLGNPFLKNHFAAAGQKPLLGGIPVTTSHSVLPSAVAVAGMTESCNSVKPSAQLTIFYAGTVNIFDDISAEKAQAIMLLAGNSLSAASNMAQPNVQVPISKLGAGAGVPVSQPANTSPGSGLSSPLSVSSHTGVQSGSGLTSTDEFLAAKTTGVPNTPICNVEPPKVVSATTMLTSAVPQARKASLARFLEKRKERVMSAAPYNLNKKSEECATAEYAGVNFSATNTVLAKQG

SEQ ID NO: 35 - protein, 427 aa, Oryza sativa
MERDFLGAIWRKEEAAGKPEEHSDYRGGGGGASAAMQWQFPATKVGAASSAFMSFRSSAAAAREEDPKEAAVFDRFSLSGFRPPPRPSPGDAFDGAAAMKQRQFGFNGRQQYAAAAQHGHREQGVDSYGVAAPHHFPSPSPSPRHPVPFGHANPMLRVHSLPNVAGGSPYRNQSFSVGNSVAGSTVGVYGGPRDLQNPKVTQMTIFYDGLVNVFDNIPVEKAQELMLLASRASIPSPPSAARKSDSPISAAAKLTVPEALPARQIVVQKPEASVPLVSGVSNPITIVSQAVTLPKSFSSSNDSAGPKSGGLPLAVTPLSQASPSQPIPVATTNASAIMPRAVPQARKASLARFLEKRKERVSSVAPYPSSKSPLESSDTIGSPSTPSKSSCTDITPSTNNCEDSLCLGQPRNISFSSQEPPSTKLQI

SEQ ID NO: 36 - protein, 157 aa, Triticum aestivum
  VARIANT 1..157: note = X = Any Amino Acid
LANGRSGMLPMSSPPANPGQLTIFYGGSVCVYDSVPPEKAQAIMLIAAAAAAASKSNGTAAVKPPAMSATNAIQAMLTRSLSLQSTSVAXGQPQAVADPGSICKLQADLPIARRHSLQRFLEKRRDRVVSKAPYGARKPFEGMGASSGMESVAEGRP

SEQ ID NO: 37 - protein, 216 aa, Zea mays
MAGHAPARDKTTSGFAATCSLLSQFLKEKKGGLQGLGGLAMAPAPAAGAGAFRPPTTMNLLSALDAAKATVGEPEGHGQRTGGNPREAAGEEAQQLTIFYGGKVVVFDRFPSAKVKDLLQIVSPPGADAVVDGAGAAVPTQNLPRPPHDSLSADLPIARRNSLHRFLEKRKDRITAKAPYQVNSSVGAEASKAEKPWLGLGQEGSDGRQAGDVIDE

SEQ ID NO: 38 - protein, 258 aa, Glycine max
MSSSSEYSEFSGQKPARSPEKSSFSQTCSLLSQYIKEKGSFGDLTLGMTSCGSPETSCQSATTMNLFPPKENNVAPKNLTAMDLLSPQASSYGPSEEIPTLVNSSAIKSVSKGAKTAQMTIFYGGQVVVFDDFPADKASEIMSYATKGGIPQSQNNSVYTYTQSQPSFPPTLIRTSADSSAPIIPSVNITNSIREHPQASSRPVVYLPIARKASLHRFLEKRKDRIASKAPYQVANGPSNKAAESMPWLGLSASSPQI

SEQ ID NO: 39 - protein, 204 aa, Triticum aestivum
MDLLERSAATIKAEAGEAQRKEAERKEQELEKEQETQQPGLTGRPPLANGRSGMLPMSSPPANPGQLTIFYGGSVCVYDSVPPEKAQAIMLIAAAAAAASKSNGTAAVKPPAMSATNAIQAMLTRSLSLQSTSVANGQPQAVADPGSICKLQADLPIARRHSLQRFLEKRRDRVVSKAPYGAGKPSEGMGASSGMEAVAEGKAQ

SEQ ID NO: 40 - protein, 235 aa, Zea mays
MAASARPGERATSFAVACSLLSRFVRQNGVAAAELGLRIKGEVEQQRTPATTSLLPGAEGEEVERRKETMELFPQSVGFSIKDAAAPPREEQGDKEKPKQLTIFYGGKVLVFDDFPADKAKDLMQLASKGSPVVQNVVLPQPSAAAAVTTDKAVLDPVISLAAAAKKPARTNASDMPIMRKASLHRFLEKRKDRLNAKTPYQTAPSDAAPVKKEPESQPWLGLGPNAVDSSLNLS

SEQ ID NO: 41 - protein, 257 aa, Glycine max
MSSSSEYSQFSGQKPARSPEKSSFSQTCSLLSQYIKEKGSFGDLTLGMTSCGSPETSCQSATTMNLFPTKENNVTPKDLTAMDLFSPQASSYRPSEEIPTLINSSAIKSVSKSAKTAQMTIFYGGQVVVFDDFPADKASEIMSYATKGIPQSQNNSVFTYTPSQPSFPANLVRTSADSSAPIIPSVNITNSIHEHPQASSRPVVYLPIARKASLHRFLEKRKDRIASKAPYQLANGSSNQPAESMPWLGLSASSPRI

SEQ ID NO: 42 - protein, 244 aa, Oryza sativa
MAASARPVGVGGERATSFAMACSLLSRYVRQNGAAAAELGLGIRGEGEAPRAAPGTMSLLPGEAERKKETMELFPQSAGFGQQDAITADSAADAREQEPEKRQLTIFYGGKVLVFNDFPADKAKGLMQLASKGSTVAPQNAVAPAPAAVTDNTKAPMAVPAPVSSLPTAQADAQKPARANASDMPIARKASLHRFLEKRKDRLNAKTPYQASPSDATPVKKEPESQPWLGLGPNAVVKPIERGQ

SEQ ID NO: 43 - protein, 213 aa, Zea mays
MDGGRDVDEGGVTGAVAAAAAQERRWRGGGGDDEESSGLSNGGGGVELSLRLRTGADDGAATAAALSPLPLPPPAEARRNMTIFYNGRVCAADVTEIQARAIISMASEETLADHRGRRRRQQQQQLTRGDGGDGRQQDGDSSSSTTTSAVALARRCARGRGLVGPAVEIDQAADAGLSMKRSLQLFLQKRKARTAAAAAPPYAGGRQAQAVRR

SEQ ID NO: 44 - protein, 133 aa, Glycine max
MRRNCNLELALFPPSDSGPPMVDNVEEEASEISPMQNLFHRQEQQQPLTIFYDGKICVADVTELQAKSILMLANRKLEERVRTPTGSEPSSPTVMQSNNQLYSPGTGPSMRKSLQRFLQKRRNRVQEASPYRH

SEQ ID NO: 45 - protein, 166 aa, Triticum aestivum
MAAASRSAPEWWRDGGSVDDGGAEVELSLRLRTGSSSTARRSMTIFYNGRVVAVDVTELQAREIITMASQQILTEQQDSGGGGGGTAVAQYGAHENPSQPAPQRWAPLLASRSLRQGAGAAAPVTSQAAAAGLSMKRSLQRFLQKRKTRVAAMGSPYAGGRRAMPS

SEQ ID NO: 46 - protein, 353 aa, Zea mays
MDWSFASKPCAAPALMSFRSAAREEPSFPQFSALDGTKNTAPRMLTHQRSFGPDSTQYAALHRAQNGARVVPVSSPFSQSNPMFRVQSSPSLPNSTAFKQPPFAISNAVASSTVGSYGGTRDAVRPRTAQLTIFYAGSVNVFNNVSAEKAQELMFLASRGSSAPVACKPEAPPTLAPAKVTAPEVLLPAKQMLFQKPQHLSPPPSSVPGILQSAALPRSASSSSNLDSPAPKSSVPLAVPPVSQAPPATLIATTTAAAIMPRAVPQARKASLARFLEKRKERVTTAAPYPSAKSPLESSDTFGSGSASANANDKSSCTDIALSSNHEESLCLGGQPRSIISFSEESPSTKLQI

SEQ ID NO: 47 - protein, 320 aa, Glycine max
MERDFMGLNLKEPLAVVKEEMNNDGCKNSGFKKGRIAQWPFSNKVSALPHLMSFKASQDDKTKNTVSDTLSSSGFMSILSQEAFDTSQKRSAGEPQMFSVPNQAISVSLGNPFLKNHFAAAGQKPLLGGIPVTTSHSVLPSAVAVAGMTESCVKPSAQLTIFYAGTVNIFDDISAEKAQAIMLLAGNSLSAASNMAQPNVQVPISKLGAGAGVPVSQPANTSPGSGLSSPLSVSSHTGVQSGSGLTSTDEFLAAKTTGVPNTPICNVEPPKVVSATTMLTSAVPQARKASLARFLEKRKERVMSAAPYNLNKKSEECATA

SEQ ID NO: 48 - protein, 308 aa, Oryza sativa
MQQASHVLARQPPHVLNGARVIPASSPFNPNNPMFRVQSSPNLPNAVGAGGGAFKQPPFAMGNAVAGSTVGVYGTRDMPKAKAAQLTIFYAGSVNVFNNVSPEKAQELMFLASRGSLPSAPTTVARMPEAHVFPPAKVTVPEVSPTKPMMLQKPQLVSSPVPAISKPISVVSQATSLPRSASSSNVDSNVTKSSGPLVVPPTSLPPPAQPETLATTTAAAIMPRAVPQARKASLARFLEKRKERVTTVAPYPLAKSPLESSDTMGSANDNKSSCTDIALSSNRDESLSLGQPRTISFCEESPSTKLQI

SEQ ID NO: 49 - protein, 218 aa, Zea mays
MAGHAPARDKTTTGFAATCSLLSQFLKEKKGGLQGLGGLAMAPAPAAGAGAFRPPTTMNLLSALDAAKATVGEPEGHGQRTGGNPREAAGEEAQQLTIFYGGKVVVFDRFPSAKVKDLLQIVSPPGADAVVDGAGAGAAVPTQNLPRPSHDSLSADLPIARRNSLHRFLEKRKDRITAKAPYQVNSSVGAEASKAEKPWLGLGQEQEGSDGRQAGEEM

SEQ ID NO: 50 - protein, 201 aa, Glycine max
MAAGVTVKSEVLESSPPEGVCSNTVENHLVQTNLSDGSPNKSVPASGLDAVIPSANQLTIFYNGSVCVYDGIPAEKVHEIMLIAAAAAKSTEMKKIGTQTTLISPAPSRPSSPHGITNNIGSSQKSSICRLQAEFPIARRHSLQRFLEKRRDRLGSKTPYPSSPTTKVADNIENNFCADNAPELISLNRSEEEFQPTVSAS

SEQ ID NO: 51 - protein, 188 aa, Oryza sativa
MSTRAPVELDFLGLRAAAADADDRHAKSGGSSASSSSSIRGMETSAIARIGPHLLRRVIAAAGPPPPPSTAPVPEEMPGAAAAAAPMTLFYNGSVAVFDVSHDKAEAIMRMATEATKAKGLARGNAIVGNFAKEPLTRTKSLQRFLSKRKERLTSLGPYQVGGPAAVGATTSTTTKSFLAKEEEHTAS

SEQ ID NO: 52 - protein, 468 aa, Zea mays
MSQQEAVDPPSATDERLGGLPRSGSTSRLNAQAPEFVPRAAAVPPPPPQQKVVRLFAPPPHAAFFVAAPRPPPPPFEYYAAVATGGGGRFGPPAAAAEQEAEAEQPPRDGSFDDPVPKIRKQVEYYFSDINLATTEHLMRFISKDPEGYVPISVVAGFKKIKALVQSNSMLASALRTSSKLVVSDDGARVKREQPFTESDLEELQARIVVAENLPDDHCYQNLMRLFSVVGSVRTIRTCYPQTPNGTGPATNRSAKLDMLFANKLHAFVEYDTIEDAARAIVELNDERNWRSGLRVRLLSTCMGGKGKKGGHESDGYGDEENVSTSDQPYDKYLEETPQMSDVPGEHMTEDSAGDMGRGRVRGRGRGGRGRGRGYHQQNNNQHHQHYQNSSHHSNSSSTRPVGTPPPSGHPVMIEQQQQQQAAQPQPLTAANKQPPGPRMPDGSRGFSMGRGKPQTLTPKVSESEPEQ

SEQ ID NO: 53 - protein, 470 aa, Arabidopsis thaliana
MGDGSSSRSNSSNSTSEKPEWLQQYNLVGKIGEGTYGLVFLARTKTPPKRPIAIKKFKQSKDGDGVSPTAIREIMLLREISHENVVKLVNVHINFADMSLYLAFDYAEYDLYEIIRHHRDKVGHSLNTYTVKSLLWQLLNGLNYLHSNWIIHRDLKPSNILVMGDAEEHGIVKIADFGLARIYQAPLKPLSDNGVVVTIWYRAPELLLGSKHYTSAVDMWAVGCIFAELLTLKPLFQGAEAKSSQNPFQLDQLDKIFKILGHPTMDKWPTLVNLPHWQNDVQHIQAHKYDSVGLHNVVHLNQKSPAYDLLSKMLEYDPLKRITASQALEHEYFRMDPLPGRNAFVASQPMEKNVNYPTRPVDTNTDFEGTTSINPPQAVAAGNVAGNMAGAHGMGSRSMPRPMVAHNMQRMQQSQGMMAYNFPAQAGLNPSVPLQQQRGMAQPHQQQQLRRKDPGMGMSGYAPPNKSRRL

SEQ ID NO: 54 - DNA, 2561 bp, Arabidopsis thaliana
  source 1..2561: mol_type = genomic DNA
gcaagtggctaaaaaaattacaaatctagtttccattctcagcgtcggctgcttggaacgtcaccgttttctggaaaacgcaatcttctcccttccgtgacgtctcaccggaattttctcgcttttgtctactctcctccatctccgaggttctccaagctcagctcctcttcccatcattcatccgaccgccttatccggtcagatcctttacgtatttctattttcctgatcgtcgatttttgagaaatgtaaaaacagatcgtataaggcctcgaagtttttaatttgaaagtggtatcgaaattttttggtctttgattaggttagggcaccgtagctctgggtattgaatttgtagggttttcctctggttattggtctttggagcttggtaatttctgctgaattgattgatcccttttccatcttttgaagtaaagtctcgagctttcgtgtctcgatgtagatgaattctattttgaatatgagatttgataagacgtcaattgctgataatttggagtctttgtgtctgaatttgttcatatgaagttttctgagggatgtgaattttattgtctgctaattttgaaacgttccttttggaatttggtttgtgaggagtcctagatctttttctgtgaagtttcttgcttgtaagttttctggatcacttgattgagtctagaatctagatagattacatgtacggtttgattcctttggctgattttccaaagttttgttcaaatttcaggagaactacaaagaggaaaccaagattgttttgttttgttagactctaccccttttccgattcacatggtaaggacattgaggtagagaataatactaaaaagcaatgggagatgggagttccagtagatccaacagctcaaacagcactagtgagaaaccagagtggctgcaacagtacaatctcgttggtaagattggtgaaggcacttatggtcttgttttcttggctagaaccaagactccgcctaaaagacctattgctatcaagaagtttaagcagtccaaagatggagatggagtttccccgactgctatccgcgagatcatgttgcttagagagatttcccatgagaacgtcgtgaagcttgtgaatgtccacatcaattttgcagacatgtctctgtatcttgcctttgattatgccgagtacgatctctatgaaatcatcaggcaccacagagacaaagtcggccattcgttaaacacatacacagttaagtctttgctctggcagcttctcaacggattgaactatcttcacagtaattggattatacacagagatttgaaaccgtcgaatatcttggttatgggtgatgcagaagagcacggaatagtgaaaatagctgatttcgggctcgcaaggatatatcaagctccgttgaaaccactatcggataacggagttgtggtcacaatctggtaccgagcaccagagctgcttcttggttcgaagcactacacgagcgctgttgatatgtgggcagttgggtgtatattcgcggagttactaactcttaaaccgttgtttcaaggagcagaagcgaaatcgtctcaaaaccctttccagttagatcaacttgacaagatattcaagatcttaggccacccgacgatggataaatggccaacactagttaaccttccacactggcaaaatgatgttcaacacattcaagctcacaaatacgacagtgtgggtctccacaacgtggttcacctgaatcagaaaagtcctgcgtatgatctgttatccaaaatgctggaatatgatcctctaaagcggatcacggcttcacaagcactagaacacgagtatttccgaatggatcctctcccaggacggaacgcatttgtagccagccaaccgatggagaagaatgtgaattacccaactcgtccagtagatacaaacaccgatttcgaaggcacgacaagcatcaatccgcctcaagcagtagcagcaggaaacgtagcagggaacatggcaggagctcatggaatgggcagtagatcgatgccaagaccaatggttgcacataacatgcagaggatgcagcaatctcaaggcatgatggcttataatttcccggcacaggcagggcttaacccgagtgttccgctgcagcagcagcgcgggatggctcaaccgcaccagcagcaacagctaagaaggaaagatcccggaatgggtatgtcaggttacgcacctcctaacaaatccagacgcctctaaaggtaaaatcgagatcatcagtctcgggttagaatctgtgtgtttgccgcagaagaaagcgttgcgatttgctttatagagtagagttagattgtaatgcagcatgtggaatgttgctattcatatggatggattggattctctgtagtttttgtataaacatcctctcaagtatttgttaattatattagatcatcatttctcttaacatcatttctcaaaacgtagtaaataggagatttgccaagtgaaaaatatatataatgagacagttattatgaac

SEQ ID NO: 55 - DNA, 2561 bp, synthetic construct; identical to SEQ ID NO: 54
  source 1..2561: mol_type = other DNA

SEQ ID NO: 56 - protein, 471 aa, Zea mays
MGDGRTGGANRPAWLQQYELIGKIGEGTYGLVFLARLKPPHPAPGRRGPPIAIKKFKQSKEGDGVSPTAIREIMLLREINHENVVKLVNVHINHADMSLYLAFDYAEHDLYEIIRHHREKLSSSINPYTVKSLLWQLLNGLNYLHSNWIIHRDLKPSNILVMGEGDEHGIIKIADFGLARIYQAPLKPLCDNGVVVTIWYRAPELLLGGKHYTSAVDMWAVGCIFAELLTLKPLFQGVEAKNPPNPFQLDQLDKIFKVLGHPTVEKWPTLANLPWWQNDHQHIQGHKYENPGFHNIVHLPPKSPAFDLLSKMLEYDPRKRITAAQALEHEYFRMDPLPGRNALLPSQPGEKIVQYPIRPVDTTTDFEGTTSLQPTQPPSGNAPPGGQSVARPMPRQMPQQPMVGGIPRVAGGVTMAAFNAASQAGMAGLNPGNMPMQRGAGGQSHPHQLRRKADQGMGMQNPGYPQQKRRF

SEQ ID NO: 57 - DNA, 1948 bp, Zea mays
  source 1..1948: mol_type = genomic DNA
gatatgttagcacttagcagcattctttggtccaacaagtcgagagaagcgggccgtacgccaccacggcaacggagaagaggactttcagctgcggcggctggccggcgcggcgacggggatgggggatgggcgcacaggcggcgccaaccgtccggcgtggctgcagcagtacgaactgattggcaagattggggaggggacctatggcctcgtcttcctcgcgcgccttaagccgccccacccggcacctggccgacgcggcccccctatcgccataaagaagtttaagcagtcaaaggagggggacggagtatcacccaccgcaattagagagatcatgctcctgcgcgagatcaaccacgagaatgtcgtcaagctcgtcaatgtgcacatcaaccacgctgacatgtccctatacctcgcattcgattacgcagagcacgacctctatgagattatcaggcatcacagggagaagctgagttcctccattaacccatacactgtcaaatccttgctgtggcaactgctcaacggcctcaactatcttcacagtaactggattatacatcgagatctaaagccttccaacatactggtcatgggagaaggagatgaacatggaattataaagatagccgattttggacttgctaggatatatcaagctccactgaaaccattatgtgataatggggttgttgtaactatctggtatcgtgctcctgagctgttacttggggggaaacactacaccagtgctgtcgatatgtgggcagttggttgcatttttgctgaactgcttacactgaaacctctattccaaggtgtggaagcaaaaaatcctccgaacccattccagcttgatcaactcgacaagatttttaaggtcttaggccaccctacagttgaaaagtggcctacccttgccaatcttccatggtggcaaaacgaccaccaacacattcaaggacataagtatgagaacccaggtttccataacattgttcatttaccaccaaagagtcctgcatttgatcttctctcaaaaatgcttgagtatgatccccgaaagcgtataacagctgcacaagctttggagcatgagaccttagtaaccaggttcccggatcgatgggatcgaggaacgggaacgtggtacgcgatactttcggatggacccactacctggacgaaacgcgcttttaccatcccagccaggggagaaaattgtacagtatcctattcgtccagtagatactacaacagattttgaaggaacaacaagccttcaaccaactcaaccgccatcagggaacgctcctcctggaggtcaatctgtagcaagacccatgccacgacaaatgccgcagcaacctatggttggggggattccaagagtggcaggtggagtaaccatggctgccttcaacgctgcctcacaggctggcatggctgggctaaatcctggtaacatgcctatgcagagaggcgcaggtggtcagtctcatccgcaccagttgagaaggaaggcggatcaagggatggggatgcagaaccctgggtatcctcagcagaagagacgattctgacgctatcaagatggagccatctgctgtatatcaggtgtttgaaacacgttgcctgtgtaagctgctgtagttttgttatcagcatccgaatgccaatgctggcacctgtaaaacacattaatcagtcgagagtccagataccagttgtccttatgggttatgatctaagctgctcgaatttggctgatttggtttgcaacagaaaggtcttgcttttgctcatggcccagtggaattatccacatgcgtaggaaatttagcatctatttggcttgagaaaagattttcattaaattctagtggcagtaaatatttttatggccacaaactacacagaattgagcagttgagct

SEQ ID NO: 58 - protein, 501 aa, Zea mays
MGDGRTGGANRPAWLQQYELIGKIGEGTYGLVFLARLKPPHPAPGRRGPPIAIKKFKQSKEGDGVSPTAIREIMLLREINHENVVKLVNVHINHADMSLYLAFDYAEHDLYEIIRHHREKLSSSINPYTVKSLLWQLLNGLNYLHSNWIIHRDLKPSNILVWCHQLYRNIIAQFLQTCPLADTYFICATKVMGEGDEHGIIKIADFGLARIYQAPLKPLCDNGVVVTIWYRAPELLLGGKHYTSAVDMWAVGCIFAELLTLKPLFQGVEAKNPPNPFQLDQLDKIFKVLGHPTVEKWPTLANLPWWQNDHQHIQGHKYENPGFHNIVHLPPKSPAFDLLSKMLEYDPRKRITAAQALEHEYFRMDPLPGRNALLPSQPGEKIVQYPIRPVDTTTDFEGTTSLQPTQPPSGNAPPGGQSVARPMPRQMPQQPMVGGIPRVAGGVTMAAFNAASQAGMAGLNPGNMPMQRGAGGQSHPHQLRRKADQGMGMQNPGYPQQKRRF

SEQ ID NO: 59 - protein, 461 aa, Glycine max
MGDGSGNRWSRAEWVQQYDLLGKIGEGTYGLVFLARTKGTPSKSIAIKKFKQSKDGDGVSPTAIREIMLLREITHENVVKLVNVHINHADMSLYLAFDYAEHDLYEIIRHHRDKLNHSINQYTVKSLLWQLLNGLSYLHSNWMIHRDLKPSNILVMGEGEEHGVVKIADFGLARIYQAPLKPLSDNGVVVTIWYRAPELLLGAKHYTSAVDMWAVGCIFAELLTLKPLFQGAEVKATSNPFQLDQLDKIFKVLGHPTLEKWPSLASLPHWQQDVQHIQGHKYDNAGLYNVVHLSPKSPAYDLLSKMLEYDPRKRLTAAQALEHEYFKIEPLPGRNALVPCQLGEKIVNYPTRPVDTTTDLEGTTNLPPSQTVNAVSGSMPGPHGSNRSVPRPVNVVGMQRMPPQAMAAYNLSSQAAMGDGMNPGGISKQRGVPQAHQPQQLRRKEQMGMPGYPAQQKSRRI

SEQ ID NO: 60 - DNA, 1874 bp, Glycine max
  source 1..1874: mol_type = genomic DNA
catttcaattttaggacacggctgcctatccccttgcgatcgaagagagatgggggacggaagtgggaaccggtggagcagggcggagtgggtgcagcagtacgatctcttaggcaaaatcggagaaggcacttacggcctcgtcttcctggcccgaaccaaaggcactccctccaaatccatcgccatcaaaaagttcaagcaatccaaggacggcgacggcgtctcccccaccgccatccgcgaaatcatgctgctcagggagattacgcacgagaacgtcgtcaagctcgtcaatgtccacatcaaccacgccgacatgtcgctctacctcgcctttgattacgccgagcacgatctctatgaaattattaggcatcacagggataaactcaaccattccattaaccaatacactgttaagtctttgctctggcagttgctcaatggactaagctatctgcacagtaattggatgatacatcgagatttgaagccatcgaatatattggttatgggagaaggagaggaacatggagttgttaagattgctgactttggacttgcgaggatatatcaagctcctctgaagccgttatctgataatggggttgttgtaaccatttggtatcgtgcacccgagttgcttcttggagcaaaacattatactagtgctgttgatatgtgggctgtgggatgcatttttgctgagttgttgaccttgaagccgctatttcaaggggcagaagtcaaagctacatcaaatccctttcagctcgaccaacttgacaagatatttaaggttttaggccatcccacattagaaaagtggccttccttagcaagtcttccacattggcaacaagatgtgcaacatatacaaggacacaaatatgataatgctggtctctataatgttgtacacctgtctccaaaaagccccgcatatgacctcttgtcaaagatgcttgaatatgatcctcgtaagcgtttaacagcagcacaagctttggagcatgagtatttcaaaattgaaccattacctggacggaatgcacttgtaccctgccaacttggagagaaaattgtaaattatcccactcgtccagtggacaccactactgatcttgaaggaacaaccaatctgccaccttcacaaacggtaaatgcagtttctggcagcatgcctggtcctcatgggtcaaatagatctgttcctcggccagtgaatgttgttggaatgcaaagaatgccccctcaagcaatggcagcttataatctctcatctcaggcagccatgggagacggaatgaatcctgggggtatctcaaagcaacgaggtgttccacaggcccatcagccgcaacagttgagaaggaaggagcaaatggggatgccgggataccctgcacaacagaagtcaagacgaatataaggtttctgctggaagagagactacgtgaagataaatttggggtcaatacttcagtgcctgaactcatgcaggacatttctggacagggtttgtctcaatacttgcaaacctctcactttattgcaatcaaagattgggtgcattcttctctggaattttgatgctaaaatgccaaatgtatgctggaacaccaatgaagccataaaagggtataaacgtatgaaagggttaagctactgtaagcacatgtatatcatgattataacaatgcaattctattgtatttctcagcttttgggcaagatcaatgtcagtgaaaccaaatgttaatcatccattgggttttcataatgaaacttttcacgattaaatttataatatgctactttgtattcgtcgaatattttgcctcacatgattgaagatagttcaaatatca

SEQ ID NO: 61 - protein, 464 aa, Glycine max
MGDGSGSRWSRAEWVQQYDLLGKIGEGTYGLVFLARTKSPVGTPSKSIAIKKFKQSKDGDGVSPTAIREIMLLREITHENVVKLVNVHINHADMSLYLAFDYAEHDLYEIIRHHRDKLNHSINQYTVKSLLWQLLNGLSYLHSNWMIHRDLKPSNILVMGEGEEHGVVKIADFGLARIYQAPLKPLSDNGVVVTIWYRAPELLLGAKHYTSAVDMWAMGCIFAELLTLKPLFQGAEVKATSNPFQLDQLDKIFKVLGHPTLEKWPSLASLPHWQQDVQHIQGHKYDNAGLYNVVHLSPKSPAYDLLSKMLEYDPRKRLTAAQALEHEYFKIEPLPGRNALVPCQLGEKIVNYPTRPVDTTTDLEGTTNLPPSQTVNAVSGSMPGPHGSNRSVPRPMNVVGMQRLPPQAMAAYNLSSQAAMGDGMNPGDISKHRGVPQAHQPQQLRRKEQMGMPGYPAQQKSRRL

SEQ ID NO: 62 - DNA, 2045 bp, Glycine max
  source 1..2045: mol_type = genomic DNA
tttcaattttcagacgctgctgcctatccccttgcgatcgaacagaacagaagagagatgggggacggaagtgggagccggtggagcagggcggagtgggtgcagcagtacgatctcttaggaaaaatcggcgaaggcacttacggcctcgtcttcctcgcccgaaccaaatcccccgttggcactccctccaaatccattgccataaaaaagttcaagcaatccaaggacggcgacggcgtctcccccaccgccatccgcgaaatcatgttgctgagggagattacgcacgagaacgtcgtcaagctcgtcaacgtacacatcaaccacgccgacatgtctctctacctcgccttcgattacgccgagcacgatctctatgaaattattaggcatcacagggacaaactcaaccattccattaatcagtacactgttaagtctttgctctggcagttgctcaatggactaagctatctgcacagtaattggatgatacatcgtgatttgaagccatcgaatatattggttatgggtgaaggagaggaacatggagttgttaagattgctgactttggacttgcgaggatatatcaagctcctctgaagccgttatctgacaatggggttgttgtaaccatttggtatcgtgcacctgagttgcttcttggagcaaaacattataccagtgctgttgatatgtgggctatgggatgcatttttgctgagttgttgaccttgaagccactatttcaaggggcagaagtcaaagctacatcaaatccctttcagcttgaccaacttgacaagatatttaaggttttaggccatcccacattagaaaagtggccttccttagcaagtcttccacattggcaacaagatgtgcaacatatacaaggacacaaatatgacaatgccggtctctataatgttgtacacctgtctccaaaaagccctgcatatgacctcttgtcaaagatgcttgaatatgatcctcgtaagcgtttaacagcagcacaagctttggagcatgagtatttcaaaattgaaccattacctggacgaaatgcacttgtaccctgccaacttggagagaaaattgtaaattatcccactcgtccagtggacactacaactgatcttgaagggacaaccaatctgccaccttcacaaacggtaaatgcagtttctggtagcatgcctggtcctcatgggtcaaatagatctgtgcctcggccaatgaatgttgttggaatgcaaagactgccccctcaagcaatggcagcttataatctctcatctcaggcagccatgggagatggaatgaatcctggggatatctcaaagcatcgaggtgttccacaggcccatcagccacaacagttgagaaggaaggagcaaatggggatgccgggataccctgcacaacagaagtcaagacgattataaggtttctgctggaagagagactaagtgaagatagatttggggtcaatacttcagtacctgaactcatgcaggacatttctggacagtgtttgccttcaatacttgcaagcctcactttattgcaatcaaagattgggtgcattcttctctggaattttgatgctaaaatgccaaatgtatgctggaacaccaatgaagccataaaagggaataaacgtatgaaagggttaagctactgtaagcacatgtatatcatgattataacaatgcaattctattgtatttcttagcttttgggcaagatcaatgtcagtaaaccaaatgttgatcatccattaggttttcataatggaacttttcttgattaaatctataacatgctactttgtatttgtagaatattttgcctcacatgattgaagatagttcaaatatcacttgcctttggtatttccgttttgaatttttctgtgatcactggaatcacagacttttcactcccaaggagattattgaagctttctgtgagtatgatgtaaactttgttcggagacgtagtagtatgaagatatgaaaagcagcaattgggagaa

SEQ ID NO: 63 - protein, 294 aa, Triticum aestivum
MEQYEKVEKIGEGTYGVVYKARDRTTNETIALKKIRLEQEDEGVPSTAIREISLLKEMQHGNIVKLHDVVHSEKRIWLVFEYLDLDLKKFMDSCPEFAKSPALIKSYLYQILRGVAYCHSHRVLHRDLKPQNLLIDRRTNALKLADFGLARAFGIPVRTFTHEVVTLWYRAPEILLGARQYSTPVDVWSVGCIFAEMVNQKPLFPGDSEIDELFKIFRVLGTPNEQTWPGVSSLPDYKSAFPRWQAEDLATVVPNLEPVGLDLLSKMLRFEPNKRITARQALEHEYFKDMEMVQ

SEQ ID NO: 64 - DNA, 1170 bp, Triticum aestivum
  source 1..1170: mol_type = genomic DNA
gcccccctctccccctcccccccacccccccaatggcggcagcagcagcagcagcagcagcagcttcgcccgccgcagccgctctcccccgcccctcctccccgtgatccccttccccttcccctcccccgcttcctcctctcccccctcccgcctcctcacccatttcccacgcccgcgccgccgccgccgccgtagcattggacgccgacccgatggagcagtacgagaaggtggagaagatcggggagggcacgtacggggtggtgtacaaggcccgggacaggaccaccaacgagaccatcgcgctcaagaagatccgcctggagcaggaggacgagggcgtcccctccaccgccatccgcgagatctcgctcctcaaggagatgcagcacggcaacatcgtcaagctgcacgatgttgtccacagcgagaagcgcatatggctcgtctttgagtacctggatctggacctgaagaagttcatggactcctgtccagagtttgccaagagccccgccttgatcaagtcatatctctatcagatactccgcggcgttgcttactgtcattctcatagagttcttcatcgagatttgaaacctcagaatttattgatagaccggcgtactaatgcactgaagcttgcagactttggtttagcaagggcatttggaattcctgtccgtacatttactcatgaggtagtaacattatggtacagagctcctgaaatccttcttggagcaaggcagtattccacaccagttgacgtgtggtcagtgggctgtatctttgcagaaatggtgaaccagaaaccactgttccctggcgattctgagattgatgagctatttaagatattcagggtactcggcactccaaatgaacaaacttggccaggcgtgagttccttgcctgactacaagtccgccttccccaggtggcaggcagaggaccttgcaaccgttgtccccaatcttgaacctgttggcctggaccttctctcgaaaatgcttcggttcgagccaaacaagaggatcacggctaggcaggctcttgagcatgagtacttcaaggacatggagatggtacagtgagctggctatgtggtagtgactggcatatgtatgagctgagctgctcgtttcattccttttgtgaacgctc

SEQ ID NO: 65 - protein, 475 aa, Oryza sativa
MGDGRVGGGTNRPAWLQQYELVGKIGEGTYGLVFLARLKQSHPHAAAGVGRRGSPIAIKKFKQSKEGDGVSPTAIREIMLLREINHENVVKLVNVHINHADMSLYLAFDYAEHDLYEIIRHHREKLNLPINPYTVKSLLWQLLNGLNYLHSNWIIHRDLKPSNILVMGEGEEHGIIKIADFGLARIYQAPLKPLSDNGVVVTIWYRAPELLLGAKHYTSAVDMWAVGCIFAELLTLKPLFQGVEAKATPNPFQLDQLDKIFKVLGHPTVEKWPTLANLPCWQNDQQHIQGHKYENTGLHNIVHLPQKSPAFDLLSKMLEYDPRKRITAAQALEHEYFRMDPLPGRNALLPSQAGEKIVQYPVRPVDTTTDFEGTTSLQPTQAPSGNAAPGNQSVVPRPIPRQMQQPMVGMSRMGGTNMAAFGAAPQGGIAGMNPGNIPMQRGAGAQSHPHQLRRKADQGMGMQNPGYPTQQKRRF

SEQ ID NO: 66 - DNA, 1832 bp, Oryza sativa
  source 1..1832: mol_type = genomic DNA
gagcgtattttggctttacgccttcgtgtggagtaaacgccctttctgttgggcgggttcggctggatcttttgttcccccttttcctttcttctccggcagcggcggcggcgatgggggacggccgcgtcggaggtggaacgaatcggccggcatggctgcagcaatacgaactagtgggcaagattggcgaggggacctacggcctcgtcttcctcgctcgcctcaaacaatcgcatccccacgctgccgctggcgttggccgccgtggctctcccatcgccatcaagaagttcaagcagtccaaggagggcgacggtgtctcgcccaccgccatcagagagatcatgcttctgcgtgagatcaaccacgagaatgttgtcaagctcgtcaatgttcacatcaaccacgccgacatgtccctctacctcgccttcgattacgccgagcacgatctctatgagattatcaggcatcacagagagaagcttaacctccccataaatccctacacagtcaaatctttgctctggcaactgctcaatggtctcaactatctccatagtaactggattatccatcgagatctcaagccttctaatatactggtcatgggagaaggagaagaacatggaattataaagattgctgattttggactcgctaggatatatcaagctccattaaagccattaagtgataacggggttgttgttaccatctggtatcgggctccagagttgttacttggggcaaagcactacacaagtgctgttgatatgtgggcagttggttgcatttttgctgaattgcttacactcaaaccactgttccaaggtgttgaagccaaagctactccaaacccgtttcaacttgatcaactagacaagatttttaaggtcttaggtcatcctaccgttgagaaatggcctaccctcgctaatcttccatgctggcaaaacgatcaacaacacattcaagggcataagtatgagaacacaggacttcataatattgttcacttgcctcagaagagtcctgcgtttgatcttctctcaaaaatgctcgagtatgatcctcgaaagcgtataacagctgcgcaagctttggaacatgagtactttcgaatggatcctctgcctggacggaatgcacttttaccatcgcaggctggagagaaaattgtgcaatatcctgtgcgtccagttgataccacaactgattttgaaggaacaacaagccttcaaccaactcaagcgccatcagggaacgcagctcctggcaaccagtctgtggtaccgagacccattccgaggcaaatgcaacaacccatggtcggtatgtcgagaatgggtggtacaaacatggcggcctttggtgcagctccgcaaggaggcatagctgggatgaatcctggtaatattccaatgcagaggggcgctggagcccaatctcatccgcatcagttgagaaggaaagctgatcaagggatggggatgcagaaccccggttatcctactcaacagaagaggcggttctgaccgactgaatttgtaattgtatatctatttggtgtgttacttgtgagcacgcttagcttttgcggtggttgctcctagtcgtacagtgagaattgtatctgttctgttgtaattgaacgccatcacaaccaacacctctactagttagttactagagtgactacgcagacagggccaggttgccgatgatgccatcaccaatggagacaggcatacccagccagagtttcgccaatactctgccccctgaacccaaccaatgaatgaattggcatcgtacgatctatttca

SEQ ID NO: 67 - DNA, 20 bp, synthetic construct
  source 1..20: mol_type = other DNA
accgagacacattcccgatt

SEQ ID NO: 68 - DNA, 20 bp, synthetic construct
  source 1..20: mol_type = other DNA
catcaggcttgcatgccatt

SEQ ID NO: 69 - DNA, 25 bp, synthetic construct
  source 1..25: mol_type = other DNA
acgaataagagcgtccattttagag

SEQ ID NO: 70 - DNA, 21 bp, synthetic construct
  source 1..21: mol_type = other DNA
tcttcctcgtgacaaaacgca

SEQ ID NO: 71 - DNA, 23 bp, synthetic construct
  source 1..23: mol_type = other DNA
ccaaacacagaaccatctccaca

SEQ ID NO: 72 - DNA, 20 bp, synthetic construct
  source 1..20: mol_type = other DNA
ccggatcgtatcggttttcg

SEQ ID NO: 73 - DNA, 23 bp, synthetic construct
  source 1..23: mol_type = other DNA
acggttcctctatgcctcaagtc

SEQ ID NO: 74 - DNA, 24 bp, synthetic construct
  source 1..24: mol_type = other DNA
gtggagtggtctaaagcaaccttc

SEQ ID NO: 75 - DNA, 23 bp, synthetic construct
  source 1..23: mol_type = other DNA
ataacgctgcggacatctacatt

SEQ ID NO: 76 - DNA, 21 bp, synthetic construct
  source 1..21: mol_type = other DNA
tcaggaagacagagtgttccc

SEQ ID NO: 77 - DNA, 21 bp, synthetic construct
  source 1..21: mol_type = other DNA
tgcgtttctctaagaaccgag

SEQ ID NO: 78 - DNA, 20 bp, synthetic construct
  source 1..20: mol_type = other DNA
ttgggtgatggttcacgtag

SEQ ID NO: 79 - DNA, 25 bp, synthetic construct
  source 1..25: mol_type = other DNA
gcttataccgaaaacccgattccag

SEQ ID NO: 80 - DNA, 25 bp, synthetic construct
  source 1..25: mol_type = other DNA
ggctcattgagatcaggaagaacca

SEQ ID NO: 81 - DNA, 20 bp, synthetic construct
  source 1..20: mol_type = other DNA
ttgggtgatggttcacgtag

SEQ ID NO: 82 - DNA, 21 bp, synthetic construct
  source 1..21: mol_type = other DNA
gacacacatcactgtcacttc

SEQ ID NO: 83 - DNA, 22 bp, synthetic construct
  source 1..22: mol_type = other DNA
agtttctgaggtctctaccttc

SEQ ID NO: 84 - DNA, 24 bp, synthetic construct
  source 1..24: mol_type = other DNA
ccgttttgtatatcccgtttccgt

SEQ ID NO: 85 - DNA, 20 bp, synthetic construct
  source 1..20: mol_type = other DNA
atgcgacttggaacttcgcc

SEQ ID NO: 86 - DNA, 19 bp, synthetic construct
  source 1..19: mol_type = other DNA
ggaggatccgaaccgtctg

SEQ ID NO: 87 - DNA, 20 bp, synthetic construct
  source 1..20: mol_type = other DNA
acgtccgcaatgtgttatta

SEQ ID NO: 88 - DNA, 22 bp, synthetic construct
  source 1..22: mol_type = other DNA
tgtcctaagagtccgccgttgt

SEQ ID NO: 89 - DNA, 22 bp, synthetic construct
  source 1..22: mol_type = other DNA
tttggaggatccgacccgtttg

SEQ ID NO: 90 - DNA, 21 bp, synthetic construct
  source 1..21: mol_type = other DNA
taccgcataatcatggtcgtc

SEQ ID NO: 91 - DNA, 21 bp, synthetic construct
  source 1..21: mol_type = other DNA
tcatgctcattgcattagtcg

SEQ ID NO: 92 - DNA, 22 bp, synthetic construct
  source 1..22: mol_type = other DNA
ctttgaagacgtggttggaacg

SEQ ID NO: 93 - DNA, 22 bp, synthetic construct
  source 1..22: mol_type = other DNA
atttctcgatcgccgtcgtagt

SEQ ID NO: 94 - DNA, 25 bp, synthetic construct
  source 1..25: mol_type = other DNA
gccaaagagctttggtcttagagtg

SEQ ID NO: 95 - DNA, 25 bp, synthetic construct
  source 1..25: mol_type = other DNA
gtctaagcgtcaatttgtttacacc

SEQ ID NO: 96 - DNA, 21 bp, synthetic construct
  source 1..21: mol_type = other DNA
gcacgtgaccaaatttgcaga

SEQ ID NO: 97 - DNA, 23 bp, synthetic construct
  source 1..23: mol_type = other DNA
tgaagagaggaggatgatgagga

SEQ ID NO: 98 - DNA, 22 bp, synthetic construct
  source 1..22: mol_type = other DNA
aaacctcctcggattccattgc

SEQ ID NO: 99 - DNA, 20 bp, synthetic construct
  source 1..20: mol_type = other DNA
atgtcgagttctatggaatg

SEQ ID NO: 100 - DNA, 20 bp, synthetic construct
  source 1..20: mol_type = other DNA
tcatatttcagctgctaaac

SEQ ID NO: 101 - DNA, 20 bp, synthetic construct
  source 1..20: mol_type = other DNA
atgtcgagtttttctgccga

SEQ ID NO: 102 - DNA, 20 bp, synthetic construct
  source 1..20: mol_type = other DNA
ttaccgtgaactgagccaag

SEQ ID NO: 103 - DNA, 21 bp, synthetic construct
  source 1..21: mol_type = other DNA
atggagagagattttctcggg

SEQ ID NO: 104 - DNA, 23 bp, synthetic construct
  source 1..23: mol_type = other DNA
ttaggttgcagagctgagagaag

SEQ ID NO: 105 - DNA, 25 bp, synthetic construct
  source 1..25: mol_type = other DNA
atggagagagattttctcgggctgg

SEQ ID NO: 106 - DNA, 25 bp, synthetic construct
  source 1..25: mol_type = other DNA
ttagtgcagatgatgagctggagga

SEQ ID NO: 107 - DNA, 20 bp, synthetic construct
  source 1..20: mol_type = other DNA
atgtcgtcgagcaatgaaaa

SEQ ID NO: 108 - DNA, 20 bp, synthetic construct
  source 1..20: mol_type = other DNA
ctatagccttagatcgagat

SEQ ID NO: 109 - DNA, 19 bp, synthetic construct
  source 1..19: mol_type = other DNA
atgtcaacgggacaagcgc

SEQ ID NO: 110 - DNA, 20 bp, synthetic construct
  source 1..20: mol_type = other DNA
ctaaagcttgagttcaaggt

SEQ ID NO: 111 - DNA, 23 bp, synthetic construct
  source 1..23: mol_type = other DNA
atgatcatcatcatcaaaaactg

SEQ ID NO: 112 - DNA, 20 bp, synthetic construct
  source 1..20: mol_type = other DNA
ctatcggtaacggtggtaag

SEQ ID NO: 113 - DNA, 20 bp, synthetic construct
  source 1..20: mol_type = other DNA
atggaaagagattttctggg

SEQ ID NO: 114 - DNA, 20 bp, synthetic construct
  source 1..20: mol_type = other DNA
ttatgtaggagaagtagaag

SEQ ID NO: 115 - DNA, 22 bp, synthetic construct
  source 1..22: mol_type = other DNA
atgtcgaaagctaccatagaac

SEQ ID NO: 116 - DNA, 30 bp, synthetic construct
  source 1..30: mol_type = other DNA
gatagtaaggagatgttgatactaatctct

SEQ ID NO: 117 - DNA, 20 bp, synthetic construct
  source 1..20: mol_type = other DNA
atgaagggttgcagcttaga

SEQ ID NO: 118 - DNA, 23 bp, synthetic construct
  source 1..23: mol_type = other DNA
ttagaaattatgaagagaggagg

SEQ ID NO: 119 - DNA, 24 bp, synthetic construct
  source 1..24: mol_type = other DNA
atggctgatggtgaagacattcaa

SEQ ID NO: 120 - DNA, 24 bp, synthetic construct
  source 1..24: mol_type = other DNA
tcagaagcacttcctgtgaacaat

SEQ ID NO: 121 - DNA, 64 bp, synthetic construct
  source 1..64: mol_type = other DNA
ccttccacactggcaaaatgatgttcaacacattcaagctcacaaatacgacagtgtgggtctc